(19) United States
(12) Patent Application Publication
(10) Pub. No.: US 2004/0186131 A1
(43) Pub. Date: Sep. 23, 2004

(54) Method of preventing or treating atherosclerosis or restenosis

(76) Inventors: Michael W. Wathen, Thousand Oaks, CA (US); Lynne K. Wathen, Thousand Oaks, CA (US)

Correspondence Address: FLYNN, THIEL, BOUTELL & TANIS, P.C., 2026 RAMBLING ROAD, KALAMAZOO, MI, 49008-1699, US

(21) Appl. No.: 10/651,309

(22) Filed: Aug. 28, 2003

Related U.S. Application Data
(60) Provisional application No. 60/407,563, filed on Aug. 30, 2002.
(60) Provisional application No. 60/467,497, filed on May 2, 2003.

(51) Int. Cl. A61K 31/47

(52) U.S. Cl. 514/312

(57) ABSTRACT

The present invention provides a method of preventing or treating atherosclerosis or restenosis in mammals, which comprises administering an effective amount of a compound selected from the group consisting of structures of Formulas I to XV:

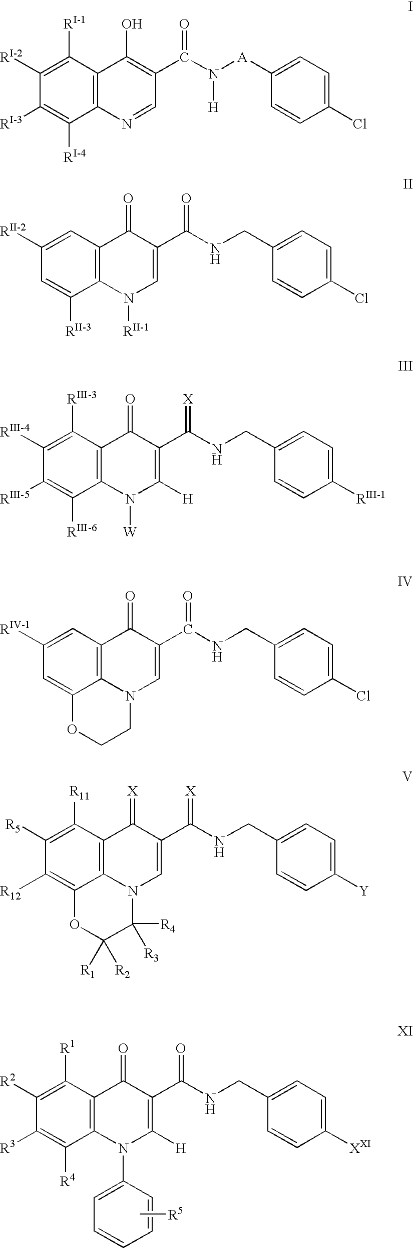

wherein the various groups in the Formulae are defined herein.

### CROSS-REFERENCE TO RELATED APPLICATIONS

This application claims the benefit of U.S. provisional application Ser. No. 60/407 563, filed Aug. 30, 2002, and U.S. provisional application Serial No. 60/467 497, filed May 2, 2003, under 35 USC 119(e)(i), which are incorporated herein by reference.

### BACKGROUND OF THE INVENTION

This invention relates to a method of preventing or treating atherosclerosis and restenosis in mammals.

Atherosclerosis is characterized by the deposition of fatty substances in and fibrosis of the inner layer of the arteries. Restenosis is an accelerated form of atherosclerosis that commonly occurs after angioplasty surgery and atherectomy.

Cardiovascular diseases (CVD) contribute substantially to illness and death worldwide and ranks second only to infectious and parasitic diseases as human affliction. Atherosclerosis, a major component of CVD, has properly been considered a public health problem of industrialized countries, accounting for an estimated one third of deaths overall. It has been reported that in the United States alone, atherosclerosis affects one in four persons, causing approximately 42% of all deaths. O'Connor et al, “Potential Infectious Etiologies of Atherosclerosis: A Multifactorial Perspective”, Emerging Infectious Disease, Vol. 7, No. 5, September-October 2001.

It has been suggested that the number of chronic infective pathogens which an individual has been exposed independently contribute to the long-term prognosis in patients with documented coronary artery disease. H J Rupprecht et al, “Impact of Viral and Bacterial Infective Burden on Long-term Prognosis in Patients with Coronary Artery Disease. (Circulation (2001) 104:25-31. Seropositivity to multiple herpesviruses is an independent risk factor for death from cardiovascular disease and risk is proportional to the number of different herpesviruses that have infected an individual. Other investigators that have suggested a connection between infectious pathogens and atherosclerosis include Espinola-Klein et al, “Impact of Infectious Burden on Extent and Long-Term Prognosis of Atherosclerosis”, Circulation (2002) 105:15-21; O'Connor et al, Supra: and Zhou et al, “Association Between Prior Cytomegalovirus Infection and the Risk of Restenosis after Coronary Atherectomy”, The New England Journal of Medicine (1996). An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994, 90:1969-1978).

Herpesviruses are believed to be a particular problem in atherosclerosis because they reside latently in an infected individual and can reactivate causing a chronic inflammatory response. The herpesvirus family contains eight known human viruses; herpes simplex virus type 1(HSV-1), herpes simplex virus type 2(HSV-2), varicella zoster virus (VZV), human cytomegalovirus (HCMV), human herpes virus 6(HHV-6), human herpes virus 7(HHV-7), Epstein-barr virus (EBV) and human herpes virus 8(HHV-8). One of the hallmarks of herpesviruses is their ability to establish latent infections in their host and to recur during times of stress or immunosuppression. The human herpesviruses are associated with a diverse set of diseases ranging in severity from mild cold sores to life-threatening illness in immunocompromised patients (Table 1).

TABLE 1
Herpesvirus diseases and treatment

| | Associated Diseases | | |
|---|---|---|---|
| | | Immunocompromised | Marketed |
| Virus | Normal Host | Host | Antivirals |
| HSV-1 | Herpes labialis | Disseminated herpes | Acyclovir |
| | (cold sores) | | Penciclovir |
| HSV-2 | Genital herpes | Disseminated herpes | Acyclovir |
| | | | Valaciclovir |
| | | | Famciclovir |
| VZV | Chicken pox | Herpes zoster | Acyclovir |
| | Herpes zoster | | Valaciclovir |
| | | | Famciclovir |
| CMV | Congenital CMV | Retinitis | Ganciclovir |
| | disease | Pneumonia | Valganciclovir |
| | | GI disease | Foscarnet |
| | | Graft rejection | Cidofovir |
| | | | Formivirsen |
| EBV | Infectious | Lymphomas (PTLD) | None |
| | mononucleosis | | |
| HHV-6 | Exanthem subitum | Graft rejection | None |
| HHV-7 | Exanthem subitum | Graft rejection | None |
| HHV-8 | Kaposi's sarcoma | Kaposi's sarcoma | None |

HSV-1, HCMV, VZV and EBV are ubiquitous viruses with seroprevalence rates in adults of 70-80% for HSV-1 and 90-100% for HCMV, VZV and EBV. Seroprevalence of HSV-2 increases from about 10% in young adults to 35% by age 60. Antibodies to HHV-8 are also found in about 33% of adults in the United States. The high seroprevalence of multiple viruses and their ability to reactivate from latent infections, make these herpesviruses prime candidates for causing chronic inflammatory responses leading to atherosclerosis.

Numerous studies and articles on the epidemiology of the herpesvirus family are in the prior art. Wathen, Michael W., “Non-nucleoside inhibitor of herpesviruses”, Rev. Med. Virol, 2002; 12: 167-178; Whitley et al, “Herpes Simplex Viruses”, Clinical Infection Diseases, 1998; 26: 541-55, Cohen, Jeffrey I., “Epstein-Barr Virus Infection”, Medical Progress, Volume 343, Number 7, The New England Journal of Medicine, Aug. 17, 2000, pp. 481-492; Blouvelt et al; “Human Herpes Virus 8 Infection Occurs Following Adolescence in the United States”, The Journal of Infectious Disease, 1997, 176: 771-4; Field, A. Kirk, “Human Cytomegalovirus: challenge opportunities and new drug development”, Antiviral Chemistry and Chemotherapy 10: 219-232.

### INFORMATION DISCLOSURE

U.S. Pat. No. 6,239,142 discloses 4-oxo-4,7-dihydro-thieno[2,3-b]pyridine-5-carboxamide derivatives, compounds of Formula I and I′ that are useful as antiviral agents. These compounds have now been found to be useful in the method of this invention.

U.S. Pat. No. 6,291,437 describes a method for preventing or retarding the development of atherosclerotic lesions or restenosis comprising administering to a subject, preferably a human, an effective amount of an anti-viral composition directed against CMV, and optionally anti-microbial composition directed against C. pneumoniae.

WO 02/48148 A2 discloses anti-viral compounds and a method of using them for the prophylaxis or treatment of atherosclerosis, coronary artery disease or restenosis.

An antiviral drug, Ganciclovir, has been shown to prevent atherosclerosis resulting from CMV infection of rats (K. B. Lemstrom et al. Cytomegalovirus infection-enhanced allograft arteriosclerosis is prevented by DHPG prophylaxis in the rat. Circulation, 1994,90:1969-1978).

U.S. Pat. No. 6,239,142 disclosed compounds and their use to treat herpesvirus infections.

WO 02/06513 disclosed method of screening 4-hydroxyquinline, 4-oxo-dihydroquinoline, and 4-oxo-dihydrothienopyridine derivatives as non-nucleoside herpesvirus DNA polymerase inhibitors.

EP 443568 disclosed fused thiophene derivatives, their production and use.

WO 02/04445 disclosed a variety of tricyclic core structures which have antiviral activity against herpesviruses.

WO 02/04444, WO 02/04443, and WO 02/04422 disclosed a variety of bicyclic core structures which have antiviral activity against herpesviruses.

U.S. Pat. No. 6,248,739 disclosed compounds in which the core structure is a quinoline and useful as antivirals against herpesviruses.

### OBJECT OF THE INVENTION

It is the object of this invention to provide a method for preventing or treating atherosclerosis or restenosis in mammals.

It is a further objective of this invention to provide a method for prophylaxis of atherosclerosis and treat patients who have atherosclerosis.

It is still a further objective of the invention to provide a method that prevents or ameliorates the occurrence of restenosis in patients anticipating coronary atheroscopy or angioplasty.

### SUMMARY OF THE INVENTION

This invention provides a method of preventing or treating atherosclerosis or restenosis in a mammal, comprising administering to said mammal an effective amount of the compound selected from the group consisting of structures of Formula I, Formula II, Formula III, Formula IV, Formula V and Formula XI; wherein Formula I is

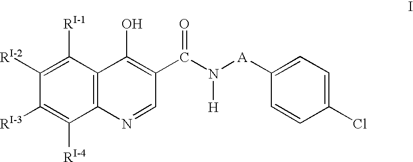

or pharmaceutically acceptable salts thereof wherein

A is:

a) —CH₂—, or

b) —NH—;

R^{I-1}, R^{I-2}, R^{I-3} and R^{I-4} are independently

a) —H,

b) halo,

c) —CN,

d) —NO₂,

e) ^{I-}aryl,

f) ^{I-}het,

g) —OR^{I-5},

h) C₁₋₁₂ alkyl,

i) C₁₋₁₂ alkyl substituted with one to three —CN, halo, —NO₂, OR^{I-5}, —C(═O)R^{I-5}, —COOR^{I-5}, het, aryl, —SR^{I-5}, —OR^{I-6}, —NR^{I-7}R^{I-8}, —OP(═O)(R⁹)₂, —OPH(═O)R^{I-9}, —OC(═O)R^{I-10}, —O-glycyl, —O-valyl, or —O-lysyl,

j) —C≡—CR^{I-11},

k) —CH═CH—R^{I-12},

l) —(CH₂)ₘ—C(═O)R^{I-13},

m) —SR^{I-14},

n) —C(═S)R^{I-15},

o) —(CH₂)ₘ—SOᵢR^{I-13},

p) —NR^{I-7}R^{I-8},

q) —NHSOᵢR^{I-13},

r) R^{I-1} and R^{I-2} taken together are ^{I-}het or C₄₋₆ cycloalkyl, or

s) R^{I-2} and R^{I-3} taken together are ^{I-}het or C₄₋₆ cycloalkyl;

R^{I-5} is

a) H,

b) C₁₋₈ alkyl, optionally substituted with one to three —OH, CN, C₁₋₄ alkoxy, halo, —NO₂, ^{I-}het or ^{I-}aryl,

c) ^{I-}aryl, or

d) ^{I-}het;

R^{I-6} is

a) —SO₂C₁₋₆ alkyl,

b) —SO₂—(CH₂)ₘ—^{I-}aryl, or

c) —SO₂—(CH₂)ₘ—^{I-}het;

R^{I-7} and R^{I-8} are independently

a) H,

b) C₁₋₈ alkyl, optionally substituted with one to three —NO₂, halo, —CN, OR⁵, ^{I-}aryl, ^{I-}het, C₃₋₆ cycloalkyl, C₁₋₆ alkynyl, C₁₋₆ alkenyl, —SR¹⁴, or —NR^{I-16}R^{I-17},

c) ^{I-}aryl,

d) ^{I-}het,

e) —(CH₂)ₘ—C(═O)OR^{I-5},

f) —(CH₂)ₘ—C(═O)R^{I-5}, or

g) R^{I-7} and R^{I-8} taken together to form ^{I-}het;

R^{I-9} is

a) —OH, or

b) —OC₁₋₈ alkyl;

R^{I-10} is

a) H,

b) C₁₋₈ alkyl,

c) —NR^{I-7}R^{I-8},

d) C₁₋₈ alkyl substituted with one to two halo, ^{I-}het, —NR^{I-7}R^{I-8}, —COOH—O(CH₂)ₘCOOH or —C(═O)N(C₁₋₄alkyl) (CH₂)ₙS(═O)₂O⁻M⁺

R^{I-11} is

a) C₁₋₈ alkyl,

b) C₁₋₈ alkyl substituted with one to three —CN, halo, —NO₂, —COOR^{I-5}, —C(═O)R^{I-5}, —SR^{I-5}, ^{I-}aryl, —OR^{I-5}, —NR^{I-7}R^{I-8},—OP(═O)(R^{I-9}) 2, —OPH (═O)R¹⁻⁹ —OC(═O)R^{I-10}, —O-glycyl, —O-valyl, —O-lysyl or —O-seluptamatyl, or

c) —(CH₂)ₘ-^{I-}het;

R^{I-12} is

a) H,

b) —CN,

c) C₁₋₈ alkyl,

d) C₁₋₈ alkyl substituted with one to three —CN, halo, —NO₂, —C(═O)R^{I-5}, —COOR^{I-5}, ^{I-}aryl, ^{I-}het —SR^{I-5} OR^{I-5}, —NR^{I-7}R^{I-8}, —OP(═O)(R^{I-9})₂ or —OPH(═O)R^{I-9},

e) —C(═O)R^{I-5}, or

f) —COOR^{I-5};

R^{I-13} is

a) C₁₋₈ alkyl,

b) C₁₋₈ alkyl substituted one to three —CN, halo, —NO₂, —C(═O)R^{I-5}, ^{I-}het, ^{I-}aryl, —COOR^{I-5}, —SR^{I-5}, —OR^{I-5} or —NR^{I-7}R^{I-8},

c) ^{I-}het,

d) ^{I-}aryl,

e) —NR^{I-7}R^{I-8},

f) OR^{I-5, or}

g) halo;

R^{I-14} is

a) C₁₋₈ alkyl, or

b) C₁₋₈ alkyl substituted with one to three —CN, halo, —NO₂, —C(═O)R^{I-5}, —COOR^{I-5}, ^{I-}het, ^{I-}aryl, —OR^{I-5}, or —NR^{I-7}R^{I-8};

R^{I-15} is

a) —NH₂, or

b) —NHNH₂;

R^{I-16} and R^{I-17} is independently

a) H,

b) C₁₋₄ alkyl,

c) —C(═O)C₁₋₄ alkyl, or

d) —C(═O)—(CH)ₘ-aryl;

aryl is phenyl or naphthyl, optionally substituted with R^{I-18};

het is a 5-, 6- or 7-membered saturated or unsaturated heterocyclic ring having 1-3 heteroatoms selected from the group consisting of nitrogen, oxygen and sulfur, wherein the heterocyclic ring is optionally fused to a benzene ring, wherein aryl, het and benzene ring are optionally substituted with R^{I-18};

R^{I-18} is

a) halo,

b) —NO₂,

c) phenyl, optionally substituted with one to five —OH, —CN, halo, —NO₂, C₁₋₆ alkyl, het, or OC₁₋₄ alkyl,

d) C₁₋₈ alkyl, optionally substituted with one to three halo, —CN, —NO₂, aryl, —SR⁵, —OR⁵ or —NR^{I-7}R^{I-8},

e) OR⁵, or

f) —SO₂NH₂;

M^{I} is sodium, potassium or lithium atom;

i^{I} is 1 or 2;

m^{I} is 0, 1, 2, or 4;

n^{I} is 1, 2, 3 or 4;

wherein Formula II is

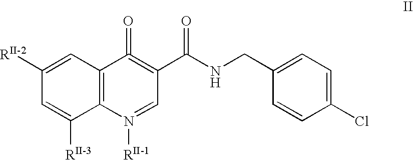

wherein

R^{II-1} is C₁₋₇ alkyl, optionally substituted by hydroxy or NR^{II-4}R^{H5};

R^{II-2} is C₁₋₇ alkyl substituted by hydroxy or NR⁴R⁵;

R^{II-3} is H, F or C₁₋₇ alkoxy;

R^{II-4} and R^{II-5} together with N are a 5- or 6-membered heterocyclic moiety having 1-3 heteroatoms selected from the group consisting of nitrogen, oxygen and sulfur in which sulfur may be substituted by one (1) or two (2) oxygen atoms;

or pharmaceutically acceptable salts thereof;

wherein, Formula III is

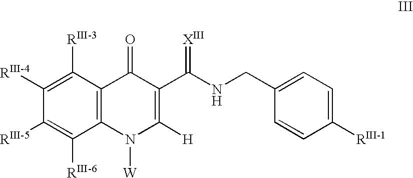

or a pharmaceutically acceptable salt thereof wherein,

X^{III} is

a) O, or

b) S;

W is

a) R^{III-2};

b) NR^{III-7}R^{III-8};

c) OR^{III-9}, or

d) SOᵢR^{III-9};

R^{III-1} is

a) Cl,

b) F,

c) Br,

d) CN, or

e) NO₂;

R^{III-2} is

a) (CH₂CH₂O)ₘR^{III-10},

b) het^{III}, wherein said het^{III}, is bonded via a carbon atom,

c) C₁₋₇ alkyl which may be partially unsaturated and is optionally substituted by one or more substituents selected from a group consisting of NR^{III-7}R^{III-8}, R^{III-11}, CN, SOᵢR^{III-9}, or OC₂₋₄ alkyl which is further substituted by het^{III}, OR^{III-10}, OC(═O)aryl^{III}, or NR^{III-7}R^{III-8}, or

d) C₃₋₈ cycloalkyl, which may be partially unsaturated and is optionally substituted by R^{III-11}, NR^{III-7}R^{III-8}, SOᵢR^{III-9}, or C₁₋₇ alkyl optionally substituted by R^{III-11}, NR^{III-7}R^{III-8}, or SOᵢR^{III-9};

R^{III-3} is

a) H,

b) halo, or

c) C₁₋₄ alkyl, optionally substituted by one to three halo;

R^{III-4} is

a) H,

b) aryl^{III},

c) het^{III},

d) SO₂NHR^{III-12},

e) CONHR^{III-12},

f) NR^{III-7}R^{III-8},

g) NHCOR^{III-12},

h) NHSO₂R^{III-12},

i) OC₂₋₇ alkyl optionally substituted by —OH,

j) SC₂₋₇ alkyl optionally substituted by OH, or

k) C₁₋₈ alkyl which may be partially unsaturated and is optionally substituted by one or more substituents selected from a group consisting of N, OR^{III-10}, NR^{III-7}R^{III-8}, halo, SOᵢR^{III-9}, OR^{III-13} or R^{III-11};

R^{III-5} is

a) H,

b) halo,

c) C≡CR^{III-14},

d) NR^{III-7}R^{III-8},

e) SO₂NHR^{III-12},

f) het^{III}, or

g) C₁₋₇ alkyl, optionally substituted by OH;

R^{III-6} is

a) H,

b) halo,

c) SC₁₋₇ alkyl,

d) C₁₋₇ alkoxy, optionally substituted by one or more halo or OH, or

e) C₁₋₇ alkyl, which may be partially unsaturated and is optionally substituted by halo, NR^{III-10}R^{III-10}, (CH₂)ₙOR^{III-13}, R^{III-11}, OC₁₋₇ alkyl which is further substituted with ^{III-}het, NR^{III-7}R^{III-8}, or SOᵢR^{III-9};

R^{III-7} and R^{III-8} are independently

a) H,

b) aryl^{III},

c) C₁₋₇alkyl which may be partially unsaturated and is optionally substituted by one or more substituents selected from a group consisting of NR^{III-10}R^{III-10}, CONP^{III-10}R^{III-10}, R^{III-11}, SOᵢR^{III-9}, halo; or

d) R^{III-7} and R^{III-8} together with the nitrogen to which they are attached to form a het^{III};

R^{III-9} is

a) aryl^{III},

b) het^{III},

c) C₃₋₈cycloalkyl, or

d) C₁₋₇alkyl which may be partially unsaturated and optionally substituted by one or more OR^{III-10}, Oaryl^{III}, het^{III}, aryl^{III}, NR^{III-10}R^{III-10}, CN, SH, SOᵢC₁₋₆ alkyl, SOᵢ aryl^{III}, halo, or CONR^{III-10}R^{III-10}.

R^{III-10} is

a) H, or

b) C₁₋₇ alkyl, optionally substituted by OH;

R^{III-11} is

a) OR^{III-10},

b) Ohet^{III},

c) Oaryl^{III},

d) CO₂R^{III-10},

e) het^{III},

f) aryl^{III}, or

g) CN;

R^{III-12} is

a) H,

b) het^{III},

c) aryl^{III},

d) C₃₋₈ cycloalkyl, or

e) C₁₋₇ alkyl optionally substituted by NR^{III-7}R^{III-8}, or R^{III-11};

R^{III-13} is

a) (P═O)(OH)₂,

b) (P═O)(C₁₋₇ alkoxy)₂,

c) CO (CH₂)ₙCON(CH₃)(CH₂)ₙSO₃⁻M⁺,

d) an amino acid,

e) C(═O)^{III-}aryl,

f) C(═O)C₁₋₆alkyl, optionally substituted by NR^{III-10}R^{III-10}, or

g) CO(CH₂)ₙCO₂H;

R^{III-14} is

a) het^{III},

b) (CH₂)ₙOR^{III-13}, or

c) C₁₋₇ alkyl substituted by one or more substituents selected from a group consisting of R^{III-11}, OC₁-7 alkyl which is further substituted with het^{III}, NR^{III-7}R^{III-8}, or SOᵢR^{III-9};

aryl^{III} is a phenyl radical or an ortho-fused bicyclic carbocyclic radical wherein at least one ring is aromatic;

het^{III} is a four- (4), five- (5), six- (6), or seven- (7) membered saturated or unsaturated heterocyclic ring having 1, 2, or 3 heteroatoms selected from the group consisting of oxygen, sulfur, and nitrogen, which is optionally fused to a benzene ring, or any bicyclic heterocycle group;

wherein any aryl^{III} is optionally substituted with one or more substituents selected from the group consisting of halo, OH, CF₃, C₁₋₆alkoxy, and C₁₋₆ alkyl which maybe further substituted by one to three SR^{III-10}, NR^{III-10}R^{III-10}, OR^{II-10}, or CO₂R^{III-10};

wherein any het^{III} is optionally substituted with one or more substituents selected from the group consisting of halo, OH, CF₃, C₁₋₆alkoxy, oxo, oxine, and C₁₋₆ alkyl which maybe further substituted by one to three SR^{III-10}, NR^{III-10}R^{III-10}, OR^{III-10}, or CO₂R^{III-10};

i^{III} is 0, 1, or 2;

m^{III} is 1, 2, or 3;

n^{III} is 1, 2, 3, 4, 5, or 6; and

M is sodium, potassium, or lithium;

wherein Formula IV is

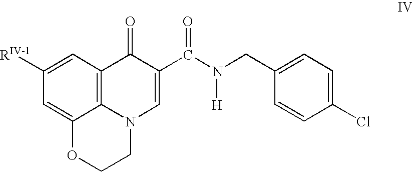

or a pharmaceutically acceptable salt, racemate, solvate, tautomer, optical isomer or prodrug derivative thereof; wherein R^{IV-1} is C₁₋₆ alkyl, optionally substituted with

—OH, —OC₁₋₄ alkyl or het^{IV};

wherein C₁₋₆ alkyl is optionally partially unsaturated;

wherein het^{IV} is a radical of a five- or six-membered heterocyclic ring having one or two heteroatoms selected from the group consisting of oxygen, sulfur and N;

wherein Formula V is

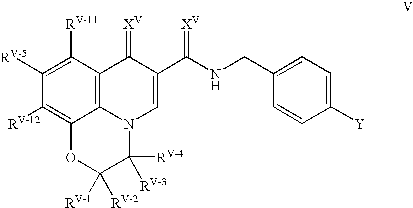

or a pharmaceutically acceptable salt, racemate, solvate, tautomer or optical isomer thereof wherein:

each X^{V} is independently O or S;

Y is Cl, F, Br, CN or NO₂;

R^{V-1}, R^{V-2}, R^{V-3} and R^{V-4} are independently

a) hydrogen,

b) N₃,

c) CN,

d) fluoro,

e) trifluoromethyl,

f) aryl^{V},

g) het^{V},

h) C_{V-1-V-8} alkyl, optionally substituted with R^{V-6} or OR^{V-7}, or

i) R^{V-1} and R^{V-2} or R^{V-3} and R^{V-4} together with the carbon to which they are attached form C₃₋₈cycloalkyl or _{V-}het;

R^{V-5} is C₁₋₈alkyl, which may be partially unsaturated and optionally substituted with one to three N₃, halo, CN, R^{V-6} or R^{V-7};

R^{V-6} is

a) aryl^{V},

b) het^{V},

c) SOᵢR^{V-8},

d) OR^{V-8},

e) C(═O)OR^{V-8},

f) C(═O)R^{V-8}, or

g) NR^{V-8}R^{V-9};

R^{V-7} is

a) P(═O)(OR^{V-10})₂,

b) CO(CH_{V-2})ⱼCON(CH₃)(CH₂)ₖSO₃⁻M^{V+},

c) an amino acid,

d) C(═O)C₁₋₆alkyl, optionally substituted by NR^{V-10}R^{V-10}, or

e) CO(CH₂)ₙCO₂H;

R^{V-8} and R^{V-9} are independently

a) hydrogen,

b) C₃₋₈cycloalkyl,

c) aryl^{V},

d) het^{V}, or

e) C₁₋₈alkyl which is further optionally substituted with one or more aryl^{V}, het^{V}, halo, CN, CO₂R^{V-10}, SOᵢR^{V-10}, OR^{V-10}, NR^{V-10}R^{V-10}, CF₃, or C₃₋₈cycloalkyl;

R^{V-10} is

a) H or

b) C₁₋₈alkyl, optionally substituted with OH or OC₁₋₄alkyl;

R^{V-11} and R^{V-12} are independently

a) hydrogen,

b) halo,

c) NO₂,

d) CN,

e) R^{V-6},

f) SOᵢNR^{V-8}R^{V-9}, or

g) C₁₋₈alkyl, which may be partially unsaturated and optionally substituted with one to three N^{V-3}, halo, CN, R^{V-6} or OR^{V-7};

aryl^{V} is

a phenyl radical, optionally fused with a saturated or unsaturated carbocyclic or heterocyclic ring; at each occurrence, aryl^{V} may be substituted with one or more halo, CN, CO₂R^{V-10}, SOᵢR^{V-10}, OR^{V-10}, NR^{V-10}, CF₃, C₃₋₈cycloalkyl, or C₁₋₄alkyl wherein C₁₋₄alkyl is optionally substituted with OR^{V-10};

het^{V} is

a four- (4), five- (5), six- (6), or seven- (7) membered saturated or unsaturated heterocyclic ring having 1, 2, or 3 heteroatoms selected from the group consisting of O, S, and NW^{V}, wherein W^{V} is hydrogen, C₁₋₄alkyl, C(═O)OC₁₋₄alkyl or absent, wherein het^{V} is optionally fused with a benzene ring, a carbocyclic or a heterocyclic ring; at each occurrence, het^{V} may be substituted with one or more halo, CN, CO₂R^{V-10}, SOᵢR^{V-10}, OR^{V-10}, NR^{V-10}R^{V-10}, C₁₋₄alkyl, CF₃, C₃₋₈cycloalkyl, oxo or oxine;

at each occurrence, a cycloalkyl may be substituted with C₁₋₄alkyl, OR^{V-10}, oxo, oxine, or a spiro fused _{V-}het;

i^{V} is 0, 1 or 2;

j^{V} is 1, 2, 3, 4, 5, or 6;

k^{V} is 1, 2, 3, 4, 5, or 6;

n^{V} is 1, 2, 3, 4, 5, or 6;

M^{V} is sodium, potassium, or lithium; and

wherein Formula XI is

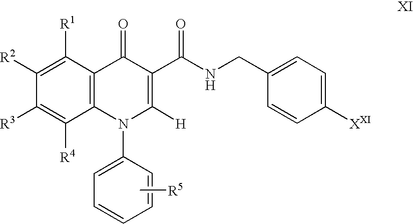

and pharmaceutically acceptable salts thereof, wherein,

X^{XI} is Cl, F, Br, CN, or NO₂;

R^{XI-1} is H, halo, or C₁₋₄alkyl optionally substituted by one to three halo;

R^{XI-2} is

a) H,

b) halo,

c) aryl^{XI},

d) het^{XI}, wherein said het^{XI}, is bound via a carbon atom,

e) C₁₋₇alkyl which may be partially unsaturated and optionally substituted by one or more substituents of the group R^{XI-10}, NR^{XI-7}R^{XI-8}, halo, (C═O)R^{XI-6}, or S(O)ₘR^{XI-6},

f) NR^{XI-7}R^{XI-8},

g) OR^{XI-11},

h) SR^{XI-11},

NHSO₂R^{XI-6},

j) S(O)ₘR^{XI-6},

k) (C═O)R^{XI-6},

l) (C═O)OR^{XI-11},

m) CHO,

n) cyano, or

o) C₃₋₈cycloalkyl which may be partially unsaturated and is optionally substituted by one or more substituents selected from halo, oxo, R^{XI-10}, C₁₋₇alkyl, or NR^{XI-7}R^{XI-8};

R^{XI-3} is

a) H,

b) halo,

c) OR¹¹, or

d) C₁₋₇alkyl which may be partially unsaturated and optionally substituted by one or more substituents of the group OR^{XI-11}, SR^{XI-11}, NR^{XI-7}R^{XI-8}, or halo, or

R^{XI-2} together with R^{XI-3} form a carbocyclic or saturated 5 or 6 membered het^{XI} which may be optionally substituted by NR^{XI-7}R^{XI-8}, het^{XI} attached through a carbon atom, or C₁₋₇alkyl which may be optionally substituted by OR^{XI-12};

R^{XI-4} is

a) H,

b) halo,

c) OR^{XI-11}, or

d) C₁₋₇alkyl which may be partially unsaturated and optionally substituted by one or more substituents of the group OR^{XI-11}, SR^{XI-11}, NR^{XI-7}R^{XI-8}, aryl^{XI}, halo, C₃₋₈cycloalkyl optionally substituted by OR^{XI-12}, or het^{XI} attached through a carbon atom, or

e) NR^{XI-7}R^{XI-8};

R^{XI-5} is

a) H,

b) halo,

c) OR^{XI-11},

d) O(CH₂CH₂O)ₙR^{XI-12},

e) C₃₋₈cycloalkyl which may be partially unsaturated and is optionally substituted by one or more substituents selected from halo, OR^{XI-12}, SR^{XI-12}, OX, C₁₋₇alkyl or NR^{XI-12}R^{XI-12},

f) het^{XI},

g) aryl^{X},

h) NHSO₂R^{XI-6},

i) S(O)ₘR^{XI-6},

j) (C═O)R^{XI-6},

k) (C═O)OR^{XI-11},

l) nitro,

m) cyano,

n) SR^{XI-11},

o) NR^{XI-7}R^{XI-8},

p) C₁₋₇alkyl which may be partially unsaturated and is optionally substituted by one or more substituents selected from NR^{XI-7}R^{XI-8}, R^{XI-10}, S(O)ₘR^{XI-6}, (P═O)(OR^{XI-12})₂, (C═O)R^{XI-6}, or halo,

q) CHO,

r) SCN,

s) Any two adjacent R^{XI-5} substituents taken with the bond connecting them form an aryl^{XI}, or het^{XI}, or

t) Any two adjacent R^{XI-5} substituents taken together constitute a C₃₋₆alkyl chain which may be optionally substituted by R^{XI-9}, NR^{XI-7}R^{XI-8}, cyano, CO₂R^{XI-12}, OR^{XI-11}, SR^{XI-11}, or (═O);

R^{XI-6} is

a) C₁₋₇alkyl,

b) NR^{XI-11}R^{XI-11},

c) aryl^{XI}, or

d) het^{XI};

R^{XI-7} and R^{XI-8} are independently

a) H,

b) aryl^{XI},

c) C₁₋₇alkyl which may be partially unsaturated and is optionally substituted by one or more substituents selected from S(O)ₘR^{XI-6}, CONR^{XI-12}, R^{XI-12}, CO₂R^{XI-12}, (C═O)R^{XI-9}, het^{XI}, aryl^{XI}, cyano, or halo,

d) C₂₋₇alkyl which may be partially unsaturated and is substituted by one or more substituents selected from NR^{XI-12}R^{XI-12}, OR^{XI-11}, or SR^{XI-11},

e) C₃₋₈cycloalkyl which may be partially unsaturated and is optionally substituted by one or more substituents selected from halo, OR^{XI-12}, SR^{XI-12}, oxo, or NR^{XI-12}R^{XI-12},

f) (C═O)R^{XI-9}, or

g) R^{XI-7} and R^{XI-8} together with the nitrogen to which they are attached for a het^{XI};

R^{XI-9} is

a) aryl^{XI},

b) het^{XI}, wherein said het^{XI} is bound through a carbon atom,

c) C₁₋₇alkyl optionally substituted by aryl^{XI}, het^{XI}, cyano, OR^{XI-12}, SR^{XI-12}, NR^{XI-12}R^{XI-12}, or halo, or

d) C₃₋₈cycloalkyl which may be partially unsaturated and is optionally substituted by one or more substituents selected from halo, OR^{XI-12}, SR^{XI-12}, or NR^{XI-12}R^{XI-12};

R^{XI-10} is

b) SR^{XI-11},

c) CO₂R^{XI-12},

d) het^{XI},

e) aryl^{XI}, or

f) cyano;

R^{XI-11} is

a) H,

b) aryl^{XI},

c) het^{XI}, wherein said het^{XI} is bound through a carbon atom,

d) C₁₋₇alkyl optionally substituted by aryl^{XI}, het^{XI} wherein said het^{XI} is bound through a carbon atom, C₃₋₈cycloalkyl optionally substituted by OR^{XI-12}, or halo,

e) C₂₋₇alkyl substituted by OR^{XI-12}, SR^{XI-12}, or NR^{XI-12}R^{XI-12}, or

f) C₃₋₈cycloalkyl which may be partially unsaturated and is optionally substituted by one or more substituents selected from halo, OR^{XI-12}, SR^{XI-12}, or NR^{XI-12}R^{XI-12},

R^{XI-12} is H, or C₁₋₇alkyl;

each m^{XI} is independently 1 or 2;

each n^{XI} is independently 1, 2, or 3;

wherein aryl^{XI} is a phenyl radical or an ortho-fused bicyclic carbocyclic radical wherein at least one ring is aromatic and is optionally substituted with one or more substituents selected from halo, OH, cyano, CO₂R^{XI-2}, CF₃, C₁₋₆alkoxy, or C₁₋₆ alkyl which may be further substituted by one to three SR^{XI-12}, NR^{XI-12}R^{XI-12}, OR^{XI-12}, or CO₂R^{XI-12} groups;

wherein het^{XI} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocyclic group and wherein any het^{XI} is optionally substituted with one or more substituents selected from halo, OH, cyano, phenyl, CO₂R^{XI-12}, CF₃, C₁₋₆alkoxy, oxo, oxime, or C₁₋₆alkyl which may be further substituted by one to three SR^{XI-12}, NR^{XI-12}R^{XI-12}, OR^{XI-12}, or CO₂R^{XI-12} groups; and

wherein halo is F, Cl, Br, I;

and pharmaceutically acceptable salts thereof.

Also provided is the use of compounds of Formula I-V and XI to prepare medicaments for preventing or treating atherosclerosis or reestenosis in mammals.

The advantage of using compounds of Formula I-V and XI in the method of our invention is their extensive activity against herpesviruses since atherosclerosis is related to the number of herpesvirus infections. Drugs containing compound of Formula I-V and XI could prevent the inflammatory response resulting from reactivation of HCMV, EBV, HSV-1, HSV-2, HHV-8 and VZV.

### DETAILED DESCRIPTION OF THE INVENTION

The compounds of Formula I, their method of preparation and formulation into pharmaceutical dosage form are described in U.S. Pat. No. 6,093,732. The disclosure of U.S. Pat. No. 6,093,732 is herein incorporated in its entirety by reference.

The compounds of Formula II, their method of preparation and formulation into pharmaceutical dosage form are described in U.S. Pat. No. 6,248,736. The disclosure of U.S. Pat. No. 6,248,736 is herein incorporated in its entirety by reference.

The compounds of Formula III, their method of preparation and formulation into pharmaceutical dosage form are described in U.S. Pat. No. 6,248,739. The disclosure of U.S. Pat. No. 6,248,739 is herein incorporated in its entirety by reference.

The compounds of Formula IV, their method of preparation and formulation into pharmaceutical dosage forms are described in U.S. Pat. No. 6,340,680. The disclosure of U.S. Pat. No. 6,340,680 is herein incorporated in its entirety by reference.

The compounds of Formula V, their method of preparation and formulation into pharmaceutical dosage forms are described in U.S. patent application Ser. No. 09/894,354, filed Jun. 28, 2001. The disclosure of U.S. patent application Ser. No. 09/894,354 is herein incorporated in its entirety by reference.

The compounds of Formula XI, their method of preparation and formulation into pharmaceutical dosage forms are described in U.S. patent application Ser. No. 09/875,432, filed Jun. 5, 2001. The disclosure of U.S. patent application Ser. No. 09/875,432 is herein incorporated in its entirety by reference.

The correspondence between the compounds utilized in the method of the invention and the compounds incorporated by reference is as follows:

Formula I corresponds to Formula I of U.S. Pat. No. 6,093,732.

Formula II corresponds to Formula I of U.S. Pat. No. 6,248,736.

Formula III corresponds to Formula I of U.S. Pat. No. 6,248,739.

Formula IV corresponds to Formula I of U.S. Pat. No. 6,340,680.

Formula V corresponds to Formula I of U.S. patent application Ser. No. 09/894,354.

Formula XI corresponds to Formula I of U.S. patent application Ser. No. 09/875,432.

The invention further provides:

A method for preventing or treating atherosclerosis or restenosis in mammals, wherein the compound administered has the Formula I and is selected from the group consisting of:

(1) N-[(4-chlorophenyl)methyl]-4-hydroxy-7-(trifluoromethyl)-3-quinolinecarboxamide;

(2) 7-amino-N-[(4-chlorophenyl)methyl]-4-hydroxy-3-quinolinecarboxamide;

(3) N-[(4-chlorophenyl)methyl]-8-fluoro-4,6-dihydroxy-3-quinolinecarboxamide;

(4) 6-bromo-N-[(4-chlorophenyl)methyl]-8-fluoro-3-quinolinecarboxamide;

(5) N-[(4-chlorophenyl)methyl]-8-fluoro-4-hydroxy-6-iodo-3-quinolinecarboxamide;

(6) N-[(4-chlorophenyl)methyl]-4-hydroxy-3-quinolinecarboxamide;

(7) N-[(4-chlorophenyl)methyl]-4-hydroxy-7-methoxy-3-quinolinecarboxamide;

(8) N-[(4-chlorophenyl)methyl]-4-hydroxy-5,7-bis(trifluoromethyl)-3-quinolinecarboxamide;

(9) N-[(4-chlorophenyl)methyl]-7-fluoro-4-hydroxy-3-quinolinecarboxamide;

(10) N-[(4-chlorophenyl)methyl]-6-fluoro-4-hydroxy-3-quinolinecarboxamide;

(11) N-[(4-chlorophenyl)methyl]-4-hydroxy-7-methyl-3-quinolinecarboxamide;

(12) N-[(4-chlorophenyl)methyl]-8-fluoro-4-hydroxy-3-quinolinecarboxamide;

(13) N-[(4-chlorophenyl)methyl]-4-hydroxy-6-nitro-3-quinolinecarboxamide;

(14) N-[(4-chlorophenyl)methyl]-5,6,7,8-tetrafluoro-4-hydroxy-3-quinolinecarboxamide;

(15) N-[(4-chlorophenyl)methyl]-6,7,8-trifluoro-4-hydroxy-3-quinolinecarboxamide;

(16) 6,7,8-trifluoro-4-hydroxy-3-quinolinecarboxylic acid 2-(4-chlorophenyl)hydrazide;

(17) N-[(4-chlorophenyl)methyl]-5,8-difluoro-4-hydroxy-3-quinolinecarboxamide;

(18) N-[(4-chlorophenyl)methyl]-7,8-difluoro-4-hydroxy-3-quinolinecarboxamide;

(19) 6-benzoyl-N-(4-chlorobenzyl)-4-hydroxy-3-quinolinecarboxamide;

(20) N-[(4-chlorophenyl)methyl]-4-hydroxy-8-methoxy-3-quinolinecarboxamide;

(21) 6-chloro-N-[(4-chlorophenyl)methyl]-4-hydroxy-3-quinolinecarboxamide;

(22) N-[(4-chlorophenyl)methyl]-4-hydroxy-6-methyl-3-quinolinecarboxamide;

(23) N-[(4-chlorophenyl)methyl]-4-hydroxy-6-methoxy-3-quinolinecarboxamide;

(24) N-[(4-chlorophenyl)methyl]-6-cyano-4-hydroxy-3-quinolinecarboxamide;

(25) 7-(acetylamino)-N-[(4-chlorophenyl)methyl]-4-hydroxy-3-quinolinecarboxamide;

(26) N-[(4-chlorophenyl)methyl]-4-hydroxy-7-[(methylsulfonyl)amino]-3-quinolinecarboxamide;

(27) N-[(4-chlorophenyl)methyl]-7-(dimethylamino)-4-hydroxy-3-quinolinecarboxamide;

(28) 6-amino-N-[(4-chlorophenyl)methyl]-4-hydroxy-3-quinolinecarboxamide;

(29) N-[(4-chlorophenyl)methyl]-4-hydroxy-6-[(methylsulfonyl)amino]-3-quinolinecarboxamide;

(30) N-[(4-chlorophenyl)methyl]-6-(dimethylamino)-4-hydroxy-3-quinolinecarboxamide;

(31) 6-(acetylamino)-N-[(4-chlorophenyl)methyl]-4-hydroxy-3-quinolinecarboxamide;

(32) N-[(4-chlorophenyl)methyl]-4-hydroxy-7-(1-pyrrolyl)-3-quinolinecarboxamide;

(33) N-[(4-chlorophenyl)methyl]-4-hydroxy-7-[(phenylsulfonyl)amino]-3-quinolinecarboxamide;

(34) N-[(4-chlorophenyl)methyl]-4-hydroxy-7-[[(phenylmethyl)sulfonyl]amino]-3-quinolinecarboxamide;

(35) N-[(4-chlorophenyl)methyl]-7-[[(4-chlorophenyl)sulfonyl]amino]-4-hydroxy-3-quinolinecarboxamide;

(36) 8-fluoro-4-hydroxy-3-quinolinecarboxylic acid 2-(4-chlorophenyl)hydrazide;

(37) N-[(4-chlorophenyl)methyl]-8-fluoro-4-hydroxy-6-methyl-3-quinolinecarboxamide;

(38) (278) N-(4-chlorobenzyl)-8-hydroxy[1,3]dioxolo[4,5-g]quinoline-7-carboxamide;

(39) N-[(4-chlorophenyl)methyl]-4-hydroxy-6-iodo-3-quinolinecarboxamide;

(40) N-[(4-chlorophenyl)methyl]-6-(cyanomethyl)-4-hydroxy-3-quinolinecarboxamide;

(41) N-[(4-chlorophenyl)methyl]-4,5-dihydroxy-3-quinolinecarboxamide;

(42) 7,8-dichloro-N-[(4-chlorophenyl)methyl]-4-hydroxy-3-quinolinecarboxamide;

(43) N-[(4-chlorophenyl)methyl]-4,6-dihydroxy-3-quinolinecarboxamide;

(44) N-[(4-chlorophenyl)methyl]-4,8-dihydroxy-3-quinolinecarboxamide;

(45) 8-chloro-N-[(4-chlorophenyl)methyl]-4-hydroxy-3-quinolinecarboxamide;

(46) N-[(4-chlorophenyl)methyl]-4-hydroxy-6-[[(1-phenyl-1H-pyrazol-5-yl)amino]sulfonyl]-3-quinoline -carboxamide;

(47) N-[(4-chlorophenyl)methyl]-8-cyano-4-hydroxy-3-quinolinecarboxamide;

(48) N-[(4-chlorophenyl)methyl]-4-hydroxy-8-nitro-3-quinolinecarboxamide;

(49) 7-amino-N-[(4-chlorophenyl)methyl]-4-hydroxy-8-methyl-3-quinolinecarboxamide;

(50) N-[(4-chlorophenyl)methyl]-6-cyano-8-fluoro-4-hydroxy-3-quinolinecarboxamide;

(51) 6-(aminothioxomethyl)-N-[(4-chlorophenyl)-methyl]-8-fluoro-4-hydroxy-3-quinolinecarboxamide;

(52) N-[(4-chlorophenyl)methyl]-8-fluoro-4-hydroxy-6-(3-hydroxy-1-propynyl)-3-quinolinecarboxamide;

(53) 8-fluoro-4-hydroxy-6-iodo-3-quinolinecarboxylic acid 2-(4-chlorophenyl)hydrazide;

(54) 8-fluoro-4-hydroxy-6-methyl-3-quinolinecarboxylic acid 2-(4-chlorophenyl)hydrazide;

(55) N-((4-chlorophenyl)methyl)-7-chloro-4-hydroxy-3-quinolinecarboxamide;

(56) N-((4-chlorophenyl)methyl)-6-bromo-4-hydroxy-3-quinolinecarboxamide;

(57) N-((4-chlorophenyl)methyl)-4-hydroxy-6-phenyl-3-quinolinecarboxamide;

(58) N-((4-chlorophenyl)methyl)-8-chloro-4-hydroxy-5-trifluoromethyl-3-quinolinecarboxamide;

(59) N-((4-chlorophenyl)methyl)-6,8-dimethoxy-4-hydroxy-3-quinolinecarboxamide;

(60) N-((4-chlorophenyl)methyl)-6,7-dimethoxy-4-hydroxy-3-quinolinecarboxamide;

(61) N-((4-chlorophenyl)methyl)-4-hydroxy-5-methyl-3-quinolinecarboxamide;

(62) N-[(4-chlorophenyl)methyl]-6-(1,1-dimethylethyl)-4-hydroxy-3-quinolinecarboxamide;

(63) N-[(4-chlorophenyl)methyl]-7,8-dihydro-4-hydroxy-6H-cyclopenta[g]quinoline-3-carboxamide;

(64) N-[(4-chlorophenyl)methyl]-1,4-dihydro-8-(methylthio)-4-oxo-3-quinolinecarboxamide;

(65) N-[(4-chlorophenyl)methyl]-9-hydroxythiazolo[5,4-f]quinoline-8-carboxamide;

(66) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;

(67) sodium 2-[(8-{[3-(3-{[(4-chlorobenzyl)amino]carbonyl}-8-fluoro-4-hydroxy-6-quinolinyl)-2-propynyl]oxy}-8-oxooctanoyl)(methyl)amino]-1-ethanesulfonate;

(68) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;

(69) N-(4-chlorobenzyl)-4-hydroxy-7-{[(1-naphthylmethyl)amino]sulfonyl}-3-quinolinecarboxamide;

(70) N-(4-chlorobenzyl)-4-hydroxy-7-(methylsulfanyl)-3-quinolinecarboxamide;

(71) N-[(4-chlorophenyl)methyl]-4-hydroxy-6-[(phenylmethyl)thio]-7-(trifluoromethyl)-3-quinolinecarboxamide;

(72) 3-(3-{[(4-chlorobenzyl)amino]carbonyl}-4-hydroxy-6-quinolinyl)propyl hydrogen phosphonate;

(73) N-[(4-chlorophenyl)methyl]-8-fluoro-4-hydroxy-6-(2-thiazolyl)-3-quinolinecarboxamide;

(74) N-[(4-chlorophenyl)methyl]-8-fluoro-4-hydroxy-6-(2-thiophenyl)-3-quinolinecarboxamide;

(75) N-((4-chlorophenyl)methyl)-4-hydroxy-5-trifluoromethyl-3-quinolinecarboxamide;

(76) N-((4-chlorophenyl)methyl)-8-fluoro-4-hydroxy-6-(2-methylphenyl)-3-quinolinecarboxamide;

(77) N-((4-chlorophenyl)methyl)-6,7-difluoro-4-hydroxy-8-(tetrahydro-2H-pyran-4-oxy)-3-quinolinecarboxamide;

(78) N-((4-chlorophenyl)methyl)-6,7-difluoro-4-hydroxy-8-methoxy-3-quinolinecarboxamide;

(79) N-((4-chlorophenyl)methyl)-7,8-dimethoxy-6-fluoro-4-hydroxy-3-quinolinecarboxamide;

(80) N-((4-chlorophenyl)methyl)-6,8-difluoro-4-hydroxy-7-(4-(hydroxymethyl)phenoxy)-3-quinolinecarboxamide;

(81) N-((4-chlorophenyl)methyl)-6,8-difluoro-4-hydroxy-7-methoxy-3-quinolinecarboxamide;

(82) N-((4-chlorophenyl)methyl)-6,8-difluoro-4-hydroxy-7-(2-(methoxy)ethoxy)-3-quinolinecarboxamide;

(83) N-((4-chlorophenyl)methyl)-6,7-difluoro-4-hydroxy-8-(2-(methoxy)ethoxy)-3-quinolinecarboxamide;

(84) N-((4-chlorophenyl)methyl)-7,8-di(2-(methoxy)ethoxy)-6-fluoro-4-hydroxy-3-quinolinecarboxamide;

(85) N-((4-chlorophenyl)methyl)-6,8-difluoro-4-hydroxy-7-(1-methylethoxy)-3-quinolinecarboxamide;

(86) N-(4-chlorobenzyl)-8-fluoro-4-hydroxy-6-(1,3-thiazol-2-yl)-3-quinolinecarboxamide;

(87) N-(4-chlorobenzyl)-6,8-difluoro-4-hydroxy-7-[(2-methoxyethyl)amino]-3-quinolinecarboxamide;

(88) N-(4-chlorobenzyl)-6-(5-cyano-1-pentynyl)-8-fluoro-4-hydroxy-3-quinolinecarboxamide;

(89) N-(4-chlorobenzyl)-8-fluoro-4-hydroxy-6-(2-pyridinyl)-3-quinolinecarboxamide;

(90) N′-(4-chlorophenyl)-4-hydroxy-6-iodo-3-quinolinecarbohydrazide;

(91) N-(4-chlorobenzyl)-8-fluoro-4-hydroxy-6-[2-(2-pyridinyl)ethynyl]-3-quinolinecarboxamide;

(92) N-(4-chlorobenzyl)-6,8-difluoro-4-hydroxy-7-[(2-hydroxyethyl)amino]-3-quinolinecarboxamide;

(93) N-(4-chlorobenzyl)-8-fluoro-4-hydroxy-6-(4-hydroxy-1-butynyl)-3-quinolinecarboxamide;

(94) N-(4-chlorobenzyl)-8-fluoro-4-hydroxy-6-(3-methoxy-1-propynyl)-3-quinolinecarboxamide;

(95) N-(4-chlorobenzyl)-8-fluoro-4-hydroxy-6-(3-hydroxy-1-butynyl)-3-quinolinecarboxamide;

(96) 6-(4-bromo-2-thienyl)-N-(4-chlorobenzyl)-8-fluoro-4-hydroxy-3-quinolinecarboxamide;

(97) N-(4-chlorobenzyl)-8-fluoro-6-(hydrazino-carbothioyl)-4-hydroxy-3-quinolinecarboxamide;

(98) N-(4-chlorobenzyl)-8-fluoro-4-hydroxy-6-(4-hydroxybutyl)-3-quinolinecarboxamide;

(99) N-(4-chlorobenzyl)-8-fluoro-4-hydroxy-6-(5-methyl-1,3,4-thiadiazol-2-yl)-3-quinolinecarboxamide;

(100) N-(4-chlorobenzyl)-4-hydroxy-7-(3-hydroxy-1-propynyl)-3-quinolinecarboxamide;

(101) 7-(aminocarbothioyl)-N-(4-chlorobenzyl)-4-hydroxy-3-quinolinecarboxamide;

(102) N-(4-chlorobenzyl)-8-fluoro-4-hydroxy-6-(3-methoxypropyl)-3-quinolinecarboxamide;

(103) N-(4-chlorobenzyl)-8-fluoro-4-hydroxy-6-[(Z)-4-hydroxy-1-butenyl]-3-quinolinecarboxamide;

(104) N-(4-chlorobenzyl)-6-(5-cyanopentyl)-8-fluoro-4-hydroxy-3-quinolinecarboxamide;

(105) N-(4-chlorobenzyl)-8-fluoro-4-hydroxy-6-(3-hydroxy-3-methylbutyl)-3-quinolinecarboxamide;

(106) N-(4-chlorobenzyl)-8-fluoro-4-hydroxy-6-(5-hydroxy-1-pentynyl)-3-quinolinecarboxamide;

(107) 6-{3-[benzyl(methyl)amino]propyl}-N-(4-chlorobenzyl)-8-fluoro-4-hydroxy-3-quinolinecarboxamide;

(108) methyl 3-{[(4-chlorobenzyl)amino]carbonyl}-8-fluoro-4-hydroxy-6-quinolinecarboxylate;

(109) N-(4-chlorobenzyl)-8-fluoro-4-hydroxy-6-(3-hydroxypropyl)-3-quinolinecarboxamide;

(110) N-(4-chlorobenzyl)-4-hydroxy-7-(3-hydroxy-propyl)-3-quinolinecarboxamide;

(111) ethyl (E)-3-(3-{[(4-chlorobenzyl)amino]carbonyl}-8-fluoro-4-hydroxy-6-quinolinyl)-2-propenoate;

(112) sodium 2-[{8-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;

(113) 3-(3-{[(4-chlorobenzyl)amino]carbonyl}-8-fluoro-4-hydroxy-6-quinolinyl)propanoic acid;

(114) 5-(3-{[(4-chlorobenzyl)amino]carbonyl}-8-fluoro-4-hydroxy-6-quinolinyl)-4-pentynoic acid;

(115) N-[(4-chlorophenyl)methyl]-9]hydroxy-3H-pyrazolo[4,3-f]quinoline-8-carboxamide;

(116) N-(4-chlorobenzyl)-4-hydroxy-6-iodo-8-methoxy-3-quinolinecarboxamide;

(117) N-(4-chlorobenzyl)-4-hydroxy-6-(3-hydroxy-1-propynyl)-8-methoxy-3-quinolinecarboxamide;

(118) N-(4-chlorobenzyl)-4-hydroxy-8-methoxy-6-(3-methoxy-1-propynyl)-3-quinolinecarboxamide;

(119) N-(4-chlorobenzyl)-4-hydroxy-6-(3-hydroxypropyl)-8-methoxy-3-quinolinecarboxamide;

(120) N-(4-chlorobenzyl)-4-hydroxy-6-(3-hydroxy-1-propynyl)-8-(trifluoromethyl)-3-quinolinecarboxamide;

(121) N-(4-chlorobenzyl)-4-hydroxy-6-(3-hydroxy-1-propynyl)-8-(trifluoromethoxy)-3-quinolinecarboxamide;

(122) N-(4-chlorobenzyl)-4-hydroxy-6-(3-hydroxypropyl)-8-(trifluoromethyl)-3-quinolinecarboxamide;

(123) N-(4-chlorobenzyl)-4-hydroxy-8-(2-hydroxyethoxy)-6-(3-hydroxypropyl)-3-quinolinecarboxamide;

(124) N-(4-chlorobenzyl)-4-hydroxy-6-(3-hydroxy-1,1-dimethylpropyl)-3-quinolinecarboxamide;

(125) N-(4-chlorobenzyl)-8-fluoro-4-hydroxy-6-[3-(methylsulfanyl)-1-propynyl]-3-quinolinecarboxamide;

(126) N-(4-chlorobenzyl)-6-[3-(ethylsulfanyl)-1-propynyl]-8-fluoro-4-hydroxy-3-quinolinecarboxamide;

(127) N-(4-chlorobenzyl)-8-fluoro-4-hydroxy-6-[(Z)-3-(methylsulfanyl)-1-propenyl]-3-quinolinecarboxamide;

(128) N-(4-chlorobenzyl)-6-[(Z)-3-(ethylsulfanyl)-1-propenyl]-8-fluoro-4-hydroxy-3-quinolinecarboxamide;

(129) N-(4-chlorobenzyl)-8-fluoro-4-hydroxy-6-[3-(methylsulfanyl)propyl]-3-quinolinecarboxamide;

(130) 3-(3-{[(4-chlorobenzyl)amino]carbonyl}-8-fluoro-4-hydroxy-6-quinolinyl)-2-propynyl formate;

(131) N-(4-chlorobenzyl)-8-fluoro-4-hydroxy-6-(3-hydroxybutyl)-3-quinolinecarboxamide;

(132) N-(4-chlorobenzyl)-6-[(E)-2-cyanoethenyl]-8-fluoro-4-hydroxy-3-quinolinecarboxamide;

(133) N-(4-chlorobenzyl)-4-hydroxy-6-(3-hydroxy-1-propynyl)-3-quinolinecarboxamide;

(134) N-(4-chlorobenzyl)-6-[(Z)-3-hydroxy-1-propenyl]-4-oxo-1,4-dihydro-3-quinolinecarboxamide;

(135) N-(4-chlorobenzyl)-6-[(E)-3-hydroxy-1-propenyl]-4-oxo-1,4-dihydro-3-quinolinecarboxamide;

(136) N-(4-chlorobenzyl)-8-fluoro-4-hydroxy-6-[(Z) -3-hydroxy-1-propenyl]-3-quinolinecarboxamide;

(137) N-(4-chlorobenzyl)-6-(2-cyanoethyl)-8-fluoro-4-hydroxy-3-quinolinecarboxamide;

(138) N-(4-chlorobenzyl)-8-fluoro-4-hydroxy-6-(3-oxopropyl)-3-quinolinecarboxamide;

(139) N-(4-chlorobenzyl)-4-hydroxy-7-(4-hydroxybutyl)-3-quinolinecarboxamide;

(140) N-(4-chlorobenzyl)-8-fluoro-4-hydroxy-6-(hydroxymethyl)-3-quinolinecarboxamide;

(141) 3-(3-{[(4-chlorobenzyl)amino]carbonyl}-8-fluoro-4-hydroxy-6-quinolinyl)propyl methanesulfonate;

(142) N-(4-chlorobenzyl)-8-fluoro-6-(3-fluoro-1-propynyl)-4-hydroxy-3-quinolinecarboxamide;

(143) N-(4-chlorobenzyl)-4-hydroxy-6-(3-hydroxypropyl)-3-quinolinecarboxamide;

(144) 3-(3-{[(4-chlorobenzyl)amino]carbonyl}-8-fluoro-4-hydroxy-6-quinolinyl)propyl 2-bromoacetate;

(145) 3-(3-{[(4-chlorobenzyl)amino]carbonyl}-8-fluoro-4-hydroxy-6-quinolinyl)propyl 2-[(tert-butoxycarbonyl)amino]-3-methylbutanoate;

(146) 3-(3-{[(4-chlorobenzyl)amino]carbonyl}-8-fluoro-4-hydroxy-6-quinolinyl)propyl 2-(4-morpholinyl)acetate;

(147) 3-(3-{[(4-chlorobenzyl)amino]carbonyl}-8-fluoro-4-hydroxy-6-quinolinyl)propyl 2-(dimethylamino)acetate;

(148) 3-(3-{[(4-chlorobenzyl)amino]carbonyl}-8-fluoro-4-hydroxy-6-quinolinyl)propyl 2-amino-3-methylbutanoate;

(149) 3-(3-{[(4-chlorobenzyl)amino]carbonyl}-8-fluoro-4-hydroxy-6-quinolinyl)-2-propynyl phenylcarbamate;

(150) N-(4-chlorobenzyl)-4-hydroxy-6-propyl-3-quinolinecarboxamide;

(151) N-(4-chlorobenzyl)-4-hydroxy-6-(3-hydroxy-1-butynyl)-3-quinolinecarboxamide;

(152) N-(4-chlorobenzyl)-8-fluoro-4-hydroxy-6-[(E)-3-oxo-1-butenyl]-3-quinolinecarboxamide;

(153) N-(4-chlorobenzyl)-8-fluoro-4-hydroxy-6-(5-hydroxypentyl)-3-quinolinecarboxamide;

(154) 3-(3-{[(4-chlorobenzyl)amino]carbonyl}-8-fluoro-4-hydroxy-6-quinolinyl)propyl 2,6-bis[(tert-butoxycarbonyl)amino]hexanoate;

(155) 3-(3-{[(4-chlorobenzyl)amino]carbonyl}-8-fluoro-4-hydroxy-6-quinolinyl)propyl 2,6-diaminohexanoate, trifluoroacetic acid salt;

(156) N-(4-chlorobenzyl)-4-hydroxy-6-(3-hydroxybutyl)-3-quinolinecarboxamide;

(157) N-(4-chlorobenzyl)-8-fluoro-4-hydroxy-6-(4-morpholinylmethyl)-3-quinolinecarboxamide;

(158) 3-(3-{[(4-chlorobenzyl)amino]carbonyl}-8-fluoro-4-hydroxy-6-quinolinyl)propyl phenylcarbamate;

(159) N-(4-chlorobenzyl)-8-fluoro-4-hydroxy-6-(3-oxobutyl)-3-quinolinecarboxamide;

(160) 3-(3-{[(4-chlorobenzyl)amino]carbonyl}-4-hydroxy-6-quinolinyl)-2-propynyl 2,6-bis[(tert-butoxycarbonyl)amino]hexanoate;

(161) 3-(3-{[(4-chlorobenzyl)amino]carbonyl}-4-hydroxy-6-quinolinyl)-2-propynyl 2,6-diaminohexanoate, trifluoroacetic acid salt;

(162) N-(4-chlorobenzyl)-4-hydroxy-6-[(2-hydroxyethoxy)methyl]-3-quinolinecarboxamide;

(163) N-(4-chlorobenzyl)-4-hydroxy-6-{[2-hydroxy-1-(hydroxymethyl)ethoxy]methyl}-3-quinolinecarboxamide;

(164) Methyl 3-{[(4-chlorobenzyl)amino]carbonyl}-4-hydroxy-6-quinolinecarboxylate;

(165) N-(4-chlorobenzyl)-4-hydroxy-6-(hydroxymethyl)-3-quinolinecarboxamide;

(166) 6-chloro-N-(4-chlorobenzyl)-4-hydroxy-8-methyl-3-quinolinecarboxamide;

(167) N-(4-chlorobenzyl)-5,6,8-trifluoro-4-hydroxy-3-quinolinecarboxamide;

(168) N-(4-chlorobenzyl)-6,7-difluoro-4-hydroxy-3-quinolinecarboxamide;

(169) N-(4-chlorobenzyl)-8-fluoro-4-hydroxy-6-(methylsulfanyl)-3-quinolinecarboxamide;

(170) N-(4-chiorobenzyl)-8-fluoro-4-hydroxy-6-[(2-hydroxyethyl)sulfanyl]-3-quinolinecarboxamide;

(171) 6-[(2-aminoethyl)sulfanyl]-N-(4-chlorobenzyl)-8-fluoro-4-hydroxy-3-quinolinecarboxamide hydrobromide;

(172) N-(4-chlorobenzyl)-8-fluoro-4-hydroxy-6-{[(2-methoxyethoxy)methyl]sulfanyl}-3-quinolinecarboxamide;

(173) N-(4-chlorobenzyl)-8-fluoro-4-hydroxy-6-{[2-(4-morpholinyl)ethyl]sulfanyl}-3-quinolinecarboxamide;

(174) N-(4-chlorobenzyl)-8-fluoro-4-hydroxy-6-(methylsulfinyl)-3-quinolinecarboxamide;

(175) N-(4-chlorobenzyl)-8-fluoro-4-hydroxy-6-(methylsulfonyl)-3-quinolinecarboxamide;

(176) N-(4-chlorobenzyl)-8-fluoro-4-hydroxy-6-[(2-hydroxyethyl)sulfinyl]-3-quinolinecarboxamide;

(177) N-(4-chlorobenzyl)-4-hydroxy-6-(2-thienyl)-3-quinolinecarboxamide;

(178) N-(4-chlorobenzyl)-4-hydroxy-6-(2-hydroxyethoxy)-3-quinolinecarboxamide;

(179) N-(4-chlorobenzyl)-8-fluoro-4-hydroxy-6-(4-morpholinylcarbonyl)-3-quinolinecarboxamide;

(180) N³-(4-chlorobenzyl)-8-fluoro-4-hydroxy-N⁶-(2-hydroxyethyl)-3,6-quinolinedicarboxamide;

(181) N³-(4-chlorobenzyl)-8-fluoro-4-hydroxy -N⁶,N˜6˜-dimethyl-3,6-quinolinedicarboxamide;

(182) N³-(4-chlorobenzyl)-8-fluoro-4-hydroxy-N⁶-(4-hydroxyphenethyl)-3,6-quinolinedicarboxamide;

(183) N³-(4-chlorobenzyl)-8-fluoro-4-hydroxy-3,6-quinolinedicarboxamide;

(184) N³,N⁶-bis(4-chlorobenzyl)-8-fluoro-4-hydroxy-3,6-quinolinedicarboxamide;

(185) 6-amino-N-(4-chlorobenzyl)-8-fluoro-4-hydroxy-3-quinolinecarboxamide;

(186) N-(4-chlorobenzyl)-8-fluoro-4-hydroxy-6-{[(4-methoxyphenyl)sulfonyl]amino}-3-quinolinecarboxamide;

(187) N-(4-chlorobenzyl)-4-hydroxy-6-[(2-hydroxyethyl)amino]-3-quinolinecarboxamide;

(188) N-(4-chlorobenzyl)-6-[ethyl(2-hydroxyethyl)amino]-4-hydroxy-3-quinolinecarboxamide;

(189) N-(4-chlorobenzyl)-4-hydroxy-6-(2-oxo-1,3-oxazolidin-3-yl)-3-quinolinecarboxamide;

(190) N-(4-chlorobenzyl)-4-hydroxy-6-(4-morpholinylmethyl)-3-quinolinecarboxamide;

(191) N-(4-chlorobenzyl)-8-fluoro-4-hydroxy-6-{[(1-naphthylmethyl)amino]sulfonyl}-3-quinolinecarboxamide;

(192) N-(4-chlorobenzyl)-8-fluoro-4-hydroxy-6-({[2-(1H-indol-3-yl)ethyl]amino}sulfonyl)-3-quinolinecarboxamide;

(193) N-(4-chlorobenzyl)-8-fluoro-6-{[(2-furylmethyl)amino]sulfonyl}-4-hydroxy-3-quinolinecarboxamide;

(194) 6-{[bis(2-hydroxyethyl)amino]sulfonyl}-N-(4-chlorobenzyl)-8-fluoro-4-hydroxy-3-quinolinecarboxamide;

(195) ethyl 2-{[(3-{[(4-chlorobenzyl)amino]carbonyl}-8-fluoro-4-hydroxy-6-quinolinyl)sulfonyl]amino}acetate;

(196) N-(4-chlorobenzyl)-8-fluoro-4-hydroxy-6-{[(2-hydroxyethyl)amino]sulfonyl}-3-quinolinecarboxamide;

(197) N-(4-chlorobenzyl)-8-fluoro-4-hydroxy-6-(4-morpholinylsulfonyl)-3-quinolinecarboxamide;

(198) N-(4-chlorobenzyl)-8-fluoro-4-hydroxy-6-{[(2-pyridinylmethyl)amino]sulfonyl}-3-quinolinecarboxamide;

(199) N-(4-chlorobenzyl)-8-fluoro-4-hydroxy-6-[(2-pyridinylamino)sulfonyl]-3-quinolinecarboxamide;

(200) N-(4-chlorobenzyl)-6-{[(cyclohexylmethyl)amino]sulfonyl}-8-fluoro-4-hydroxy-3-quinolinecarboxamide;

(201) N-(4-chlorobenzyl)-8-fluoro-4-hydroxy-6-({[2-(1-methyl-2-pyrrolidinyl)ethyl]amino}sulfonyl)-3-quinolinecarboxamide;

(202) N-(4-chlorobenzyl)-8-fluoro-4-hydroxy-6-({[2-(1-pyrrolidinyl)ethyl]amino}sulfonyl)-3-quinolinecarboxamide;

(203) N-(4-chlorobenzyl)-8-fluoro-6-{[(2-furylmethyl)amino]sulfonyl}-4-hydroxy-3-quinolinecarboxamide;

(204) N-(4-chlorobenzyl)-6-({[3-(cyclohexylamino)propyl]amino}sulfonyl)-8-fluoro-4-hydroxy-3-quinolinecarboxamide;

(205) N-(4-chlorobenzyl)-8-fluoro-4-hydroxy-6-{[(1-naphthylmethyl)amino]sulfonyl}-3-quinolinecarboxamide;

(206) N-(4-chlorobenzyl)-8-fluoro-4-hydroxy-6-({[2-(1H-imidazol-4-yl)ethyl]amino}-sulfonyl)-3-quinolinecarboxamide;

(207) N-(4-chlorobenzyl)-8-fluoro-4-hydroxy-6-{[(tetrahydro-2-furanylmethyl)amino]-sulfonyl}-3-quinolinecarboxamide;

(208) N-(4-chlorobenzyl)-8-fluoro-4-hydroxy-6-{[(2-thienylmethyl)amino]sulfonyl}-3-quinolinecarboxamide;

(209) N-(4-chlorobenzyl)-8-fluoro-4-hydroxy-6-({[2-(1H-indol-3-yl)ethyl]amino}sulfonyl)-3-quinolinecarboxamide;

(210) N-(4-chlorobenzyl)-8-fluoro-4-hydroxy-6-({[2-(5-methoxy-1H-indol-3-yl)ethyl]amino}sulfonyl)-3-quinolinecarboxamide;

(211) 6-{[(1,3-benzodioxol-5-ylmethyl)amino]sulfonyl}-N-(4-chlorobenzyl)-8-fluoro-4-hydroxy-3-quinolinecarboxamide;

(212) N-(4-chlorobenzyl)-8-fluoro-4-hydroxy-6-({[2-(4-morpholinyl)ethyl]amino}-sulfonyl)-3-quinolinecarboxamide;

(213) N-(4-chlorobenzyl)-8-fluoro-4-hydroxy-6-({[3-(4-morpholinyl)propyl]amino}-sulfonyl)-3-quinolinecarboxamide;

(214) N-(4-chlorobenzyl)-8-fluoro-4-hydroxy-6-[({2-[(5-nitro-2-pyridinyl)amino]ethyl}-amino)sulfonyl]-3-quinolinecarboxamide;

(215) N-(4-chlorobenzyl)-8-fluoro-4-hydroxy-6-{[(2-pyridinylmethyl)amino]sulfonyl}-3-quinolinecarboxamide;

(216) N-(4-chlorobenzyl)-8-fluoro-4-hydroxy-6-({[2-(2-pyridinyl)ethyl]amino}sulfonyl)-3-quinolinecarboxamide;

(217) N-(4-chlorobenzyl)-8-fluoro-4-hydroxy-6-{[(3-pyridinylmethyl)amino]sulfonyl}-3-quinolinecarboxamide;

(218) N-(4-chlorobenzyl)-8-fluoro-4-hydroxy-6-{[(4-pyridinylmethyl)amino]sulfonyl}-3-quinolinecarboxamide;

(219) N-(4-chlorobenzyl)-6-{[(4-chlorobenzyl)amino]sulfonyl}-8-fluoro-4-hydroxy-3-quinolinecarboxamide;

(220) N-(4-chlorobenzyl)-8-fluoro-4-hydroxy-6-{[(4-methoxybenzyl)amino]sulfonyl}-3-quinolinecarboxamide;

(221) N-(4-chlorobenzyl)-8-fluoro-4-hydroxy-6-[(neopentylamino)sulfonyl]-3-quinolinecarboxamide;

(222) N-(4-chlorobenzyl)-8-fluoro-4-hydroxy-6-{[(2-hydroxypropyl)amino]sulfonyl}-3-quinolinecarboxamide;

(223) N-(4-chlorobenzyl)-6-{[(2,3-dihydroxypropyl)amino]sulfonyl}-8-fluoro-4-hydroxy-3-quinolinecarboxamide;

(224) N-(4-chlorobenzyl)-6-{[(2,2-diphenylethyl)amino]sulfonyl}-8-fluoro-4-hydroxy-3-quinolinecarboxamide;

(225) 11-{[(3-{[(4-chlorobenzyl)amino]carbonyl}-8-fluoro-4-hydroxy-6-quinolinyl)sulfonyl]aminolundecanoic acid;

(226) 6-({[2-(acetylamino)ethyl]amino}sulfonyl)-N -(4-chlorobenzyl)-8-fluoro-4-hydroxy-3-quinolinecarboxamide;

(227) N-(4-chlorobenzyl)-8-fluoro-4-hydroxy-6-({[2-(2-hydroxyethoxy)ethyl]amino}-sulfonyl)-3-quinolinecarboxamide;

(228) N-(4-chlorobenzyl)-8-fluoro-4-hydroxy-6-{[(2-hydroxyethyl)amino]sulfonyl}-3-quinolinecarboxamide;

(229) N-(4-chlorobenzyl)-8-fluoro-4-hydroxy-6-[(phenethylamino)sulfonyl]-3-quinolinecarboxamide;

(230) N-(4-chlorobenzyl)-6-{[(4-chlorophenethyl)amino]sulfonyl}-8-fluoro-4-hydroxy-3-quinolinecarboxamide;

(231) N-(4-chlorobenzyl)-8-fluoro-4-hydroxy-6-[(2-propynylamino)sulfonyl]-3-quinolinecarboxamide;

(232) N-(4-chlorobenzyl)-8-fluoro-4-hydroxy-6-[(isopentylamino)sulfonyl]-3-quinolinecarboxamide;

(233) N-(4-chlorobenzyl)-8-fluoro-4-hydroxy-6-{[(3-phenylpropyl)amino]sulfonyl}-3-quinolinecarboxamide;

(234) N-(4-chlorobenzyl)-8-fluoro-4-hydroxy-6-[(pentylamino)sulfonyl]-3-quinolinecarboxamide;

(235) 6-({[3,5-bis(trifluoromethyl)benzyl]amino}sulfonyl)-N-(4-chlorobenzyl)-8-fluoro-4-hydroxy-3-quinolinecarboxamide;

(236) N-(4-chlorobenzyl)-6-({[2-(1-cyclohexen-1-yl)ethyl]amino}sulfonyl)-8-fluoro-4-hydroxy-3-quinolinecarboxamide;

(237) N-(4-chlorobenzyl)-8-fluoro-4-hydroxy-6-({[2-(1-naphthylamino)ethyl]amino}-sulfonyl)-3-quinolinecarboxamide;

(238) N-(4-chlorobenzyl)-8-fluoro-4-hydroxy-6-[(methylamino)sulfonyl]-3-quinolinecarboxamide;

(239) N-(4-chlorobenzyl)-6-{[(cyanomethyl)amino]sulfonyl}-8-fluoro-4-hydroxy-3-quinolinecarboxamide;

(240) N-(4-chlorobenzyl)-6-{[(2,4-dimethoxybenzyl)amino]sulfonyl}-8-fluoro-4-hydroxy-3-quinolinecarboxamide;

(241) N-(4-chlorobenzyl)-8-fluoro-4-hydroxy-6-{[(3-iodobenzyl)amino]sulfonyl}-3-quinolinecarboxamide; -quinolinecarboxamide;

(243) 6-{[(2-bromoethyl)amino]sulfonyl}-N-(4-chlorobenzyl)-8-fluoro-4-hydroxy-3-quin

(243) 6-{[(2-bromoethyl)amino]sulfonyl}-N-(4-chlorobenzyl)-8-fluoro-4-hydroxy-3-quinolinecarboxamide;

(244) N-(4-chlorobenzyl)-6-{[(2-chloroethyl)amino]sulfonyl}-8-fluoro-4-hydroxy-3-quinolinecarboxamide;

(245) N-(4-chlorobenzyl)-6-{[(3,4-dihydroxyphenethyl)amino]sulfonyl}-8-fluoro-4-hydroxy-3-quinolinecarboxamide;

(246) N-(4-chlorobenzyl)-6-({[2-(ethylsulfanyl)ethyl]amino}sulfonyl)-8-fluoro-4-hydroxy-3-quinolinecarboxamide;

(247) 6-{[(3-bromopropyl)amino]sulfonyl}-N-(4-chlorobenzyl)-8-fluoro-4-hydroxy-3-quinolinecarboxamide;

(248) 6-({[4-(aminosulfonyl)benzyl]amino}sulfonyl)-N-(4-chlorobenzyl)-8-fluoro-4-hydroxy-3-quinolinecarboxamide;

(249) 6-[({2-[bis(2-hydroxyethyl)amino]ethyl}amino)sulfonyl]-N-(4-chlorobenzyl)-8-fluoro-4-hydroxy-3-quinolinecarboxamide;

(250) N-(4-chlorobenzyl)-6-({[2-(ethylsulfanyl)ethyl]amino}sulfonyl)-8-fluoro-4-hydroxy-3-quinolinecarboxamide;

(251) N-(4-chlorobenzyl)-6-{[(3,4-dimethylbenzyl)amino]sulfonyl}-8-fluoro-4-hydroxy-3-quinolinecarboxamide;

(252) N-(4-chlorobenzyl)-6-{[(cyclopropylmethyl)amino]sulfonyl}-8-fluoro-4-hydroxy-3-quinolinecarboxamide;

(253) 6-{[(4-bromobenzyl)amino]sulfonyl}-N-(4-chlorobenzyl)-8-fluoro-4-hydroxy-3-quinolinecarboxamide;

(254) N-(4-chlorobenzyl)-8-fluoro-4-hydroxy-6-({[2-(2-thienyl)ethyl]amino}sulfonyl)-3-quinolinecarboxamide;

(255) N-(4-chlorobenzyl)-8-fluoro-4-hydroxy-6-{[(2-phenoxyethyl)amino]sulfonyl}-3-quinolinecarboxamide;

(256) tert-butyl 2-{[(3-{[(4-chlorobenzyl)amino]carbonyl}-8-fluoro-4-hydroxy-6-quinolinyl)sulfonyl]amino}acetate;

(257) tert-butyl 3-{[(3-{[(4-chlorobenzyl)amino]carbonyl}-8-fluoro-4-hydroxy-6-quinolinyl)sulfonyl]amino}propanoate;

(258) N-(4-chlorobenzyl)-8-fluoro-4-hydroxy-6-({[3-(trifluoromethoxy)benzyl]amino}-sulfonyl)-3-quinolinecarboxamide;

(259) N-(4-chlorobenzyl)-8-fluoro-4-hydroxy-6-{[(2-{[2-(hydroxymethyl)phenyl]sulfanyl}-benzyl)amino]sulfonyl}-3-quinolinecarboxamide;

(260) N-(4-chlorobenzyl)-8-fluoro-4-hydroxy-6-({[4-(1,2,3-thiadiazol-4-yl)benzyl]amino}sulfonyl)-3-quinolinecarboxamide;

(261) N-(4-chlorobenzyl)-6-{[(4-chloro-2-fluorobenzyl)amino]sulfonyl}-8-fluoro-4-hydroxy-3-quinolinecarboxamide;

(262) N-(4-chlorobenzyl)-8-fluoro-4-hydroxy-6-[({2-[(2-hydroxyethyl)sulfanyl]ethyl}-amino)sulfonyl]-3-quinolinecarboxamide;

(263) 6-{[(2-amino-2-methylpropyl)amino]sulfonyl}-N-(4-chlorobenzyl)-8-fluoro-4-hydroxy-3-quinolinecarboxamide;

(264) 6-{[(2-amino-2-oxoethyl)amino]sulfonyl}-N-(4-chlorobenzyl)-8-fluoro-4-hydroxy-3-quinolinecarboxamide;

(265) 6-{[(4-aminobenzyl)amino]sulfonyl}-N-(4-chlorobenzyl)-8-fluoro-4-hydroxy-3-quinolinecarboxamide;

(266) di(tert-butyl) 3-(3-{[(4-chlorobenzyl)amino]carbonyl}-8-fluoro-4-hydroxy-6-quinolinyl)propyl phosphate;

(267) 3-(3-{[(4-chlorobenzyl)amino]carbonyl}-8-fluoro-4-hydroxy-6-quinolinyl)propyl dihydrogen phosphate;

(268) 3-(3-{[(4-chlorobenzyl)amino]carbonyl}-4-hydroxy-6-quinolinyl)propyl dihydrogen phosphate;

(269) tert-butyl 3-(3-{[(4-chlorobenzyl)amino]carbonyl}-4-hydroxy-6-quinolinyl)propyl phosphonate;

(270) tert-butyl 3-(3-{[(4-chlorobenzyl)amino]carbonyl}-8-fluoro-4-hydroxy-6-quinolinyl)propyl phosphonate;

(271) (E)-3-(3-{[(4-chlorobenzyl)amino]carbonyl}-8-fluoro-4-hydroxy-6-quinolinyl)-2-propenoic acid; or (272) N-[(4-chlorophenyl)methyl]-4-hydroxy-7-iodo-3-quinolinecarboamide.

A method for preventing or treating atherosclerosis or restenosis in mammals, wherein the compound administered has the Formula I and is selected from the group consisting of:

(1) 7-amino-N-(4-chlorobenzyl)-4-hydroxy-3-quinolinecarboxamide;

(2) N-(4-chlorobenzyl)-4-hydroxy-7-methoxy-3-quinolinecarboxamide;

(3) N-(4-chlorobenzyl)-7-fluoro-4-hydroxy-3-quinolinecarboxamide;

(4) N-(4-chlorobenzyl)-8-fluoro-4-hydroxy-3-quinolinecarboxamide;

(5) 6-chloro-N-(4-chlorobenzyl)-4-hydroxy-3-quinolinecarboxamide;

(6) N-(4-chlorobenzyl)-4-hydroxy-6-methyl-3-quinolinecarboxamide;

(7) N-(4-chlorobenzyl)-4-hydroxy-6-methoxy-3-quinolinecarboxamide;

(8) N-(4-chlorobenzyl)-8-fluoro-4-hydroxy-6-methyl-3-quinolinecarboxamide;

(9) N-(4-chlorobenzyl)-4-hydroxy-6-iodo-3-quinolinecarboxamide;

(10) N-(4-chlorobenzyl)-4-hydroxy-6-phenyl-3-quinolinecarboxamide;

(11) N-(4-chlorobenzyl)-4-hydroxy-6,8-dimethoxy-3-quinolinecarboxamide;

(12) 6-(tert-butyl)-N-(4-chlorobenzyl)-4-hydroxy-3-quinolinecarboxamide;

(13) N-(4-chlorobenzyl)-6-(cyanomethyl)-4-hydroxy-3-quinolinecarboxamide;

(14) N-(4-chlorobenzyl)-9-hydroxy[1,3]thiazolo[5,4-f]quinoline-8-carboxamide;

(15) N-(4-chlorobenzyl)-8-fluoro-4-hydroxy-6-(3-hydroxy-1-propynyl)-3-quinolinecarboxamide;

(16) N-(4-chlorobenzyl)-8-fluoro-4-hydroxy-6-(1,3-thiazol-2-yl)-3-quinolinecarboxamide;

(17) N-(4-chlorobenzyl)-8-fluoro-4-hydroxy-6-(2-thienyl)-3-quinolinecarboxamide;

(18) N-(4-chlorobenzyl)-8-fluoro-4-hydroxy-6-(3-methoxy-1-propynyl)-3-quinolinecarboxamide;

(19) 6-(4-bromo-2-thienyl)-N-(4-chlorobenzyl)-8-fluoro-4-hydroxy-3-quinolinecarboxamide;

(20) N-(4-chlorobenzyl)-8-fluoro-4-hydroxy-6-(4-hydroxybutyl)-3-quinolinecarboxamide;

(21) N-(4-chlorobenzyl)-8-fluoro-4-hydroxy-6-[(Z)-4-hydroxy-1-butenyl]-3-quinolinecarboxamide;

(22) N-((4-chlorobenzyl)-6,8-difluoro-4-hydroxy-7-[4-(hydroxymethyl)phenoxy]-3-quinolinecarboxamide;

(23) N-((4-chlorobenzyl)-6,8-difluoro-4-hydroxy-7-methoxy-3-quinolinecarboxamide;

(24) N-(4-chlorobenzyl)-8-fluoro-4-hydroxy-6-(5-hydroxy-1-pentynyl)-3-quinolinecarboxamide;

(25) 3-(3-{[(4-chlorobenzyl)amino]carbonyl}-8-fluoro-4-hydroxy-6-quinolinyl)-2-propynyl formate;

(26) N-(4-chlorobenzyl)-8-fluoro-4-hydroxy-6-(3-hydroxypropyl)-3-quinolinecarboxamide;

(27) N-(4-chlorobenzyl)-4-hydroxy-7-(3-hydroxypropyl)-3-quinolinecarboxamide;

(28) N-((4-chlorobenzyl)-8-fluoro-4-hydroxy-6-(3-hydroxybutyl)-3-quinolinecarboxamide;

(29) N-((4-chlorobenzyl)-6-[(E)-2-cyanoethenyl]-8-fluoro-4-hydroxy-3-quinolinecarboxamide;

(30) N-((4-chlorobenzyl)-4-hydroxy-6-(3-hydroxy-1-propynyl)-8-methoxy-3-quinolinecarboxamide;

(31) N-(4-chlorobenzyl)-4-hydroxy-6-(3-hydroxy-1-propynyl)-3-quinolinecarboxamide;

(32) N-(4-chlorobenzyl)-4-hydroxy-8-methoxy-6-(3-methoxy-l-propynyl)-3-quinolinecarboxamide;

(33) N-(4-chlorobenzyl)-8-fluoro-4-hydroxy-6-[(Z)-3-hydroxy-1-propenyl]-3-quinolinecarboxamide;

(34) N-(4-chlorobenzyl)-8-fluoro-4-hydroxy-6-(3-oxopropyl)-3-quinolinecarboxamide;

(35) N-(4-chlorobenzyl)-4-hydroxy-7-(4-hydroxybutyl)-3-quinolinecarboxamide;

(36) N-(4-chlorobenzyl)-4-hydroxy-6-iodo-8-methoxy-3-quinolinecarboxamide;

(37) N-(4-chlorobenzyl)-8-fluoro-6-(3-fluoro-1-propynyl)-4-hydroxy-3-quinolinecarboxamide;

(38) N-(4-chlorobenzyl)-4-hydroxy-6-(3-hydroxypropyl)-3-quinolinecarboxamide;

(39) 3-(3-{[(4-chlorobenzyl)amino]carbonyl}-8-fluoro-4-hydroxy-6-quinolinyl)propyl 2-bromoacetate;

(40) 3-(3-{[(4-chlorobenzyl)amino]carbonyl}-8-fluoro-4-hydroxy-6-quinolinyl)propyl 2-[(tert-butoxycarbonyl)amino]-3-methylbutanoate;

(41) 3-(3-{[(4-chlorobenzyl)amino]carbonyl}-8-fluoro-4-hydroxy-6-quinolinyl)propyl 2-amino-3-methylbutanoate;

(42) 3-(3-{[(4-chlorobenzyl)amino]carbonyl}-8-fluoro-4-hydroxy-6-quinolinyl)-2-propynyl phenylcarbamate;

(43) N-((4-chlorobenzyl)-4-hydroxy-6-propyl-3-quinolinecarboxamide;

(44) N-((4-chlorobenzyl)-4-hydroxy-6-(3-hydroxypropyl)-8-methoxy-3-quinolinecarboxamide;

(45) N-(4-chlorobenzyl)-8-fluoro-4-hydroxy-6-(methylsulfanyl)-3-quinolinecarboxamide;

(46) N-(4-chlorobenzyl)-4-hydroxy-7-({[(1-naphthylmethyl)amino]sulfonyl}-3-quinolinecarboxamide;

(47) N-(4-chlorobenzyl)-8-fluoro-4-hydroxy-6-[(E)-3-oxo-1-butenyl]-3-quinolinecarboxamide;

(48) N-(4-chlorobenzyl)-4-hydroxy-6-(3-hydroxy-1-propynyl)-8-(trifluoromethoxy)-3-quinolinecarboxamide;

(49) sodium 2-[{8-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;

(50) 3-(3-{[(4-chlorobenzyl)amino]carbonyl}-8-fluoro-4-hydroxy-6-quinolinyl)propyl dihydrogen phosphate;

(51) 3-(3-{[(4-chlorobenzyl)amino]carbonyl}-8-fluoro-4-hydroxy-6-quinolinyl)propyl 2,6-bis[(tert-butoxycarbonyl)amino]hexanoate;

(52) tert-butyl 3-(3-{[(4-chlorobenzyl)amino]carbonyl}-8-fluoro-4-hydroxy-6-quinolinyl)propyl phosphonate;

(53) sodium 2-[(8-{[3-(3-{[(4-chlorobenzyl)amino]carbonyl}-8-fluoro-4-hydroxy-6-quinolinyl)-2-propynyl]oxy}-8-oxooctanoyl)(methyl)amino]-1-ethanesulfonate;

(54) N-(4-chlorobenzyl)-4-hydroxy-6-(2-hydroxyethoxy)-3-quinolinecarboxamide;

(55) 3-(3-{[(4-chlorobenzyl)amino]carbonyl}-8-fluoro-4-hydroxy-6-quinolinyl)propyl 2,6-diaminohexanoate trifluoroacetic acid salt;

(56) N-(4-chlorobenzyl)-4-hydroxy-6-[(2-hydroxyethyl)amino]-3-quinolinecarboxamide;

(57) N-(4-chlorobenzyl)-4-hydroxy-6-(3-hydroxybutyl)-3-quinolinecarboxamide;

(58) sodium 2-[(8-{[3-(3-{[(4-chlorobenzyl)amino]carbonyl}-4-hydroxy-6- quinolinyl)-2-propynyl]oxy}-8-oxooctanoyl)(methyl)amino]-1-ethanesulfonate;

(59) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;

(60) tert-butyl 3-(3-{[(4-chlorobenzyl)amino]carbonyl}-4-hydroxy-6-quinolinyl)propyl phosphonate;

(61) 3-(3-{[(4-chlorobenzyl)amino]carbonyl}-4-hydroxy-6-quinolinyl)propyl dihydrogen phosphate;

(62) N-(4-chlorobenzyl)-4-hydroxy-8-(2-hydroxyethoxy)-6-(3-hydroxypropyl)-3-quinolinecarboxamide;

(63) N-(4-chlorobenzyl)-8-fluoro-4-hydroxy-6-(4-morpholinylmethyl)-3-quinolinecarboxamide;

(64) N-(4-chlorobenzyl)-8-fluoro-4-hydroxy-6-(3-oxobutyl)-3-quinolinecarboxamide;

(65) N-(4-chlorobenzyl)-4-hydroxy-6-(2-oxo-1,3-oxazolidin-3-yl)-3-quinolinecarboxamide;

(66) 3-(3-{[(4-chlorobenzyl)amino]carbonyl}-4-hydroxy-6-quinolinyl)-2-propynyl 2,6-bis[(tert-butoxycarbonyl)amino]hexanoate;

(67) N-(4-chlorobenzyl)-6-[(Z)-3-hydroxy-1-propenyl]-4-oxo-1,4-dihydro-3-quinolinecarboxamide;

(68) N-(4-chlorobenzyl)-6-[(E)-3-hydroxy-1-propenyl]-4-oxo-1,4-dihydro-3-quinolinecarboxamide;

(69) 3-(3-{[(4-chlorobenzyl)amino]carbonyl}-4-hydroxy-6-quinolinyl)-2-propynyl 2,6-diaminohexanoate trifluoroacetic acid salt;

(70) 3-(3-{[(4-chlorobenzyl)amino]carbonyl}-4-hydroxy-6-quinolinyl)propyl hydrogen phosphonate;

(71) N-(4-chlorobenzyl)-4-hydroxy-6-(2-thienyl)-3-quinolinecarboxamide;

(72) N-((4-chlorobenzyl)-4-hydroxy-6-[(2-hydroxyethoxy)methyl]-3-quinolinecarboxamide;

(73) N-(4-chlorobenzyl)-4-hydroxy-6-(3-hydroxy-1,1-dimethylpropyl)-3-quinolinecarboxamide;

(74) N-(4-chlorobenzyl)-4-hydroxy-6-{[2-hydroxy-1-(hydroxymethyl)ethoxy]methyl}-3-quinolinecarboxamide;

(75) N-((4-chlorobenzyl)-4-hydroxy-6-(hydroxymethyl)-3-quinolinecarboxamide;

(76) Methyl 3-{[(4-chlorobenzyl)amino]carbonyl}-4-hydroxy-6-quinolinecarboxylate; or

(77) N-(4-chlorobenzyl)-4-hydroxy-6-(4-morpholinylmethyl)-3-quinolinecarboxamide.

A method for preventing or treating atherosclerosis or restenosis in mammals, wherein the compound administered has the Formula I and is selected from the group consisting of:

(1) N-(4-chlorobenzyl)-4-hydroxy-7-methoxy-3-quinolinecarboxamide;

(2) N-(4-chlorobenzyl)-7-fluoro-4-hydroxy-3-quinolinecarboxamide;

(3) N-(4-chlorobenzyl)-8-fluoro-4-hydroxy-3-quinolinecarboxamide;

(4) N-(4-chlorobenzyl)-4-hydroxy-6-methoxy-3-quinolinecarboxamide;

(5) N-(4-chlorobenzyl)-8-fluoro-4-hydroxy-6-(2-thienyl)-3-quinolinecarboxamide;

(6) N-(4-chlorobenzyl)-8-fluoro-4-hydroxy-6-(4-hydroxybutyl)-3-quinolinecarboxamide;

(7) N-(4-chlorobenzyl)-8-fluoro-4-hydroxy-6-[(Z)-4-hydroxy-1-butenyl]-3-quinolinecarboxamide;

(8) N-(4-chlorobenzyl)-8-fluoro-4-hydroxy-6-(3-hydroxypropyl)-3-quinolinecarboxamide;

(9) N-(4-chlorobenzyl)-4-hydroxy-7-(3-hydroxypropyl)-3-quinolinecarboxamide;

(10) N-(4-chlorobenzyl)-4-hydroxy-6-(3-hydroxy-1-propynyl)-8-methoxy-3-quinolinecarboxamide;

(11) N-(4-chlorobenzyl)-4-hydroxy-6-(3-hydroxy-1-propynyl)-3-quinolinecarboxamide;

(12) N-(4-chlorobenzyl)-4-hydroxy-8-methoxy-6-(3-methoxy-1-propynyl)-3-quinolinecarboxamide;

(13) N-(4-chlorobenzyl)-4-hydroxy-7-(4-hydroxybutyl)-3-quinolinecarboxamide;

(14) N-(4-chlorobenzyl)-4-hydroxy-6-(3-hydroxypropyl)-3-quinolinecarboxamide;

(15) 3-(3-{[(4-chlorobenzyl)amino]carbonyl}-8-fluoro-4-hydroxy-6-quinolinyl)propyl 2-bromoacetate;

(16) 3-(3-{[(4-chlorobenzyl)amino]carbonyl}-8-fluoro-4-hydroxy-6-quinolinyl)propyl 2-amino-3-methylbutanoate;

(17) N-(4-chlorobenzyl)-4-hydroxy-6-(3-hydroxypropyl)-8-methoxy-3-quinolinecarboxamide;

(18) sodium 2-[18-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;

(19) 3-(3-{[(4-chlorobenzyl)amino]carbonyl}-8-fluoro-4-hydroxy-6-quinoiinyl)propyl dihydrogen phosphate;

(20) 3-(3-{[(4-chlorobenzyl)amino]carbonyl}-8-fluoro-4-hydroxy-6-quinolinyl)propyl 2,6-bis[(tert-butoxycarbonyl)amino]hexanoate;

(21) tert-butyl 3-(3-{[(4-chlorobenzyl)amino]carbonyl}-8-fluoro-4-hydroxy-6-quinolinyl)propyl phosphonate;

(22) 3-(3-{[(4-chlorobenzyl)amino]carbonyl}-8-fluoro-4-hydroxy-6-quinolinyl)propyl 2,6-diaminohexanoate trifluoroacetic acid salt;

(23) N-(4-chlorobenzyl)-4-hydroxy-6-[(2-hydroxyethyl)amino]-3-quinolinecarboxamide;

(24) N-(4-chlorobenzyl)-4-hydroxy-6-(3-hydroxybutyl)-3-quinolinecarboxamide;

(25) sodium 2-[(8-{[3-(3-{[(4-chlorobenzyl)amino]carbonyl}-4-hydroxy-6- quinolinyl)-2-propynyl]oxy}-8-oxooctanoyl)(methyl)amino]-1-ethanesulfonate;

(26) sodium 2-[{8-[3-(3-{[(4-chlorobenzyl)amino]carbonyl}-4-hydroxy-6-qluinolinyl)propoxy]-8-oxooctanoyl}(methyl)amino]-1-ethanesulfonate;

(27) tert-butyl 3-(3-{[(4-chlorobenzyl)amino]carbonyl}-4-hydroxy-6-quinolinyl)propyl phosphonate;

(28) 3-(3-{[(4-chlorobenzyl)amino]carbonyl}-4-hydroxy-6-quinolinyl)propyl dihydrogen phosphate;

(29) N-(4-chlorobenzyl)-4-hydroxy-8-(2-hydroxyethoxy)-6-(3-hydroxypropyl)-3-quinolinecarboxamide;

(30) N-(4-chlorobenzyl)-8-fluoro-4-hydroxy-6-(4-morpholinylmethyl)-3-quinolinecarboxamide;

(31) 3-(3-{[(4-chlorobenzyl)amino]carbonyl}-4-hydroxy-6-quinolinyl)-2-propynyl 2,6-bis[(tert-butoxycarbonyl)amino]hexanoate;

(32) N-(4-chlorobenzyl)-6-[(Z)-3-hydroxy-1-propenyl]-4-oxo-1,4-dihydro-3-quinolinecarboxamide;

(33) N-(4-chlorobenzyl)-6-[(E)-3-hydroxy-1-propenyl]-4-oxo-1,4-dihydro-3-quinolinecarboxamide;

(34) 3-(3-{[(4-chlorobenzyl)amino]carbonyl}-4-hydroxy-6-quinolinyl)-2-propynyl 2,6-diaminohexanoate trifluoroacetic acid salt;

(35) 3-(3-{[(4-chlorobenzyl)amino]carbonyl}-4-hydroxy-6-quinolinyl)propyl hydrogen phosphonate;

(36) N-(4-chlorobenzyl)-4-hydroxy-6-(2-thienyl)-3-quinolinecarboxamide;

(37) N-(4-chlorobenzyl)-4-hydroxy-6-[(2-hydroxyethoxy)methyl]-3-quinolinecarboxamide;

(38) N-(4-chlorobenzyl)-4-hydroxy-6-(3-hydroxy-1,1-dimethylpropyl)-3-quinolinecarboxamide;

(39) methyl 3-{[(4-chlorobenzyl)amino]carbonyl}-4-hydroxy-6-quinolinecarboxylate; or

(40) N-(4-chlorobenzyl)-4-hydroxy-6-(4-morpholinylmethyl)-3-quinolinecarboxamide.

A method for preventing or treating atherosclerosis or restenosis in mammals, wherein the compound administered has the Formula I and is selected from the group consisting of:

(1) N-(4-chlorobenzyl)-8-fluoro-4-hydroxy-6-(3-hydroxypropyl)-3-quinolinecarboxamide;

(2) N-(4-chlorobenzyl)-4-hydroxy-6-(3-hydroxy-1-propynyl)-8-methoxy-3-quinolinecarboxamide;

(3) N-(4-chlorobenzyl)-4-hydroxy-8-methoxy-6-(3-methoxy-1-propynyl)-3-quinolinecarboxamide;

(4) N-(4-chlorobenzyl)-4-hydroxy-6-(3-hydroxypropyl)-3-quinolinecarboxamide;

(5) N-(4-chlorobenzyl)-4-hydroxy-6-(3-hydroxypropyl)-8-methoxy-3-quinolinecarboxamide;

(6) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;

(7) sodium 2-[{8-[3-(3-{[(4-chlorobenzyl)amino]carbonyl}-4-hydroxy-6-quinolinyl)propoxy]

8-oxooctanoyl}(methyl)amino]-1-ethanesulfonate;

(8) 3-(3-{[(4-chlorobenzyl)amino]carbonyl}-4-hydroxy-6-quinolinyl)propyl dihydrogen phosphate;

(9) N-(4-chlorobenzyl)-6-[(Z)-3-hydroxy-1-propenyl]-4-oxo-1,4-dihydro-3-quinolinecarboxamide;

(10) N-(4-chlorobenzyl)-6-[(E)-3-hydroxy-1-propenyl]-4-oxo-1,4-dihydro-3-quinolinecarboxamide;

(11) 3-(3-{[(4-chlorobenzyl)amino]carbonyl}-4-hydroxy-6-quinolinyl)-2-propynyl 2,6-diaminohexanoate trifluoroacetic acid salt;

(12) 3-(3-{[(4-chlorobenzyl)amino]carbonyl}-4-hydroxy-6-quinolinyl)propyl hydrogen phosphonate;

(13) N-(4-chlorobenzyl)-4-hydroxy-6-(2-thienyl)-3-quinolinecarboxamide;

(14) N-((4-chlorobenzyl)-4-hydroxy-6-[(2-hydroxyethoxy)methyl]-3-quinolinecarboxamide; or (15) N-(4-chlorobenzyl)-4-hydroxy-6-(4-morpholinylmethyl)-3-quinolinecarboxamide.

A method for preventing or treating atherosclerosis or restenosis in mammals, wherein the compound administered has the Formula II and is selected from the group consisting of:

(a) N-(4-chlorobenzyl)-6-(3-hydroxy-1,1-dimethylpropyl)-1-methyl-4-oxo-1,4-dihydro-3-quinolinecarboxamide;

(b) N-(4-chlorobenzyl)-1-methyl-6-(4-morpholinylmethyl)-4-oxo-1,4-dihydro-3-quinolinecarboxamide;

(c) N-(4-chlorobenzyl)-1-(2-hydroxyethyl)-6-(3-hydroxypropyl)-4-oxo-1,4-dihydro-3-quinolinecarboxamide;

(d) N-(4-chlorobenzyl)-6-(3-hydroxypropyl)-1-methyl-4-oxo-1,4-dihydro-3-quinolinecarboxamide;

(e) 1-(tert-butyl)-N-(4-chlorobenzyl)-6-(3-hydroxypropyl)-4-oxo-1,4-dihydro-3-quinolinecarboxamide;

(f) N-(4-chlorobenzyl)-6-[(1,1-dioxo-1′,4-thiazinan-4-yl)methyl]-1-methyl-4-oxo-1,4-dihydro-3-quinolinecarboxamide;

(g) N-(4-chlorobenzyl)-1-methyl-4-oxo-6-[(1-oxo-1′,4-thiazinan-4-yl)methyl]-1,4-dihydro-3-quinolinecarboxamide;

(h) N-(4-chlorobenzyl)-8-fluoro-1-methyl-6-(4-morpholinylmethyl)-4-oxo-1,4-dihydro-3-quinolinecarboxamide;

or a pharmaceutically acceptable salt thereof.

A method of preventing or treating atherosclerosis or restenosis, wherein the compound administered has the Formula II and is selected from the group consisting of:

(a) N-(4-chlorobenzyl)-6-(3-hydroxy-1,1-dimethylpropyl)-1-methyl-4-oxo-1,4-dihydro-3-quinolinecarboxamide;

(b) N-(4-chlorobenzyl)-1-methyl-6-(4-morpholinylmethyl)-4-oxo-1,4-dihydro-3-quinolinecarboxamide;

(c) N-(4-chlorobenzyl)-1-(2-hydroxyethyl)-6-(3-hydroxypropyl)-4-oxo-1,4-dihydro-3-quinolinecarboxamide;

(d) N-(4-chlorobenzyl)-6-(3-hydroxypropyl)-1-methyl-4-oxo-1,4-dihydro-3-quinolinecarboxamide;

(e) N-(4-chlorobenzyl)-6-[(1,1-dioxo-1′,4-thiazinan-4-yl)methyl]-1-methyl-4-oxo-1,4-dihydro-3-quinolinecarboxamide;

(f) N-(4-chlorobenzyl)-1-methyl-4-oxo-6-[(1-oxo-1′,4-thiazinan-4-yl)methyl]-1,4-dihydro-3-quinolinecarboxamide;

(g) N-(4-chlorobenzyl)-8-fluoro-1-methyl-6-(4-morpholinylmethyl)-4-oxo-1,4-dihydro-3-quinolinecarboxamide;

or a pharmaceutically acceptable salt thereof.

A method of preventing or treating atherosclerosis or restenosis in mammals, wherein the compound administered has the Formula II and is selected from the group consisting of:

(a) N-(4-chlorobenzyl)-1-methyl-6-(4-morpholinylmethyl)-4-oxo-1,4-dihydro-3-quinolinecarboxamide;

(b) N-(4-chlorobenzyl)-6-(3-hydroxypropyl)-1-methyl-4-oxo-1,4-dihydro-3-quinolinecarboxamide;

(c) N-(4-chlorobenzyl)-6-[(1,1-dioxo-1′,4-thiazinan-4-yl)methyl]-1-methyl-4-oxo-1,4-dihydro-3-quinolinecarboxamide;

(d) N-(4-chlorobenzyl)-1-methyl-4-oxo-6-[(1-oxo-1′,4-thiazinan-4-yl)methyl]-1,4-dihydro-3-quinolinecarboxamide;

(e) N-(4-chlorobenzyl)-8-fluoro-1-methyl-6-(4-morpholinylmethyl)-4-oxo-1,4-dihydro-3-quinolinecarboxamide;

or a pharmaceutically acceptable salt thereof.

A method of preventing or treating atherosclerosis or restenosis in mammals, wherein the compound administered has the Formula II and is N-(4-chlorobenzyl)-1-methyl-6-(4-morpholinylmethyl)-4-oxo-1,4-dihydro-3-quinolinecarboxamide, or a pharmaceutically acceptable salt thereof.

A method of preventing or treating atherosclerosis or restenosis in mammals, wherein the compound administered has the Formula III and R^{III-1} is Cl.

A method of preventing or treating atherosclerosis or restenosis in mammals, wherein the compound administered has the Formula III and R^{III-1} is F.

A method of preventing or treating atherosclerosis or restenosis in mammals, wherein the compound administered has the Formula III and R^{III-1} is CN, or NO_{2. administered has the Formula III and R}^{III-1} is CN, or NO₂. or restenosis in mammals, wherein the compound administered has the Formula III and R^{III-2} is (CH₂CH₂O)ₘH, or (CH₂CH₂O)ₘC₁₋₄ alkyl, wherein m^{III} is 2, or 3.

A method of preventing or treating atherosclerosis or restenosis in mammals, wherein the compound administered has the Formula III and R^{III-2} is C₃₋₈cycloalkyl optionally substituted by R^{III-11}, NR^{III-7}R^{III-8}, SOᵢR^{III-9}, or C₁₋₇ alkyl optionally substituted by R^{III-11}, NR^{III-7}R^{III-8}, or SOᵢR^{III-9}; wherein R^{III-7}, R^{III-8}, R^{III-9}, R^{III-11} and i^{III} are the same as defined in the summary of invention.

A method of preventing or treating atherosclerosis or restenosis in mammals, wherein the compound administered has the Formula III and R^{III-2} is cyclopropyl.

A method of preventing or treating atherosclerosis or restenosis in mammals, wherein the compound administered has the Formula III and R^{III-2} is het^{III} wherein said het^{III} is bonded via a carbon atom and is the same as defined in the summary of invention.

A method of preventing or treating atherosclerosis or restenosis in mammals, wherein the compound administered has the Formula III and het^{III} is tetrahydro-2H-pyranyl, piperdinyl, 1-methyl-piperidinyl, or 1,1-dioxo-tetrahydro-2H-thiopyran.

A method of preventing or treating atherosclerosis or restenosis in mammals, wherein the compound administered has the Formula III and R^{III-2} is C₂₋₇ alkyl which is partially unsaturated and optionally substituted by NR^{III-7}R^{III-8}, R^{III-11}, SOᵢR^{III-9}, or OC₂₋₄ alkyl which is further substituted by het^{III}, OR^{III-10}, or OC(═O)aryl^{III}; wherein R^{III-7}, R^{III-8}, R^{III-9}, R^{III-10} are the same as defined in the summary of invention.

A method of preventing or treating atherosclerosis or restenosis in mammals, wherein the compound administered has the Formula III and R^{III-2} is (Z or E) —CH═CHR^{III-10}, or —C—C≡CR^{III-10}; wherein said R^{III-10} is H, or C₁₋₇ alkyl optionally substituted by OH.

A method of preventing or treating atherosclerosis or restenosis in mammals, wherein the compound administered has the Formula III and R^{III-2} is C₁₋₇ alkyl substituted by NR^{III-7}R^{III-8}, R^{III-11}, SOᵢR^{III-9}, or OC₂₋₄ alkyl which is further substituted by het^{III}, OR^{III-10}, or OC(═O)aryl^{III} wherein R^{III-7}, R^{III-8}, R^{III-9}, R^{III-10} and R^{III-11} are the same as defined in the summary of invention.

A method of preventing or treating atherosclerosis or restenosis in mammals, wherein the compound administered has the Formula III and R^{III-2} is C₁₋₇ alkyl substituted by OC₂₋₄ alkyl which is further substituted by het^{III}, OH, OC₁₋₄ alkyl, or OC(═O)aryl^{III}.

A method of preventing or treating atherosclerosis or restenosis in mammals, wherein the compound administered has the Formula III and R^{III-2} is C₁₋₇ alkyl substituted by SOᵢR^{III-9} wherein R^{III-9} and I are the same as defined in the summary of invention.

A method of preventing or treating atherosclerosis or restenosis in mammals, wherein the compound administered has the Formula III and R^{III-2} is C₁₋₇ alkyl substituted by SOᵢR^{III-9}; wherein R^{III-9} is C₁₋₄ alkyl, optionally substituted by OH, or R^{III-9} is phenyl, optionally substituted by Cl; wherein i^{III} is 0, 1, or 2.

A method of preventing or treating atherosclerosis or restenosis in mammals, wherein the compound administered has the Formula III and R^{III-2} is methyl.

A method of preventing or treating atherosclerosis or restenosis in mammals, wherein the compound administered has the Formula III and W is NR^{III-7}R^{III-8}, wherein R^{III-7} and R^{III-8} are the same as defined in the summary of invention.

A method of preventing or treating atherosclerosis or restenosis in mammals, wherein the compound administered has the Formula III and W is NR^{III-7}R^{III-8}, wherein R^{III-7} and R^{III-8} together with the nitrogen to which they are attached to form a het^{III}, wherein said het^{III} is the same as defined in the summary of invention. A method of preventing or treating atherosclerosis or restenosis in mammals, wherein the compound administered has the Formula III and het^{III} is morpholine, piperidine, pyrrolidine, piperazine, or 4-methyl-piperazine.

A method of preventing or treating atherosclerosis or restenosis in mammals, wherein the compound administered has the Formula III and W is NR^{III-}7^{III-8}, wherein R^{III-7} and R^{III-8} are independently H or C₁₋₄ alkyl optionally substituted by OH.

A method of preventing or treating atherosclerosis or restenosis in mammals, wherein the compound administered has the Formula III and het^{III} is morpholine.

A method of preventing or treating atherosclerosis or restenosis in mammals, wherein the compound administered has the Formula III and W is OR^{III-9}, or SOᵢR^{III-9} wherein R^{III-9} is C₁₋₆alkyl which may be partially unsaturated and optionally substituted by OR^{III-10}, Oaryl^{III}, het^{III}, aryl^{III}, NR^{III-10}R^{III-10}, CN, CONR^{III-10}R^{III-10}, or halo; wherein R^{III-10} is H or C₁₋₄ alkyl.

A method of preventing or treating atherosclerosis or restenosis in mammals, wherein the compound administered has the Formula III and R^{III-3} is H.

A method of preventing or treating atherosclerosis or restenosis in mammals, wherein the compound administered has the Formula III and R^{III-3} is CF₃, or halo.

A method of preventing or treating atherosclerosis or restenosis in mammals, wherein the compound administered has the Formula III and R^{III-4} is aryl^{III} or het^{III}.

A method of preventing or treating atherosclerosis or restenosis in mammals, wherein the compound administered has the Formula III and R^{III-4} is SO₂NHR^{III-12}, CONHR^{III-12}, NHCOR^{III-12}, or NHSO₂R^{III-12}, wherein R^{III-12} is the same as defined in the summary of invention.

A method of preventing or treating atherosclerosis or restenosis in mammals, wherein the compound administered has the Formula III and R^{III-4} is C₂₋₈ alkyl which is partially unsaturated and optionally substituted by OR^{III-10}, NR^{III-7}R^{III-8}, halo, SOᵢR^{III-9}, OR^{III-13} or R^{III-11}, wherein R^{III-7}, R^{III-8}, R^{III-9}, R^{III-10}, R^{III-11} and R^{III-13} are the same as defined in the summary of invention.

A method of preventing or treating atherosclerosis or restenosis in mammals, wherein the compound administered has the Formula III and R^{III-4} is (Z or E)—CH═CHC₁₋₄ alkyl, optionally substituted by OH.

A method of preventing or treating atherosclerosis or restenosis in mammals, wherein the compound administered has the Formula III and R^{III-4} is —C≡CC₁₋₄ alkyl, optionally substituted by OH or OR^{III-13}, wherein R^{III-13} is (P═O)(OH)₂, (P═O)(C₁₋₇ alkoxy)₂, or CO(CH₂)₆CON(CH₃)(CH₂)ₙSO₃⁻M^{III+}.

A method of preventing or treating atherosclerosis or restenosis in mammals, wherein the compound administered has the Formula III and R^{III-4} is C₁₋₈ alkyl substituted by OR^{III-3} wherein R^{III-13} is (P═O)(OH)₂, (P═O)(C₁₋₇ alkoxy)₂, or CO(CH₂)ₙCON(CH₃)(CH₂)₆SO₃⁻M⁺.

A method of preventing or treating atherosclerosis or restenosis in mammals, wherein the compound administered has the Formula III and R^{III-4} is C₁₋₈ alkyl substituted by SOᵢR^{III-9}, wherein R^{III-9} is the same as defined in the summary of invention.

A method of preventing or treating atherosclerosis or restenosis in mammals, wherein the compound administered has the Formula III and R^{III-4} is NR^{III-7}R^{III-8}, wherein R^{III-7} and R^{III-8} are the same as defined in the summary of invention.

A method of preventing or treating atherosclerosis or restenosis in mammals, wherein the compound administered has the Formula III and R^{III-4} is C₁₋₈ alkyl substituted by NR^{III-7}R^{III-8}, wherein R^{III-7} and R^{III-8} are the same as defined in the summary of invention.

A method of preventing or treating atherosclerosis or restenosis in mammals, wherein the compound administered has the Formula III and R^{III-7} and R^{III-8} together with the nitrogen to which they are attached to form a het^{III}, wherein het^{III} is the same as defined in the summary of invention.

A method of preventing or treating atherosclerosis or restenosis in mammals, wherein the compound administered has the Formula III and R^{III-7} and R^{III-8} are independently C₁₋₆ alkyl, optionally substituted by one or more substituents selected from a group consisting of OH, aryl^{III} or CN wherein aryl^{III} is the same as defined in the summary of invention.

A method of preventing or treating atherosclerosis or restenosis in mammals, wherein the compound administered has the Formula III and R^{III-4} is C₁₋₈ alkyl substituted by N₃.

A method of preventing or treating atherosclerosis or restenosis in mammals, wherein the compound administered has the Formula III and R^{III-4} is C₁₋₈ alkyl substituted by het^{III} wherein het^{III} is the same as defined in the summary of invention.

A method of preventing or treating atherosclerosis or restenosis in mammals, wherein the compound administered has the Formula III and R^{III-4} is 4-morpholine methyl.

A method of preventing or treating atherosclerosis or restenosis in mammals, wherein the compound administered has the Formula III and R^{III-4} is C₁₋₇ alkyl substituted by R^{III-11}, wherein R^{III-11} is the same as defined in the summary of invention.

A method of preventing or treating atherosclerosis or restenosis in mammals, wherein the compound administered has the Formula III and R^{III-5} is H or C₁₋₇ alkyl optionally substituted by OH.

A method of preventing or treating atherosclerosis or restenosis in mammals, wherein the compound administered has the Formula III and R^{III-6} is OC₁₋₇ alkyl optionally substituted by one or more OH.

A method of preventing or treating atherosclerosis or restenosis in mammals, wherein the compound administered has the Formula III and R^{III-6} is halo.

A method of preventing or treating atherosclerosis or restenosis in mammals, wherein the compound administered has the Formula III and R^{III-6} is C≡CC₁₋₇ alkyl substituted by one or more OH, or C₂₋₇ alkoxy substituted by one or more OH.

A method of preventing or treating atherosclerosis or restenosis in mammals, wherein the compound administered has the Formula III and R^{III-6} is H or C₁₋₇ alkyl, optionally substituted by halo, NR^{III-10}R^{III-10}, OH, CO₂R^{III-10}, or het^{III}; wherein R^{III-10} and het^{III} are the same as defined in the summary of invention.

A method of preventing or treating atherosclerosis or restenosis in mammals, wherein the compound administered has the Formula III and M^{III} is sodium, potassium, or lithium.

A method of preventing or treating atherosclerosis or restenosis in mammals, wherein the compound administered has the Formula III and X^{III} is S; W, R^{III-1}, R^{III-2}, R^{III-3}, R^{III-4}, R^{III-5}, R^{III-6} are the same as defined in the summary of invention.

A method of preventing or treating atherosclerosis or restenosis in mammals, wherein the compound administered has the Formula III and XIII, is O; R^{III-1}, R^{III-3}, R^{III-4}, R^{III-5}, R^{III-6} are the same as defined in claim 1, W is NR^{II-7}R^{III-8}, OR^{III-9}, SOᵢR^{III-9} or R^{III-2}; wherein R^{III-2} is:

a) (CH₂CH₂O)ₙR^{III-10},

b) het^{III}, wherein said het^{III}, is bonded via a carbon atom,

c) C₁₋₇ alkyl which is partially unsaturated and optionally substituted by OH,

d) C₃₋₈ cycloalkyl, or

e) C₁₋₇ alkyl which is optionally substituted by one or more substituents selected from a group consisting of Ohet^{III}, Oaryl^{III}, SOᵢR^{III-9}, or OC₂₋₄ alkyl which is further substituted by het^{III}, OR^{III-10}, or OC(═O)aryl^{III}; wherein R^{III-7}, R^{III-8}, R^{III-9}, R^{III-10} and n^{III} are the same as defined in the summary of invention.

A method of preventing or treating atherosclerosis or restenosis in mammals, wherein the compound administered has the Formula III and X^{III} is O or S; R^{III-1} is Cl; R^{III-3} is H; R^{III-5} is H; R^{III-6} is H or F; R^{III-4} is 4-morpholinylmethyl; and R^{III-2} is:

a) C₁₋₄ alkyl substituted by SOᵢR^{III-9} or C₁₋₄ alkoxy which is further substituted by OH, het^{III}, OC₁₋₄ alkyl, or OC(═O)phenyl,

b) (CH₂CH₂O)₂C₁₋₄alkyl,

c) C₁₋₆alkyl which is partially unsaturated and optionally substituted by OH,

d) cyclopropyl,

e) tetrahydro-2H-pyranyl,

f) piperdinyl,

g) mopholinyl,

h) 1-methyl-piperidinyl, or

i) 1,1-dioxo-tetrahydro-2H-thiopyran;

wherein R^{III-9} is phenyl optionally substituted by Cl, or R^{III-9} is C₁₋₆alkyl optionally substituted by OH. A method of preventing or treating atherosclerosis or restenosis in mammals, wherein the compound administered has the Formula III and X^{III} is O or S; R^{III-1} is Cl; R^{III-3} is H; R^{III-5} is H; R^{III-6} is H or F; R^{III-4} is C₁₋₆alkyl which is partially unsaturated and optionally substituted by OH or OR^{III-13}; or R^{III-4} is C₁₋₄alkyl substituted with OR^{III-13}; W is NR^{III-10}R^{III-10}, cyclopropyl, (CH₂CH₂O)₂OR^{III-10}, or C₁₋₆ alkyl which may be partially unsaturated and is optionally substituted by OH, mopholinyl, NR^{III-10}R^{III-10}; C(═O)OC₁₋₄alkyl, wherein R^{III-10} is H or C₁₋₄alkyl; R^{III-13} is (P═O)(C₁₋₇ alkoxy)₂, CO(CH₂)ₙCON(CH₃)(CH₂)ₙSO₃⁻M^{III+}, or (P═O)(OH)₂.

A method of preventing or treating atherosclerosis or restenosis in mammals, wherein the compound administered has the Formula III and X^{III}, is O or S; R^{III-1} is Cl; R^{III-3} is H; R^{III-5} is H; R^{III-6} is C≡CC₁₋₄ alkyl optionally substituted by OH; R^{III-4} is H or C₁₋₄alkyl which may be partially unsaturated and optionally substituted by OH, and W is C₁₋₄ alkyl optionally substituted by OH.

A method of preventing or treating atherosclerosis or restenosis in mammals, wherein the compound administered has the Formula III and M^{III} is sodium, potassium, or lithium.

X is S; W, R^{III-1}, R^{III-2}, R^{III-3}, R^{III-4}, R^{III-5}, R^{III-6} are the same as defined in X^{III} is O; R^{III-1}, R^{III-3}, R^{III-4}, R^{III-5}, R^{III-6} are the same as defined in claim 1, W is NR^{III-7}R^{III-8}, OR^{III-9}, SOᵢR^{III-9} or R^{III-2}; wherein R^{III-2} is:

a) (CH₂CH₂O)ₙR^{III-10},

b) het^{III}, wherein said het^{III}, is bonded via a carbon atom,

c) C₁₋₇ alkyl which is partially unsaturated and optionally substituted by OH,

d) C₃₋₈ cycloalkyl, or

e) C₁₋₇ alkyl which is optionally substituted by one or more substituents selected from a group consisting of Ohet^{III}, Oaryl^{III}, SOᵢR^{III-9}, or OC₂₋₄ alkyl which is further substituted by het^{III}, OR^{III-10}, or OC(═O)aryl^{III};

wherein R^{III-7}, R^{III-8}, R^{III-9}, R^{III-10} and n^{III} are the same as defined in X^{III} is O or S; R^{III-1} is Cl; R^{III-3} is H; R^{III-5} is H; R^{III-6} is H or F; R^{III-4} is 4-morpholinylmethyl; and R^{III-2} is:

a) C₁₋₄ alkyl substituted by SOᵢR^{III-9}, or C₁₋₄ alkoxy which is further substituted by OH, het^{III}, OC₁₋₄ alkyl, or OC(═O)phenyl,

b) (CH₂CH₂O)₂C₁₋₄alkyl,

c) C₁₋₆alkyl which is partially unsaturated and optionally substituted by OH,

d) cyclopropyl,

e) tetrahydro-2H-pyranyl,

f) piperdinyl,

g) mopholinyl,

h) 1-methyl-piperidinyl, or

i) 1,1-dioxo-tetrahydro-2H-thiopyran;

wherein R^{III-9} is phenyl optionally substituted by Cl, or R^{III-9} is C₁₋₆alkyl optionally substituted by OH. X^{III} is O or S; R^{III-1} is Cl; R^{III-3} is H; R^{III-5} is H; R^{III-6} is H or F; R^{III-4} is C₁₋₆alkyl which is partially unsaturated and optionally substituted by OH or OR^{III-13}; or R^{III-4} is C₁₋₄alkyl substituted with OR^{III-13}; W is NR^{III-10}R^{III-10}, cyclopropyl, (CH₂CH₂O)₂OR^{III-10}, or C₁₋₆ alkyl which may be partially unsaturated and is optionally substituted by OH, mopholinyl, NR^{III-10}R^{III-10}; C(═O)OC₁₋₄alkyl, wherein R^{III-10} is H or C₁₋₄alkyl; R^{III-13} is (P═O)(C₁₋₇ alkoxy)₂, CO(CH₂)ₙCON(CH₃)(CH₂)ₙSO₃⁻M^{III+}, or (P═O)(OH)₂.

A method of preventing or treating atherosclerosis or restenosis in mammals, wherein the compound administered has the Formula III and X^{III} is O or S; R^{III-1} is Cl; R^{III-3} is H; R^{III-5} is H; R^{III-6} is C≡CC₁₋₄ alkyl optionally substituted by OH; R^{III-4} is H or C₁₋₄alkyl which may be partially unsaturated and optionally substituted by OH, and W is C₁₋₄ alkyl optionally substituted by OH.

A method of preventing or treating atherosclerosis or restenosis, wherein the compound administered has the Formula III and the compound administered is

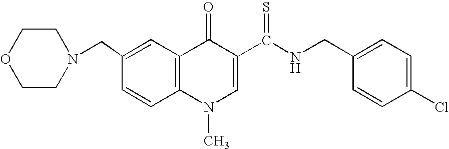

or a pharmaceutically acceptable salt thereof.

A method of preventing or treating atherosclerosis or restenosis, wherein the compound administered has the Formula III and the compound administered is

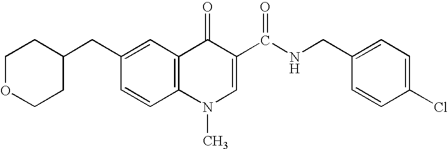

or a pharmaceutically acceptable salt thereof.

A method of preventing or treating atherosclerosis or restenosis, wherein the compound administered has the Formula III and the compound administered is

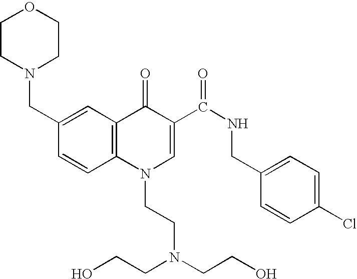

or a pharmaceutically acceptable salt thereof.

A method of preventing or treating atherosclerosis or restenosis, wherein the compound administered has the Formula III and the compound administered is

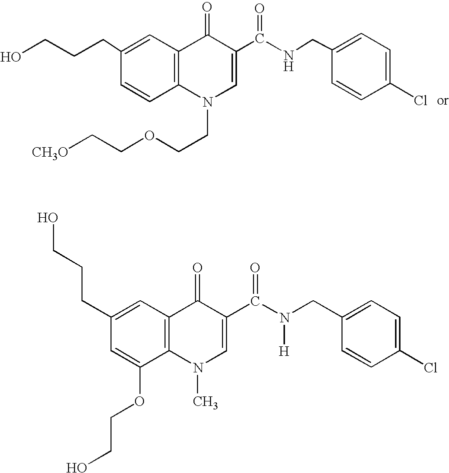

or a pharmaceutically acceptable salt thereof.

A method of preventing or treating atherosclerosis or restenosis, wherein the compound administered has the Formula III and the compound administered is

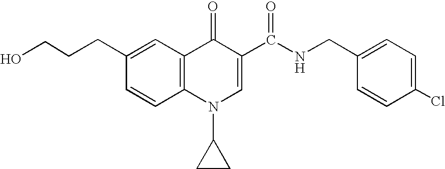

or a pharmaceutically acceptable salt thereof.

A method of preventing or treating atherosclerosis or restenosis, wherein the compound administered has the Formula III and the compound administered is

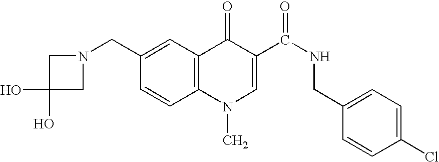

or a pharmaceutically acceptable salt thereof.

A method of preventing or treating atherosclerosis or restenosis, wherein the compound administered has the Formula III and the compound administered is

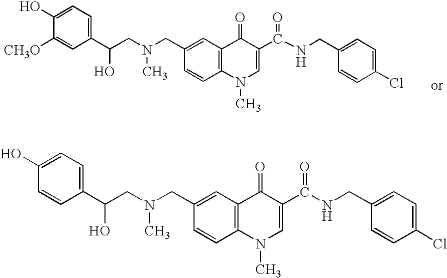

or a pharmaceutically acceptable salt thereof.

A method of preventing or treating atherosclerosis or restenosis, wherein the compound administered has the Formula III and the compound administered is

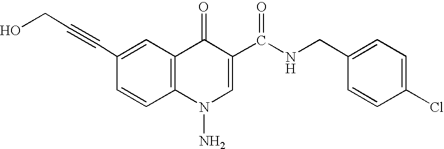

or a pharmaceutically acceptable salt thereof.

A method of preventing or treating atherosclerosis or restenosis, wherein the compound administered has the Formula III and the compound administered is

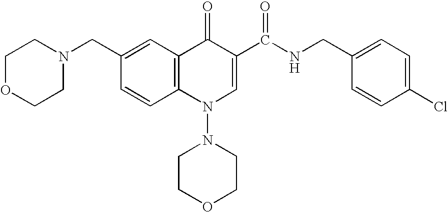

or a pharmaceutically acceptable salt thereof.

A method of preventing or treating atherosclerosis or restenosis in mammals, wherein the compound administered has the Formula III and R^{III-2} is methyl, ethyl, propyl, isopropyl, butyl, tert-butyl, cyclopropyl, carboxymethyl, (C₁₋₇ alkoxy)carbonylmethyl, 2-hydroxyethyl, 2-(2-methoxyethoxy)ethyl, 3-(2-tetrahydropyranyloxy)propyl, 2-morpholinoethyl, 2-(diethylamino)ethyl, 2-(dimethylamino)ethyl, 2-piperidinoethyl, 3-piperidinopropyl, 2-(1-methylpyrrolidin-2-yl)ethyl, 2-(diisopropylamino)ethyl, 2-pyrrolidin-1-ylethyl, 3-(dimethylamino)propyl, or vinyl.

A method of preventing or treating atherosclerosis or restenosis in mammals, wherein the compound administered has the Formula III and R^{III-4} is 3-hydroxy-1-propynyl, 3-hydroxypropyl, hydroxymethyl, cis-4-hydroxy-1-butenyl, trans-4-hydroxy-1-butenyl, 2-hydroxyethyl)(ethyl)amino, morpholinomethyl, (CH₂)₂O(P═O)(OH)₂, (CH₂)₃O(P═O)((tert-butoxy)₂, 3-[di(tert-butyl)phosphoryl]propyl, 3-phosphorylpropyl, Na⁺⁻OS(O)₂CH₂CH₂N—CH₃)C(═O)(CH₂)₆C(═O)O(CH₂)₃, or Na⁺⁻OS(O)₂CH₂CH₂N(CH₃)C(═O)—(CH₂)₆C(═O)OCH₂C≡C—.

A method for preventing or treating atherosclerosis or restenosis in mammals, wherein the compound administered has the Formula III and is selected from the group consisting of

(1) N-(4-chlorobenzyl)-6-(3-hydroxy-1-propynyl)-1-isopropyl-4-oxo-1,4-dihydro-3-quinolinecarboxamide;

(2) 1-(sec-butyl)-N-(4-chlorobenzyl)-6-(3-hydroxy-1-propynyl)-4-oxo-1,4-dihydro-3-quinolinecarboxamide;

(3) 1-(sec-butyl)-N-(4-chlorobenzyl)-6-(3-hydroxy-1-propynyl)-8-methoxy-4-oxo-1,4-dihydro-3-quinolinecarboxamide;

(4) N-(4-chlorobenzyl)-6-[3-hydroxy-1-propenyl]-1-[2-(4-morpholinyl)ethyl]-4-oxo-1,4-dihydro-3-quinolinecarboxamide;

(5) N-(4-chlorobenzyl)-8-fluoro-6-(3-hydroxy-1-propynyl)-1-methyl-4-oxo-1,4-dihydro-3-quinolinecarboxamide;

(6) N-(4-chlorobenzyl)-8-fluoro-6-[(Z)-3-hydroxy-1-propenyl]-1-methyl-4-oxo-1,4-dihydro-3-quinolinecarboxamide;

(7) N-(4-chlorobenzyl)-1-[2-(diethylamino)ethyl]-8-fluoro-6-(3-hydroxy-1-propynyl)-4-oxo-1,4-dihydro-3-quinolinecarboxamide;

(8) N-(4-chlorobenzyl)-6-(3-hydroxy-1-propynyl)-4-oxo-1-propyl-1,4-dihydro-3-quinolinecarboxamide;

(9) N-(4-chlorobenzyl)-1-[2-(diethylamino)ethyl]-6-(3-hydroxy-1-propynyl)-4-oxo-1,4-dihydro-3-quinolinecarboxamide;

(10) N-(4-chlorobenzyl)-1-[2-(dimethylamino)ethyl]-6-(3-hydroxy-1-propynyl)-4-oxo-1,4-dihydro-3-quinolinecarboxamide hydrochloride;

(11) N-(4-chlorobenzyl)-6-(3-hydroxy-1-propynyl)-4-oxo-1-[2-(1-piperidinyl)ethyl]-1,4-dihydro-3-quinolinecarboxamide;

(12) N-(4-chlorobenzyl)-6-(3-hydroxy-1-propynyl)-4-oxo-1-[3-(1-piperidinyl)propyl]-1,4-dihydro-3-quinolinecarboxamide;

(13) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;

(14) N-(4-chlorobenzyl)-1-[2-(diisopropylamino)ethyl]-6-(3-hydroxy-1-propynyl)-4-oxo-1,4-dihydro-3-quinolinecarboxamide;

(15) N-(4-chlorobenzyl)-6-(3-hydroxy-1-propynyl)-4-oxo-1-[2-(1-pyrrolidinyl)ethyl]-1,4-dihydro-3-quinolinecarboxamide;

(16) N-(4-chlorobenzyl)-6-(3-hydroxy-1-propynyl)-1-[2-(4-morpholinyl)ethyl]-4-oxo-1,4-dihydro-3-quinolinecarboxamide;

(17) N-(4-chlorobenzyl)-1-[3-(dimethylamino)propyl]-6-(3-hydroxy-1-propynyl)-4-oxo-1,4-dihydro-3-quinolinecarboxamide;

(18) N-(4-chlorobenzyl)-6-(3-hydroxy-1-propynyl)-4-oxo-1-vinyl-1,4-dihydro-3-quinolinecarboxamide;

(19) N-(4-chlorobenzyl)-6-[(E)-3-hydroxy-1-propenyl]-1-methyl-4-oxo-1,4-dihydro-3-quinolinecarboxamide;

(20) N-(4-chlorobenzyl)-6-[(Z)-3-hydroxy-1-propenyl]-1-methyl-4-oxo-1,4-dihydro-3-quinolinecarboxamide;

(21) N-(4-chlorobenzyl)-1-cyclopropyl-6-(3-hydroxy-1-propynyl)-4-oxo-1,4-dihydro-3-quinolinecarboxamide;

(22) tert-butyl 2-[3-{[(4-chlorobenzyl)amino]carbonyl}-6-(3-hydroxy-1-propynyl)-4-oxo-1(4H)-quinolinyl]acetate;

(23) 2-[3-{[(4-chlorobenzyl)amino]carbonyl}-6-(3-hydroxy-1-propynyl)-4-oxo-1(4H)-quinolinyl]acetic acid;

(24) N-(4-chlorobenzyl)-1-(2-hydroxyethyl)-6-(3-hydroxy-1-propynyl)-4-oxo-1,4-dihydro-3-quinolinecarboxamide;

(25) N-(4-chlorobenzyl)-6-(3-hydroxy-1-propynyl)-1-methyl-4-oxo-1,4-dihydro-3-quinolinecarboxamide;

(26) di(tert-butyl) 3-(3-{[(4-chlorobenzyl)amino]carbonyl}-1-methyl-4-oxo-1,4-dihydro-6-quinolinyl)propyl phosphate;

(27) 3-(3-{[(4-chlorobenzyl)amino]carbonyl}-1-methyl-4-oxo-1,4-dihydro-6-quinolinyl)propyl dihydrogen phosphate;

(28) di(tert-butyl) 3-(3-{[(4-chlorobenzyl)amino]carbonyl}-1-cyclopropyl-4-oxo-1,4-dihydro-6-quinolinyl)propyl phosphate;

(29) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;

(30) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;

(31) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;

(32) sodium 2-[(8-{[3-(3-{[(4-chlorobenzyl)amino]carbonyl}-1-cyclopropyl-4-oxo-1,4-dihydro-6-quinolinyl)-2-propynyl]oxy}-8-oxooctanoyl)(methyl)amino]-1-ethanesulfonate;

(33) 1-(tert-butyl)-N-(4-chlorobenzyl)-6-(3-hydroxy-1-propynyl)-4-oxo-1,4-dihydro-3-quinolinecarboxamide;

(34) sodium 2-[{8-[3-(1-(tert-butyl)-3-{[(4-chlorobenzyl)amino]-carbonyl}-4-oxo-1,4-dihydro-6-quinolinyl)propoxy]-8-oxooctanoyl}(methyl)amino]-1-ethanesulfonate;

(35) sodium 2-[(8-{[3-(1-(tert-butyl)-3-{[(4-chlorobenzyl)amino]-carbonyl}-4-oxo-1,4-dihydro-6-quinolinyl)-2-propynyl]oxy}-8-oxooctanoyl)(methyl)amino]-1-ethanesulfonate;

(36) N-(4-chlorobenzyl)-6-(3-hydroxy-1-propynyl)-1-[2-(2-methoxyethoxy)-ethyl]-4-oxo-1,4-dihydro-3-quinolinecarboxamide;

(37) N-(4-cyanobenzyl)-6-(3-hydroxy-1-propynyl)-1-methyl-4-oxo-1,4-dihydro-3-quinolinecarboxamide;

(38) N-(4-chlorobenzyl)-1-methyl-6-(1,4-oxazepan-4-ylmethyl)-4-oxo-1,4-dihydro-3-quinolinecarboxamide;

(39) N-(4-chlorobenzyl)-1-methyl-4-oxo-6-(1,4-thiazepan-4-ylmethyl)-1,4-dihydro-3-quinolinecarboxamide;

(40) N-(4-chlorobenzyl)-1-methyl-6-(2-oxa-5-azabicyclo[2.2.1]hept-5-ylmethyl)-4-oxo-1,4-dihydro-3-quinolinecarboxamide;

(41) N-(4-chlorobenzyl)-6-(2,3-dihydro-4H-1,4-benzoxazin-4-ylmethyl)-1-methyl-4-oxo-1,4-dihydro-3-quinolinecarboxamide;

(42) 6-(azidomethyl)-N-(4-chlorobenzyl)-1-methyl-4-oxo-1,4-dihydro-3-qulnoline-carboxamide;

(43) N-(4-chlorobenzyl)-1-methyl-4-oxo-6-vinyl-1,4-dihydro-3-quinoline-carboxamide;

(44) N-(4-chlorobenzyl)-1-[2-(2-hydroxyethoxy)ethyl]-6-(3-hydroxy-1-propynyl)-4-oxo-1,4-dihydro-3-quinolinecarboxamide;

(45) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;

(46) N-(4-chlorobenzyl)-1-[2-(2-hydroxyethoxy)ethyl]-6-(4-morpholinylmethyl)-4-oxo-1,4-dihydro-3-quinolinecarboxamide;

(47) N-(4-chlorobenzyl)-1-[2-(2-ethoxyethoxy)ethyl]-6-(4-morpholinylmethyl)-4-oxo-1,4-dihydro-3-quinolinecarboxamide;

(48) N-(4-chlorobenzyl)-6-(4-morpholinylmethyl)-4-oxo-1-(2-propynyl)-1,4-dihydro-3-quinolinecarboxamide;

(49) N-(4-chlorobenzyl)-1-[2-(ethylsulfanyl)ethyl]-6-(4-morpholinylmethyl)-4-oxo-1,4-dihydro-3-quinolinecarboxamide;

(50) N-(4-chlorobenzyl)-1-[3-(methylsulfanyl)propyl]-6-(4-morpholinylmethyl)-4-oxo-1,4-dihydro-3-quinolinecarboxamide;

(51) N-(4-chlorobenzyl)-1-(4-hydroxy-2-butynyl)-6-(4-morpholinylmethyl)-4-oxo-1,4-dihydro-3-quinolinecarboxamide;

(52) N-(4-chlorobenzyl)-6-{[(2-hydroxy-2-phenylethyl)(methyl)amino]methyl}-1-methyl-4-oxo-1,4-dihydro-3-quinolinecarboxamide;

(53) N-(4-chlorobenzyl)-1-[3-(methylsulfinyl)propyl]-6-(4-morpholinylmethyl)-4-oxo-1,4-dihydro-3-quinolinecarboxamide;

(54) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;

(55) N-(4-chlorobenzyl)-1-[3-(methylsulfonyl)propyl]-6-(4-morpholinylmethyl)-4-oxo-1,4-dihydro-3-quinolinecarboxamide;

(56) N-(4-chlorobenzyl)-1-[2-(ethylsulfinyl)ethyl]-6-(4-morpholinylmethyl)-4-oxo-1,4-dihydro-3-quinolinecarboxamide;

(57) N-(4-chlorobenzyl)-1-[2-(ethylsulfonyl)ethyl]-6-(4-morpholinylmethyl)-4-oxo-1,4-dihydro-3-quinolinecarboxamide;

(58) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;

(59) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;

(60) N-(4-chlorobenzyl)-6-(4-morpholinylmethyl)-4-oxo-1-[2-(phenylsulfanyl)ethyl]-1,4-dihydro-3-quinolinecarboxamide;

(61) N-(4-chlorobenzyl)-1-[(methylsulfanyl)methyl]-6-(4-morpholinylmethyl)-4-oxo-1,4-dihydro-3-quinolinecarboxamide;

(62) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;

(63) N-(4-chlorobenzyl)-6-[(3-hydroxy-1-azetidinyl)methyl]-1-methyl-4-oxo-1,4-dihydro-3-quinolinecarboxamide;

(64) N-(4-chlorobenzyl)-6-(4-morpholinylmethyl)-4-oxo-1-[(phenylsulfanyl)-methyl]-1,4-dihydro-3-quinolinecarboxamide;

(65) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;

(66) N-(4-chlorobenzyl)-6-[(3,3-dihydroxy-1-azetidinyl)methyl]-1-methyl-4-oxo-1,4-dihydro-3-quinolinecarboxamide;

(67) N-(4-chlbrobenzyl)-1-[(methylsulfinyl)methyl]-6-(4-morpholinylmethyl)-4-oxo-1,4-dihydro-3-quinolinecarboxamide;

(68) N-(4-chlorobenzyl)-1-[(methylsulfonyl)methyl]-6-(4-morpholinylmethyl)-4-oxo-1,4-dihydro-3-quinolinecarboxamide;

(69) N-(4-chlorobenzyl)-6-(4-morpholinylmethyl)-4-oxo-1-[(phenylsulfinyl)-methyl]-1,4-dihydro-3-quinolinecarboxamide;

(70) N-(4-chlorobenzyl)-6-(4-morpholinylmethyl)-4-oxo-1-[(phenylsulfonyl)-methyl]-1,4-dihydro-3-quinolinecarboxamide;

(71) N-(4-chlorobenzyl)-6-(3-hydroxypropyl)-1-[2-(2-methoxyethoxy)ethyl]-4-oxo-1,4-dihydro-3-quinolinecarboxamide;

(72) N-(4-chlorobenzyl)-1-[2-(2-methoxyethoxy)ethyl]-6-(4-morpholinylmethyl)-4-oxo-1,4-dihydro-3-quinolinecarboxamide;

(73) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;

(74) N-(4-chlorobenzyl)-6-{[(cyanomethyl)(methyl)amino]methyl}-1-[2-(2-methoxyethoxy)ethyl]-4-oxo-1,4-dihydro-3-quinolinecarboxamide;

(75) N-(4-chlorobenzyl)-6-{[(3R)-3-hydroxypyrrolidinyl]methyl}-1-[2-(2-methoxyethoxy)ethyl]-4-oxo-1,4-dihydro-3-quinolinecarboxamide;

(76) N-(4-chlorobenzyl)-1-[2-(2-methoxyethoxy)ethyl]-6-[(methylsulfanyl)methyl]-4-oxo-1,4-dihydro-3-quinolinecarboxamide;

(77) N-(4-chlorobenzyl)-6-{[[(1R,2S)-2-hydroxy-1-methyl-2-phenylethyl](methyl)-amino]methyl}-1-[2-(2-methoxyethoxy)ethyl]-4-oxo-1,4-dihydro-3-quinoline-carboxamide;

(78) N-(4-chlorobenzyl)-6-{[(2-hydroxy-2-phenylethyl)(methyl)amino]methyl}-1-[2-(2-methoxyethoxy)ethyl]-4-oxo-1,4-dihydro-3-quinolinecarboxamide;

(79) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;

(80) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;

(81) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinoline-carboxamide;

(82) N-(4-chlorobenzyl)-6-(3-hydroxy-1-propynyl)-1-methyl-4-oxo-1,4-dihydro-3-quinolinecarbothioamide;

(83) N-(4-chlorobenzyl)-8-(3-hydroxy-1-propynyl)-1-methyl-4-oxo-1,4-dihydro-3-quinolinecarboxamide;

(84) N-(4-chlorobenzyl)-8-(4-hydroxy-1-butynyl)-1-methyl-4-oxo-1,4-dihydro-3-quinolinecarboxamide;

(85) N-(4-chlorobenzyl)-6-{[3-(hydroxyimino)-1-azetidinyl]methyl}-1-methyl-4-oxo-1,4-dihydro-3-quinolinecarboxamide;

(86) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;

(87) N-(4-chlorobenzyl)-1-([(4-chlorophenyl)sulfanyl]methyl)-6-(4-morpholinylmethyl)-4-oxo-1,4-dihydro-3-quinolinecarboxamide;

(88) N-(4-chlorobenzyl)-1-([(4-chlorophenyl)sulfinyl]methyl)-6-(4-morpholinylmethyl)-4-oxo-1,4-dihydro-3-quinolinecarboxamide;

(89) N-(4-chlorobenzyl)-1-([(4-chlorophenyl)sulfonyl]methyl)-6-(4-morpholinylmethyl)-4-oxo-1,4-dihydro-3-quinolinecarboxamide;

(90) N-(4-chlorobenzyl)-1-[(4-chlorophenoxy)methyl]-6-(4-morpholinylmethyl)-4-oxo-1,4-dihydro-3-quinolinecarboxamide;

(91) N-(4-chlorobenzyl)-1-[(2-methoxyethoxy)methyl]-6-(4-morpholinylmethyl)-4-oxo-1,4-dihydro-3-quinolinecarboxamide;

(92) 2-{[3-{[(4-chlorobenzyl)amino]carbonyl}-6-(4-morpholinylmethyl)-4-oxo-1(4H)-quinolinyl]methoxy}ethyl benzoate;

(93) N-(4-chlorobenzyl)-1-[(2-hydroxyethoxy)methyl]-6-(4-morpholinylmethyl)-4-oxo-1,4-dihydro-3-quinolinecarboxamide;

(94) N-(4-chlorobenzyl)-6-(4-morpholinylmethyl)-4-oxo-1-tetrahydro-2H-pyran-4-yl-1,4-dihydro-3-quinolinecarboxamide;

(95) N-(4-chlorobenzyl)-1-(1-methyl-4-piperidinyl)-6-(4-morpholinylmethyl)-4-oxo-1,4-dihydro-3-quinolinecarboxamide;

(96) N-(4-chlorobenzyl)-6-(4-morpholinylmethyl)-4-oxo-1-(4-piperidinyl)-1,4-dihydro-3-quinolinecarboxamide;

(97) N-(4-chlorobenzyl)-1-(1,1-dioxohexahydrothiopyran-4-yl)-6-(4-morpholinylmethyl)-4-oxo-1,4-dihydro-3-quinolinecarboxamide;

(98) N-(4-chlorobenzyl)-1-(4-morpholinyl)-6-(4-morpholinylmethyl)-4-oxo-1,4-dihydro-3-quinolinecarboxamlde;

(99) N-(4-chlorobenzyl)-1-(4-methyl-1-piperazinyl)-6-(4-morpholinylmethyl)-4-oxo-1,4-dihydro-3-quinolinecarboxamide;

(100) N-(4-chlorobenzyl)-6-(4-morpholinylmethyl)-4-oxo-1-(l-piperidinyl)-1,4-dihydro-3-quinolinecarboxamide;

(101) N-(4-chlorobenzyl)-6-(4-morpholinylmethyl)-4-oxo-1-(l-pyrrolidinyl)-1,4-dihydro-3-quinolinecarboxamide;

(102) N-(4-chlorobenzyl)-1-[(2R)-2-(methoxymethyl)pyrrolidinyl]-6-(4-morpholinylmethyl)-4-oxo-1,4-dihydro-3-quinolinecarboxamide;

(103) N-(4-chlorobenzyl)-1-(dimethylamino)-6-(4-morpholinylmethyl)-4-oxo-1,4-dihydro-3-quinolinecarboxamide;

(104) 1-Amino-N-(4-chlorobenzyl)-6-(3-hydroxy-1-propynyl)-4-oxo-1,4-dihydro-3-quinolinecarboxamide;

(105) 1-Amino-N-(4-chlorobenzyl)-6-(3-hydroxypropyl)-4-oxo-1,4-dihydro-3-quinolinecarboxamide;

(106) N-(4-chlorobenzyl)-1-(dimethylamino)-6-(3-hydroxy-1-propynyl)-4-oxo-1,4-dihydro-3-quinolinecarboxamide;

(107) N-(4-chlorobenzyl)-1-(dimethylamino)-4-oxo-1,4-dihydro-3-quinolinecarboxamide;

(108) 1-(allyloxy)-N-(4-chlorobenzyl)-4-oxo-1,4-dihydro-3-quinolinecarboxamide;

(109) N-(4-chlorobenzyl)-1-methoxy-4-oxo-1,4-dihydro-3-quinolinecarboxamide;

(110) N-(4-bromobenzyl)-1-(4-morpholinyl)-6-(4-morpholinylmethyl)-4-oxo-1,4-dihydro-3-quinolinecarboxamide;

(111) N-(4-fluorobenzyl)-1-(4-morpholinyl)-6-(4-morpholinylmethyl)-4-oxo-1,4-dihydro-3-quinolinecarboxamide;

(112) N-(4-chlorobenzyl)-1-{[2-(4-morpholinyl)ethoxy]methyl}-6-(4-morpholinylmethyl)-4-oxo-1,4-dihydro-3-quinolinecarboxamide;

(113) N-(4-chlorobenzyl)-1-{[2-(dimethylamino)ethoxy]methyl}-6-(4-morpholinylmethyl)-4-oxo-1,4-dihydro-3-quinolinecarboxamide;

(114) N-(4-chlorobenzyl)-1-{[2-(4-methyl-1-piperazinyl)ethoxy]methyl}-6-(4-morpholinylmethyl)-4-oxo-1,4-dihydro-3-quinolinecarboxamide;

(115) N-(4-chlorobenzyl)-6-(4-morpholinylmethyl)-4-oxo-1-{[2-(1-piperidinyl)ethoxylmethyl}-1,4-dihydro-3-quinolinecarboxamide;

(116) N-(4-chlorobenzyl)-6-(4-morpholinylmethyl)-4-oxo-1-{[2-(1-pyrrolidinyl)ethoxy]methyl}-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) 1-(sec-butyl)-N-(4-chlorobenzyl)-6-(3-hydroxy-1-propynyl)-4-oxo-1,4-dihydro-3-quinolinecarboxamide;

(2) 1-(sec-butyl)-N-(4-chlorobenzyl)-6-(3-hydroxy-1-propynyl)-8-methoxy-4-oxo-1,4-dihydro-3-quinolinecarboxamide;

(3) N-(4-chlorobenzyl)-6-[3-hydroxy-1-propenyl]-1-[2-(4-morpholinyl)ethyl]-4-oxo-1,4-dihydro-3-quinolinecarboxamide;

(4) N-(4-chlorobenzyl)-8-fluoro-6-(3-hydroxy-1-propynyl)-1-methyl-4-oxo-1,4-dihydro-3-quinolinecarboxamide;

(5) N-(4-chlorobenzyl)-8-fluoro-6-[(Z)-3-hydroxy-1-propenyl]-1-methyl-4-oxo-1,4-dihydro-3-quinolinecarboxamide;

(6) N-(4-chlorobenzyl)-1-[2-(diethylamino)ethyl]-6-(3-hydroxy-1-propynyl)-4-oxo-1,4-dihydro-3-quinolinecarboxamide;

(7) N-(4-chlorobenzyl)-1-[2-(dimethylamino)ethyl]-6-(3-hydroxy-1-propynyl)-4-oxo-1,4-dihydro-3-quinolinecarboxamide hydrochloride;

(8) N-(4-chlorobenzyl)-6-(3-hydroxy-1-propynyl)-4-oxo-1-[2-(1-piperidinyl)ethyl]-1,4-dihydro-3-quinolinecarboxamide;

(9) N-(4-chlorobenzyl)-6-(3-hydroxy-1-propynyl)-4-oxo-1-[3-(1-piperidinyl)propyl]-1,4-dihydro-3-quinolinecarboxamide;

(10) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;

(11) N-(4-chlorobenzyl)-1-[2-(diisopropylamino)ethyl]-6-(3-hydroxy-1-propynyl)-4-oxo-1,4-dihydro-3-quinolinecarboxamide;

(12) N-(4-chlorobenzyl)-6-(3-hydroxy-1-propynyl)-4-oxo-1-[2-(1-pyrrolidinyl)ethyl]-1,4-dihydro-3-quinolinecarboxamide;

(13) N-(4-chlorobenzyl)-6-(3-hydroxy-1-propynyl)-1-[2-(4-morpholinyl)ethyl]-4-oxo-1,4-dihydro-3-quinolinecarboxamide;

(14) N-(4-chlorobenzyl)-1-[3-(dimethylamino)propyl]-6-(3-hydroxy-1-propynyl)-4-oxo-1,4-dihydro-3-quinolinecarboxamide;

(15) N-(4-chlorobenzyl)-6-(3-hydroxy-1-propynyl)-4-oxo-1-vinyl-1,4-dihydro-3-quinolinecarboxamide;

(16) N-(4-chlorobenzyl)-6-[(E)-3-hydroxy-1-propenyl]-1-methyl-4-oxo-1,4-dihydro-3-quinolinecarboxamide;

(17) N-(4-chlorobenzyl)-6-[(Z)-3-hydroxy-1-propenyl]-1-methyl-4-oxo-1,4-dihydro-3-quinolinecarboxamide;

(18) N-(4-chlorobenzyl)-1-cyclopropyl-6-(3-hydroxy-1-propynyl)-4-oxo-1,4-dihydro-3-quinolinecarboxamide;

(19) tert-butyl 2-[3-{[(4-chlorobenzyl)amino]carbonyl}-6-(3-hydroxy-1-propynyl)-4-oxo-1(4H)-quinolinyl]acetate;

(20) N-(4-chlorobenzyl)-1-(2-hydroxyethyl)-6-(3-hydroxy-1-propynyl)-4-oxo-1,4-dihydro-3-quinolinecarboxamide;

(21) N-(4-chlorobenzyl)-6-(3-hydroxy-1-propynyl)-1-methyl-4-oxo-1,4-dihydro-3-quinolinecarboxamide;

(22) 3-(3-{[(4-chlorobenzyl)amino]carbonyl}-1-methyl-4-oxo-1,4-dihydro-6-quinolinyl)propyl dihydrogen phosphate;

(23) di(tert-butyl) 3-(3-{[(4-chlorobenzyl)amino]carbonyl}-1-cyclopropyl-4-oxo-1,4-dihydro-6-quinolinyl)propyl phosphate;

(24) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;

(25) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;

(26) N-(4-chlorobenzyl)-6-(3-hydroxy-1-propynyl)-1-[2-(2-methoxyethoxy)-ethyl]-4-oxo-1,4-dihydro-3-quinolinecarboxamide;

(27) N-(4-cyanobenzyl)-6-(3-hydroxy-1-propynyl)-1-methyl-4-oxo-1,4-dihydro-3-quinolinecarboxamide;

(28) N-(4-chlorobenzyl)-6-(3-hydroxy-1-propynyl)-4-oxo-1-propyl-1,4-dihydro-3-quinolinecarboxamide;

(29) N-(4-chlorobenzyl)-1-methyl-6-(1,4-oxazepan-4-ylmethyl)-4-oxo-1,4-dihydro-3-quinolinecarboxamide;

(30) N-(4-chlorobenzyl)-1-methyl-4-oxo-6-(1,4-thiazepan-4-ylmethyl)-1,4-dihydro-3-quinolinecarboxamide;

(31) 6-(azidomethyl)-N-(4-chlorobenzyl)-1-methyl-4-oxo-1,4-dihydro-3-quinolinecarboxamide;

(32) N-(4-chlorobenzyl)-6-[(4,4-difluoro-1-piperidinyl)methyl]-1-methyl-4-oxo-1,4-dihydro-3-quinolinecarboxamide;

(33) N-(4-chlorobenzyl)-4-hydroxy-6-iodo-3-quinolinecarbothioamide;

(34) N-(4-chlorobenzyl)-6-(2,3-dihydro-4H-1,4-benzoxazin-4-ylmethyl)-1-methyl-4-oxo-1,4-dihydro-3-quinolinecarboxamide.

(35) N-(4-chlorobenzyl)-1-methyl-4-oxo-6-vinyl-1,4-dihydro-3-quinoline-carboxamide;

(36) N-(4-chlorobenzyl)-1-[2-(2-hydroxyethoxy)ethyl]-6-(3-hydroxy-1-propynyl)-4-oxo-1,4-dihydro-3-quinolinecarboxamide;

(37) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;

(38) N-(4-chlorobenzyl)-1-[2-(2-hydroxyethoxy)ethyl]-6-(4-morpholinylmethyl)-4-oxo-1,4-dihydro-3-quinolinecarboxamide;

(39) N-(4-chlorobenzyl)-1-[2-(2-ethoxyethoxy)ethyl]-6-(4-morpholinylmethyl)-4-oxo-1,4-dihydro-3-quinolinecarboxamide;

(40) N-(4-chlorobenzyl)-1-[2-(ethylsulfanyl)ethyl]-6-(4-morpholinylmethyl)-4-oxo-1,4-dihydro-3-quinolinecarboxamide;

(41) N-(4-chlorobenzyl)-1-[3-(methylsulfanyl)propyl]-6-(4-morpholinylmethyl)-4-oxo-1,4-dihydro-3-quinolinecarboxamide;

(42) N-(4-chlorobenzyl)-1-(4-hydroxy-2-butynyl)-6-(4-morpholinylmethyl)-4-oxo-1,4-dihydro-3-quinolinecarboxamide;

(43) N-(4-chlorobenzyl)-6-{[(2-hydroxy-2-phenylethyl)(methyl)amino]methyl}-1-methyl-4-oxo-1,4-dihydro-3-quinolinecarboxamide;

(44) N-(4-chlorobenzyl)-1-[3-(methylsulfinyl)propyl]-6-(4-morpholinylmethyl)-4-oxo-1,4-dihydro-3-quinolinecarboxamide;

(45) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;

(46) N-(4-chlorobenzyl)-1-[3-(methylsulfonyl)propyl]-6-(4-morpholinylmethyl)-4-oxo-1,4-dihydro-3-quinolinecarboxamide;

(47) N-(4-chlorobenzyl)-1-[2-(ethylsulfinyl)ethyl]-6-(4-morpholinylmethyl)-4-oxo-1,4-dihydro-3-quinolinecarboxamide;

(48) N-(4-chlorobenzyl)-1-[2-(ethylsulfonyl)ethyl]-6-(4-morpholinylmethyl)-4-oxo-1,4-dihydro-3-quinolinecarboxamide;

(49) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;

(50) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;

(51) N-(4-chlorobenzyl)-6-(4-morpholinylmethyl)-4-oxo-1-[2-(phenylsulfanyl)ethyl]-1,4-dihydro-3-quinolinecarboxamide;

(52) N-(4-chlorobenzyl)-1-[(methylsulfanyl)methyl]-6-(4-morpholinylmethyl)-4-oxo-1,4-dihydro-3-quinolinecarboxamide;

(53) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;

(54) N-(4-chlorobenzyl)-6-[(3-hydroxy-1-azetidinyl)methyl]-1-methyl-4-oxo-1,4-dihydro-3-quinolinecarboxamide;

(55) N-(4-chlorobenzyl)-6-(4-morpholinylmethyl)-4-oxo-1-[(phenylsulfanyl)-methyl]-1,4-dihydro-3-quinolinecarboxamide

(56) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;

(57) N-(4-chlorobenzyl)-6-[(3,3-dihydroxy-1-azetidinyl)methyl]-1-methyl-4-oxo-1,4-dihydro-3-quinolinecarboxamide;

(58) N-(4-chlorobenzyl)-1-[(methylsulfinyl)methyl]-6-(4-morpholinylmethyl)-4-oxo-1,4-dihydro-3-quinolinecarboxamide;

(59) N-(4-chlorobenzyl)-1-[(methylsulfonyl)methyl]-6-(4-morpholinylmethyl)-4-oxo-1,4-dihydro-3-quinolinecarboxamide;

(60) N-(4-chlorobenzyl)-6-(4-morpholinylmethyl)-4-oxo-1-[(phenylsulfinyl)-methyl]-1,4-dihydro-3-quinolinecarboxamide;

(61) N-(4-chlorobenzyl)-6-(4-morpholinylmethyl)-4-oxo-1-[(phenylsulfonyl)-methyl]-1,4-dihydro-3-quinolinecarboxamide;

(62) N-(4-chlorobenzyl)-6-(3-hydroxypropyl)-1-[2-(2-methoxyethoxy)ethyl]-4-oxo-1,4-dihydro-3-quinolinecarboxamide;

(63) N-(4-chlorobenzyl)-1-[2-(2-methoxyethoxy)ethyl]-6-(4-morpholinylmethyl)-4-oxo-1,4-dihydro-3-quinolinecarboxamide;

(64) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;

(65) N-(4-chlorobenzyl)-6-{[(3R)-3-hydroxypyrrolidinyl]methyl}-1-92-(2-methoxyethoxy)ethyl]-4-oxo-1,4-dihydro-3-quinolinecarboxamide;

(66) N-(4-chlorobenzyl)-6-{[[(1R,2S)-2-hydroxy-1-methyl-2-phenylethyl](methyl)-amino]methyl}-1-[2-(2-methoxyethoxy)ethyl]-4-oxo-1,4-dihydro-3-quinoline-carboxamide;

(67) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;

(68) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;

(69) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinoline-carboxamide;

(70) N-(4-chlorobenzyl)-6-(3-hydroxy-1-propynyl)-1-methyl-4-oxo-1,4-dihydro-3-quinolinecarbothioamide;

(71) N-(4-chlorobenzyl)-8-(3-hydroxy-1-propynyl)-1-methyl-4-oxo-1,4-dihydro-3-quinolinecarboxamide;

(72) N-(4-chlorobenzyl)-8-(4-hydroxy-1-butynyl)-1-methyl-4-oxo-1,4-dihydro-3-quinolinecarboxamide;

(73) N-(4-chlorobenzyl)-6-{[3-(hydroxyimino)-1-azetidinyl]methyl}-1-methyl-4-oxo-1,4-dihydro-3-quinolinecarboxamide;

(74) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;

(75) N-(4-chlorobenzyl)-1-([(4-chlorophenyl)sulfanyl]methyl)-6-(4-morpholinylmethyl)-4-oxo-1,4-dihydro-3-quinolinecarboxamide;

(76) N-(4-chlorobenzyl)-1-([(4-chlorophenyl)sulfinyl]methyl)-6-(4-morpholinylmethyl)-4-oxo-1,4-dihydro-3-quinolinecarboxamide;

(77) N-(4-chlorobenzyl)-1-([(4-chlorophenyl)sulfonyl]methyl)-6-(4-morpholinylmethyl)-4-oxo-1,4-dihydro-3-quinolinecarboxamide;

(78) N-(4-chlorobenzyl)-1-[(4-chlorophenoxy) methyl]-6-(4-morpholinylmethyl)-4-oxo-1,4-dihydro-3-quinolinecarboxamide;

(79) N-(4-chlorobenzyl)-1-[(2-methoxyethoxy)methyl]-6-(4-morpholinylmethyl)-4-oxo-1,4-dihydro-3-quinolinecarboxamide;

(80) 2-{[3-{[(4-chlorobenzyl)amino]carbonyl}-6-(4-morpholinylmethyl)-4-oxo-1(4H)-quinolinyl]methoxy}ethyl benzoate;

(81) N-(4-chlorobenzyl)-1-[(2-hydroxyethoxy)methyl]-6-(4-morpholinylmethyl)-4-oxo-1,4-dihydro-3-quinolinecarboxamide;

(82) N-(4-chlorobenzyl)-6-(4-morpholinylmethyl)-4-oxo-1-tetrahydro-2H-pyran-4-yl-1,4-dihydro-3-quinolinecarboxamide;

(83) N-(4-chlorobenzyl)-1-(1-methyl-4-piperidinyl)-6-(4-morpholinylmethyl)-4-oxo-1,4-dihydro-3-quinolinecarboxamide;

(84) N-(4-chlorobenzyl)-6-(4-morpholinylmethyl)-4-oxo-1-(4-piperidinyl)-1,4-dihydro-3-quinolinecarboxamide;

(85) N-(4-chlorobenzyl)-1-(1,1-dioxohexahydrothiopyran-4-yl)-6-(4-morpholinylmethyl)-4-oxo-1,4-dihydro-3-quinolinecarboxamide;

(86) N-(4-chlorobenzyl)-1-(4-morpholinyl)-6-(4-morpholinylmethyl)-4-oxo-1,4-dihydro-3-quinolinecarboxamide;

(87) N-(4-chlorobenzyl)-1-(4-methyl-1-piperazinyl)-6-(4-morpholinylmethyl)-4-oxo-1,4-dihydro-3-quinolinecarboxamide;

(88) N-(4-chlorobenzyl)-6-(4-morpholinylmethyl)-4-oxo-1-(1-piperidinyl)-1,4-dihydro-3-quinolinecarboxamide;

(89) N-(4-chlorobenzyl)-6-(4-morpholinylmethyl)-4-oxo-1-(1-pyrrolidinyl)-1,4-dihydro-3-quinolinecarboxamide;

(90) N-(4-chlorobenzyl)-1-[(2R)-2-(methoxymethyl)pyrrolidinyl]-6-(4-morpholinylmethyl)-4-oxo-1,4-dihydro-3-quinolinecarboxamide;

(91) N-(4-chlorobenzyl)-1-(dimethylamino)-6-(4-morpholinylmethyl)-4-oxo-1,4-dihydro-3-quinolinecarboxamide;

(92) 1-amino-N-(4-chlorobenzyl)-6-(3-hydroxy-1-propynyl)-4-oxo-1,4-dihydro-3-quinolinecarboxamide;

(93) 1-amino-N-(4-chlorobenzyl)-6-(3-hydroxypropyl)-4-oxo-1,4-dihydro-3-quinolinecarboxamide;

(94) N-(4-chlorobenzyl)-1-(dimethylamino)-6-(3-hydroxy-1-propynyl)-4-oxo-1,4-dihydro-3-quinolinecarboxamide;

(95) 1-(allyloxy)-N-(4-chlorobenzyl)-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) 1-(sec-butyl)-N-(4-chlorobenzyl)-6-(3-hydroxy-1-propynyl)-4-oxo-1,4-dihydro-3-quinolinecarboxamide;

(2) 1-(sec-butyl)-N-(4-chlorobenzyl)-6-(3-hydroxy-1-propynyl)-8-methoxy-4-oxo-1,4-dihydro-3-quinolinecarboxamide;

(3) N-(4-chlorobenzyl)-6-[3-hydroxy-1-propenyl]-1-[2-(4-morpholinyl)ethyl]-4-oxo-1,4-dihydro-3-quinolinecarboxamide;

(4) N-(4-chlorobenzyl)-8-fluoro-6-(3-hydroxy-1-propynyl)-1-methyl-4-oxo-1,4-dihydro-3-quinolinecarboxamide;

(5) N-(4-chlorobenzyl)-8-fluoro-6-[(Z)-3-hydroxy-1-propenyl]-1-methyl-4-oxo-1,4-dihydro-3-quinolinecarboxamide;

(6) N-(4-chlorobenzyl)-1-[2-(dimethylamino)ethyl]-6-(3-hydroxy-1-propynyl)-4-oxo-1,4-dihydro-3-quinolinecarboxamide hydrochloride;

(7) N-(4-chlorobenzyl)-6-(3-hydroxy-1-propynyl)-4-oxo-1-[2-(1-piperidinyl)ethyl]-1,4-dihydro-3-quinolinecarboxamide;

(8) N-(4-chlorobenzyl)-6-(3-hydroxy-1-propynyl)-4-oxo-1-[3-(1-piperidinyl)propyl]-1,4-dihydro-3-quinolinecarboxamide;

(9) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;

(10) N-(4-chlorobenzyl)-1-[2-(diisopropylamino)ethyl]-6-(3-hydroxy-1-propynyl)-4-oxo-1,4-dihydro-3-quinolinecarboxamide;

(11) N-(4-chlorobenzyl)-6-(3-hydroxy-1-propynyl)-4-oxo-1-[2-(1-pyrrolidinyl)ethyl]-1,4-dihydro-3-quinolinecarboxamide;

(12) N-(4-chlorobenzyl)-6-(3-hydroxy-1-propynyl)-1-[2-(4-morpholinyl)ethyl]-4-oxo-1,4-dihydro-3-quinolinecarboxamide;

(13) N-(4-chlorobenzyl)-1-[3-(dimethylamino)propyl]-6-(3-hydroxy-1-propynyl)-4-oxo-1,4-dihydro-3-quinolinecarboxamide;

(14) N-(4-chlorobenzyl)-6-(3-hydroxy-1-propynyl)-4-oxo-1-vinyl-1,4-dihydro-3-quinolinecarboxamide;

(15) N-(4-chlorobenzyl)-6-[(E)-3-hydroxy-1-propenyl]-1-methyl-4-oxo-1,4-dihydro-3-quinolinecarboxamide;

(16) N-(4-chlorobenzyl)-6-[(Z)-3-hydroxy-1-propenyl]-1-methyl-4-oxo-1,4-dihydro-3-quinolinecarboxamide;

(17) N-(4-chlorobenzyl)-1-cyclopropyl-6-(3-hydroxy-1-propynyl)-4-oxo-1,4-dihydro-3-quinolinecarboxamide;

(18) tert-butyl 2-[3-{[(4-chlorobenzyl)amino]carbonyl}-6-(3-hydroxy-1-propynyl)-4-oxo-1(4H)-quinolinyl]acetate;

(19) N-(4-chlorobenzyl)-1-(2-hydroxyethyl)-6-(3-hydroxy-1-propynyl)-4-oxo-1,4-dihydro-3-quinolinecarboxamide;

(20) N-(4-chlorobenzyl)-6-(3-hydroxy-1-propynyl)-1-methyl-4-oxo-1,4-dihydro-3-quinolinecarboxamide;

(21) 3-(3-{[(4-chlorobenzyl)aminogcarbonyl}-1-methyl-4-oxo-1,4-dihydro-6-quinolinyl)propyl dihydrogen phosphate;

(22) di(tert-butyl) 3-(3-{[(4-chlorobenzyl)amino]carbonyl)-1-cyclopropyl-4-oxo-1,4-dihydro-6-quinolinyl)propyl phosphate;

(23) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;

(24) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;

(25) N-(4-chlorobenzyl)-6-(3-hydroxy-1-propynyl)-1-[2-(2-methoxyethoxy)ethyl]-4-oxo-1,4-dihydro-3-quinolinecarboxamide;

(26) N-(4-cyanobenzyl)-6-(3-hydroxy-1-propynyl)-1-methyl-4-oxo-1,4-dihydro-3-quinolinecarboxamide;

(27) N-(4-chlorobenzyl)-6-(3-hydroxy-1-propynyl)-4-oxo-1-propyl-1,4-dihydro-3-quinolinecarboxamide;

(28) 6-(azidomethyl)-N-(4-chlorobenzyl)-1-methyl-4-oxo-1,4-dihydro-3-quinolinecarboxamide;

(29) N-(4-chlorobenzyl)-6-(2,3-dihydro-4H-1,4-benzoxazin-4-ylmethyl)-1-methyl-4-oxo-1,4-dihydro-3-quinolinecarboxamide;

(30) N-(4-chlorobenzyl)-1-methyl-4-oxo-6-vinyl-1,4-dihydro-3-quinoline-carboxamide;

(31) N-(4-chlorobenzyl)-1-[2-(2-hydroxyethoxy)ethyl]-6-(3-hydroxy-1-propynyl)-4-oxo-1,4-dihydro-3-quinolinecarboxamide;

(32) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;

(33) N-(4-chlorobenzyl)-1-[2-(2-hydroxyethoxy)ethyl]-6-(4-morpholinylmethyl)-4-oxo-1,4-dihydro-3-quinolinecarboxamide;

(34) N-(4-chlorobenzyl)-1-[2-(2-ethoxyethoxy)ethyl]-6-(4-morpholinylmethyl)-4-oxo-1,4-dihydro-3-quinolinecarboxamide;

(35) N-(4-chlorobenzyl)-1-[2-(ethylsulfanyl)ethyl]-6-(4-morpholinylmethyl)-4-oxo-1,4-dihydro-3-quinolinecarboxamide;

(36) N-(4-chlorobenzyl)-1-[3-(methylsulfanyl)propyl]-6-(4-morpholinylmethyl)-4-oxo-1,4-dihydro-3-quinolinecarboxamide;

(37) N-(4-chlorobenzyl)-1-(4-hydroxy-2-butynyl)-6-(4-morpholinylmethyl)-4-oxo-1,4-dihydro-3-quinolinecarboxamide;

(38) N-(4-chlorobenzyl)-1-[3-(methylsulfinyl)propyl]-6-(4-morpholinylmethyl)-4-oxo-1,4-dihydro-3-quinolinecarboxamide;

(39) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;

(40) N-(4-chlorobenzyl)-1-[3-(methylsulfonyl)propyl]-6-(4-morpholinylmethyl)-4-oxo-1,4-dihydro-3-quinolinecarboxamide;

(41) N-(4-chlorobenzyl)-1-[2-(ethylsulfinyl)ethyl]-6-(4-morpholinylmethyl)-4-oxo-1,4-dihydro-3-quinolinecarboxamide;

(42) N-(4-chlorobenzyl)-1-[2-(ethylsulfonyl)ethyl]-6-(4-morpholinylmethyl)-4-oxo-1,4-dihydro-3-quinolinecarboxamide;

(43) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;

(44) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;

(45) N-(4-chlorobenzyl)-6-(4-morpholinylmethyl)-4-oxo-1-[2-(phenylsulfanyl)ethyl]-1,4-dihydro-3-quinolinecarboxamide;

(46) N-(4-chlorobenzyl)-1-[(methylsulfanyl)methyl]-6-(4-morpholinylmethyl)-4-oxo-1,4-dihydro-3-quinolinecarboxamide;

(47) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;

(48) N-(4-chlorobenzyl)-6-[(3-hydroxy-1-azetidinyl)methyl]-1-methyl-4-oxo-1,4-dihydro-3-quinolinecarboxamide;

(49) N-(4-chlorobenzyl)-6-(4-morpholinylmethyl)-4-oxo-1-[(phenylsulfanyl)-methyl]-1,4-dihydro-3-quinolinecarboxamide;

(50) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;

(51) N-(4-chlorobenzyl)-6-[(3,3-dihydroxy-1-azetidinyl)methyl]-1-methyl-4-oxo-1,4-dihydro-3-quinolinecarboxamide;

(52) N-(4-chlorobenzyl)-1-[(methylsulfinyl)methyl]-6-(4-morpholinylmethyl)-4-oxo-1,4-dihydro-3-quinolinecarboxamide;

(53) N-(4-chlorobenzyl)-1-[(methylsulfonyl)methyl]-6-(4-morpholinylmethyl)-4-oxo-1,4-dihydro-3-quinolinecarboxamide;

(54) N-(4-chlorobenzyl)-6-(4-morpholinylmethyl)-4-oxo-1-[(phenylsulfinyl)-methyl]-1,4-dihydro-3-quinolinecarboxamide;

(55) N-(4-chlorobenzyl)-6-(4-morpholinylmethyl)-4-oxo-1-[(phenylsulfonyl)-methyl]-1,4-dihydro-3-quinolinecarboxamide;

(56) N-(4-chlorobenzyl)-6-(3-hydroxypropyl)-1-[2-(2-methoxyethoxy)ethyl]-4-oxo-1,4-dihydro-3-quinolinecarboxamide;

(57) N-(4-chlorobenzyl)-1-[2-(2-methoxyethoxy)ethyl]-6-(4-morpholinylmethyl)-4-oxo-1,4-dihydro-3-quinolinecarboxamide;

(58) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;

(59) N-(4-chlorobenzyl)-6-{[(3R)-3-hydroxypyrrolidinyl]methyl}-1-[2-(2-methoxyethoxy)ethyl]-4-oxo-1,4-dihydro-3-quinolinecarboxamide;

(60) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;

(61) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;

(62) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinoline-carboxamide;

(63) N-(4-chlorobenzyl)-6-(3-hydroxy-1-propynyl)-1-methyl-4-oxo-1,4-dihydro-3-quinolinecarbothioamide;

(64) N-(4-chlorobenzyl)-8-(3-hydroxy-1-propynyl)-1-methyl-4-oxo-1,4-dihydro-3-quinolinecarboxamide;

(65) N-(4-chlorobenzyl)-8-(4-hydroxy-1-butynyl)-1-methyl-4-oxo-1,4-dihydro-3-quinolinecarboxamide;

(66) N-(4-chlorobenzyl)-6-{[3-(hydroxyimino)-1-azetidinyl]methyl}-1-methyl-4-oxo-1,4-dihydro-3-quinolinecarboxamide;

(67) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;

(68) N-(4-chlorobenzyl)-1-([(4-chlorophenyl)sulfanyl]methyl)-6-(4-morpholinylmethyl)-4-oxo-1,4-dihydro-3-quinolinecarboxamide;

(69) N-(4-chlorobenzyl)-1-([(4-chlorophenyl)sulfinyl]methyl)-6-(4-morpholinylmethyl)-4-oxo-1,4-dihydro-3-quinolinecarboxamide;

(70) N-(4-chlorobenzyl)-1-([(4-chlorophenyl)sulfonyl]methyl)-6-(4-morpholinylmethyl)-4-oxo-1,4-dihydro-3-quinolinecarboxamide;

(71) N-(4-chlorobenzyl)-1-[(2-methoxyethoxy)methyl]-6-(4-morpholinylmethyl)-4-oxo-1,4-dihydro-3-quinolinecarboxamide;

(72) 2-{[3-{[(4-chlorobenzyl)amino]carbonyl}-6-(4-morpholinylmethyl)-4-oxo-1(4H)-quinolinyl]methoxy}ethyl benzoate;

(73) N-(4-chlorobenzyl)-1-[(2-hydroxyethoxy)methyl]-6-(4-morpholinylmethyl)-4-oxo-1,4-dihydro-3-quinolinecarboxamide;

(74) N-(4-chlorobenzyl)-6-(4-morpholinylmethyl)-4-oxo-1-tetrahydro-2H-pyran-4-yl-1,4-dihydro-3-quinolinecarboxamide;

(75) N-(4-chlorobenzyl)-1-(1-methyl-4-piperidinyl)-6-(4-morpholinylmethyl)-4-oxo-1,4-dihydro-3-quinolinecarboxamide;

(76) N-(4-chlorobenzyl)-6-(4-morpholinylmethyl)-4-oxo-1-(4-piperidinyl)-1,4-dihydro-3-quinolinecarboxamide;

(77) N-(4-chlorobenzyl)-1-(1,1-dioxohexahydrothiopyran-4-yl)-6-(4-morpholinylmethyl)-4-oxo-1,4-dihydro-3-quinolinecarboxamide;

(78) N-(4-chlorobenzyl)-1-(4-morpholinyl)-6-(4-morpholinylmethyl)-4-oxo-1,4-dihydro-3-quinolinecarboxamide;

(79) N-(4-chlorobenzyl)-1-(4-methyl-1-piperazinyl)-6-(4-morpholinylmethyl)-4-oxo-1,4-dihydro-3-quinolinecarboxamide;

(80) N-(4-chlorobenzyl)-6-(4-morpholinylmethyl)-4-oxo-1-(1-piperidinyl)-1,4-dihydro-3-quinolinecarboxamide;

(81) N-(4-chlorobenzyl)-1-(dimethylamino)-6-(4-morpholinylmethyl)-4-oxo-1,4-dihydro-3-quinolinecarboxamide;

(82) 1-amino-N-(4-chlorobenzyl)-6-(3-hydroxy-1-propynyl)-4-oxo-1,4-dihydro-3-quinolinecarboxamide;

(83) 1-amino-N-(4-chlorobenzyl)-6-(3-hydroxypropyl)-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-8-fluoro-6-[(Z)-3-hydroxy-1-propenyl]-1-methyl-4-oxo-1,4-dihydro-3-quinolinecarboxamide;

(2) N-(4-chlorobenzyl)-6-(3-hydroxy-1-propynyl)-1-[2-(4-morpholinyl)ethyl]-4-oxo-1,4-dihydro-3-quinolinecarboxamide;

(3) N-(4-chlorobenzyl)-6-(3-hydroxy-1-propynyl)-4-oxo-1-vinyl-1,4-dihydro-3-quinolinecarboxamide;

(4) N-(4-chlorobenzyl)-6-[(Z)-3-hydroxy-1-propenyl]-1-methyl-4-oxo-1,4-dihydro-3-quinolinecarboxamide;

(5) N-(4-chlorobenzyl)-1-cyclopropyl-6-(3-hydroxy-1-propynyl)-4-oxo-1,4-dihydro-3-quinolinecarboxamide;

(6) N-(4-chlorobenzyl)-1-(2-hydroxyethyl)-6-(3-hydroxy-1-propynyl)-4-oxo-1,4-dihydro-3-quinolinecarboxamide;

(7) N-(4-chlorobenzyl)-6-(3-hydroxy-1-propynyl)-1-methyl-4-oxo-1,4-dihydro-3-quinolinecarboxamide;

(8) 3-(3-{[(4-chlorobenzyl)amino]carbonyl}-1-methyl-4-oxo-1,4-dihydro-6-quinolinyl)propyl dihydrogen phosphate;

(9) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;

(10) N-(4-chlorobenzyl)-6-(3-hydroxy-1-propynyl)-1-[2-(2-methoxyethoxy)-ethyl]-4-oxo-1,4-dihydro-3-quinolinecarboxamide;

(11) N-(4-chlorobenzyl)-1-methyl-4-oxo-6-vinyl-1,4-dihydro-3-quinoline-carboxamide;

(12) N-(4-chlorobenzyl)-1-[2-(2-hydroxyethoxy)ethyl]-6-(3-hydroxy-1-propynyl)-4-oxo-1,4-dihydro-3-quinolinecarboxamide;

(13) N-(4-chlorobenzyl)-1-[2-(2-hydroxyethoxy)ethyl]-6-(4-morpholinylmethyl)-4-oxo-1,4-dihydro-3-quinolinecarboxamide;

(14) N-(4-chlorobenzyl)-1-[2-(2-ethoxyethoxy)ethyl]-6-(4-morpholinylmethyl)-4-oxo-1,4-dihydro-3-quinolinecarboxamide;

(15) N-(4-chlorobenzyl)-1-[2-(ethylsulfanyl)ethyl]-6-(4-morpholinylmethyl)-4-oxo-1,4-dihydro-3-quinolinecarboxamide;

(16) N-(4-chlorobenzyl)-1-[3-(methylsulfanyl)propyl]-6-(4-morpholinylmethyl)-4-oxo-1,4-dihydro-3-quinolinecarboxamide;

(17) N-(4-chlorobenzyl)-1-(4-hydroxy-2-butynyl)-6-(4-morpholinylmethyl)-4-oxo-1,4-dihydro-3-quinolinecarboxamide;

(18) N-(4-chlorobenzyl)-1-[3-(methylsulfinyl)propyl]-6-(4-morpholinylmethyl)-4-oxo-1,4-dihydro-3-quinolinecarboxamide;

(19) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;

(20) N-(4-chlorobenzyl)-1-[3-(methylsulfonyl)propyl]-6-(4-morpholinylmethyl)-4-oxo-1,4-dihydro-3-quinolinecarboxamide;

(21) N-(4-chlorobenzyl)-1-[2-(ethylsulfinyl)ethyl]-6-(4-morpholinylmethyl)-4-oxo-1,4-dihydro-3-quinolinecarboxamide;

(22) N-(4-chlorobenzyl)-1-[2-(ethylsulfonyl)ethyl]-6-(4-morpholinylmethyl)-4-oxo-1,4-dihydro-3-quinolinecarboxamide;

(23) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;

(24) N-(4-chlorobenzyl)-6-(4-morpholinylmethyl)-4-oxo-1-[2-(phenylsulfanyl)ethyl]-1,4-dihydro-3-quinolinecarboxamide;

(25) N-(4-chlorobenzyl)-1-[(methylsulfanyl)methyl]-6-(4-morpholinylmethyl)-4-oxo-1,4-dihydro-3-quinolinecarboxamide;

(26) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;

(27) N-(4-chlorobenzyl)-6-(4-morpholinylmethyl)-4-oxo-1-[(phenylsulfanyl)-methyl]-1,4-dihydro-3-quinolinecarboxamide;

(28) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;

(29) N-(4-chlorobenzyl)-6-[(3,3-dihydroxy-1-azetidinyl)methyl]-1-methyl-4-oxo-1,4-dihydro-3-quinolinecarboxamide;

(30) N-(4-chlorobenzyl)-1-[(methylsulfinyl)methyl]-6-(4-morpholinylmethyl)-4-oxo-1,4-dihydro-3-quinolinecarboxamide;

(31) N-(4-chlorobenzyl)-1-[(methylsulfonyl)methyl]-6-(4-morpholinylmethyl)-4-oxo-1,4-dihydro-3-quinolinecarboxamide;

(32) N-(4-chlorobenzyl)-6-(4-morpholinylmethyl)-4-oxo-1-[(phenylsulfinyl)-methyl]-1,4-dihydro-3-quinolinecarboxamide;

(33) N-(4-chlorobenzyl)-6-(4-morpholinylmethyl)-4-oxo-1-[(phenylsulfonyl)-methyl]-1,4-dihydro-3-quinolinecarboxamide;

(34) N-(4-chlorobenzyl)-6-(3-hydroxypropyl)-1-[2-(2-methoxyethoxy)ethyl]-4-oxo-1,4-dihydro-3-quinolinecarboxamide;

(35) N-(4-chlorobenzyl)-1-[2-(2-methoxyethoxy)ethyl]-6-(4-morpholinylmethyl)-4-oxo-1,4-dihydro-3-quinolinecarboxamide;

(36) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;

(37) N-(4-chlorobenzyl)-6-(3-hydroxy-1-propynyl)-1-methyl-4-oxo-1,4-dihydro-3-quinolinecarbothioamide;

(38) N-(4-chlorobenzyl)-8-(3-hydroxy-1-propynyl)-1-methyl-4-oxo-1,4-dihydro-3-quinolinecarboxamide;

(39) N-k(4-chlorobenzyl)-8-(4-hydroxy-1-butynyl)-1-methyl-4-oxo-1,4-dihydro-3-quinolinecarboxamide;

(40) N-(4-chlorobenzyl)-1-([(4-chlorophenyl)sulfanyl]methyl)-6-(4-morpholinylmethyl)-4-oxo-1,4-dihydro-3-quinolinecarboxamide;

(41) N-(4-chlorobenzyl)-1-([(4-chlorophenyl)sulfinyl]methyl)-6-(4-morpholinylmethyl)-4-oxo-1,4-dihydro-3-quinolinecarboxamide;

(42) N-(4-chlorobenzyl)-1-[(2-methoxyethoxy)methyl]-6-(4-morpholinylmethyl)-4-oxo-1,4-dihydro-3-quinolinecarboxamide;

(43) 2-{[3-{[(4-chlorobenzyl)amino]carbonyl}-6-(4-morpholinylmethyl)-4-oxo-1(4H)-quinolinyl]methoxy}ethyl benzoate;

(44) N-(4-chlorobenzyl)-1-[(2-hydroxyethoxy)methyl]-6-(4-morpholinylmethyl)-4-oxo-1,4-dihydro-3-quinolinecarboxamide;

(45) N-(4-chlorobenzyl)-6-(4-morpholinylmethyl)-4-oxo-1-tetrahydro-2H-pyran-4-yl-1,4-dihydro-3-quinolinecarboxamide;

(46) N-(4-chlorobenzyl)-1-(1-methyl-4-piperidinyl)-6-(4-morpholinylmethyl)-4-oxo-1,4-dihydro-3-quinolinecarboxamide;

(47) N-(4-chlorobenzyl)-6-(4-morpholinylmethyl)-4-oxo-1-(4-piperidinyl)-1,4-dihydro-3-quinolinecarboxamide;

(48) N-(4-chlorobenzyl)-1-(1,1-dioxohexahydrothiopyran-4-yl)-6-(4-morpholinylmethyl)-4-oxo-1,4-dihydro-3-quinolinecarboxamide;

(49) N-(4-chlorobenzyl)-1-(4-morpholinyl)-6-(4-morpholinylmethyl)-4-oxo-1,4-dihydro-3-quinolinecarboxamide;

(50) 1-amino-N-(4-chlorobenzyl)-6-(3-hydroxy-1-propynyl)-4-oxo-1,4-dihydro-3-quinolinecarboxamide;

(51) 1-amino-N-(4-chlorobenzyl)-6-(3-hydroxypropyl)-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-1-methyl-4-oxo-6-(tetrahydro-2H-pyran-4-ylmethyl)-1,4-dihydro-3-quinolinecarboxamide;

(2) N-(4-chlorobenzyl)-1-methyl-6-(4-morpholinylmethyl)-4-oxo-1,4-dihydro-3-quinolinecarbothioamide;

(3) N-(4-chlorobenzyl)-8-(2-hydroxyethoxy)-6-(3-hydroxypropyl)-1-methyl-4-oxo-1,4-dihydro-3-quinolinecarboxamide;

(4) N-(4-chlorobenzyl)-1-cyclopropyl-6-(3-hydroxypropyl)-4-oxo-1,4-dihydro-3-quinolinecarboxamid;

(5) 1-{2-[bis(2-hydroxyethyl)amino]ethyl}-N-(4-chlorobenzyl)-6-(4-morpholinyl-methyl)-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-8-[2-hydroxy-1-(hydroxymethyl)ethoxy]-6-(3-hydroxypropyl)-1-methyl-4-oxo-1,4-dihydro-3-quinolinecarboxamide;

(2) N-(4-chlorobenzyl)-8-fluoro-6-(hydroxymethyl)-4-oxo-1-[3-(tetrahydro-2H-pyran-2-yloxy)propyl]-1,4-dihydro-3-quinolinecarboxamide;

(3) N-(4-chlorobenzyl)-6-[ethyl(2-hydroxyethyl)amino]-1-methyl-4-oxo1,4-dihydro-3-quinolinecarboxamide;

(4) N-(4-chlorobenzyl)-1-cyclopropyl-6-(4-morpholinylmethyl)-4-oxo-1,4-dihydro-3-quinolinecarboxamide;

(5) 6-{[bis(2-hydroxyethyl)amino]methyl}-N-(4-chlorobenzyl)-1-methyl-4-oxo-1,4-dihydro-3-quinolinecarboxamide;

(6) N-(4-chlorobenzyl)-6-{[(2-hydroxyethyl)(methyl)amino]methyl}-1-methyl-4-oxo-1,4-dihydro-3-quinolinecarboxamide;

(7) 6-((benzyl(2-hydroxyethyl)amino)methyl)-N-(4-chlorobenzyl)-1-methyl-4-oxo-1,4-dihydro-3-quinolinecarboxamide;

(8) N-(4-chlorobenzyl)-6-[(4,4-difluoro-1-piperidinyl)methyl]-1-methyl-4-oxo-1,4-dihydro-3-quinolinecarboxamide;

(9) N-(4-chlorobenzyl)-6-{[4-fluoro-3,6-dihydro-1(2H)-pyridinyl]methyl}-1-methyl-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-1-[2-(2-hydroxyethoxy)ethyl]-6-(3-hydroxy-1-propynyl)-4-oxo-1,4-dihydro-3-quinolinecarboxamide;

(2) N-(4-chlorobenzyl)-6-{[[2-hydroxy-2-(4-hydroxyphenyl)ethyl](methyl)amino]methyl}-1-methyl-4-oxo-1,4-dihydro-3-quinolinecarboxamide;

(3) N-(4-chlorobenzyl)-6-{[[2-hydroxy-2-(4-hydroxy-3-methoxyphenyl)ethyl](methyl)amino]methyl}-1-methyl-4-oxo-1,4-dihydro-3-quinolinecarboxamide;

(4) N-(4-chlorobenzyl)-6-[(3,3-dihydroxy-1-azetidinyl)methyl]-1-methyl-4-oxo-1,4-dihydro-3-quinolinecarboxamide;

(5) N-(4-chlorobenzyl)-8-fluoro-6-[(Z)-3-hydroxy-1-propenyl]-1-methyl-4-oxo-1,4-dihydro-3-quinolinecarboxamide;

(6) N-(4-chlorobenzyl)-6-(3-hydroxy-1-propynyl)-1-[2-(4-morpholinyl)ethyl]-4-oxo-1,4-dihydro-3-quinolinecarboxamide;

(7) N-(4-chlorobenzyl)-6-[(Z)-3-hydroxy-1-propenyl]-1-methyl-4-oxo-1,4-dihydro-3-quinolinecarboxamide;

(8) N-(4-chlorobenzyl)-1-cyclopropyl-6-(3-hydroxy-1-propynyl)-4-oxo-1,4-dihydro-3-quinolinecarboxamide;

(9) N-(4-chlorobenzyl)-1-(2-hydroxyethyl)-6-(3-hydroxy-1-propynyl)-4-oxo-1,4-dihydro-3-quinolinecarboxamide;

(10) N-(4-chlorobenzyl)-6-(3-hydroxy-1-propynyl)-1-methyl-4-oxo-1,4-dihydro-3-quinolinecarboxamide;

(11) N-(4-chlorobenzyl)-6-(3-hydroxy-1-propynyl)-1-[2-(2-methoxyethoxy)ethyl]-4-oxo-1,4-dihydro-3-quinolinecarboxamide;

(12) N-(4-chlorobenzyl)-6-(3-hydroxy-1-propynyl)-1-methyl-4-oxo-1,4-dihydro-3-quinolinecarbothioamide;

(13) N-(4-chlorobenzyl)-8-(3-hydroxy-1-propynyl)-1-methyl-4-oxo-1,4-dihydro-3-quinolinecarboxamide;

(14) N-(4-chlorobenzyl)-8-(4-hydroxy-1-butynyl)-1-methyl-4-oxo-1,4-dihydro-3-quinolinecarboxamide;

(15) N-(4-chlorobenzyl)-1-methyl-4-oxo-6-(tetrahydro-2H-pyran-4-ylmethyl)-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-1-(4-morpholinyl)-6-(4-morpholinylmethyl)-4-oxo-1,4-dihydro-3-quinolinecarboxamide;

(2) 1-amino-N-(4-chlorobenzyl)-6-(3-hydroxy-1-propynyl)-4-oxo-1,4-dihydro-3-quinolinecarboxamide;

(3) 1-amino-N-(4-chlorobenzyl)-6-(3-hydroxypropyl)-4-oxo-1,4-dihydro-3-quinolinecarboxamide;

(4) N-(4-bromobenzyl)-1-(4-morpholinyl)-6-(4-morpholinylmethyl)-4-oxo-1,4-dihydro-3-quinolinecarboxamide;

(5) N-(4-fluorobenzyl)-1-(4-morpholinyl)-6-(4-morpholinylmethyl)-4-oxo-1,4-dihydro-3-quinolinecarboxamide;

or a pharmaceutically acceptable salt thereof.

A method for preventing or treating atherosclerosis or restenosis in mammals, wherein the compound administered has the Formula III and is selected from the group consisting of

(1) N-(4-chlorobenzyl)-1-{[(4-chlorophenyl)sulfanyl]methyl}-6-(4-morpholinylmethyl)-4-oxo-1,4-dihydro-3-quinolinecarboxamide;

(2) N-(4-chlorobenzyl)-1-{[(4-chlorophenyl)sulfinyl]methyl}-6-(4-morpholinylmethyl)-4-oxo-1,4-dihydro-3-quinolinecarboxamide;

(3) N-(4-chlorobenzyl)-1-[(2-methoxyethoxy)methyl]-6-(4-morpholinylmethyl)-4-oxo-1,4-dihydro-3-quinolinecarboxamide;

(4) 2-{[3-{[(4-chlorobenzyl)amino]carbonyl}-6-(4-morpholinylmethyl)-4-oxo-1(4H)-quinolinyl]methoxy}ethyl benzoate;

(5) N-(4-chlorobenzyl)-1-[(2-hydroxyethoxy)methyl]-6-(4-morpholinylmethyl)-4-oxo-1,4-dihydro-3-quinolinecarboxamide;

(6) N-(4-chlorobenzyl)-6-(4-morpholinylmethyl)-4-oxo-1-tetrahydro-2H-pyran-4-yl-1,4-dihydro-3-quinolinecarboxamide;

(7) N-(4-chlorobenzyl)-1-(1-methyl-4-piperidinyl)-6-(4-morpholinylmethyl)-4-oxo-1,4-dihydro-3-quinolinecarboxamide;

(8) N-(4-chlorobenzyl)-6-(4-morpholinylmethyl)-4-oxo-1-(4-piperidinyl)-1,4-dihydro-3-quinolinecarboxamide;

(9) N-(4-chlorobenzyl)-1-(1,1-dioxohexahydro-llambda˜6˜-thiopyran-4-yl)-6-(4-morpholinylmethyl)-4-oxo-1,4-dihydro-3-quinolinecarboxamide;

(10) N-(4-chlorobenzyl)-1-(4-morpholinyl)-6-(4-morpholinylmethyl)-4-oxo-1,4-dihydro-3-quinolinecarboxamide;

(11) N-(4-chlorobenzyl)-1-[2-(2-hydroxyethoxy)ethyl]-6-(4-morpholinylmethyl)-4-oxo-1,4-dihydro-3-quinolinecarboxamide;

(12) N-(4-chlorobenzyl)-1-[2-(2-ethoxyethoxy)ethyl]-6-(4-morpholinylmethyl)-4-oxo-1,4-dihydro-3-quinolinecarboxamide;

(13) N-(4-chlorobenzyl)-1-[2-(ethylsulfanyl)ethyl]-6-(4-morpholinylmethyl)-4-oxo-1,4-dihydro-3-quinolinecarboxamide;

(14) N-(4-chlorobenzyl)-1-[3-(methylsulfanyl)propyl]-6-(4-morpholinylmethyl)-4-oxo-1,4-dihydro-3-quinolinecarboxamide;

(15) N-(4-chlorobenzyl)-1-(4-hydroxy-2-butynyl)-6-(4-morpholinylmethyl)-4-oxo-1,4-dihydro-3-quinolinecarboxamide;

(16) 1-{2-[bis(2-hydroxyethyl)amino]ethyl}-N-(4-chlorobenzyl)-6-(4-morpholinylmethyl)-4-oxo-1,4-dihydro-3-quinolinecarboxamide;

(17) N-(4-chlorobenzyl)-1-[3-(methylsulfinyl)propyl]-6-(4-morpholinylmethyl)-4-oxo-1,4-dihydro-3-quinolinecarboxamide;

(18) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;

(19) N-(4-chlorobenzyl)-1-[3-(methylsulfonyl)propyl]-6-(4-morpholinylmethyl)-4-oxo-1,4-dihydro-3-quinolinecarboxamide;

(20) N-(4-chlorobenzyl)-1-[2-(ethylsulfinyl)ethyl]-6-(4-morpholinylmethyl)-4-oxo-1,4-dihydro-3-quinolinecarboxamide;

(21) N-(4-chlorobenzyl)-1-[2-(ethylsulfonyl)ethyl]-6-(4-morpholinylmethyl)-4-oxo-1,4-dihydro-3-quinolinecarboxamide;

(22) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;

(23) N-(4-chlorobenzyl)-6-(4-morpholinylmethyl)-4-oxo-1-[2-(phenylsulfanyl)ethyl]-1,4-dihydro-3-quinolinecarboxamide;

(24) N-(4-chlorobenzyl)-1-[(methylsulfanyl)methyl]-6-(4-morpholinylmethyl)-4-oxo-1,4-dihydro-3-quinolinecarboxamide;

(25) N-(4-chlorobenzyl)-6-(4-morpholinylmethyl)-4-oxo-1-[(phenylsulfanyl)methyl]-1,4-dihydro-3-quinolinecarboxamide;

(26) N-(4-chlorobenzyl)-1-[(methylsulfinyl)methyl]-6-(4-morpholinylmethyl)-4-oxo-1,4-dihydro-3-quinolinecarboxamide;

(27) N-(4-chlorobenzyl)-1-[(methylsulfonyl)methyl]-6-(4-morpholinylmethyl)-4-oxo-1,4-dihydro-3-quinolinecarboxamide;

(28) N-(4-chlorobenzyl)-6-(4-morpholinylmethyl)-4-oxo-1-[(phenylsulfinyl)methyl]-1,4-dihydro-3-quinolinecarboxamide;

(29) N-(4-chlorobenzyl)-6-(4-morpholinylmethyl)-4-oxo-1-[(phenylsulfonyl)methyl]-1,4-dihydro-3-quinolinecarboxamide;

(30) N-(4-chlorobenzyl)-1-[2-(2-methoxyethoxy)ethyl]-6-(4-morpholinylmethyl)-4-oxo-1,4-dihydro-3-quinolinecarboxamide;

(31) 1-12-[2-(tert-butoxy)ethoxy]ethyl}-N-(4-chlorobenzyl)-6-(4-morpholinylmethyl)-4-oxo-1,4-dihydro-3-quinolinecarboxamide;

(32) N-(4-chlorobenzyl)-1-cyclopropyl-6-(4-morpholinylmethyl)-4-oxo-1,4-dihydro-3-quinolinecarboxamide;

or a pharmaceutically acceptable salt thereof.

A method for preventing or treating atherosclerosis in mammals, wherein the compound administered has the Formula IV and R^{IV-1} is propyl.

A method for preventing or treating atherosclerosis in mammals, wherein the compound administered has the Formula IV and R^{IV-1} is 3-hydroxypropyl.

A method for preventing or treating atherosclerosis in mammals, wherein the compound administered has the Formula IV and R^{IV-1} is 3-hydroxy-1-propynyl.

A method for preventing or treating atherosclerosis in mammals, wherein the compound administered has the Formula IV and het^{IV} is morpholine, thiomorpholine, piperidine, piperazine or pyrrolidine.

A method for preventing or treating atherosclerosis in mammals, wherein the compound administered has the Formula IV and R^{IV-1} is 4-morpholinylmethyl.

A method for preventing or treating atherosclerosis in mammals, wherein the compound administered has the Formula IV and is selected from the group consisting of

(a) N-(4-Chlorobenzyl)-9-(4-morpholinylmethyl)-7-oxo-2,3-dihydro-7H-[1,4]oxazino[2,3,4-ij]quinoline-6-carboxamide,

(b) N-(4-Chlorobenzyl)-9-(3-hydroxy-1-propynyl)-7-oxo-2,3-dihydro-7H-[1,4]oxazino[2,3,4-ij]quinoline-6-carboxamide,

(c) N-(4-Chlorobenzyl)-9-(3-hydroxypropyl)-7-oxo-2,3-dihydro-7H-[1,4]oxazino[2,3,4-ij]quinoline-6-carboxamide, or

(d) N-(4-Chlorobenzyl)-7-oxo-9-propyl-2,3-dihydro-7H-[1,4]oxazino[2,3,4-ij]quinoline-6-carboxamide.

A method for preventing or treating atherosclerosis in mammals, wherein the compound administered has the Formula IV and is N-(4-Chlorobenzyl)-9-(4-morpholinylmethyl)-7-oxo-2,3-dihydro-7H-[1,4]oxazino[2,3,4-ij]quinoline-6-carboxamide.

A method for preventing or treating atherosclerosis or restenosis, wherein the compound administered has the Formula V-A

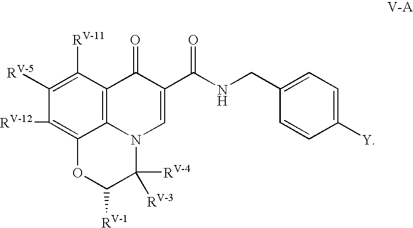

A method for preventing or treating atherosclerosis or restenosis, wherein the compound administered has the Formula V-B

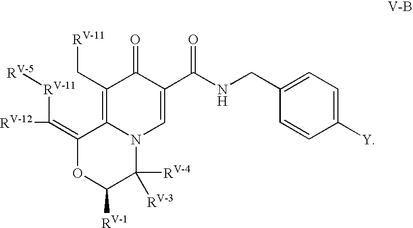

A method for preventing or treating atherosclerosis or restenosis, wherein the compound administered has the Formula V-C

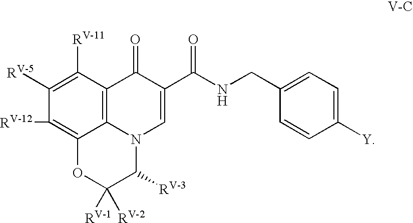

A method for preventing or treating atherosclerosis or restenosis, wherein the compound administered has the Formula V-D

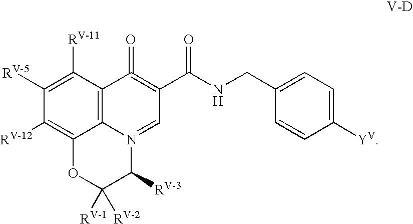

A method for preventing or treating atherosclerosis or restenosis, wherein the compound administered has the Formula V-E

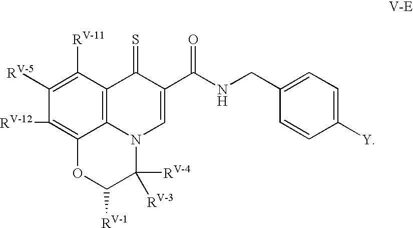

A method for preventing or treating atherosclerosis or restenosis, wherein the compound administered has the Formula V-F

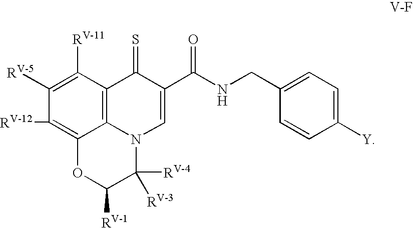

A method for preventing or treating atherosclerosis or restenosis, wherein the compound administered has the Formula V-G

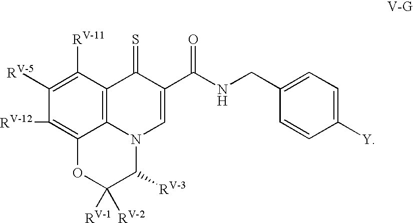

A method for preventing or treating atherosclerosis or restenosis, wherein the compound administered has the Formula V-H

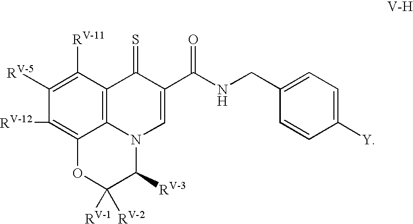

A method for preventing or treating atherosclerosis or restenosis, wherein the compound administered has the Formula V-I

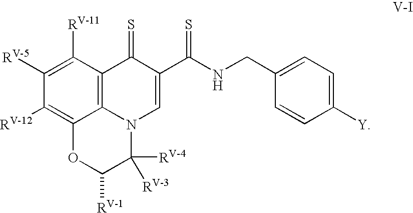

A method for preventing or treating atherosclerosis or restenosis, wherein the compound administered has the Formula V-J

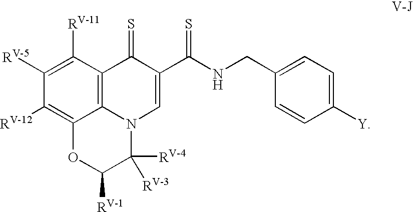

A method for preventing or treating atherosclerosis or restenosis, wherein the compound administered has the Formula V-K

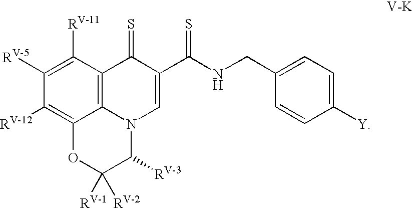

A method for preventing or treating atherosclerosis or restenosis, wherein the compound administered has the Formula V-L

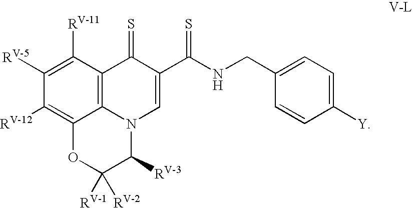

A method for preventing or treating atherosclerosis or restenosis, wherein the compound administered has the Formula V-M

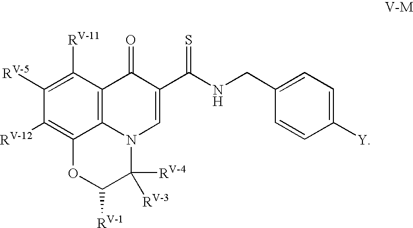

A method for preventing or treating atherosclerosis or restenosis, wherein the compound administered has the Formula V-N

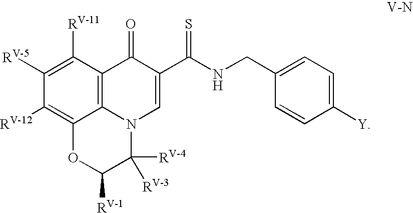

A method for preventing or treating atherosclerosis or restenosis, wherein the compound administered has the Formula V-O

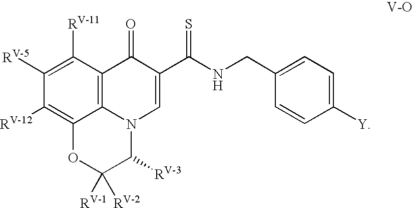

A method for preventing or treating atherosclerosis or restenosis, wherein the compound administered has the Formula V-P

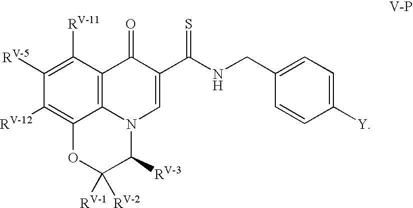

A method for preventing or treating atherosclerosis or restenosis in mammals, wherein the compound administered has the Formula V and R^{V-11} is H, halo, or C₁₋₄alkyl optionally substituted with one to three halo; and

R^{V-12} is

a) H,

b) SOᵢR^{V-8},

c) OR^{V-}8,

d) C(═O)OR^{V-8},

e) C(═O)R^{V-8},

f) NR^{V-8}R^{V-9},

g) SOᵢR^{V-8}R^{V-9}, or

h) C₁₋₈alkyl, which may be partially unsaturated and optionally substituted with one to three N₃, halo, CN, or R^{V-6}.

A method for preventing or treating atherosclerosis or restenosis in mammals, wherein the compound administered has the Formula V and R^{V-11} and R^{V-12} are hydrogen.

A method for preventing or treating atherosclerosis or restenosis in mammals, wherein the compound administered has the Formula V and R^{V-5} is C₁₋₈alkyl substituted with OR₈ or het^{V}.

A method for preventing or treating atherosclerosis or restenosis in mammals, wherein the compound administered has the Formula V and R^{V-5} is C₁₋₆alkyl substituted with OH.

A method for preventing or treating atherosclerosis or restenosis in mammals, wherein the compound administered has the Formula V and R^{V-5} is C₁₋₄alkyl substituted with het^{V}.

A method for preventing or treating atherosclerosis or restenosis in mammals, wherein the compound administered has the Formula V and het^{V} is morpholinyl or thiomorpholinyl.

A method for preventing or treating atherosclerosis or restenosis in mammals, wherein the compound administered has the Formula V and R^{V-5} is 4-morpholinylmethyl.

A method for preventing or treating atherosclerosis or restenosis in mammals, wherein the compound administered has the Formula V and R^{V-5} is C₁₋₈alkyl which is partially unsaturated and optionally substituted with OR^{V-8}.

A method for preventing or treating atherosclerosis or restenosis in mammals, wherein the compound administered has the Formula V and R^{V-5} is propynyl.

A method for preventing or treating atherosclerosis or restenosis in mammals, wherein the compound administered has the Formula V and propynyl is substituted with OH.

A method for preventing or treating atherosclerosis or restenosis in mammals, wherein the compound administered has the Formula V and Y is Cl.

A method for preventing or treating atherosclerosis or restenosis in mammals, wherein the compound administered has the Formula V and R^{V-3} and R^{V-4} are independently hydrogen.

A method for preventing or treating atherosclerosis or restenosis in mammals, wherein the compound administered has the Formula V and R^{V-1} and R^{V-2} are independently

a) hydrogen,

b) fluoro,

c) C₁₋₈ alkyl substituted with R^{V-6} or OR^{V-7};

d) aryl^{V},

e) het^{V}, or

f) R^{V-1} and R^{V-2} together with the carbon to which they are attached form a six- (6) membered cycloalkyl or a het^{V};

wherein R^{V-6} is

a) het^{V},

b) SOᵢR^{V-8},

c) OR^{V-8} or

d) NR^{V-8}R^{V-9};

wherein R^{V-7} is

a) P(═O)(OR₁₀)₂,

b) CO(CH₂)ₙCON(CH₃)(CH₂)ₙSO₃⁻M^{V+}, or

c) C(═O)C₁₋₆alkyl,

wherein R^{V-8} and R^{V-9} are independently

a) hydrogen,

b) aryl^{V},

c) het^{V}, or

d) C₁₋₈alkyl which is further optionally substituted with one or more aryl^{V}, het^{V}, halo, CO₂R^{V-10}, SOᵢR^{V-10}, or OR^{V-10};

wherein R^{V-10} is

a) H or

b) C₁₋₄alkyl, optionally substituted with OH.

A method for preventing or treating atherosclerosis or restenosis in mammals, wherein the compound administered has the Formula V and R^{V-1} and R^{V-2} are independently H, C₁₋₄alkyl substituted with OR^{V-8} wherein R^{V-8} is H, or C₁₋₄alkyl substituted with OR^{V-10}.

A method for preventing or treating atherosclerosis or restenosis in mammals, wherein the compound administered has the Formula V and R^{V-1} is H; R^{V-2} is aryl^{V} wherein aryl^{V} is optionally substituted with one or two halo, CN, OR^{V-10}, or C₁₋₄alkyl substituted with OR^{V-10}.

A method for preventing or treating atherosclerosis or restenosis in mammals, wherein the compound administered has the Formula V and R^{V-1} is H; R^{V-2} is aryl^{V} wherein aryl^{V} is fused with a heterocyclic ring.

A method for preventing or treating atherosclerosis or restenosis in mammals, wherein the compound administered has the Formula V and R^{V-2} is 1,3-benzodioxolyl or 1,4-benxodioxinyl.

A method for preventing or treating atherosclerosis or restenosis in mammals, wherein the compound administered has the Formula V and R^{V-1} is H; R^{V-2} is het^{V}.

A method for preventing or treating atherosclerosis or restenosis in mammals wherein the compound administered has the Formula V and het^{V} is a five- (5) or six- (6) membered saturated or unsaturated heterocyclic ring having 1, 2, or 3 heteroatoms selected from the group consisting of O, S, and NW^{V}, wherein W^{V} is hydrogen, C₁₋₄alkyl, C(═O)OC₁₋₄alkyl or absent, wherein het^{V} may be substituted with one or more halo, C₁₋₄alkyl, CF₃, oxo or oxine.

A method for preventing or treating atherosclerosis or restenosis in mammals, wherein the compound administered has the Formula V and het^{V} is pyridinyl.

A method for preventing or treating atherosclerosis or restenosis in mammals, wherein the compound administered has the Formula V and het^{V} is a five- (5) membered heterocyclic ring.

A method for preventing or treating atherosclerosis or restenosis in mammals, wherein the compound administered has the Formula V and R^{V-1} and R^{V-2} together with the carbon to which they are attached form a het^{V}.

A method for preventing or treating atherosclerosis or restenosis in mammals, wherein the compound administered has the Formula V and het^{V} is a five- (5) or six- (6) membered heterocyclic ring having 1, 2, or 3 heteroatoms selected from the group consisting of O, S, and NW^{V}, wherein W^{V} is hydrogen, C₁₋₄alkyl, or C(═O)OC₁₋₄alkyl, wherein v-het may be substituted with one or more halo, OR^{V-10}, C₁₋₄alkyl, CF₃, oxo or oxine.

A method for preventing or treating atherosclerosis or restenosis in mammals, wherein the compound administered has the Formula V and het^{V} is a (6) membered heterocyclic ring.

A method for preventing or treating atherosclerosis or restenosis in mammals, wherein the compound administered has the Formula V and het^{V} is pyran, piperdine, or thiopyran.

A method for preventing or treating atherosclerosis or restenosis in mammals, wherein the compound administered has the Formula V and R^{V-1} and R^{V-2} together with the carbon to which they are attached form a six- (6) membered cycloalkyl.

A method for preventing or treating atherosclerosis or restenosis in mammals, wherein the compound administered has the Formula V and cycloalkyl is optionally substituted with oxo, or OR^{V-10}.

A method for preventing or treating atherosclerosis or restenosis in mammals wherein the compound administered has the Formula V and R^{V-2} is hydrogen;

R^{V-1} is C₁₋₈ alkyl substituted with R^{V-6} or OR^{V-7};

where R^{V-6} is

a) het^{V},

b) SR^{V-8},

c) OR^{V-8} or

d) NR^{V-8}R^{V-9};

wherein R^{V-7} is

a) (P═O)(OCH₃)₂,

b) CO(CH₂)ₙCON(CH₃)(CH₂)ₙSO₃⁻M^{V+}, or

c) C(═O)CH₃,

wherein R^{V-8} and R^{V-9} are independently

a) hydrogen,

b) het^{V}, or

c) C₁₋₈alkyl, which is optionally substituted with one or two het^{V}, CO₂R^{V-10}, SOᵢR^{V-10}, or OR^{V-10}; and

wherein R^{V-10} is

a) H or

b) C₁₋₄alkyl, optionally substituted with OH, or OC₁₋₄alkyl.

A method for preventing or treating atherosclerosis or restenosis in mammals, wherein the compound administered has the Formula V and R₁ is C₁₋₈ alkyl substituted with het^{V}.

A method for preventing or treating atherosclerosis or restenosis in mammals, wherein the compound administered has the Formula V and het^{V} is piperidinyl, morpholinyl, thiomorpholinyl, piperazinyl, N—C₁₋₄alkyl substituted piperazinyl, pyrrolidinyl, pyridyl, imidazolyl, or N—C₁₋₄alky substituted imidazol.

A method for preventing or treating atherosclerosis or restenosis in mammals, wherein the compound administered has the Formula V and het^{V} is morpholinyl.

A method for preventing or treating atherosclerosis or restenosis in mammals, wherein the compound administered has the Formula V and het^{V} is pyridinyl.

A method for preventing or treating atherosclerosis or restenosis in mammals, wherein the compound administered has the Formula V and R^{V-1} is C₁₋₈ alkyl substituted with OH or OC₁₋₄alkyl.

A method for preventing or treating atherosclerosis or restenosis in mammals, wherein the compound administered has the Formula V and R^{V-1} is C₁₋₄ alkyl substituted with OH.

A method for preventing or treating atherosclerosis or restenosis in mammals, wherein the compound administered has the Formula V and R^{V-1} is C₁₋₈ alkyl substituted with SR^{V-8}.

A method for preventing or treating atherosclerosis or restenosis in mammals, wherein the compound administered has the Formula V and R^{V-8} is het^{V}.

A method for preventing or treating atherosclerosis or restenosis in mammals, wherein the compound administered has the Formula V and R^{V-8} is C₁₋₄alkyl optionally substituted with one or two OR^{V-10}.

A method for preventing or treating atherosclerosis or restenosis in mammals, wherein the compound administered has the Formula V and R^{V-1} is C₁₋₄ alkyl substituted with NR^{V-8}R^{V-9}.

A method for preventing or treating atherosclerosis or restenosis in mammals, wherein the compound administered has the Formula V and R^{V-8} is H, and R^{V-9} is het^{V}.

A method for preventing or treating atherosclerosis or restenosis in mammals, wherein the compound administered has the Formula V and het^{V} is a six- (6) membered heterocyclic ring having 1, 2, or 3 heteroatoms selected from the group consisting of O, S, and NW, wherein W is hydrogen, C₁₋₄alkyl, or absent.

A method for preventing or treating atherosclerosis or restenosis in mammals, wherein the compound administered has the Formula V and het^{V} is pyridinyl.

A method for preventing or treating atherosclerosis or restenosis in mammals, wherein the compound administered has the Formula V and R^{V-8} is H, and R^{V-9} is C₁₋₈alkyl optionally substituted with het^{V}.

A method for preventing or treating atherosclerosis or restenosis in mammals, wherein the compound administered has the Formula V and het^{V} is a six- (6) membered heterocyclic ring having 1, 2, or 3 heteroatoms selected from the group consisting of O, S, and NW, wherein W is hydrogen, C₁₋₄alkyl, or absent.

A method for preventing or treating atherosclerosis or restenosis in mammals, wherein the compound administered has the Formula V and het^{V} is pyridinyl.

A method for preventing or treating atherosclerosis or restenosis in mammals, wherein the compound administered has the Formula V and R^{V-8} is H, and R^{V-9} is C₁₋₈alkyl optionally substituted with one or two OR^{V-10}.

A method for preventing or treating atherosclerosis or restenosis in mammals, wherein the compound administered has the Formula V and R^{V-1} is hydroxymethyl, morpholinylmethyl, (pyridinylmethyl)aminomethyl, (dimethylamino)methyl, (hydroxyethyl)sulfanylmethyl, (1-methyl-1H-imidazol-2-yl)sulfanylmethyl, —CH₂OCO(CH₂)₆CON(CH₃)(CH₂)₂SO₃⁻M^{V+}, —CH₂OC(═O)CH₃, CH₂OP(═O)(OMe)₂, (4-methyl-1-piperazinyl)methyl, 1-pyrrolidinylmethyl, (2,3-dihydroxypropyl)aminomethyl, (2-hydroxyethyl)aminomethyl, 1-piperidinylmethyl, bis(2-hydroxyethyl)aminomethyl, 1H-imidazol-1-ylmethyl, (methylsulfanyl)methyl, (tert-butylsulfanyl)methyl, methylsulfanyl acetate, (2,3-dihydroxypropyl)sulfanylmethyl, phenyl or fluoro.

A method for preventing or treating atherosclerosis or restenosis in mammals, wherein the compound administered has the Formula V and R^{V-1} is hydroxymethyl, morpholinylmethyl, (2-pyridinylmethyl)aminomethyl, (3-pyridinylmethyl)aminomethyl, (dimethylamino)methyl, (2-hydroxyethyl)sulfanylmethyl, (1-methyl-1H-imidazol-2-yl)sulfanylmethyl, OP(═O)(OCH₃)₂, —CH₂OCO(CH₂)₆CON(CH₃)(CH₂)₂SO₃⁻M⁺, or —CH₂OC(═O)CH₃. ₁₋₈alkyl optionally substituted with one or two OR^{V-10}.

A method for preventing or treating atherosclerosis or restenosis in mammals, wherein the compound administered has the Formula V R^{V-1} and R^{V-2} are independently hydrogen, R^{V-3} and R^{V-4} are independently

a) hydrogen,

b) fluoro, or

c) C₁₋₈ alkyl substituted with R^{V-6} or OR^{V-7};

where R^{V-6} is

a) het^{V},

b) SOᵢR^{V-8},

c) OR^{V-8} or

d) NR^{V-8}R^{V-9};

wherein R^{V-7} is

a) P═O)(OH)₂,

b) (P═O)(C₁₋₄alkoxy)₂,

c) CO(CH₂)ₙCON(CH₃)(CH₂)ₙSO₃⁻M⁺, or

d) C(═O)C₁₋₆alkyl,

wherein R^{V-8} and R^{V-9} are independently

a) hydrogen,

b) aryl^{V},

c) het^{V}, or

d) C₁₋₈alkyl which is further optionally substituted with one or more aryl^{V}, het^{V}, halo, CO₂R^{V-10}, SOᵢR^{V-10}, or OR^{V-10};

wherein R^{V-10} is

a) H or

b) C₁₋₄alkyl, optionally substituted with OH. ₁₋₈alkyl optionally substituted with one or two OR^{V-10},

A method for preventing or treating atherosclerosis or restenosis in mammals, wherein the compound administered has the Formula V R^{V-3} and R^{V-4} are independently fluoro or hydroxymethyl. ₁₋₈alkyl optionally substituted with one or two OR^{V-10}, A method for preventing or treating atherosclerosis or restenosis in mammals, wherein the compound administered has the Formula V R^{V-3} is hydrogen and R^{V-4} is phenyl, morpholinylmethyl, or hydroxymethyl. ₁₋₈-alkyl optionally substituted with one or two OR^{V-10}.

A method for preventing or treating atherosclerosis or restenosis in mammals, wherein the compound administered has the Formula V R^{V-4} is morpholinylmethyl. ₁₋₈alkyl optionally substituted with one or two OR^{V-10}.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxamide,

(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-oxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-oxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-oxo-2,3-dihydro-7H-[1,4]oxazino[2,3,4-ij]quinoline-6-carboxamide,

(j) N-(4-Chlorobenzyl)-2-{[(1-methyl-1H-imidazol-2-yl)sulfanyl]methyl}-9-(morpholin-4-ylmethyl)-7-oxo-2,3-dihydro-7H-[1,4]oxazino[2,3,4-ij]quinoline-6-carboxamide,

(k) N-(4-Chlorobenzyl)-9-(morpholin-4-ylmethyl)-7-oxo-2-{[(3-pyridinylmethyl)amino]methyl}-2,3-dihydro-7H-[1,4]oxazino[2,3,4-ij]quinoline-6-carboxamide,

(l) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-oxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-oxo-2,3-dihydro-7H-[1,4]oxazino[2,3,4-ij]quinoline-6-carboxamide,

(o) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-oxo-2,3-dihydro-7H-[1,4]oxazino[2,3,4-ij]quinoline-6-carboxamide,

(q) N-(4-Chlorobenzyl)-2-[2-(hydroxymethyl)phenyl]-9-(morpholin-4-ylmethyl)-7-oxo-2,3-dihydro-7H-[1,4]oxazino[2,3,4-ij]quinoline-6-carboxamide,

(r) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-oxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-oxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-oxo-2,3-dihydro-7H-[1,4]oxazino[2,3,4-ij]quinoline-6-carboxamide,

(v) N-(4-Chlorobenzyl)-9-(morpholin-4-ylmethyl)-7-oxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-oxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-oxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,

(bb) N-(4-Chlorobenzyl)-9-(morpholin-4-ylmethyl)-7-oxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-oxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4,7′-dioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-oxospiro[cyclohexane-1,2′(3′H)-[7H]pyrido[1,2,3-de] [1,4]benzoxazine]-6′-carboxamide,

(ff) N-(4-Chlorobenzyl)-3,9-bis(morpholin-4-ylmethyl)-7-oxo-2,3-dihydro-7H-[1,4]oxazino[2,3,4-ij]quinoline-6-carboxamide,

(gg) N-(4-Chlorobenzyl)-9-(morpholin-4-ylmethyl)-7-oxo-2-phenyl-2,3-dihydro-7H-[1,4]oxazino[2,3,4-ij]quinoline-6-carboxamide,

(hh) N-(4-Chlorobenzyl)-2,2-difluoro-9-(morpholin-4-ylmethyl)-7-oxo-2,3-dihydro-7H-[1,4]oxazino[2,3,4-ij]quinoline-6-carboxamide,

(ii) N-(4-Chlorobenzyl)-2-[(methylsulfanyl)methyl]-9-(morpholin-4-ylmethyl)-7-oxo-2,3-dihydro-7H-[1,4]oxazino[2,3,4-ij]quinoline-6-carboxamide,

(jj) N-(4-Chlorobenzyl)-2-[(dimethylamino)methyl]-9-(morpholin-4-ylmethyl)-7-oxo-2,3-dihydro-7H-[1,4]oxazino[2,3,4-ij]quinoline-6-carboxamide,

(kk) N-(4-Chlorobenzyl)-2-[(4-methyl-1-piperazinyl)methyl]-9-(morpholin-4-ylmethyl)-7-oxo-2,3-dihydro-7H-[1,4]oxazino[2,3,4-ij]quinoline-6-carboxamide,

(ll) Methyl ({[6-{[(4-chlorobenzyl)amino]carbonyl}-9-(morpholin-4-ylmethyl)-7-oxo-2,3-dihydro-7H-[1,4]oxazino[2,3,4-ij]quinolin-2-yl]methyl}thio)acetate,

(mm) N-(4-Chlorobenzyl)-9-(morpholin-4-ylmethyl)-7-oxo-2-(1-pyrrolidinylmethyl)-2,3-dihydro-7H-[1,4]oxazino[2,3,4-ij]quinoline-6-carboxamide,

(nn) N-(4-Chlorobenzyl)-2-{[(2,3-dihydroxypropyl)sulfanyl]methyl}-9-(morpholin-4-ylmethyl)-7-oxo-2,3-dihydro-7H-[1,4]oxazino[2,3,4-ij]quinoline-6-carboxamide,

(oo) N-(4-Chlorobenzyl)-2-{[(2,3-dihydroxypropyl)amino]methyl}-9-(morpholin-4-ylmethyl)-7-oxo-2,3-dihydro-7H-[1,4]oxazino[2,3,4-ij]quinoline-6-carboxamide,

(pp) N-(4-Chlorobenzyl)-2-{[(2-hydroxyethyl)amino]methyl}-9-(morpholin-4-ylmethyl)-7-oxo-2,3-dihydro-7H-[1,4]oxazino[2,3,4-ij]quinoline-6-carboxamide,

(qq) N-(4-Chlorobenzyl)-9-(morpholin-4-ylmethyl)-7-oxo-2-(1-piperidinylmethyl)-2,3-dihydro-7H-[1,4]oxazino[2,3,4-ij]quinoline-6-carboxamide,

(rr) 2-{[bis(2—Hydroxyethyl)amino]methyl}-N-(4-chlorobenzyl)-9-(morpholin-4-ylmethyl)-7-oxo-2,3-dihydro-7H-[1,4]oxazino[2,3,4-ij]quinoline-6-carboxamide,

(ss) N-(4-Chlorobenzyl)-9-(morpholin-4-ylmethyl)-7-oxo-2-{[(2-pyridinylmethyl)amino]methyl}-2,3-dihydro-7H-[1,4]oxazino[2,3,4-ij]quinoline-6-carboxamide,

(tt) 2-[(8-{[6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methoxy}-8-oxooctanoyl)(methyl)amino]ethanesulfonic acid sodium salt,

(uu) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl dimethyl phosphate,

(vv) N-(4-Chlorobenzyl)-9-(morpholin-4-ylmethyl)-7-oxo-2-{[(4pyridinylmethyl)amino]methyl}-2,3-dihydro-7H-[1,4]oxazino[2,3,4-ij]quinoline-6-carboxamide,

(ww) N-(4-Chlorobenzyl)-2-(1H-imidazol-1-ylmethyl)-9-(morpholin-4-ylmethyl)-7-oxo-2,3-dihydro-7H-[1,4]oxazino[2,3,4-ij]quinoline-6-carboxamide,

(xx) N-(4-Chlorobenzyl)-2-{[(4-chlorobenzyl)amino]methyl}-9-(morpholin-4-ylmethyl)-7-oxo-2,3-dihydro-7H-[1,4]oxazino[2,3,4-ij]quinoline-6-carboxamide,

(yy) N-(4-Chlorobenzyl)-3-(hydroxymethyl)-9-(morpholin-4-ylmethyl)-7-oxo-2,3-dihydro-7H-[1,4]oxazino[2,3,4-ij]quinoline-6-carboxamide,

(zz) N-(4-Chlorobenzyl)-2-(4-hydroxyphenyl)-9-(morpholin-4-ylmethyl)-7-oxo-2,3-dihydro-7H-[1,4]oxazino[2,3,4-ij]quinoline-6-carboxamide,

(aaa) N-(4-Chlorobenzyl)-2-{3-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,

(bbb) N-(4-Chlorobenzyl)-2-{2-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,

(ccc) N-(4-Chlorobenzyl)-2-(2-hydroxyphenyl)-9-(morpholin-4-ylmethyl)-7-oxo-2,3-dihydro-7H-[1,4]oxazino[2,3,4-ij]quinoline-6-carboxamide,

(ddd) N-[(4-Chlorophenyl)methyl]-2,3,5,6-tetrahydro-9′-(4-morpholinylmethyl)-7′-oxospiro[4H-pyran-4,2′(3′H)-[7H]pyrido[1,2,3-de] [1,4]benzoxazine]-6′-carboxamide,

(eee) 1,1-Dimethylethyl 6-[[[(4-chlorophenyl)methyl]amino]carbonyl]-9′-(4-morpholinylmethyl)-7′-oxospiro[piperidine-4,2′(3′H)-[7H]pyrido[1,2,3-de] [1,4]benzoxazine]-1-carboxylate,

(fff) N-[(4-Chlorophenyl)methyl]-9′-(4-morpholinylmethyl)-7′-oxospiro[piperidine-4,2′(3′H)-[7H]pyrido[1,2,3-de] [1,4]benzoxazine]-6′-carboxamide,

(ggg) N-(4-Chlorobenzyl)-2,2-bis(hydroxymethyl)-9-(morpholin-4-ylmethyl)-7-oxo-2,3-dihydro-7H-[1,4]oxazino[2,3,4-ij]quinoline-6-carboxamide,

(hhh) N-[(4-Chlorophenyl)methyl]-2′,3′,5′,6′-tetrahydro-9-(4-morpholinylmethyl)-7-oxospiro[7H-pyrido[1,2,3-de]-1,4-benzoxazine-2(3H),4′-[4H]thiopyran]-6-carboxamide,

(iii) N-(4-Chlorobenzyl)-9-(morpholin-4-ylmethyl)-7-oxo-3-phenyl-2,3-dihydro-7H-[1,4]oxazino[2,3,4-ij]quinoline-6-carboxamide,

(jjj) N-(4-Chlorobenzyl)-3,3-bis(hydroxymethyl)-9-(3-hydroxy-1-propynyl)-7-oxo-2,3-dihydro-7H-[1,4]oxazino[2,3,4-ij]quinoline-6-carboxamide,

(kkk) N-(4-Chlorobenzyl)-3,3-bis(hydroxymethyl)-9-(3-hydroxypropyl)-7-oxo-2,3-dihydro-7H-[1,4]oxazino[2,3,4-ij]quinoline-6-carboxamide,

(lll) N-(4-Chlorobenzyl)-2-[2-(methoxymethoxy)phenyl]-9-(morpholin-4-ylmethyl)-7-oxo-2,3-dihydro-7H-[1,4]oxazino[2,3,4-ij]quinoline-6-carboxamide,

(mmm) N-(4-Chlorobenzyl)-2-{4-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,

(nnn) 2-[2,3-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,

(ooo) N-[(4-Chlorophenyl)methyl]-1-methyl-9′-(4-morpholinylmethyl)-7′-oxospiro[piperidine-4,2′(3′H)-[7H]pyrido[1,2,3-de] [1,4]benzoxazine]-6′-carboxamide,

(ppp) N-[(4-Chlorophenyl)methyl]-9″-(4-morpholinylmethyl)dispiro[1,3-dioxolane-2,1′-cyclohexane-4′,2″(3″H)-[7H] pyrido[1,2,3-de] [1,4]benzoxazine]-6″-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,

(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-thioxo-2-pyridin-2-yl-2,3-dihydro-7 H-[1,4]oxazino[2,3,4-ij]quinoline-6-carboxamide,

(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-thioxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-thioxo-2,3-dihydro-7H-[1,4]oxazino[2,3,4-ij]quinoline-6-carboxamide,

(j) N-(4-Chlorobenzyl)-2-{[(1-methyl-1H-imidazol-2-yl)sulfanyl]methyl}-9-(morpholin-4-ylmethyl)-7-thioxo-2,3-dihydro-7H-[1,4]oxazino[2,3,4-ij]quinoline-6-carboxamide,

(k) N-(4-Chlorobenzyl)-9-(morpholin-4-ylmethyl)-7-thioxo-2-{[(3-pyridinylmethyl)amino]methyl}-2,3-dihydro-7H-[1,4]oxazino[2,3,4-ij]quinoline-6-carboxamide,

(l) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-thioxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-thioxo-2,3-dihydro-7H-[1,4]oxazino[2,3,4-ij]quinoline-6-carboxamide,

(o) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-thioxo-2,3-dihydro-7H-[1,4]oxazino[2,3,4-ij]quinoline-6-carboxamide,

(q) N-(4-Chlorobenzyl)-2-[2-(hydroxymethyl)phenyl]-9-(morpholin-4-ylmethyl)-7-thioxo-2,3-dihydro-7H-[1,4]oxazino[2,3,4-ij]quinoline-6-carboxamide,

(r) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-thioxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-thioxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-thioxo-2,3-dihydro-7H-[1,4]oxazino[2,3,4-ij]quinoline-6-carboxamide,

(v) N-(4-Chlorobenzyl)-9-(morpholin-4-ylmethyl)-7-thioxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-thioxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-thloxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-thioxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-thioxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(bb) N-(4-Chlorobenzyl)-9-(morpholin-4-ylmethyl)-7-thioxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-thioxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4-oxo-7′-thioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-thioxospiro[cyclohexane-1,2′(3′H)-[7H]pyrido[1,2,3-de] [1,4]benzoxazine]-6′-carboxamide,

(ff) N-(4-Chlorobenzyl)-3,9-bis(morpholin-4-ylmethyl)-7-thioxo-2,3-dihydro-7H-[1,4]oxazino[2,3,4-ij]quinoline-6-carboxamide,

(gg) N-(4-Chlorobenzyl)-9-(morpholin-4-ylmethyl)-7-thioxo-2-phenyl-2,3-dihydro-7H-[1,4]oxazino[2,3,4-ij]quinoline-6-carboxamide,

(hh) N-(4-Chlorobenzyl)-2,2-difluoro-9-(morpholin-4-ylmethyl)-7-thioxo-2,3-dihydro-7H-[1,4]oxazino[2,3,4-ij]quinoline-6-carboxamide,

(ii) N-(4-Chlorobenzyl)-2-[(methylsulfanyl)methyl]-9-(morpholin-4-ylmethyl)-7-thioxo-2,3-dihydro-7H-[1,4]oxazino[2,3,4-ij]quinoline-6-carboxamide,

(jj) N-(4-Chlorobenzyl)-2-[(dimethylamino)methyl]-9-(morpholin-4-ylmethyl)-7-thioxo-2,3-dihydro-7H-[1,4]oxazino[2,3,4-ij]quinoline-6-carboxamide,

(kk) N-(4-Chlorobenzyl)-2-[(4-methyl-1-piperazinyl)methyl]-9-(morpholin-4-ylmethyl)-7-thioxo-2,3-dihydro-7H-[1,4]oxazino[2,3,4-ij]quinoline-6-carboxamide,

(ll) Methyl ({[6-{[(4-chlorobenzyl)amino]carbonyl}-9-(morpholin-4-ylmethyl)-7-thioxo-2,3-dihydro-7H-[1,4]oxazino[2,3,4-ij]quinolin-2-yl]methyl}thio)acetate,

(mm) N-(4-Chlorobenzyl)-9-(morpholin-4-ylmethyl)-7-thioxo-2-(1-pyrrolidinylmethyl)-2,3-dihydro-7H-[1,4]oxazino[2,3,4-ij]quinoline-6-carboxamide,

(nn) N-(4-Chlorobenzyl)-2-{[(2,3-dihydroxypropyl)sulfanyl]methyl}-9-(morpholin-4-ylmethyl)-7-thioxo-2,3-dihydro-7H-[1,4]oxazino[2,3,4-ij]quinoline-6-carboxamide,

(oo) N-(4-Chlorobenzyl)-2-{[(2,3-dihydroxypropyl)amino]methyl}-9-(morpholin-4-ylmethyl)-7-thioxo-2,3-dihydro-7H-[1,4]oxazino[2,3,4-ij]quinoline-6-carboxamide,

(pp) N-(4-Chlorobenzyl)-2-{[(2-hydroxyethyl)amino]methyl}-9-(morpholin-4-ylmethyl)-7-thioxo-2,3-dihydro-7H-[1,4]oxazino[2,3,4-ij]quinoline-6-carboxamide,

(qq) N-(4-Chlorobenzyl)-9-(morpholin-4-ylmethyl)-7-thioxo-2-(1-piperidinylmethyl)-2,3-dihydro-7H-[1,4]oxazino[2,3,4-ij]quinoline-6-carboxamide,

(rr) 2-{[bis(2-Hydroxyethyl)amino]methyl}-N-(4-chlorobenzyl)-9-(morpholin-4-ylmethyl)-7-thioxo-2,3-dihydro-7H-[1,4]oxazino[2,3,4-ij]quinoline-6-carboxamide,

(ss) N-(4-Chlorobenzyl)-9-(morpholin-4-ylmethyl)-7-thioxo-2-{[(2-pyridinylmethyl)amino]methyl}-2,3-dihydro-7H-[1,4]oxazino[2,3,4-ij]quinoline-6-carboxamide,

(tt) 2-[(8-{[6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methoxy}-8-oxooctanoyl)(methyl)amino]ethanesulfonic acid sodium salt,

(uu) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl dimethyl phosphate,

(vv) N-(4-Chlorobenzyl)-9-(morpholin-4-ylmethyl)-7-thioxo-2-{[(4-pyridinylmethyl)amino]methyl}-2,3-dihydro-7H-[1,4]oxazino[2,3,4-ij]quinoline-6-carboxamide,

(ww) N-(4-Chlorobenzyl)-2-(1H-imidazol-1-ylmethyl)-9-(morpholin-4-ylmethyl)-7-thioxo-2,3-dihydro-7H-[1,4]oxazino[2,3,4-ij]quinoline-6-carboxamide,

(xx) N-(4-Chlorobenzyl)-2-{[(4-chlorobenzyl)amino]methyl}-9-(morpholin-4-ylmethyl)-7-thioxo-2,3-dihydro-7H-[1,4]oxazino[2,3,4-ij]quinoline-6-carboxamide,

(yy) N-(4-Chlorobenzyl)-3-(hydroxymethyl)-9-(morpholin-4-ylmethyl)-7-thioxo-2,3-dihydro-7H-[1,4]oxazino[2,3,4-ij]quinoline-6-carboxamide,

(zz) N-(4-Chlorobenzyl)-2-(4-hydroxyphenyl)-9-(morpholin-4-ylmethyl)-7-thioxo-2,3-dihydro-7H-[1,4]oxazino[2,3,4-ij]quinoline-6-carboxamide,

(aaa) N-(4-Chlorobenzyl)-2-{3-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,

(bbb) N-(4-Chlorobenzyl)-2-{2-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,

(ccc) N-(4-Chlorobenzyl)-2-(2-hydroxyphenyl)-9-(morpholin-4-ylmethyl)-7-thioxo-2,3-dihydro-7H-[1,4]oxazino[2,3,4-ij]quinoline-6-carboxamide,

(ddd) N-[(4-Chlorophenyl)methyl]-2,3,5,6-tetrahydro-9′-(4-morpholinylmethyl)-7′-thioxospiro[4H-pyran-4,2′(3′H)-[7H]pyrido[1,2,3-de] [1,4]benzoxazine]-6′-carboxamide,

(eee) 1,1-Dimethylethyl 6-[[[(4-chlorophenyl)methyl]amino]carbonyl]-9′-(4-morpholinylmethyl)-7′-thioxospiro[piperidine-4,2′(3′H)-[7H]pyrido[1,2,3-de] [1,4]benzoxazine]-1-carboxylate,

(fff) N-[(4-Chlorophenyl)methyl]-9′-(4-morpholinylmethyl)-7′-thioxospiro[piperidine-4,2′(3′H)-[7H]pyrido[1,2,3-de] [1,4]benzoxazine]-6′-carboxamide,

(ggg) N-(4-Chlorobenzyl)-2,2-bis(hydroxymethyl)-9-(morpholin-4-ylmethyl)-7-thioxo-2,3-dihydro-7H-[1,4]oxazino[2,3,4-ij]quinoline-6-carboxamide,

(hhh) N-[(4-Chlorophenyl)methyl]-2′,3′,5′,6′-tetrahydro-9-(4-morpholinylmethyl)-7-thioxospiro[7H-pyrido[1,2,3-de]-1,4-benzoxazine-2(3H),4′-[4H]thiopyran]-6-carboxamide,

(iii) N-(4-Chlorobenzyl)-9-(morpholin-4-ylmethyl)-7-thioxo-3-phenyl-2,3-dihydro-7H-[1,4]oxazino[2,3,4-ij]quinoline-6-carboxamide,

(jjj) N-(4-Chlorobenzyl)-3,3-bis(hydroxymethyl)-9-(3-hydroxy-1-propynyl)-7-thioxo-2,3-dihydro-7H-[1,4]oxazino[2,3,4-ij]quinoline-6-carboxamide,

(kkk) N-(4-Chlorobenzyl)-3,3-bis(hydroxymethyl)-9-(3-hydroxypropyl)-7-thioxo-2,3-dihydro-7H-[1,4]oxazino[2,3,4-ij]quinoline-6-carboxamide,

(lll) N-(4-Chlorobenzyl)-2-[2-(methoxymethoxy)phenyl]-9-(morpholin-4-ylmethyl)-7-thioxo-2,3-dihydro-7H-[1,4]oxazino[2,3,4-ij]quinoline-6-carboxamide,

(mmm) N-(4-Chlorobenzyl)-2-{4-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,

(nnn) 2-[2,3-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(ooo) N-[(4-Chlorophenyl)methyl]-1-methyl-9′-(4-morpholinylmethyl)-7′-thioxospiro[piperidine-4,2′(3′H)-[7H]pyrido[1,2,3-de] [1,4]benzoxazine]-6′-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxamide,

(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-oxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-oxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-oxo-2,3-dihydro-7H-[1,4]oxazino[2,3,4-ij]quinoline-6-carboxamide,

(j) N-(4-Chlorobenzyl)-2-{[(1-methyl-1H-imidazol-2-yl)sulfanyl]methyl}-9-(morpholin-4-ylmethyl)-7-oxo-2,3-dihydro-7H-[1,4]oxazino[2,3,4-ij]quinoline-6-carboxamide,

(k) N-(4-Chlorobenzyl)-9-(morpholin-4-ylmethyl)-7-oxo-2-{[(3-pyridinylmethyl)amino]methyl}-2,3-dihydro-7H-[1,4]oxazino[2,3,4-ij]quinoline-6-carboxamide,

(l) [6-{[(4-Chlorobenzyl)amino]carbonyl)-9-(morpholin-4-ylmethyl)-7-oxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-oxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-oxo-2,3-dihydro-7H-[1,4]oxazino[2,3,4-ij]quinoline-6-carboxamide,

(o) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-oxo-2,3-dihydro-7H-[1,4]oxazino[2,3,4-ij]quinoline-6-carboxamide,

(q) N-(4-Chlorobenzyl)-2-(2-(hydroxymethyl)phenyl]-9-(morpholin-4-ylmethyl)-7-oxo-2,3-dihydro-7H-[1,4]oxazino[2,3,4-ij]quinoline-6-carboxamide,

(r) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-oxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-oxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-oxo-2,3-dihydro-7H-[1,4]oxazino[2,3,4-ij]quinoline-6-carboxamide,

(v) N-(4-Chlorobenzyl)-9-(morpholin-4-ylmethyl)-7-oxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-oxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-oxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,

(bb) N-(4-Chlorobenzyl)-9-(morpholin-4-ylmethyl)-7-oxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-oxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4,7′-dioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-oxospiro[cyclohexane-1,2′(3′H)-[7H]pyrido[1,2,3-de] [1,4]benzoxazine]-6′-carboxamide,

(ff) N-(4-Chlorobenzyl)-3,9-bis(morpholin-4-ylmethyl)-7-oxo-2,3-dihydro-7H-[1,4]oxazino[2,3,4-ij]quinoline-6-carboxamide,

(gg) N-(4-Chlorobenzyl)-9-(morpholin-4-ylmethyl)-7-oxo-2-phenyl-2,3-dihydro-7H-[1,4]oxazino[2,3,4-ij]quinoline-6-carboxamide,

(hh) N-(4-Chlorobenzyl)-2,2-difluoro-9-(morpholin-4-ylmethyl)-7-oxo-2,3-dihydro-7H-[1,4]oxazino[2,3,4-ij]quinoline-6-carboxamide,

(ii) N-(4-Chlorobenzyl)-2-[(methylsulfanyl)methyl]-9-(morpholin-4-ylmethyl)-7-oxo-2,3-dihydro-7H-[1,4]oxazino[2,3,4-ij]quinoline-6-carboxamide,

(jj) N-(4-Chlorobenzyl)-2-[(dimethylamino)methyl]-9-(morpholin-4-ylmethyl)-7-oxo-2,3-dihydro-7H-[1,4]oxazino[2,3,4-ij]quinoline-6-carboxamide,

(kk) N-(4-Chlorobenzyl)-2-[(4-methyl-1-piperazinyl)methyl]-9-(morpholin-4-ylmethyl)-7-oxo-2,3-dihydro-7H-[1,4]oxazino[2,3,4-ij]quinoline-6-carboxamide,

(ll) Methyl({[6-{[(4-chlorobenzyl)amino]carbonyl}-9-(morpholin-4-ylmethyl)-7-oxo-2,3-dihydro-7H-[1,4]oxazino[2,3,4-ij]quinolin-2-yl]methyl)thio)acetate,

(mm) N-(4-Chlorobenzyl)-9-(morpholin-4-ylmethyl)-7-oxo-2-(1-pyrrolidinylmethyl)-2,3-dihydro-7H-[1,4]oxazino[2,3,4-ij]quinoline-6-carboxamide,

(nn) N-(4-Chlorobenzyl)-2-{[(2,3-dihydroxypropyl)sulfanyl]methyl}-9-l(morpholin-4-ylmethyl)-7-oxo-2,3-dihydro-7H-[1,4]oxazino[2,3,4-ij]quinoline-6-carboxamide,

(oo) N-(4-Chlorobenzyl)-2-{[(2,3-dihydroxypropyl)amino]methyl}-9-(morpholin-4-ylmethyl)-7-oxo-2,3-dihydro-7H-[1,4]oxazino[2,3,4-ij]quinoline-6-carboxamide,

(pp) N-(4-Chlorobenzyl)-2-{[(2-hydroxyethyl)amino]methyl}-9-(morpholin-4-ylmethyl)-7-oxo-2,3-dihydro-7H-[1,4]oxazino[2,3,4-ij]quinoline-6-carboxamide,

(qq) N-(4-Chlorobenzyl)-9-(morpholin-4-ylmethyl)-7-oxo-2-(1-piperidinylmethyl)-2,3-dihydro-7H-[1,4]oxazino[2,3,4-ij]quinoline-6-carboxamide,

(rr) 2-{[bis(2-Hydroxyethyl)amino]methyl}-N-(4-chlorobenzyl)-9-(morpholin-4-ylmethyl)-7-oxo-2,3-dihydro-7H-[1,4]oxazino[2,3,4-ij]quinoline-6-carboxamide,

(ss) N-(4-Chlorobenzyl)-9-(morpholin-4-ylmethyl)-7-oxo-2-{[(2-pyridinylmethyl)amino]methyl}-2,3-dihydro-7H-[1,4]oxazino[2,3,4-ij]quinoline-6-carboxamide,

(tt) 2-[(8-{[6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methoxy}-8-oxooctanoyl)(methyl)amino]ethanesulfonic acid sodium salt,

(uu) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl dimethyl phosphate,

(vv) N-(4-Chlorobenzyl)-9-(morpholin-4-ylmethyl)-7-oxo-2-{[(4-pyridinylmethyl)amino]methyl}-2,3-dihydro-7H-[1,4]oxazino[2,3,4-ij]quinoline-6-carboxamide,

(ww) N-(4-Chlorobenzyl)-2-(1H-imidazol-1-ylmethyl)-9-(morpholin-4-ylmethyl)-7-oxo-2,3-dihydro-7H-[1,4]oxazino[2,3,4-ij]quinoline-6-carboxamide,

(xx) N-(4-Chlorobenzyl)-2-{[(4-chlorobenzyl)amino]methyl}-9-(morpholin-4-ylmethyl)-7-oxo-2,3-dihydro-7H-[1,4]oxazino[2,3,4-ij]quinoline-6-carboxamide,

(yy) N-(4-Chlorobenzyl)-3-(hydroxymethyl)-9-(morpholin-4-ylmethyl)-7-oxo-2,3-dihydro-7H-[1,4]oxazino[2,3,4-ij]quinoline-6-carboxamide,

(zz) N-(4-Chlorobenzyl)-2-(4-hydroxyphenyl)-9-(morpholin-4-ylmethyl)-7-oxo-2,3-dihydro-7H-[1,4]oxazino[2,3,4-ij]quinoline-6-carboxamide,

(aaa) N-(4-Chlorobenzyl)-2-{3-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,

(bbb) N-(4-Chlorobenzyl)-2-{2-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,

(ccc) N-(4-Chlorobenzyl)-2-(2-hydroxyphenyl)-9-(morpholin-4-ylmethyl)-7-oxo-2,3-dihydro-7H-[1,4]oxazino[2,3,4-ij]quinoline-6-carboxamide,

(ddd) N-[(4-Chlorophenyl)methyl]-2,3,5,6-tetrahydro-9′-(4-morpholinylmethyl)-7′-oxospiro[4H-pyran-4,2′(3′H)-[7H]pyrido[1,2,3-de] [1,4]benzoxazine]-6′-carboxamide,

(eee) 1,1-Dimethylethyl 6-[[[(4-chlorophenyl)methyl]amino]carbonyl]-9′-(4-morpholinylmethyl)-7′-oxospiro[piperidine-4,2′(3′H)-[7H]pyrido[1,2,3-de] [1,4]benzoxazine]-1-carboxylate,

(fff) N-[(4-Chlorophenyl)methyl]-9′-(4-morpholinylmethyl)-7′-oxospiro[piperidine-4,2′(3′H)-[7H]pyrido[1,2,3-de] [1,4]benzoxazine]-6′-carboxamide,

(ggg) N-(4-Chlorobenzyl)-2,2-bis(hydroxymethyl)-9-(morpholin-4-ylmethyl)-7-oxo-2,3-dihydro-7H-[1,4]oxazino[2,3,4-ij]quinoline-6-carboxamide,

(hhh) N-[(4-Chlorophenyl)methyl]-2′,3′,5′,6′-tetrahydro-9-(4-morpholinylmethyl)-7-oxospiro[7H-pyrido[1,2,3-de]-1,4-benzoxazine-2(3H),4′-[4H]thiopyran]-6-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,

(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-thioxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-thioxo-2,3-dihydro-7H-[1,4]oxazino[2,3,4-ij]quinoline-6-carboxamide,

(j) N-(4-Chlorobenzyl)-2-{[(1-methyl-1H-imidazol-2-yl)sulfanyl]methyl}-9-(morpholin-4-ylmethyl)-7-thioxo-2,3-dihydro-7H-[1,4]oxazino[2,3,4-ij]quinoline-6-carboxamide,

(k) N-(4-Chlorobenzyl)-9-(morpholin-4-ylmethyl)-7-thioxo-2-{[(3-pyridinylmethyl)amino]methyl}-2,3-dihydro-7H-[1,4]oxazino[2,3,4-ij]quinoline-6-carboxamide,

(l) [6-([(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-thioxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-thioxo-2,3-dihydro-7H-[1,4]oxazino[2,3,4-ij]quinoline-6-carboxamide,

(o) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-thioxo-2,3-dihydro-7H-[1,4]oxazino[2,3,4-ij]quinoline-6-carboxamide,

(q) N-(4-Chlorobenzyl)-2-[2-(hydroxymethyl)phenyl]-9-(morpholin-4-ylmethyl)-7-thioxo-2,3-dihydro-7H-[1,4]oxazino[2,3,4-ij]quinoline-6-carboxamide, p1 (r) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-thioxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-thioxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-thioxo-2,3-dihydro-7H-[1,4]oxazino[2,3,4-ij]quinoline-6-carboxamide,

(v) N-(4-Chlorobenzyl)-9-(morpholin-4-ylmethyl)-7-thioxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-thioxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-thioxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-thioxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-thioxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(bb) N-(4-Chlorobenzyl)-9-(morpholin-4-ylmethyl)-7-thioxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-thioxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4-oxo-7′-thioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-thioxospiro[cyclohexane-1,2′(3′H)-[7H]pyrido[1,2,3-de] [1,4]benzoxazine]-6′-carboxamide,

(ff) N-(4-Chlorobenzyl)-3,9-bis(morpholin-4-ylmethyl)-7-thioxo-2,3-dihydro-7H-[1,4]oxazino[2,3,4-ij]quinoline-6-carboxamide,

(gg) N-(4-Chlorobenzyl)-9-(morpholin-4-ylmethyl)-7-thioxo-2-phenyl-2,3-dihydro-7H-[1,4]oxazino[2,3,4-ij]quinoline-6-carboxamide,

(hh) N-(4-Chlorobenzyl)-2,2-difluoro-9-(morpholin-4-ylmethyl)-7-thioxo-2,3-dihydro-7H-[1,4]oxazino[2,3,4-ij]quinoline-6-carboxamide,

(ii) N-(4-Chlorobenzyl)-2-[(methylsulfanyl)methyl]-9-(morpholin-4-ylmethyl)-7-thioxo-2,3-dihydro-7H-[1,4]oxazino[2,3,4-ij]quinoline-6-carboxamide,

(jj) N-(4-Chlorobenzyl)-2-[(dimethylamino)methyl]-9-(morpholin-4-ylmethyl)-7-thioxo-2,3-dihydro-7H-[1,4]oxazino[2,3,4-ij]quinoline-6-carboxamide,

(kk) N-(4-Chlorobenzyl)-2-[(4-methyl-1-piperazinyl)methyl]-9-(morpholin-4-ylmethyl)-7-thioxo-2,3-dihydro-7H-[1,4]oxazino[2,3,4-ij]quinoline-6-carboxamide,

(ll) Methyl({[6-{[(4-chlorobenzyl)amino]carbonyl}-9-(morpholin-4-ylmethyl)-7-thioxo-2,3-dihydro-7H-[1,4]oxazino[2,3,4-ij]quinolin-2-yl]methyl}thio)acetate,

(mm) N-(4-Chlorobenzyl)-9-(morpholin-4-ylmethyl)-7-thioxo-2-(1-pyrrolidinylmethyl)-2,3-dihydro-7H-[1,4]oxazino[2,3,4-ij]quinoline-6-carboxamide,

(nn) N-(4-Chlorobenzyl)-2-{[(2,3-dihydroxypropyl)sulfanyl]methyl}-9-(morpholin-4-ylmethyl)-7-thioxo-2,3-dihydro-7H-[1,4]oxazino[2,3,4-ij]quinoline-6-carboxamide,

(oo) N-(4-Chlorobenzyl)-2-{[(2,3-dihydroxypropyl)amino]methyl}-9-(morpholin-4-ylmethyl)-7-thioxo-2,3-dihydro-7H-[1,4]oxazino[2,3,4-ij]quinoline-6-carboxamide,

(pp) N-(4-Chlorobenzyl)-2-{[(2-hydroxyethyl)amino]methyl}-9-(morpholin-4-ylmethyl)-7-thioxo-2,3-dihydro-7H-[1,4]oxazino[2,3,4-ij]quinoline-6-carboxamide,

(qq) N-(4-Chlorobenzyl)-9-(morpholin-4-ylmethyl)-7-thioxo-2-(1-piperidinylmethyl)-2,3-dihydro-7H-[1,4]oxazino[2,3,4-ij]quinoline-6-carboxamide,

(rr) 2-{[bis(2-Hydroxyethyl)amino]methyl}-N-(4-chlorobenzyl)-9-(morpholin-4-ylmethyl)-7-thioxo-2,3-dihydro-7H-[1,4]oxazino[2,3,4-ij]quinoline-6-carboxamide,

(ss) N-(4-Chlorobenzyl)-9-(morpholin-4-ylmethyl)-7-thioxo-2-{[(2-pyridinylmethyl)amino]methyl)-2,3-dihydro-7H-[1,4]oxazino[2,3,4-ij]quinoline-6-carboxamide,

(tt) 2-[(8-{[6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methoxy}-8-oxooctanoyl)(methyl)amino]ethanesulfonic acid sodium salt,

(uu) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl dimethyl phosphate,

(vv) N-(4-Chlorobenzyl)-9-(morpholin-4-ylmethyl)-7-thioxo-2-{[(4-pyridinylmethyl)amino]methyl}-2,3-dihydro-7H-[1,4]oxazino[2,3,4-ij]quinoline-6-carboxamide,

(ww) N-(4-Chlorobenzyl)-2-(1H-imidazol-1-ylmethyl)-9-(morpholin-4-ylmethyl)-7-thioxo-2,3-dihydro-7H-[1,4]oxazino[2,3,4-ij]quinoline-6-carboxamide,

(xx) N-(4-Chlorobenzyl)-2-{[(4-chlorobenzyl)amino]methyl}-9-(morpholin-4-ylmethyl)-7-thioxo-2,3-dihydro-7H-[1,4]oxazino[2,3,4-ij]quinoline-6-carboxamide,

(yy) N-(4-Chlorobenzyl)-3-(hydroxymethyl)-9-(morpholin-4-ylmethyl)-7-thioxo-2,3-dihydro-7H-[1,4]oxazino[2,3,4-ij]quinoline-6-carboxamide,

(zz) N-(4-Chlorobenzyl)-2-(4-hydroxyphenyl)-9-(morpholin-4-ylmethyl)-7-thioxo-2,3-dihydro-7H-[1,4]oxazino[2,3,4-ij]quinoline-6-carboxamide,

(aaa) N-(4-Chlorobenzyl)-2-{3-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,

(bbb) N-(4-Chlorobenzyl)-2-{2-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,

(ccc) N-(4-Chlorobenzyl)-2-(2-hydroxyphenyl)-9-(morpholin-4-ylmethyl)-7-thioxo-2,3-dihydro-7H-[1,4]oxazino[2,3,4-ij]quinoline-6-carboxamide,

(ddd) N-[(4-Chlorophenyl)methyl]-2,3,5,6-tetrahydro-9′-(4-morpholinylmethyl)-7′-thioxospiro[4H-pyran-4,2′(3′H)-[7H]pyrido[1,2,3-de] [1,4]benzoxazine]-6′-carboxamide,

(eee) 1,1-Dimethylethyl 6-[[[(4-chlorophenyl)methyl]amino]carbonyl]-9′-(4-morpholinylmethyl)-7′-thioxospiro[piperidine-4,2′(3′H)-[7H]pyrido[1,2,3-de] [1,4]benzoxazine]-1-carboxylate,

(fff) N-[(4-Chlorophenyl)methyl]-9′-(4-morpholinylmethyl)-7′-thioxospiro[piperidine-4,2′(3′H)-[7H]pyrido[1,2,3-de] [1,4]benzoxazine]-6′-carboxamide,

(ggg) N-(4-Chlorobenzyl)-2,2-bis[(hydroxymethoxy)methyl]-9-(morpholin-4-ylmethyl)-7-thioxo-2,3-dihydro-7H-[1,4]oxazino[2,3,4-ij]quinoline-6-carboxamide,

(hhh) N-[(4-Chlorophenyl)methyl]-2′,3′,5′,6′-tetrahydro-9-(4-morpholinylmethyl)-7-thioxospiro[7H-pyrido[1,2,3-de]-1,4-benzoxazine-2(3H), 4′-[4H]thiopyran]-6-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxamide,

(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-oxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-oxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-oxo-2,3-dihydro-7H-[1,4]oxazino[2,3,4-ij]quinoline-6-carboxamide,

(j) N-(4-Chlorobenzyl)-2-{[(1-methyl-1H-1-midazol-2-yl)sulfanyl]methyl}-9-(morpholin-4-ylmethyl)-7-oxo-2,3-dihydro-7H-[1,4]oxazino[2,3,4-ij]quinoline-6-carboxamide,

(k) N-(4-Chlorobenzyl)-9-(morpholin-4-ylmethyl)-7-oxo-2-{[(3-pyridinylmethyl)amino]methyl}-2,3-dihydro-7H-[1,4]oxazino[2,3,4-ij]quinoline-6-carboxamide,

(l) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-oxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-oxo-2,3-dihydro-7H-[1,4]oxazino[2,3,4-ij]quinoline-6-carboxamide,

(o) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-oxo-2,3-dihydro-7H-[1,4]oxazino[2,3,4-ij]quinoline-6-carboxamide,

(q) N-(4-Chlorobenzyl)-2-[2-(hydroxymethyl)phenyl]-9-(morpholin-4-ylmethyl)-7-oxo-2,3-dihydro-7H-[1,4]oxazino[2,3,4-ij]quinoline-6-carboxamide,

(r) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-oxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-oxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-oxo-2,3-dihydro-7H-[1,4]oxazino[2,3,4-ij]quinoline-6-carboxamide,

(v) N-(4-Chlorobenzyl)-9-(morpholin-4-ylmethyl)-7-oxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-oxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-oxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-oxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,

(bb) N-(4-Chlorobenzyl)-9-(morpholin-4-ylmethyl)-7-oxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-oxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4,7′-dioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-oxospiro[cyclohexane-1,2′(3′H)-[7H]pyrido[1,2,3-de] [1,4]benzoxazine]-6′-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,

(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxamide,

(g) N-(4-Chlorobenzyl)-2,9-bis(morpholin-4-ylmethyl)-7-thioxo-2,3-dihydro-7H-[1,4]oxazino[2,3,4-ij]quinoline-6-carboxamide,

(h) 2-[(tert-Butylsulfanyl)methyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(i) N-(4-Chlorobenzyl)-2-{[(2-hydroxyethyl)sulfanyl]methyl}-9-(morpholin-4-ylmethyl)-7-thioxo-2,3-dihydro-7H-[1,4]oxazino[2,3,4-ij]quinoline-6-carboxamide,

(j) N-(4-Chlorobenzyl)-2-{[(′-methyl-!H-imidazol-2-yl)sulfanyl]methyl}-9-(morpholin-4-ylmethyl)-7-thioxo-2,3-dihydro-7H-[1,4]oxazino[2,3,4-ij]quinoline-6-carboxamide,

(k) N-(4-Chlorobenzyl)-9-(morpholin-4-ylmethyl)-7-thioxo-2-{[(3-pyridinylmethyl)amino]methyl}-2,3-dihydro-7H-[1,4]oxazino[2,3,4-ij]quinoline-6-carboxamide,

(l) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl acetate,

(m) N-(4-Chlorobenzyl)-9-(morpholin-4-ylmethyl)-7-thioxo-2-(R or S)-{[(3-pyridinylmethyl)amino]methyl}-2,3-dihydro-7H-[1,4]oxazino[2,3,4-ij]quinoline-6-carboxamide,

(n) N-(4-Chlorobenzyl)-2-(3-hydroxyphenyl)-9-(morpholin-4-ylmethyl)-7-thioxo-2,3-dihydro-7H-[1,4]oxazino[2,3,4-ij]quinoline-6-carboxamide,

(o) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,

(p) N-(4-Chlorobenzyl)-2-[3-(hydroxymethyl)phenyl]-9-(morpholin-4-ylmethyl)-7-thioxo-2,3-dihydro-7H-[1,4]oxazino[2,3,4-ij]quinoline-6-carboxamide,

(q) N-(4-Chlorobenzyl)-2-[2-(hydroxymethyl)phenyl]-9-(morpholin-4-ylmethyl)-7-thioxo-2,3-dihydro-7H-[1,4]oxazino[2,3,4-ij]quinoline-6-carboxamide,

(r) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide,

(s) N-(4-Chlorobenzyl)-2-(2-furyl)-9-(morpholin-4-ylmethyl)-7-thioxo-2,3-dihydro-7H-[1,4]oxazino[2,3,4-ij]quinoline-6-carboxamide,

(t) N-(4-Chlorobenzyl)-2-(3-cyanophenyl)-9-(morpholin-4-ylmethyl)-7-thioxo-2,3-dihydro-7H-[1,4]oxazino[2,3,4-ij]quinoline-6-carboxamide,

(u) N-(4-Chlorobenzyl)-2-(3-furyl)-9-(morpholin-4-ylmethyl)-7-thioxo-2,3-dihydro-7H-[1,4]oxazino[2,3,4-ij]quinoline-6-carboxamide,

(v) N-(4-Chlorobenzyl)-9-(morpholin-4-ylmethyl)-7-thioxo-2-thien-2-yl-2,3-dihydro-7H-[1,4]oxazino[2,3,4-ij]quinoline-6-carboxamide,

(w) N-(4-Chlorobenzyl)-2-(3,5-difluorophenyl)-9-(morpholin-4-ylmethyl)-7-thioxo-2,3-dihydro-7H-[1,4]oxazino[2,3,4-ij]quinoline-6-carboxamide,

(x) 2-(1,3-Benzodioxol-5-yl)-N-(4-chlorobenzyl)-9-(morpholin-4-ylmethyl)-7-thioxo-2,3-dihydro-7H-[1,4]oxazino[2,3,4-ij]quinoline-6-carboxamide,

(y) N-(4-Chlorobenzyl)-2-(2,3-dihydro-1,4-benzodioxin-6-yl)-9-(morpholin-4-ylmethyl)-7-thioxo-2,3-dihydro-7H-[1,4]oxazino[2,3,4-ij]quinoline-6-carboxamide,

(z) 2-(1,3-Benzodioxol-4-yl)-N-(4-chlorobenzyl)-9-(morpholin-4-ylmethyl)-7-thioxo-2,3-dihydro-7H-[1,4]oxazino[2,3,4-ij]quinoline-6-carboxamide,

(aa) 2-[3,5-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,

(bb) N-(4-Chlorobenzyl)-9-(morpholin-4-ylmethyl)-7-thioxo-2-thien-3-yl-2,3-dihydro-7H-[1,4]oxazino[2,3,4-ij]quinoline-6-carboxamide,

(cc) N-(4-Chlorobenzyl)-2,2-bis[(methoxymethoxy)methyl]-9-(morpholin-4-ylmethyl)-7-thioxo-2,3-dihydro-7H-[1,4]oxazino[2,3,4-ij]quinoline-6-carboxamide,

(dd) N-[(4-Chlorophenyl)methyl]-9′-(4-morpholinylmethyl)-4-oxo-7′-thioxospiro[cyclohexane-1,2′(3′H)-[7H]pyrido[1,2,3-de] [1,4]benzoxazine]-6′-carboxamide,

(ee) N-[(4-Chlorophenyl)methyl]-4-hydroxy-9′-(4-morpholinylmethyl)-7′-thioxospiro[cyclohexane-1,2′(3′H)-[7H]pyrido[1,2,3-de] [1,4]benzoxazine]-6′-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxamide,

(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxamide,

(g) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxamide,

(h) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxamide,

(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,

(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,

(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,

(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxamide,

(f) N-(4-Chlorobenzyl)-9-(morpholin-4-ylmethyl)-2-thioxo-2-(R or S)-pyridin-3-yl-2,3-dihydro-7H-[1,4]oxazino[2,3,4-ij]quinoline-6-carboxamide,

(g) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,

(h) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxthioamide,

(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxthioamide,

(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxthioamide,

(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxthioamide,

(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxthioamide,

(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxthioamide,

(g) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxthioamide,

(h) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxthioamide, or a pharmaceutically acceptable salt.

A method for preventing or treating atherosclerosis or restenosis in mammals, wherein the compound administered has the Formula V and is selected from the group consisting of

(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxthioamide,

(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxthioamide,

(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxthioamide,

(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxthioamide,

(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxthioamide,

(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxthioamide,

(g) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxthioamide,

(h) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxthioamide, or a pharmaceutically acceptable salt.

A method for preventing or treating atherosclerosis or restenosis in mammals, wherein the compound administered has the Formula V and is N-(4-chlorobenzyl)-9-(morpholin-4-ylmethyl)-7-oxo-2-(S)-pyridin-2-yl-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is N-(4-chlorobenzyl)-9-(morpholin-4-ylmethyl)-7-oxo-2-(R)-pyridin-2-yl-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is N-(4-chlorobenzyl)-9-(morpholin-4-ylmethyl)-7-oxo-2-(R)-pyridin-3-yl-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is N-(4-chlorobenzyl)-9-(morpholin-4-ylmethyl)-7-oxo-2-(S)-pyridin-3-yl-2,3-dihydro-7H-[1,4]oxazino[2,3,4-ij]quinoline-6-carboxamide, or a pharmaceutically acceptable salt.

A method for preventing or treating atherosclerosis or restenosis in mammals, wherein the compound administered has the Formula V and is N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide, or a pharmaceutically acceptable salt thereof.

A method for preventing or treating atherosclerosis or restenosis in mammals, wherein the compound administered has the Formula XI, and wherein X^{XI} is Cl;

R^{XI-1} is H;

R^{XI-2} is C₁₋₇alkyl which may be partially unsaturated and is substituted by one or more substituents of the group OH, NR^{XI-7}R^{XI-8}, or ^{XI-}het bound through a carbon atom; and

R^{XI-3} is H.

A method for preventing or treating atherosclerosis or restenosis in mammals, wherein the compound administered has the Formula XI and R^{XI-2} is C₁₋₇alkyl which may be partially unsaturated and is substituted by one or more substituents of the group OH, NR^{XI-7}R^{XI-8}, or het^{XI} bound through a carbon atom.

A method for preventing or treating atherosclerosis or restenosis in mammals, wherein the compound administered has the Formula XI and R^{XI-2} is C₁₋₇alkyl which is fully saturated and is substituted by one or more substituents of the group OH or NR^{XI-7}R^{XI-8}.

A method for preventing or treating atherosclerosis or restenosis in mammals, wherein the compound administered has the Formula XI and R^{XI-2} is 3-hydroxypropyl.

A method for preventing or treating atherosclerosis or restenosis in mammals, wherein the compound administered has the Formula XI and R^{XI-2} is 3-hydroxy-1-propynyl.

A method for preventing or treating atherosclerosis or restenosis in mammals, wherein the compound administered has the Formula XI and R^{XI-2} is tetrahydro-2H-pyran-4-ylmethyl.

A method for preventing or treating atherosclerosis or restenosis in mammals, wherein the compound administered has the Formula XI and R^{XI-2} is 4-morpholinylmethyl.

A method for preventing or treating atherosclerosis or restenosis in mammals, wherein the compound administered has the Formula XI and is selected from the group consisting of

(1) N-(4-chlorobenzyl)-6-(4-morpholinylmethyl)-4-oxo-1-phenyl-1,4-dihydro-3-quinolinecarboxamide;

(2) N-(4-chlorobenzyl)-6-(3-hydroxypropyl)-4-oxo-1-phenyl-1,4-dihydro-3-quinoline-carboxamide;

(3) N-(4-chlorobenzyl)-6-(3-hydroxy-1-propynyl)-4-oxo-1-phenyl-1,4-dihydro-3-quinolinecarboxamide;

(4) N-(4-chlorobenzyl)-4-oxo-1-phenyl-6-(tetrahydro-2H-pyran-4-ylmethyl)-1,4-dihydro-3-quinolinecarboxamide;

(5) N-(4-chlorobenzyl)-1-(2-methylphenyl)-6-(4-morpholinylmethyl)-4-oxo-1,4-dihydro-3-quinolinecarboxamide;

(6) N-(4-chlorobenzyl)-1-(3-iodophenyl)-6-(4-morpholinylmethyl)-4-oxo-1,4-dihydro-3-quinolinecarboxamideN-(4-chlorobenzyl)-1-(4-chlorophenyl)-6-(4-morpholinylmethyl)-4-oxo-1,4-dihydro-3-quinolinecarboxamide;

(7) N-(4-chlorobenzyl)-1-(4-isopropylphenyl)-6-(4-morpholinylmethyl)-4-oxo-1,4-dihydro-3-quinolinecarboxamide;

(8) N-(4-chlorobenzyl)-1-(4-methoxyphenyl)-6-(4-morpholinylmethyl)-4-oxo-1,4-dihydro-3-quinolinecarboxamide;

(9) N-(4-fluorobenzyl)-1-(4-chlorophenyl)-6-(4-morpholinylmethyl)-4-oxo-1,4-dihydro-3-quinolinecarboxamide;

(10) N-(4-chlorobenzyl)-6-(4-morpholinylmethyl)-4-oxo-1-(2,4-difluorophenyl)-1,4-dihydro-3-quinolinecarboxamide;

(11) N-(4-chlorobenzyl)-6-(3-hydroxypropyl)-4-oxo-1-(2,4-difluorophenyl)-1,4-dihydro-3-quinolinecarboxamide;

(12) N-(4-chlorobenzyl)-6-(3-hydroxy-1-propynyl)-4-oxo-1-(2,4-difluorophenyl)-1,4-dihydro-3-quinolinecarboxamide;

(13) N-(4-chlorobenzyl)-4-oxo-1-(2,4-difluorophenyl)-6-(tetrahydro-2H-pyran-4-yl-methyl)-1,4-dihydro-3-quinolinecarboxamide;

(14) N-(4-Chlorobenzyl)-1-(2-(hydroxymethyl)phenyl)-6-(4-morpholinylmethyl)-4-oxo-1,4-dihydro-3-quinolinecarboxamide;

(15) N-(4-Chlorobenzyl)-1-(2,3-dihydro-1H-inden-5-yl)-6-(4-morpholinylmethyl)-4-oxo-1,4-dihydro-3-quinolinecarboxamide;

(16) 1-(1,3-Benzodioxol-5-yl)-N-(4-chlorobenzyl)-6-(4-morpholinylmethyl)-4-oxo-1,4-dihydro-3-quinolinecarboxamide;

(17) N-(4-Chlorobenzyl)-1-(1H-indol-5-yl)-6-(4-morpholinylmethyl)-4-oxo-1,4-dihydro-3-quinolinecarboxamide;

(18) N-(4-Fluorobenzyl)-1-(1H-indol-5-yl)-6-(4-morpholinylmethyl)-4-oxo-1,4-dihydro-3-quinolinecarboxamide;

(19) N-(4-Chlorobenzyl)-1-(3-hydroxyphenyl)-6-(4-morpholinylmethyl)-4-oxo-1,4-dihydro-3-quinolinecarboxamide;

(20) N-(4-Chlorobenzyl)-1-(3-(2-hydroxyethyl)phenyl)-6-(4-morpholinylmethyl)-4-oxo-1,4-dihydro-3-quinolinecarboxamide;

(21) N-(4-Fluorobenzyl)-1-(3-(2-hydroxyethyl)phenyl)-6-(4-morpholinylmethyl)-4-oxo-1,4-dihydro-3-quinolinecarboxamide;

(22) N-(4-chlorobenzyl)-1-(3-methoxyphenyl)-6-(4-morpholinylmethyl)-4-oxo-1,4-dihydro-3-quinolinecarboxamide;

(23) N-(4-chlorobenzyl)-1-(3-(hydroxymethyl)phenyl)-6-(4-morpholinylmethyl)-4-oxo-1,4-dihydro-3-quinolinecarboxamide;

(24) N-(4-chlorobenzyl)-6-(4-morpholinylmethyl)-1-(4-(4-morpholinyl)phenyl)-4-oxo-1,4-dihydro-3-quinolinecarboxamide;

(25) N-(4-chlorobenzyl)-1-(3,4-difluorophenyl)-6-(4-morpholinylmethyl)-4-oxo-1,4-dihydro-3-quinolinecarboxamide;

(26) N-(4-chlorobenzyl)-1-(3-(3-hydroxy-1-propynyl)phenyl)-6-(4-morpholinylmethyl)-4-oxo-1,4-dihydro-3-quinolinecarboxamide;

(27) N-(4-chlorobenzyl)-1-(3-(4-hydroxy-1-butynyl)phenyl)-6-(4-morpholinylmethyl)-4-oxo-1,4-dihydro-3-quinolinecarboxamide;

(28) N-(4-chlorobenzyl)-1-(3-(4-hydroxy-1-butynyl)phenyl)-6-(4-morpholinylmethyl)-4-oxo-1,4-dihydro-3-quinolinecarboxamide;

(29) N-(4-chlorobenzyl)-1-(3-(5-hydroxypentyl)phenyl)-6-(4-morpholinylmethyl)-4-oxo-1,4-dihydro-3-quinolinecarboxamide;

(30) N-(4-chlorobenzyl)-1-(3-(4-hydroxybutyl)phenyl)-6-(4-morpholinylmethyl)-4-oxo-1,4-dihydro-3-quinolinecarboxamide;

(31) N-(4-chlorobenzyl)-1-[3-(3-hydroxypropyl)phenyl]-6-(4-morpholinylmethyl)-4-oxo-1,4-dihydro-3-quinolinecarboxamide;

and pharmaceutically acceptable salts thereof.

A method for preventing or treating atherosclerosis or restenosis in mammals, wherein the compound administered has the Formula XI and is N-(4-chlorobenzyl)-6-(4-morpholinylmethyl)-4-oxo-1-phenyl-1,4-dihydro-3-quinolinecarboxamide or a pharmaceutically acceptable salt thereof.

A method for preventing or treating atherosclerosis or restenosis in mammals, wherein the compound administered has the Formula XI and ^{XI-}X is Cl.

A method for preventing or treating atherosclerosis or restenosis in mammals, wherein the compound administered has the Formula XI and either R^{XI-2} or R^{XI-4} or both R^{XI-2} and R^{XI-4} do not represent H.

### Dosages and Dosage Forms

By the term “effective amount” of a compound as provided herein is meant a nontoxic but sufficient amount of one or more anti-atherosclerosis or anti-restenosis agents to provide the desired effect. The desired effect may be to prevent, give relief from, or ameliorate atherosclerosis or restenosis.

As pointed out below, the exact amount of the anti-atherosclerosis or anti-restenosis agent required to treat atherosclerosis or restenosis will vary from subject to subject, depending on the species, age, and general condition of the subject, the severity of the disease that is being treated, the particular compound(s) used, the mode of administration, such as the route and frequency of administration, and the particular compound(s) employed, and the like. Thus, it is not possible to specify an exact “effective amount.” However, an appropriate effective amount may be determined by one of ordinary skill in the art using only routine experimentation.

Pharmaceutical compositions including one or more anti-atherosclerosis or anti-restenosis agents of Formula I-V or XI can be administered orally or parenterally at dose levels, calculated as the free base, of each of the anti-atherosclerosis or anti-restenosis agent at 0.1 to 300 mg/kg of mammal body weight, preferably 1.0 to 30 mg/kg of mammal body weight, and can be used in a human in a unit dosage form, administered one to four times daily in the amount of 1 to 1000 mg per unit dose. The desired dosage may conveniently be presented in a single dose or as divided into multiple doses administered at appropriate intervals, for example, as two, three, four or more sub-doses per day. The sub-dose itself may be further divided, e.g., into a number of discrete loosely spaced aministrations.

Initial treatment of a patient suffering from atherosclerosis or restenosis can begin with a dosage regimen as indicated above. Treatment is generally continued as necessary over a period of several weeks to several months or years until the condition or disorder has been controlled or eliminated. Patents undergoing treatment with a composition of the invention can be routinely monitored by any of the methods well known in the art to determine the effectiveness of therapy. Continuous analysis of data from such monitoring permits modification of the treatment regin during therapy so that optimally effective amounts of drug are administered at any point in time, and so that the duration of treatment can be determined. In this way, the treatment regimen and dosing schedule can be rationally modified over the course of therapy so that the lowest amount of the compounds of this invention exhibiting satisfactory effectiveness is administered, and so that administration is continued only for so long as is necessary to successfully treat the condition or disorder.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In a combination therapy, the anti-atherosclerosis or anti-restenosis agent compound(s) and other inhibitor compound(s) can be administered simultaneously or at separate intervals. When administered simultaneously the anti-atherosclerosis or anti-restenosis agent compound(s) and the other inhibitor compound(s) can be incorporated into a single pharmaceutical composition or into separate compositions, e.g., anti-atherosclerosis or anti-restenosis agent compound(s) in one composition and the other inhibitor compound(s) in another composition. For instance the combination therapy, the anti-atherosclerosis or anti-restenosis agent compound(s) may be administered concurrently or concomitantly with the other inhibitor compound(s). The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

When separately administered, therapeutically effective amounts of anti-atherosclerosis or anti-restenosis agent compound(s) and the other inhibitor compound(s) are administered on a different schedule. One may be administered before the other as long as the time between the two administrations falls within a therapeutically effective interval. A therapeutically effective interval is a period of time beginning when one of either (a) the anti-atherosclerosis or anti-restenosis agent compound(s), or (b) the other inhibitor compound(s) is aministered to a mammal and ending at the limit of the beneficial effect in the treatment of atherosclerosis or restenosis of the combination of (a) and (b). The methods of administration of the anti-atherosclerosis or anti-restenosis agent compound(s) and the other inhibitor compound(s) may vary. Thus, one agent may be administered orally, while the other is administered by injection.

A specific active agent may have more than one recommended dosage range, particularly for different routes of administration. Generally, an effective amount of dosage of anti-atherosclerosis or anti-restenosis agent compound(s), either administered individually or in combination with other inhibitor compound(s), will be in the range of about 0.1 to about 300 mg/kg of body weight/day, preferably about 1 to about 30 mg/kg of body weight/day. It is to be understood that the dosages of active component(s) may vary depending upon the requirements of each subject being treated and the severity of the atherosclerosis or restenosis.

In addition to the anti-atherosclerosis or anti-restenosis agents, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion or active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Generally, the concentration of each of the anti-atherosclerosis or anti-restenosis agents in a liquid composition, such as a lotion, will be from about 0.1 wt. % to about 20 wt. %, preferably from about 0.5 wt. % to about 10 wt. %. The solution may contain other ingredients, such as emulsifiers, antioxidants or buffers. The concentration in a semi-solid or solid composition, such as a gel or a powder, will be about 0.1 wt. % to about 5 wt. %, preferably about 0.5 wt. % to about 2.5 wt. %. When the topically deliverable, pharmaceutical composition of the present invention is utilized to effect targeted treatment of a specific internal site, each of the anti-atherosclerosis or anti-restenosis agent is preferably contained in the composition in an amount of from 0.05-10 wt. %., more preferably 0.5-5 wt. %.

### Routes of Administration

In therapeutic use for treating, or combating, viral infections in a mammal (i.e., human and animals) the pharmaceutical composition including the anti-atherosclerosis or anti-restenosis agent(s) can be administered orally, parenterally, topically, rectally, or intranasally.

Parenteral administrations include injections to generate a systemic effect or injections directly to the afflicted ara. Examples to parenteral administrations are subcutaneous, intravenous, intramuscular, intradermal, intrathecal, intraocular, intraventricular, and general infusion techniques.

Topical administrations includes transdermal delivery to generate a system effect.

The rectal administration includes the form of suppositories.

The intranasally administration includes nasal aerosol or inhalation applications.

Pharmaceutical compositions including the anti-atherosclerosis or anti-restenosis agent(s) may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the anti-atherosclerosis or anti-restenosis agent(s) can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a bonder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of pharmaceutical compositions with the anti-atherosclerosis or anti-restenosis agent(s) dissolved in water and water-propylene glycol and water-polyethylene glycol systems, optionally containing suitable conventional coloring agents, flavoring agents, stabilizers and thickening agents.

The anti-atherosclerosis or anti-restenosis agent(s) may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oil or aqueous vehicles, and may contain formulating materials such as suspending, stabilizing and/or dispersing agents.

For injection, the anti-atherosclerosis or anti-restenosis agent(s) may be formulated in aqueous solution, preferably in physiologically compatible buffers or physiological saline buffer. Suitable buffering agents include tri-sodium orthophosphate, sodium bicarbonate, sodium citrate, N-methyl-glucamine, L(+)-lysine and L(+)-arginine.

The compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the anti-atherosclerosis or anti-restenosis agent(s). Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the anti-atherosclerosis or anti-restenosis agent(s) may be in a powder form for constitution with a suitable vehicle, e.g., sterile, pyrogen-free water, before use.

For suppository administration, the pharmaceutical compositions may also be formulated by mixing the anti-atherosclerosis or anti-restenosis agent(s) with a suitable non-irritating excipient which is solid at room temperature but liquid at rectal temperature and therefore will melt in the rectum to release the drug. Such materials include cocoa butter, beeswax and other glycerides.

For administration by inhalation, the anti-atherosclerosis or anti-restenosis agent(s) can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a powder base such as lactose or starch.

In addition to the formulations described previously, the anti-atherosclerosis or anti-restenosis agent(s) may also be formulated as depot preparations. Such long acting formulations may be in the form of implants. The anti-atherosclerosis or anti-restenosis agent(s) may be formulated for this route of administration with suitable polymers, hydrophobic materials, or as a sparing soluble derivative such as, without limitation, a sparingly soluble salt.

Additionally, the anti-atherosclerosis or anti-restenosis agent(s) may be delivered using a sustained-release system. Various sustained-release materials have been estabilshed and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

In certain embodiments, the anti-atherosclerosis or anti-restenosis agent(s) are applied topically. For topical applications, the pharmaceutical composition may be formulated in a suitable ointment containing the anti-atherosclerosis or anti-restenosis agent(s) suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

Several different animal models are available to evaluate reduction of atherosclerosis or restinosis by antiviral drug treatment. In these models histological changes in the atherosclerotic lesions of aortic arteries are measured in animals infected with a herpesvirus and treated or untreated with an antiviral drug capable of inhibiting replication of the herpesvirus. The models include murine CMV infection of apoE deficient mice and rat CMV infection of rats. These models would mimic the effects of human CMV infection. MHV-68 is a murine gammaherpesvirus related to EBV. Antiviral treatment has been shown to reduce atherosclerosis caused by HMV-68 infection in apoE deficient mice. Drugs containing compounds of Formula I and II inhibit replication of these animal viruses so the models could be used to show an effect of drugs containing compounds of Formula I and II on development of atherosclerosis. Lemstrom, et al, “Cytomegalovirus Infection-Enhanced Allograft Atherosclerosis is prevented by DHPG Prophylaxis in the Rat”, Circulation Vol. 90, No. 4, October 1994, pp 1969-1978; Burnell et al, “Atherosclerosis in a poE Knockout Mice Infected with Multiple Pathogens”. Both of these references are herein incorporated by reference.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited by the claims which follow.

All published documents are incorporated by reference herein.

What is claimed is:

1. A method of preventing or treating atherosclerosis or restenosis in a mammal, comprising administering to said mammal an effective amount of the compound selected from the group consisting of structures of Formula I, Formula II, Formula III, Formula IV, Formula V and Formula XI; wherein Formula I is 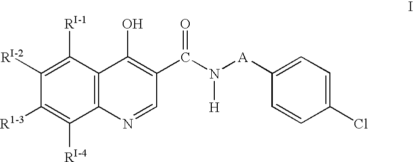
or a pharmaceutically acceptable salt thereof wherein
A is:
a) —CH₂—, or
b) —NH—;
R^{I-1}, R^{I-2}, R^{I-3} and R^{I-4} are independently
a) —H,
b) halo,
c) —CN,
d) —NO₂,
e) ^{I-}aryl,
f) ^{I-}het,
g) —OR^{I-5},
h) C₁₋₁₂ alkyl,
i) C₁₋₁₂ alkyl substituted with one to three —CN, halo, —NO₂, OR^{I-5}, —C(═O)R^{I-5}, —COOR^{I-5}, het, aryl, —SR^{I-5}, —OR^{I-6}, —NR^{I-7}R^{I-8}, —OP(═O)(R⁹)₂, —OPH(═O)R^{I-9}, —OC(═O)R^{I-10}, —O-glycyl, —O-valyl, or —O-lysyl,
j) —C≡CR^{I-11},
k) —CH═CH—R^{I-12},
l) —(CH₂)ₘ—C(═O)R^{I-13},
m) —SR^{I-14},
n) —C(═S)R^{I-5},
o) —(CH₂)ₘ—SOᵢR^{I-13},
p) —NR^{I-7}R^{I-8},
q) —NHSOᵢR^{I-13},
q) —NHSOᵢR^{I-13},
r) R^{I-1} and R^{I-2} taken together are ^{I-}het or C₄₋₆ cycloalkyl, or
s) R^{I-2} and R^{I-3} taken together are ^{I-}het or C₄₋₆ cycloalkyl;
R^{I-5} is
a) H,
b) C₁₋₈ alkyl, optionally substituted with one to three —OH, CN, C₁₋₄ alkoxy, halo, —NO₂, ^{I-}het or ^{I-}aryl,
c) ^{I-}aryl, or
d) ^{I-}het;
R^{I-6} is
a) —SO₂C₁₋₆ alkyl,
b) —SO₂—(CH₂)ₘ-^{I-}-aryl, or
c) —SO₂—(CH₂)ₘ-^{I-}het;
R^{I-7} and R^{I-8} are independently
a) H,
b) C₁₋₈ alkyl, optionally substituted with one to three —NO₂, halo, —CN, OR⁵, ^{I-}aryl, ^{I-}het, C₃₋₆ cycloalkyl, C₁₋₆ alkynyl, C₁₋₆ alkenyl, —SR¹⁴, or —NR^{I-16}R^{I-17},
c) ^{I-}aryl,
d) ^{I-}het,
e) —(CH₂)ₘ—C(═O)OR^{I-5},
f) —(CH₂)ₘ—C(═O)R^{I-5}, or
g) R^{I-7} and R^{I-8} taken together to form ^{I-}het;
R^{I-9} is
a) —OH, or
b) —OC₁₋₈ alkyl;
R^{I-10} is
a) H,
b) C₁₋₈ alkyl,
c) —NR^{I-7}R^{I-8},
d) C₁₋₈ alkyl substituted with one to two halo, ^{I-}het, —NR^{I-7}R^{I-8}, —COOH—O(CH₂)ₘCOOH or —C(═O)N(C₁₋₄alkyl)(CH₂)ₙS(═O)₂O⁻M⁺
R^{I-11} is
a) C₁₋₈ alkyl,
b) C₁₋₈ alkyl substituted with one to three —CN, halo, —NO₂, —COOR^{I-5}, —C(═O)R^{I-5}, —SR^{I-5}, ^{I-}aryl, —OR^{I-5}, —NR^{I-7}R^{I-8}, —OP(═O)(R^{I-9})₂, —OPH)(═O)R^{I-9} —OC(═O)R¹⁰, —O-glycyl, —O-valyl, —O-lysyl or —O-seluptamatyl, or
c) —(CH₂)ₘ-^{I-}het;
R^{I-12} is
a) H,
b) —CN,
c) C₁₋₈ alkyl,
d) C₁₋₈ alkyl substituted with one to three —CN, halo, —NO₂, —C(═O)R^{I-5}, —COOR^{I-5}, ^{I-}aryl, ^{I-}het, —SR^{I-5}, —OR^{I-5}, —NR^{I-7}R^{I-8}, —OP(═O)(R^{I-9})₂ or —OPH (═O)R^{I-9},
e) —C(═O)R^{I-5}, or
f) —COOR^{I-5};
R^{I-13} is
a) C₁₋₈ alkyl,
b) C₁₋₈ alkyl substituted one to three —CN, halo, —NO₂, —C(═O)R^{I-5}, ^{I-}het, ^{I-}aryl, —COOR^{I-5}, —SR^{I-5}, —OR^{I-5} or —NR^{I-7}R^{I-8},
c) ^{I-}het,
d) ^{I-}aryl,
e) —NR^{I-7}R^{I-8},
f) OR^{I-5}, or
g) halo;
R^{I-14} is
a) C₁₋₈ alkyl, or
b) C₁₋₈ alkyl substituted with one to three —CN, halo, —NO₂, —C(═O)R^{I-5}, —COOR^{I-5}, ^{I-}het, ^{I-}aryl —OR^{I-5}, or —NR^{I-7}R^{I-8};
R^{I-15} is
a) —NH₂, or
b) —NHNH₂;
R^{I-16} and R^{I-17} is independently
a) H,
b) C₁₋₄ alkyl,
c) —C(═O)C₁₋₄ alkyl, or
d) —C(═O)—(CH)ₘ-aryl;
aryl is phenyl or naphthyl, optionally substituted with R^{I-18};
het is a 5-, 6- or 7-membered saturated or unsaturated heterocyclic ring having 1-3 heteroatoms selected from the group consisting of nitrogen, oxygen and sulfur, wherein the heteroclyclic ring is optionally fused to a benzene ring, wherein aryl, het and benzene ring are optionally substituted with R^{I-18};
R^{I-18} is
a) halo,
b) —NO₂,
c) phenyl, optionally substituted with one to five —OH, —CN, halo, —NO₂, C₁₋₆ alkyl, het, or OC₁₋₄ alkyl,
d) C₁₋₈ alkyl, optionally substituted with one to three halo, —CN, —NO₂, aryl, —SR⁵, —OR⁵ or —NR^{I-7}R^{I-8},
e) OR⁵, or
f) —SO₂NH₂;
M^{I} is sodium, potassium or lithium atom;
i^{I} is 1 or 2;
m^{I} is 0, 1, 2, or 4;
n^{I} is 1, 2, 3 or 4;
wherein Formula II is 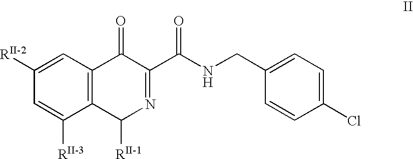
wherein
R^{II-1} is C₁₋₇ alkyl, optionally substituted by hydroxy or NR^{II-4}R⁵;
R^{II-2} is C₁₋₇ alkyl substituted by hydroxy or NR⁴R⁵;
R^{II-3} is H, F or C₁₋₇ alkoxy;
R^{II-4} and R^{II-5} together with N are a 5- or 6-membered heterocyclic moiety having 1-3 heteroatoms selected from the group consisting of nitrogen, oxygen and sulfur in which sulfur may be substituted by one (1) or two (2) oxygen atoms;
or a pharmaceutically acceptable salt thereof;
wherein, Formula III is 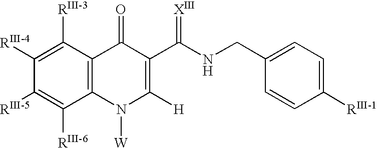
or a pharmaceutically acceptable salt thereof wherein,
X^{III} is
a) O, or
b) S;
W is
a) R^{III-2};
b) NR^{III-7}R^{III-8},
c) OR^{III-9}, or
d) SOᵢR^{III-9};
R^{III-2} is
a) Cl,
b) F,
c) Br,
d) CN, or
e) NO₂;
R^{III-2} is
a) (CH₂CH₂O)ₘR^{III-10},
b) het^{III}, wherein said het^{III} is bonded via a carbon atom,
c) C₁₋₇ alkyl which may be partially unsaturated and is optionally substituted by one or more substituents selected from a group consisting of NR^{III-7}R^{III-8}, R^{III-11}, CN, SOᵢR^{III-9}, or OC₂₋₄ alkyl which is further substituted by het^{III}, OR^{III-10}, OC(═O)aryl^{III}, or NR^{III-7}R^{III-8}, or
d) C₃₋₈ cycloalkyl, which may be partially unsaturated and is optionally substituted by R^{III-11}, NR^{III-7}R^{III-8}, SOᵢR^{III-9}, or C₁₋₇ alkyl optionally substituted by R^{III-11}, NR^{III-7}R^{III-8}, or SOᵢR^{III-9};
R^{III-3} is
a) H,
b) halo, or
c) C₁₋₄ alkyl, optionally substituted by one to three halo;
R^{III-4} is
a) H,
b) aryl^{III},
c) het^{III},
d) SO₂NHR^{III-12},
e) CONHR^{III-12},
f) NR^{III-7}R^{III-8},
g) NHCOR^{III-12},
h) NHSO₂R^{III-12},
i) OC₂₋₇ alkyl optionally substituted by —OH,
j) SC₂₋₇ alkyl optionally substituted by OH, or
k) C₁₋₈ alkyl which may be partially unsaturated and is optionally substituted by one or more substituents selected from a group consisting of N₃, OR^{III-10}, NR^{III-7}R^{III-8}, halo, SOᵢR^{III-9}, OR^{III-13} or R^{III-11},
R^{III-5} is
a) H,
b) halo,
c) C≡CR^{III-14},
d) NR^{III-7}R^{III-8},
e) SO₂NHR^{III-12},
f) het^{III}, or
g) C₁₋₇ alkyl, optionally substituted by OH;
R^{III-6} is
a) H,
b) halo,
c) SC₁₋₇ alkyl,
d) C₁₋₇ alkoxy, optionally substituted by one or more halo or OH, or
e) C₁₋₇ alkyl, which may be partially unsaturated and is optionally substituted by halo, NR^{III-10}R^{III-10}, (CH₂)ₙOR^{III-13}, R^{III-11}, OC₁₋₇ alkyl which is further substituted with ^{III-}het, NR^{III-7}R^{III-8}, or SOᵢR^{III-9};
R^{III-7} and R^{III-8} are independently
a) H,
b) aryl^{III},
c) C₁₋₇alkyl which may be partially unsaturated and is optionally substituted by one or more substituents selected from a group consisting of NR^{III-10}R^{III-10}, CONR^{III-10}R^{III-10}, R^{III-11}, SOᵢR^{III-9}, halo; or
d) R^{III-7} and R^{III-8} together with the nitrogen to which they are attached to form a het^{III};
R^{III-9} is
a) aryl^{III},
b) het^{III},
c) C₃₋₈cycloalkyl, or
d) C₁₋₇alkyl which may be partially unsaturated and optionally substituted by one or more OR^{III-10}, Oaryl^{III}, het^{III}, aryl^{III}, NR^{III-10}R^{III-10}, CN, SH, SOᵢC₁₋₆ alkyl, SOᵢ aryl^{III}, halo, or CONR^{III-10}R^{III-10};
R^{III-10} is
a) H, or
b) C₁₋₇ alkyl, optionally substituted by OH;
R^{III-11} is
a) OR^{III-10},
b) Ohet^{III},
c) Oaryl^{III},
d) CO₂R^{III-10},
e) het^{III},
f) aryl^{III}, or
g) CN;
R^{III-12} is
a) H,
b) het^{III},
c) aryl^{III},
d) C₃₋₈ cycloalkyl, or
e) C₁₋₇ alkyl optionally substituted by NR^{III-7}R^{III-8}, or R^{III-11};
R^{III-13} is
a) (P═O)(OH)₂,
b) (P═O)(C₁₋₇ alkoxy)₂,
c) CO(CH₂)ₙCON(CH₃)(CH₂)ₙSO₃⁻M⁺,
d) an amino acid,
e) C(═O)^{III-}aryl,
f) C(═O)C₁₋₆alkyl, optionally substituted by NR^{III-10}R^{III-10}, or
g) CO(CH₂)ₙCO₂H;
R^{III-14} is
a) het^{III},
b) (CH₂)ₙOR^{III-13}, or
c) C₁₋₇ alkyl substituted by one or more substituents selected from a group consisting of R^{III-11}, OC₁₋₇ alkyl which is further substituted with het^{III}, NR^{III-7}R^{III-8}, or SOᵢR^{III-9};
aryl^{III} is a phenyl radical or an ortho-fused bicyclic carbocyclic radical wherein at least one ring is aromatic;
het^{III} is a four- (4), five- (5), six- (6), or seven- (7) membered saturated or unsaturated heterocyclic ring having 1, 2, or 3 heteroatoms selected from the group consisting of oxygen, sulfur, and nitrogen, which is optionally fused to a benzene ring, or any bicyclic heterocycle group;
wherein any aryl^{III} is optionally substituted with one or more substituents selected from the group consisting of halo, OH, CF₃, C₁₋₆alkoxy, and C₁₋₆ alkyl which maybe further substituted by one to three SR^{III-10}, NR^{III-10}R^{III-10}, OR^{III-10}, or CO₂R^{III-10};
wherein any het^{III} is optionally substituted with one or more substituents selected from the group consisting of halo, OH, CF₃, C₁₋₆alkoxy, oxo, oxine, and C₁₋₆ alkyl which maybe further substituted by one to three SR^{III-10}, NR^{III-10} R^{III-10}, OR^{III-10}, or CO₂R^{III-10};
i^{III} is 0, 1, or 2;
m^{III} is 1, 2, or 3;
n^{III} is 1, 2, 3, 4, 5, or 6; and
M is sodium, potassium, or lithium;
wherein Formula IV is 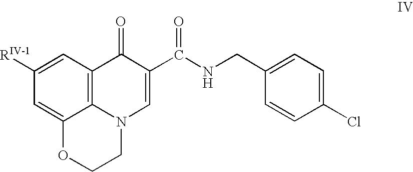
or a pharmaceutically acceptable salt, racemate, solvate, tautomer, optical isomer or prodrug derivative thereof;
wherein R^{IV-1} is C₁₋₆ alkyl, optionally substituted with —OH, —OC₁₋₄ alkyl or het^{IV};
wherein C₁₋₆ alkyl is optionally partially unsaturated;
wherein het^{IV} is a radical of a five- or six-membered heterocyclic ring having one or two heteroatoms selected from the group consisting of oxygen, sulfur and N;
wherein Formula V is 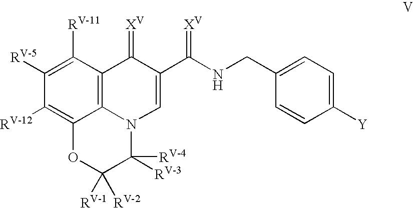
or a pharmaceutically acceptable salt, racemate, solvate, tautomer or optical isomer thereof
wherein:
each X^{V} independently O or S;
Y is Cl, F, Br, CN or NO₂;
R^{V-1}, R^{V-2}, R^{V-3} and R^{V-4} are independently
a) hydrogen,
b) N₃,
c) CN,
d) fluoro,
e) trifluoromethyl,
f) aryl^{V},
g) het^{V},
h) C_{V-1-V-8} alkyl, optionally substituted with R^{V-6} or OR^{V-7}, or
i) R^{V-1} and R^{V-2} or R^{V-3} and R^{V-4} together with the carbon to which they are attached form C₃₋₈cycloalkyl or _{V-}het;
R^{V-5} is C₁₋₈alkyl, which may be partially unsaturated and optionally substituted with one to three N₃, halo, CN, R^{V-6} or R^{V-7};
R^{V-6} is
a) aryl^{V},
b) het^{V},
c) SOᵢR^{V-8},
d) OR^{V-8},
e) C(═O)OR^{V-8},
f) C(═O)R^{V-8}, or
g) NR^{V-8}R^{V-9};
R^{V-7} is
a) P(═O)(OR^{V-10})₂,
b) CO(CH_{V-2})ⱼCON(CH₃)(CH₂)ₖSO₃⁻M^{V+},
c) an amino acid,
d) C(═O)C₁₋₆alkyl, optionally substituted by NR^{V-10}R^{V-10}, or
e) CO(CH₂)ₙCO₂H;
R^{V-8} and R^{V-9} are independently
a) hydrogen,
b) C₃₋₈cycloalkyl,
c) aryl^{V},
d) het^{V}, or
e) C₁₋₈alkyl which is further optionally substituted with one or more aryl^{V}, het^{V}, halo, CN, CO₂R^{V-10}, SOᵢR^{V-10}, OR^{V-10}, NR^{V-10}R^{V-10}, CF₃, or C₃₋₈ cycloalkyl;
R^{V-10} is
a) H or
b) C₁₋₈alkyl, optionally substituted with OH or OC₁₋₄alkyl;
R^{V-11} and R^{V-12} are independently
a) hydrogen,
b) halo,
c) NO₂,
d) CN,
e) R^{V-6},
f) SOᵢNR^{V-8}R^{V-9}, or
g) C₁₋₈alkyl, which may be partially unsaturated and optionally substituted with one to three N^{V-3}, halo, CN, R^{V-6} or OR^{V-7};
aryl^{V} is
a phenyl radical, optionally fused with a saturated or unsaturated carbocyclic or heterocyclic ring; at each occurrence, aryl^{V} may be substituted with one or more halo, CN, CO₂R^{V-10}, SOᵢR^{V-10}, OR^{V-10}, NR^{V-10}R^{V-10}, CF₃, C₃₋₈cycloalkyl, or C₁₋₄alkyl wherein C₁₋₄alkyl is optionally substituted with OR^{V-10};
het^{V} is
a four- (4), five- (5), six- (6), or seven- (7) membered saturated or unsaturated heterocyclic ring having 1, 2, or 3 heteroatoms selected from the group consisting of O, S, and NW^{V}, wherein W^{V} is hydrogen, C₁₋₄alkyl, C(═O)OC₁₋₄alkyl or absent, wherein het^{V} is optionally fused with a benzene ring, a carbcyclic or a heterocyclic ring; at each occurrence, het^{V} may be substituted with one or more halo, CN, CO₂R^{V-10}, SOᵢR^{V-10}, OR^{V-10}, NR^{V-10}R^{V-10}, C₁₋₄alkyl, CF₃, C₃₋₈cycloalkyl, oxo or oxine;
at each occurrence, a cycloalkyl may be substituted with C₁₋₄alkyl, OR^{V-10}, oxo, oxine, or a spiro fused _{V-}het;
i^{V} is 0, 1 or 2;
j^{V} is 1, 2, 3, 4, 5, or 6;
k^{V} is 1, 2, 3, 4, 5, or 6;
n^{V} is 1, 2, 3, 4, 5, or 6;
M^{V} is sodium, potassium, or lithium; and
wherein Formula XI is 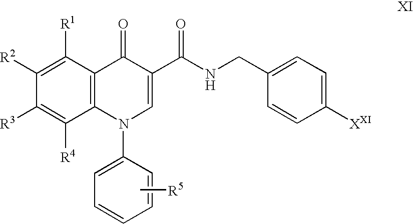
and pharmaceutically acceptable salts thereof,
wherein,
X^{XI} is Cl, F, Br, CN, or NO₂;
R^{XI-1} is H, halo, or C₁₋₄alkyl optionally substituted by one to three halo;
R^{XI-2} is
a) H,
b) halo,
c) aryl^{XI},
d) het^{XI}, wherein said het^{XI} is bound via a carbon atom,
e) C₁₋₇alkyl which may be partially unsaturated and optionally substituted by one or more substituents of the group R^{XI-10}, NR^{XI-7}R^{XI}^{I-8}, halo, (C═O)R^{XI-6}, or S(O)ₘR^{XI-6},
f) NR^{XI-7}R^{XI-8},
g) OR^{XI-11},
h) SR^{XI-11},
i) NHSO₂R^{XI-6},
j) S(O)ₘR^{XI-6},
k) (C═O)R^{XI-6},
l) (C═O) OR^{XI-11},
m) CHO,
n) cyano, or
o) C₃₋₈cycloalkyl which may be partially unsaturated and is optionally substituted by one or more substituents selected from halo, oxo, R^{XI-10}, C₁₋₇alkyl, or NR^{XI-7}R^{XI-8};
R^{XI-3} is
a) H,
b) halo,
c) OR¹¹, or
d) C₁₋₇alkyl which may be partially unsaturated and optionally substituted by one or more substituents of the group OR^{XI-11}, SR^{XI-11}, NR^{XI-7}R^{XI-8}, or halo, or
R^{XI-2} together with R^{XI-3} form a carbocyclic or saturated 5 or 6 membered het^{XI} which may be optionally substituted by NR^{XI-7}R^{XI-8}, het^{XI} attached through a carbon atom, or C₁₋₇alkyl which may be optionally substituted by OR^{XI-12};
R^{XI-4} is
a) H,
b) halo,
c) OR^{XI-11}, or
d) C₁₋₇alkyl which may be partially unsaturated and optionally substituted by one or more substituents of the group OR^{XI-11}, SR^{XI-11}, NR^{XI-7}R^{XI-8}, aryl^{XI}, halo, C₃₋₈cycloalkyl optionally substituted by OR^{XI-12}, or het^{XI} attached through a carbon atom, or
e) NR^{XI-7}R^{XI-8};
R^{XI-5} is
a) H,
b) halo,
c) OR^{XI-11},
d) O(CH₂CH₂O)ₙR^{XI-12},
e) C₃₋₈cycloalkyl which may be partially unsaturated and is optionally substituted by one or more substituents selected from halo, OR^{XI-12}, SR^{XI-12}, oxo, C₁₋₇alkyl or NR^{XI-12}R^{XI-12},
f) het^{XI},
g) aryl^{XI},
h) NHSO₂R^{XI-6},
i) S(O)ₘR^{XI-6},
j) (C═O)R^{XI-6},
k) (C═O)OR^{XI-11},
l) nitro,
m) cyano,
n) SR^{XI-11},
o) NR^{XI-7}R^{XI-8},
p) C₁₋₇alkyl which may be partially unsaturated and is optionally substituted by one or more substituents selected from NR^{XI-7}R^{XI-8}, R^{XI-10}, S(O)ₘR^{XI-6}, (P═O)(OR^{XI-12})₂, (C═O)R^{XI-6}, or halo,
q) CHO,
r) SCN,
s) Any two adjacent R^{XI-5} substituents taken with the bond connecting them form an aryl^{XI}, or het^{XI}, or
t) Any two adjacent R^{XI-5} substituents taken together constitute a C₃₋₆alkyl chain which may be optionally substituted by R^{XI-9}, NR^{XI-7}R^{XI-8}, cyano, CO₂R^{XI-12}, OR^{XI-11}, SR^{XI-11}, or (═O);
R^{XI-6} is
a) C₁₋₇alkyl,
b) NR^{XI-11}R^{XI-11},
c) aryl^{XI}, or
d) het^{XI};
R^{XI-7} and R^{XI-8} are independently
a) H,
b) aryl^{XI},
c) C₁₋₇alkyl which may be partially unsaturated and is optionally substituted by one or more substituents selected from S(O)ₘR^{XI-6}, CONR^{XI-12}R^{XI-12}, CO₂R^{XI-12}, (C═O)R^{XI-9}, het^{XI}, aryl^{XI}, cyano, or halo,
d) C₂₋₇alkyl which may be partially unsaturated and is substituted by one or more substituents selected from NR^{XI-12}R^{XI-12}, OR^{XI-11}, or SR^{XI-11},
e) C₃₋₈cycloalkyl which may be partially unsaturated and is optionally substituted by one or more substituents selected from halo, OR^{XI-12}, SR^{XI-12}, oxo, or NR^{XI-12}R^{XI-12},
f) (C═O)R^{XI-9}, or
g) R^{XI-7} and R^{XI-8} together with the nitrogen to which they are attached for a het^{XI};
R^{XI-9} is
a) aryl^{XI},
b) het^{XI}, wherein said het^{XI} is bound through a carbon atom,
c) C₁₋₇alkyl optionally substituted by aryl^{XI}, het^{XI}, cyano, OR^{XI-12}, SR^{XI-12}, NR^{XI-12}R^{XI-12}, or halo, or
d) C₃₋₈cycloalkyl which may be partially unsaturated and is optionally substituted by one or more substituents selected from halo, OR^{XI-12}, SR^{XI-12}, or NR^{XI-12}R^{XI-12};
R^{XI-10} is
a) OR^{XI-11},
b) SR^{XI-11},
c) CO₂R^{XI-12},
d) het^{XI},
e) aryl^{XI}, or
f) cyano;
R^{XI-11} is
a) H,
b) aryl^{XI},
c) het^{XI}, wherein said het^{XI} is bound through a carbon atom,
d) C₁₋₇alkyl optionally substituted by aryl^{XI}, het^{XI} wherein said het^{XI} is bound through a carbon atom, C₃₋₈cycloalkyl optionally substituted by OR^{XI-12}, or halo,
e) C₂₋₇alkyl substituted by OR^{XI-12}, SR^{XI-12}, or NR^{XI-12}R^{XI-12}, or
f) C₃₋₈cycloalkyl which may be partially unsaturated and is optionally substituted by one or more substituents selected from halo, -OR^{XI-12}, SR^{XI-12}, or NR^{XI-12}R^{XI-12},
R^{XI-12} is H, or C₁₋₇alkyl;
each m^{XI} is independently 1 or 2;
each n^{XI} is independently 1, 2, or 3;
wherein aryl^{XI} is a phenyl radical or an ortho-fused bicyclic carbocyclic radical wherein at least one ring is aromatic and is optionally substituted with one or more substituents selected from halo, OH, cyano, CO₂R^{XI-12}, CF₃, C₁₋₆alkoxy, or C₁₋₆ alkyl which may be further substituted by one to three SR^{XI-12}, NR^{XI-12}R^{XI-12}, OR^{XI-12}, or CO₂R^{XI-12} groups;
wherein het^{XI} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocyclic group and wherein any het^{XI} is optionally substituted with one or more substituents selected from halo, OH, cyano, phenyl, CO₂R^{XI-12}, CF₃, C₁₋₆alkoxy, oxo, oxime, or C₁₋₆ alkyl which may be further substituted by one to three SR^{XI-12}, NR^{XI-12}R^{XI-12}, OR^{XI-12}, or CO₂R^{XI-12}, groups; and
wherein halo is F, Cl, Br, I;
or a pharmaceutically acceptable salt thereof.

2. The method of claim 1, wherein the compound administered has the Formula I 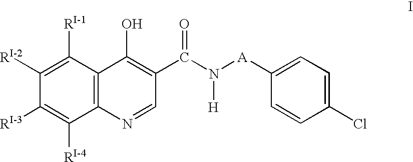
or a pharmaceutically acceptable salt thereof wherein A is:
a) —CH₂—, or
b) —NH—;
R^{I-1}, R^{I-2}, R^{I-3} and R^{I-4} are independently
a) —H,
b) halo,
c) —CN,
d) —NO₂,
e) aryl^{I},
f) het^{I},
g) —OR^{I-5},
h) C₁₋₁₂ alkyl,
i) C₁₋₁₂ alkyl substituted with one to three —CN, halo, —NO₂, OR^{I-5}, —C(═O)R^{I-5}, —COOR^{I-5}, ^{I-}het, ^{I-}aryl, —SR^{I-5}, —OR^{I-6}, —NR^{I-7}R^{I-8}, —OP(═O)(R^{I-9})₂, —OPH(═O)R^{I-9}, —OC(═O)R^{I-10}, —O-glycyl, —O-valyl, or —O-lysyl,
j) —C≡CR^{I-11},
k) —CH═CH—R^{I-12},
l) —(CH₂)ₘ—C(═O)R^{I-13},
m) —SR^{I-14},
n) —C(═S)R^{I-15},
o) (CH₂)ₘ—SOᵢR^{I-13},
p) —NR^{I-7}R^{I-8},
q) —NHSOᵢR^{I-13},
r) R^{I-1} and R^{I-2} taken together are het^{I} or C₄₋₆ cycloalkyl, or
s) R^{I-2} and R^{I-3} taken together are het^{I} or C₄₋₆ cycloalkyl;
R^{I-5} is
a) H,
b) C₁₋₈ alkyl, optionally substituted with one to three —OH, CN, C₁₋₄ alkoxy, halo, —NO₂, het^{I} or aryl^{I},
c) aryl^{I}, or
d) het^{I};
R^{I-6} is
a) —SO₂C₁₋₆ alkyl,
b) —SO₂—(CH₂)ₘ-aryl^{I}, or
c) —SO₂—(CH₂)ₘ-het^{I};
R^{I-7} and R^{I-8} are independently
a) H,
b) C₁₋₈ alkyl, optionally substituted with one to three —NO₂, halo, —CN, OR^{I-5}, aryl, het, C₃₋₆ cycloalkyl, C₁₋₆ alkynyl, C₁₋₆ alkenyl, —SR^{I-14}, or —NR^{I-16}R^{I-17},
c) aryl^{I},
d) het^{I},
e) —(CH₂)ₘ—C(═O)OR^{I-5},
f) —(CH₂)ₘ—C(═O)R^{I-5}, or
g) R^{I-7} and R^{I-8} taken together to form het^{I};
R^{I-9} is
a) —OH, or
b) —OC₁₋₈ alkyl;
R^{I-10} is
a) H,
b) C₁₋₈ alkyl,
c) —NR^{I-7}R^{I-8},
d) C₁₋₈ alkyl substituted with one to two halo, het^{I}, —NR^{I-7}R^{I-8}, —COOH—O(CH₂)ₘCOOH or —C(═O)N(C₁₋₄ alkyl)(CH₂)ₙS(═O)₂O⁻M^{I+}
R^{I-11} is
a) C₁₋₈ alkyl,
b) C₁₋₈ alkyl substituted with one to three —CN, halo, —NO₂, —COOR^{I-5}, —C(═O)R^{I-5}, —SR^{I-5}, aryl^{I}, —OR^{I-5}, NR^{I-7}R^{I-8}, —OP(═O)(R^{I-9})₂, —OPH(═O)R^{I-9} —OC(═O)R¹⁰, —O-glycyl, —O-valyl, —O-lysyl or —O-seluptamatyl, or
c) —(CH₂)ₘ-het^{I};
R^{I-12} is
a) H,
b) —CN,
c) C₁₋₈ alkyl,
d) C₁₋₈ alkyl substituted with one to three —CN, halo, —NO₂, —C(═O)R^{I-5}, —COOR^{I-5}, aryl^{I}, het^{I}, —SR^{I-5}, —OR^{I-5}, —NR^{I-7}R^{I-8}, —OP(═O)(R^{I-9})₂ or —OPH(═O)R^{I-9},
e) —C(═O)R^{I-5}, or
f) —COOR^{I-5};
R^{I-13} is
a) C₁₋₈ alkyl,
b) C₁₋₈ alkyl substituted one to three —CN, halo, —NO₂, —C(═O)R^{I-5}, het^{I}, aryl^{I}, —COOR^{I-5}, —SR^{I-5}, —OR^{I-5} or —NR^{I-7}R^{I-8},
c) het^{I},
d) aryl^{I},
e) —NR^{I-7}R^{I-8},
f) OR^{I-5}, or
g) halo;
R^{I-14} is
a) C₁₋₈ alkyl, or
b) C₁₋₈ alkyl substituted with one to three —CN, halo, —NO₂, —C(═O)R^{I-5}, —COOR^{I-5}, het_{I}, aryl^{I}, —OR^{I-5}, or —NR^{I-7}R^{I-8};
R^{I-15} is
a) —NH₂, or
b) —NHNH₂;
R^{I-16} and R^{I-17} is independently
a) H,
b) C₁₋₄ alkyl,
c) —C(═O)C₁₋₄ alkyl, or
d) —C(═O)—(CH)ₘ-^{I-}aryl;
aryl^{I} is phenyl or naphthyl, optionally substituted with R^{I-18};
het^{I} is a 5-, 6- or 7-membered saturated or unsaturated heterocyclic ring having 1-3 heteroatoms selected from the group consisting of nitrogen, oxygen and sulfur, wherein the heteroclyclic ring is optionally fused to a benzene ring, wherein aryl^{I}, het^{I}, and benzene ring are optionally substituted with R^{I-18};
R^{I-18} is
a) halo,
b) —NO₂,
c) phenyl, optionally substituted with one to five —OH, —CN, halo, —NO₂, C₁₋₆ alkyl, het^{I}, or OC₁₋₄ alkyl,
d) C₁₋₈ alkyl, optionally substituted with one to three halo, —CN, —NO₂, aryl^{I}, —SR^{I-5}, —OR^{I-5} or —NR^{I-7}R^{I-8},
e) OR^{I-5}, or
f) —SO₂NH₂;
m^{I} is sodium, potassium or lithium atom;
i^{I} is 1 or 2;
M^{I} is 0, 1, 2, or 4;
n^{I} is 1, 2, 3 or 4;

3. The method of claim 2, wherein the compound administered is
(1) N-[(4-chlorophenyl)methyl]-4-hydroxy-7-(trifluoromethyl)-3-quinolinecarboxamide;
(2) 7-amino-N-[(4-chlorophenyl)methyl]-4-hydroxy-3-quinolinecarboxamide;
(3) N-[(4-chlorophenyl)methyl]-8-fluoro-4,6-dihydroxy-3-quinolinecarboxamide;
(4) 6-bromo-N-[(4-chlorophenyl)methyl]-8-fluoro-3-quinolinecarboxamide;
(5) N-[(4-chlorophenyl)methyl]-8-fluoro-4-hydroxy-6-iodo-3-quinolinecarboxamide;
(6) N-[(4-chlorophenyl)methyl]-4-hydroxy-3-quinolinecarboxamide;
(7) N-[(4-chlorophenyl)methyl]-4-hydroxy-7-methoxy-3-quinolinecarboxamide;
(8) N-[(4-chlorophenyl)methyl]-4-hydroxy-5,7-bis(trifluoromethyl)-3-quinolinecarboxamide;
(9) N-[(4-chlorophenyl)methyl]-7-fluoro-4-hydroxy-3-quinolinecarboxamide;
(10) N-[(4-chlorophenyl)methyl]-6-fluoro-4-hydroxy-3-quinolinecarboxamide;
(11) N-[(4-chlorophenyl)methyl]-4-hydroxy-7-methyl-3-quinolinecarboxamide;
(12) N-[(4-chlorophenyl)methyl]-8-fluoro-4-hydroxy-3-quinolinecarboxamide;
(13) N-[(4-chlorophenyl)methyl]-4-hydroxy-6-nitro-3-quinolinecarboxamide;
(14) N-[(4-chlorophenyl)methyl]-5,6,7,8-tetrafluoro-4-hydroxy-3-quinolinecarboxamide;
(15) N-[(4-chlorophenyl)methyl]-6,7,8-trifluoro-4-hydroxy-3-quinolinecarboxamide;
(16) 6,7,8-trifluoro-4-hydroxy-3-quinolinecarboxylic acid 2-(4-chlorophenyl)hydrazide;
(17) N-[(4-chlorophenyl)methyl]-5,8-difluoro-4-hydroxy-3-quinolinecarboxamide;
(18) N-[(4-chlorophenyl)methyl]-7,8-difluoro-4-hydroxy-3-quinolinecarboxamide;
(19) 6-benzoyl-N-(4-chlorobenzyl)-4-hydroxy-3-quinolinecarboxamide;
(20) N-[(4-chlorophenyl)methyl]-4-hydroxy-8-methoxy-3-quinolinecarboxamide;
(21) 6-chloro-N-[(4-chlorophenyl)methyl]-4-hydroxy-3-quinolinecarboxamide;
(22) N-[(4-chlorophenyl)methyl]-4-hydroxy-6-methyl-3-quinolinecarboxamide;
(23) N-[(4-chlorophenyl)methyl]-4-hydroxy-6-methoxy-3-quinolinecarboxamide;
(24) N-[(4-chlorophenyl)methyl]-6-cyano-4-hydroxy-3-quinolinecarboxamide;
(25) 7-(acetylamino)-N-[(4-chlorophenyl)methyl]-4-hydroxy-3-quinolinecarboxamide;
(26) N-[(4-chlorophenyl)methyl]-4-hydroxy-7-[(methylsulfonyl)amino]-3-quinolinecarboxamide;
(27) N-[(4-chlorophenyl)methyl]-7-(dimethylamino)-4-hydroxy-3-quinolinecarboxamide;
(28) 6-amino-N-[(4-chlorophenyl)methyl]-4-hydroxy-3-quinolinecarboxamide;
(29) N-[(4-chlorophenyl)methyl]-4-hydroxy-6-[(methylsulfonyl)amino]-3-quinolinecarboxamide;
(30) N-[(4-chlorophenyl)methyl]-6-(dimethylamino)-4-hydroxy-3-quinolinecarboxamide;
(31) 6-(acetylamino)-N-[(4-chlorophenyl)methyl]-4-hydroxy-3-quinolinecarboxamide;
(32) N-[(4-chlorophenyl)methyl]-4-hydroxy-7-(1-pyrrolyl)-3-quinolinecarboxamide;
(33) N-[(4-chlorophenyl)methyl]-4-hydroxy-7-[(phenylsulfonyl)amino]-3-quinolinecarboxamide;
(34) N-[(4-chlorophenyl)methyl]-4-hydroxy-7-[[(phenylmethyl)sulfonyl]amino]-3-quinolinecarboxamide;
(35) N-[(4-chlorophenyl)methyl]-7-[[(4-chlorophenyl)sulfonyl]amino]-4-hydroxy-3-quinolinecarboxamide;
(36) 8-fluoro-4-hydroxy-3-quinolinecarboxylic acid 2-(4-chlorophenyl)hydrazide;
(37) N-[(4-chlorophenyl)methyl]-8-fluoro-4-hydroxy-6-methyl-3-quinolinecarboxamide;
(38) (278) N-(4-chlorobenzyl)-8-hydroxy[1,3]dioxolo[4,5-g]quinoline-7-carboxamide;
(39) N-[(4-chlorophenyl)methyl]-4-hydroxy-6-iodo-3-quinolinecarboxamide;
(40) N-[(4-chlorophenyl)methyl]-6-(cyanomethyl)-4-hydroxy-3-quinolinecarboxamide;
(41) N-[(4-chlorophenyl)methyl]-4,5-dihydroxy-3-quinolinecarboxamide;
(42) 7,8-dichloro-N-[(4-chlorophenyl)methyl]-4-hydroxy-3-quinolinecarboxamide;
(43) N-[(4-chlorophenyl)methyl]-4,6-dihydroxy-3-quinolinecarboxamide;
(44) N-[(4-chlorophenyl)methyl]-4,8-dihydroxy-3-quinolinecarboxamide;
(45) 8-chloro-N-[(4-chlorophenyl)methyl]-4-hydroxy-3-quinolinecarboxamide;
(46) N-[(4-chlorophenyl)methyl]-4-hydroxy-6-[[(1-phenyl-1H-pyrazol-5-yl)amino]sulfonyl]-3-quinolinecarboxamide;
(47) N-[(4-chlorophenyl)methyl]-8-cyano-4-hydroxy-3-quinolinecarboxamide;
(48) N-[(4-chlorophenyl)methyl]-4-hydroxy-8-nitro-3-quinolinecarboxamide;
(49) 7-amino-N-[(4-chlorophenyl)methyl]-4-hydroxy-8-methyl-3-quinolinecarboxamide;
(50) N-[(4-chlorophenyl)methyl]-6-cyano-8-fluoro-4-hydroxy-3-quinolinecarboxamide;
(51) 6-(aminothioxomethyl)-N-[(4-chlorophenyl)-methyl]8-fluoro-4-hydroxy-3-quinolinecarboxamide;
(52) N-[(4-chlorophenyl)methyl]-8-fluoro-4-hydroxy-6-(3-hydroxy-1-propynyl)-3-quinolinecarboxamide;
(53) 8-fluoro-4-hydroxy-6-iodo-3-quinolinecarboxylic acid 2-(4-chlorophenyl)hydrazide;
(54) 8-fluoro-4-hydroxy-6-methyl-3-quinolinecarboxylic acid 2-(4-chlorophenyl)hydrazide;
(55) N-((4-chlorophenyl)methyl)-7-chloro-4-hydroxy-3-quinolinecarboxamide;
(56) N-((4-chlorophenyl)methyl)-6-bromo-4-hydroxy-3-quinolinecarboxamide;
(57) N-((4-chlorophenyl)methyl)-4-hydroxy-6-phenyl-3-quinolinecarboxamide;
(58) N-((4-chlorophenyl)methyl)-8-chloro-4-hydroxy-5-trifluoromethyl-3-quinolinecarboxamide;
(59) N-((4-chlorophenyl)methyl)-6,8-dimethoxy-4-hydroxy-3-quinolinecarboxamide;
(60) N-((4-chlorophenyl)methyl)-6,7-dimethoxy-4-hydroxy-3-quinolinecarboxamide;
(61) N-((4-chlorophenyl)methyl)-4-hydroxy-5-methyl-3-quinolinecarboxamide;
(62) N-[(4-chlorophenyl)methyl]-6-(1,1-dimethylethyl)-4-hydroxy-3-quinolinecarboxamide;
(63) N-[(4-chlorophenyl)methyl]-7,8-dihydro-4-hydroxy-6H-cyclopenta[g]quinoline-3-carboxamide;
(64) N-[(4-chlorophenyl)methyl]-1,4-dihydro-8-(methylthio)-4-oxo-3-quinolinecarboxamide;
(65) N-[(4-chlorophenyl)methyl]-9-hydroxythiazolo[5,4-f]quinoline-8-carboxamide;
(66) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;
(67) sodium 2-[(8-{[3-(3-{[(4-chlorobenzyl)amino]carbonyl}-8-fluoro-4-hydroxy-6-quinolinyl)-2-propynyl]oxy}-8-oxooctanoyl)(methyl)amino]-1-ethanesulfonate;
(68) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;
(69) N-(4-chlorobenzyl)-4-hydroxy-7-{[(1-naphthylmethyl)amino]sulfonyl}-3-quinolinecarboxamide;
(70) N-(4-chlorobenzyl)-4-hydroxy-7-(methylsulfanyl)-3-quinolinecarboxamide;
(71) N-[(4-chlorophenyl)methyl]-4-hydroxy-6-[(phenylmethyl)thio]-7-(trifluoromethyl)-3-quinolinecarboxamide;
(72) 3-(3-{[(4-chlorobenzyl)amino]carbonyl}-4-hydroxy-6-quinolinyl)propyl hydrogen phosphonate;
(73) N-[(4-chlorophenyl)methyl]-8-fluoro-4-hydroxy-6-(2-thiazolyl)-3-quinolinecarboxamide;
(74) N-[(4-chlorophenyl)methyl]-8-fluoro-4-hydroxy-6-(2-thiophenyl)-3-quinolinecarboxamide;
(75) N-((4-chlorophenyl)methyl)-4-hydroxy-5-trifluoromethyl-3-quinolinecarboxamide;
(76) N-((4-chlorophenyl)methyl)-8-fluoro-4-hydroxy-6-(2-methylphenyl)-3-quinolinecarboxamide;
(77) N-((4-chlorophenyl)methyl)-6,7-difluoro-4-hydroxy-8-(tetrahydro-2H-pyran-4-oxy)-3-quinolinecarboxamide;
(78) N-((4-chlorophenyl)methyl)-6,7-difluoro-4-hydroxy-8-methoxy-3-quinolinecarboxamide;
(79) N-((4-chlorophenyl)methyl)-7,8-dimethoxy-6-fluoro-4-hydroxy-3-quinolinecarboxamide;
(80) N-((4-chlorophenyl)methyl)-6,8-difluoro-4-hydroxy-7-(4-(hydroxymethyl)phenoxy)-3-quinolinecarboxamide;
(81) N-((4-chlorophenyl)methyl)-6,8-difluoro-4-hydroxy-7-methoxy-3-quinolinecarboxamide;
(82) N-((4-chlorophenyl)methyl)-6,8-difluoro-4-hydroxy-7-(2-(methoxy)ethoxy)-3-quinolinecarboxamide;
(83) N-((4-chlorophenyl)methyl)-6,7-difluoro-4-hydroxy-8-(2-(methoxy)ethoxy)-3-quinolinecarboxamide;
(84) N-((4-chlorophenyl)methyl)-7,8-di(2-(methoxy)ethoxy)-6-fluoro-4-hydroxy-3-quinolinecarboxamide;
(85) N-((4-chlorophenyl)methyl)-6,8-difluoro-4-hydroxy-7-(1-methylethoxy)-3-quinolinecarboxamide;
(86) N-(4-chlorobenzyl)-8-fluoro-4-hydroxy-6-(1,3-thiazol-2-yl)-3-quinolinecarboxamide;
(87) N-(4-chlorobenzyl)-6,8-difluoro-4-hydroxy-7-[(2-methoxyethyl)amino]-3-quinolinecarboxamide;
(88) N-(4-chlorobenzyl)-6-(5-cyano-1-pentynyl)-8-fluoro-4-hydroxy-3-quinolinecarboxamide;
(89) N-(4-chlorobenzyl)-8-fluoro-4-hydroxy-6-(2-pyridinyl)-3-quinolinecarboxamide;
(90) N′-(4-chlorophenyl)-4-hydroxy-6-iodo-3-quinolinecarbohydrazide;
(91) N-(4-chlorobenzyl)-8-fluoro-4-hydroxy-6-[2-(2-pyridinyl)ethynyl]-3-quinolinecarboxamide;
(92) N-(4-chlorobenzyl)-6,8-difluoro-4-hydroxy-7-[(2-hydroxyethyl)amino]-3-quinolinecarboxamide;
(93) N-(4-chlorobenzyl)-8-fluoro-4-hydroxy-6-(4-hydroxy-1-butynyl)-3-quinolinecarboxamide;
(94) N-(4-chlorobenzyl)-8-fluoro-4-hydroxy-6-(3-methoxy-1-propynyl)-3-quinolinecarboxamide;
(95) N-(4-chlorobenzyl)-8-fluoro-4-hydroxy-6-(3-hydroxy-1-butynyl)-3-quinolinecarboxamide;
(96) 6-(4-bromo-2-thienyl)-N-(4-chlorobenzyl)-8-fluoro-4-hydroxy-3-quinolinecarboxamide;
(97) N-(4-chlorobenzyl)-8-fluoro-6-(hydrazino-carbothioyl)-4-hydroxy-3-quinolinecarboxamide;
(98) N-(4-chlorobenzyl)-8-fluoro-4-hydroxy-6-(4-hydroxybutyl)-3-quinolinecarboxamide;
(99) N-(4-chlorobenzyl)-8-fluoro-4-hydroxy-6-(5-methyl-1,3,4-thiadiazol-2-yl)-3-quinolinecarboxamide;
(100) N-(4-chlorobenzyl)-4-hydroxy-7-(3-hydroxy-1-propynyl)-3-quinolinecarboxamide;
(101) 7-(aminocarbothioyl)-N-(4-chlorobenzyl)-4-hydroxy-3-quinolinecarboxamide;
(102) N-(4-chlorobenzyl)-8-fluoro-4-hydroxy-6-(3-methoxypropyl)-3-quinolinecarboxamide;
(103) N-(4-chlorobenzyl)-8-fluoro-4-hydroxy-6-[(Z)-4-hydroxy-1-butenyl]-3-quinolinecarboxamide;
(104) N-(4-chlorobenzyl)-6-(5-cyanopentyl)-8-fluoro-4-hydroxy-3-quinolinecarboxamide;
(105) N-(4-chlorobenzyl)-8-fluoro-4-hydroxy-6-(3-hydroxy-3-methylbutyl)-3-quinolinecarboxamide;
(106) N-(4-chlorobenzyl)-8-fluoro-4-hydroxy-6-(5-hydroxy-1-pentynyl)-3-quinolinecarboxamide;
(107) 6-{3-[benzyl(methyl)amino]propyl}-N-(4-chlorobenzyl)-8-fluoro-4-hydroxy-3-quinolinecarboxamide;
(108) methyl 3-{[(4-chlorobenzyl)amino]carbonyl}-8-fluoro-4-hydroxy-6-quinolinecarboxylate;
(109) N-(4-chlorobenzyl)-8-fluoro-4-hydroxy-6-(3-hydroxypropyl)-3-quinolinecarboxamide;
(110) N-(4-chlorobenzyl)-4-hydroxy-7-(3-hydroxypropyl)-3-quinolinecarboxamide;
(111) ethyl (E)-3-(3-{[(4-chlorobenzyl)amino]carbonyl}-8-fluoro-4-hydroxy-6-quinolinyl)-2-propenoate;
(112) sodium 2-[{8-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;
(113) 3-(3-{[(4-chlorobenzyl)amino]carbonyl}-8-fluoro-4-hydroxy-6-quinolinyl)propanoic acid;
(114) 5-(3-{[(4-chlorobenzyl)amino]carbonyl}-8-fluoro-4-hydroxy-6-quinolinyl)-4-pentynoic acid;
(115) N-[(4-chlorophenyl)methyl]-9]hydroxy-3H-pyrazolo[4,3-f]quinoline-8-carboxamide;
(116) N-(4-chlorobenzyl)-4-hydroxy-6-iodo-8-methoxy-3-quinolinecarboxamide;
(117) N-(4-chlorobenzyl)-4-hydroxy-6-(3-hydroxy-1-propynyl)-8-methoxy-3-quinolinecarboxamide;
(118) N-(4-chlorobenzyl)-4-hydroxy-8-methoxy-6-(3-methoxy-1-propynyl)-3-quinolinecarboxamide;
(119) N-(4-chlorobenzyl)-4-hydroxy-6-(3-hydroxypropyl)-8-methoxy-3-quinolinecarboxamide;
(120) N-(4-chlorobenzyl)-4-hydroxy-6-(3-hydroxy-1-propynyl)-8-(trifluoromethyl)-3-quinolinecarboxamide;
(121) N-(4-chlorobenzyl)-4-hydroxy-6-(3-hydroxy-1-propynyl)-8-(trifluoromethoxy)-3-quinolinecarboxamide;
(122) N-(4-chlorobenzyl)-4-hydroxy-6-(3-hydroxypropyl)-8-(trifluoromethyl)-3-quinolinecarboxamide;
(123) N-(4-chlorobenzyl)-4-hydroxy-8-(2-hydroxyethoxy)-6-(3-hydroxypropyl)-3-quinolinecarboxamide;
(124) N-(4-chlorobenzyl)-4-hydroxy-6-(3-hydroxy-1,1-dimethylpropyl)-3-quinolinecarboxamide;
(125) N-(4-chlorobenzyl)-8-fluoro-4-hydroxy-6-[3-(methylsulfanyl)-1-propynyl]-3-quinolinecarboxamide;
(126) N-(4-chlorobenzyl)-6-[3-(ethylsulfanyl)-1-propynyl]-8-fluoro-4-hydroxy-3-quinolinecarboxamide;
(127) N-(4-chlorobenzyl)-8-fluoro-4-hydroxy-6-[(Z)-3-(methylsulfanyl)-1-propenyl]-3-quinolinecarboxamide;
(128) N-(4-chlorobenzyl)-6-[(Z)-3-(ethylsulfanyl)-1-propenyl]-8-fluoro-4-hydroxy-3-quinolinecarboxamide;
(129) N-(4-chlorobenzyl)-8-fluoro-4-hydroxy-6-[3-(methylsulfanyl)propyl]-3-quinolinecarboxamide;
(130) 3-(3-{[(4-chlorobenzyl)amino]carbonyl}-8-fluoro-4-hydroxy-6-quinolinyl)-2-propynyl formate;
(131) N-(4-chlorobenzyl)-8-fluoro-4-hydroxy-6-(3-hydroxybutyl)-3-quinolinecarboxamide;
(132) N-(4-chlorobenzyl)-6-[(E)-2-cyanoethenyl]-8-fluoro-4-hydroxy-3-quinolinecarboxamide;
(133) N-(4-chlorobenzyl)-4-hydroxy-6-(3-hydroxy-1-propynyl)-3-quinolinecarboxamide;
(134) N-(4-chlorobenzyl)-6-[(Z)-3-hydroxy-1-propenyl]-4-oxo-1,4-dihydro-3-quinolinecarboxamide;
(135) N-(4-chlorobenzyl)-6-[(E)-3-hydroxy-1-propenyl]-4-oxo-1,4-dihydro-3-quinolinecarboxamide;
(136) N-(4-chlorobenzyl)-8-fluoro-4-hydroxy-6-[(Z)-3-hydroxy-1-propenyl]-3-quinolinecarboxamide;
(137) N-(4-chlorobenzyl)-6-(2-cyanoethyl)-8-fluoro-4-hydroxy-3-quinolinecarboxamide;
(138) N-(4-chlorobenzyl)-8-fluoro-4-hydroxy-6-(3-oxopropyl)-3-quinolinecarboxamide;
(139) N-(4-chlorobenzyl)-4-hydroxy-7-(4-hydroxybutyl)-3-quinolinecarboxamide;
(140) N-(4-chlorobenzyl)-8-fluoro-4-hydroxy-6-(hydroxymethyl)-3-quinolinecarboxamide;
(141) 3-(3-{[(4-chlorobenzyl)amino]carbonyl}-8-fluoro-4-hydroxy-6-quinolinyl)propyl methanesulfonate;
(142) N-(4-chlorobenzyl)-8-fluoro-6-(3-fluoro-1-propynyl)-4-hydroxy-3-quinolinecarboxamide;
(143) N-(4-chlorobenzyl)-4-hydroxy-6-(3-hydroxypropyl)-3-quinolinecarboxamide;
(144) 3-(3-{[(4-chlorobenzyl)amino]carbonyl}-8-fluoro-4-hydroxy-6-quinolinyl)propyl 2-bromoacetate;
(145) 3-(3-{[(4-chlorobenzyl)amino]carbonyl}-8-fluoro-4-hydroxy-6-quinolinyl)propyl 2-[(tert-butoxycarbonyl)amino]-3-methylbutanoate;
(146) 3-(3-{[(4-chlorobenzyl)amino]carbonyl}-8-fluoro-4-hydroxy-6-quinolinyl)propyl 2-(4-morpholinyl)acetate;
(147) 3-(3-{[(4-chlorobenzyl)amino]carbonyl}-8-fluoro-4-hydroxy-6-quinolinyl)propyl 2-(dimethylamino)acetate;
(148) 3-(3-{[(4-chlorobenzyl)amino]carbonyl}-8-fluoro-4-hydroxy-6-quinolinyl)propyl 2-amino-3-methylbutanoate;
(149) 3-(3-{[(4-chlorobenzyl)amino]carbonyl}-8-fluoro-4-hydroxy-6-quinolinyl)-2-propynyl phenylcarbamate;
(150) N-(4-chlorobenzyl)-4-hydroxy-6-propyl-3-quinolinecarboxamide;
(151) N-(4-chlorobenzyl)-4-hydroxy-6-(3-hydroxy-1-butynyl)-3-quinolinecarboxamide;
(152) N-(4-chlorobenzyl)-8-fluoro-4-hydroxy-6-[(E)-3-oxo-1-butenyl]-3-quinolinecarboxamide;
(153) N-(4-chlorobenzyl)-8-fluoro-4-hydroxy-6-(5-hydroxypentyl)-3-quinolinecarboxamide;
(154) 3-(3-{[(4-chlorobenzyl)amino]carbonyl}-8-fluoro-4-hydroxy-6-quinolinyl)propyl 2,6-bis[(tert-butoxycarbonyl)amino]hexanoate;
(155) 3-(3-{[(4-chlorobenzyl)amino]carbonyl}-8-fluoro-4-hydroxy-6-quinolinyl)propyl 2,6-diaminohexanoate, trifluoroacetic acid salt;
(156) N-(4-chlorobenzyl)-4-hydroxy-6-(3-hydroxybutyl)-3-quinolinecarboxamide;
(157) N-(4-chlorobenzyl)-8-fluoro-4-hydroxy-6-(4-morpholinylmethyl)-3-quinolinecarboxamide;
(158) 3-(3-{[(4-chlorobenzyl)amino]carbonyl}-8-fluoro-4-hydroxy-6-quinolinyl)propyl phenylcarbamate;
(159) N-(4-chlorobenzyl)-8-fluoro-4-hydroxy-6-(3-oxobutyl)-3-quinolinecarboxamide;
(160) 3-(3-{[(4-chlorobenzyl)amino]carbonyl}-4-hydroxy-6-quinolinyl)-2-propynyl 2,6-bis[(tert-butoxycarbonyl)amino]hexanoate;
(161) 3-(3-{[(4-chlorobenzyl)amino]carbonyl}-4-hydroxy-6-quinolinyl)-2-propynyl 2,6-diaminohexanoate, trifluoroacetic acid salt;
(162) N-(4-chlorobenzyl)-4-hydroxy-6-[(2-hydroxyethoxy)methyl]-3-quinolinecarboxamide;
(163) N-(4-chlorobenzyl)-4-hydroxy-6-{[2-hydroxy-1-(hydroxymethyl)ethoxy]methyl}-3-quinolinecarboxamide;
(164) Methyl 3-{[(4-chlorobenzyl)amino]carbonyl}-4-hydroxy-6-quinolinecarboxylate;
(165) N-(4-chlorobenzyl)-4-hydroxy-6-(hydroxymethyl)-3-quinolinecarboxamide;
(166) 6-chloro-N-(4-chlorobenzyl)-4-hydroxy-8-methyl-3-quinolinecarboxamide;
(167) N-(4-chlorobenzyl)-5,6,8-trifluoro-4-hydroxy-3-quinolinecarboxamide;
(168) N-(4-chlorobenzyl)-6,7-difluoro-4-hydroxy-3-quinolinecarboxamide;
(169) N-(4-chlorobenzyl)-8-fluoro-4-hydroxy-6-(methylsulfanyl)-3-quinolinecarboxamide;
(170) N-(4-chlorobenzyl)-8-fluoro-4-hydroxy-6-[(2-hydroxyethyl)sulfanyl]-3-quinolinecarboxamide;
(171) 6-[(2-aminoethyl)sulfanyl]-N-(4-chlorobenzyl)-8-fluoro-4-hydroxy-3-quinolinecarboxamide hydrobromide;
(172) N-(4-chlorobenzyl)-8-fluoro-4-hydroxy-6-{[(2-methoxyethoxy)methyl]sulfanyl}-3-quinolinecarboxamide;
(173) N-(4-chlorobenzyl)-8-fluoro-4-hydroxy-6-{[2-(4-morpholinyl)ethyl]sulfanyl}-3-quinolinecarboxamide;
(174) N-(4-chlorobenzyl)-8-fluoro-4-hydroxy-6-(methylsulfinyl)-3-quinolinecarboxamide;
(175) N-(4-chlorobenzyl)-8-fluoro-4-hydroxy-6-(methylsulfonyl)-3-quinolinecarboxamide;
(176) N-(4-chlorobenzyl)-8-fluoro-4-hydroxy-6-[(2-hydroxyethyl)sulfinyl]-3-quinolinecarboxamide;
(177) N-(4-chlorobenzyl)-4-hydroxy-6-(2-thienyl)-3-quinolinecarboxamide;
(178) N-(4-chlorobenzyl)-4-hydroxy-6-(2-hydroxyethoxy)-3-quinolinecarboxamide;
(179) N-(4-chlorobenzyl)-8-fluoro-4-hydroxy-6-(4-morpholinylcarbonyl)-3-quinolinecarboxamide;
(180) N-³-(4-chlorobenzyl)-8-fluoro-4-hydroxy-N⁶-(2-hydroxyethyl)-3,6-quinolinecarboxamide;
(181) N³-(4-chlorobenzyl)-8-fluoro-4-hydroxy-N⁶,N˜6˜-dimethyl-3,6-quinolinecarboxamide;
(182) N³-(4-chlorobenzyl)-8-fluoro-4-hydroxy-N-⁶-(4-hydroxyphenethyl)-3,6-quinolinecarboxamide;
(183) N³-(4-chlorobenzyl)-8-fluoro-4-hydroxy-3,6-quinolinecarboxamide;
(184) N³,N⁶-bis(4-chlorobenzyl)-8-fluoro-4-hydroxy-3,6-quinolinecarboxamide;
(185) 6-amino-N-(4-chlorobenzyl)-8-fluoro-4-hydroxy-3-quinolinecarboxamide;
(186) N-(4-chlorobenzyl)-8-fluoro-4-hydroxy-6-{[(4-methoxyphenyl)sulfonyl]amino}-3-quinolinecarboxamide;
(187) N-(4-chlorobenzyl)-4-hydroxy-6-[(2-hydroxyethyl)amino]-3-quinolinecarboxamide;
(188) N-(4-chlorobenzyl)-6-[ethyl(2-hydroxyethyl)amino]-4-hydroxy-3-quinolinecarboxamide;
(189) N-(4-chlorobenzyl)-4-hydroxy-6-(2-oxo-1,3-oxazolidin-3-yl)-3-quinolinecarboxamide;
(190) N-(4-chlorobenzyl)-4-hydroxy-6-(4-morpholinylmethyl)-3-quinolinecarboxamide;
(191) N-(4-chlorobenzyl)-8-fluoro-4-hydroxy-6-{[(1-naphthylmethyl)amino]sulfonyl}-3-quinolinecarboxamide;
(192) N-(4-chlorobenzyl)-8-fluoro-4-hydroxy-6-({[2-(1H-indol-3-yl)ethyl]amino}sulfonyl)-3-quinolinecarboxamide;
(193) N-(4-chlorobenzyl)-8-fluoro-6-{[(2-furylmethyl)amino]sulfonyl}-4-hydroxy-3-quinolinecarboxamide;
(194) 6-{[bis(2-hydroxyethyl)amino]sulfonyl}-N-(4-chlorobenzyl)-8-fluoro-4-hydroxy-3-quinolinecarboxamide;
(195) ethyl 2-{[(3-{[(4-chlorobenzyl)amino]carbonyl}-8-fluoro-4-hydroxy-6-quinolinyl)sulfonyl]amino}acetate;
(196) N-(4-chlorobenzyl)-8-fluoro-4-hydroxy-6-{[(2-hydroxyethyl)amino]sulfonyl}-3-quinolinecarboxamide;
(197) N-(4-chlorobenzyl)-8-fluoro-4-hydroxy-6-(4-morpholinylsulfonyl)-3-quinolinecarboxamide;
(198) N-(4-chlorobenzyl)-8-fluoro-4-hydroxy-6-{[(2-pyridinylmethyl)amino]sulfonyl}-3-quinolinecarboxamide;
(199) N-(4-chlorobenzyl)-8-fluoro-4-hydroxy-6-[(2-pyridinylamino)sulfonyl]-3-quinolinecarboxamide;
(200) N-(4-chlorobenzyl)-6-{[(cyclohexylmethyl)amino]sulfonyl}-8-fluoro-4-hydroxy-3-quinolinecarboxamide;
(201) N-(4-chlorobenzyl)-8-fluoro-4-hydroxy-6-({[2-(1-methyl-2-pyrrolidinyl)ethyl]amino}sulfonyl)-3-quinolinecarboxamide;
(202) N-(4-chlorobenzyl)-8-fluoro-4-hydroxy-6-({[2-(1-pyrrolidinyl)ethyl]amino}sulfonyl)-3-quinolinecarboxamide;
(203) N-(4-chlorobenzyl)-8-fluoro-6-{[(2-furylmethyl)amino]sulfonyl}-4-hydroxy-3-quinolinecarboxamide;
(204) N-(4-chlorobenzyl)-6-({[3-(cyclohexylamino)propyl]amino}sulfonyl)-8-fluoro-4-hydroxy-3-quinolinecarboxamide;
(205) N-(4-chlorobenzyl)-8-fluoro-4-hydroxy-6-{[(1-naphthylmethyl)amino]sulfonyl}-3-quinolinecarboxamide;
(206) N-(4-chlorobenzyl)-8-fluoro-4-hydroxy-6-({[2-(1H-imidazol-4-yl)ethyl]amino}-sulfonyl)-3-quinolinecarboxamide;
(207) N-(4-chlorobenzyl)-8-fluoro-4-hydroxy-6-{[(tetrahydro-2-furanylmethyl)amino]-sulfonyl}-3-quinolinecarboxamide;
(208) N-(4-chlorobenzyl)-8-fluoro-4-hydroxy-6-{[(2-thienylmethyl)amino]sulfonyl}-3-quinolinecarboxamide;
(209) N-(4-chlorobenzyl)-8-fluoro-4-hydroxy-6-({[2-(1H-indol-3-yl)ethyl]amino}sulfonyl)-3-quinolinecarboxamide;
(210) N-(4-chlorobenzyl)-8-fluoro-4-hydroxy-6-({[2-(5-methoxy-1H-indol-3-yl)ethyl]amino}sulfonyl)-3-quinolinecarboxamide;
(211) 6-{[(1,3-benzodloxol-5-ylmethyl)amino]sulfonyl}-N-(4-chlorobenzyl)-8-fluoro-4-hydroxy-3-quinolinecarboxamide;
(212) N-(4-chlorobenzyl)-8-fluoro-4-hydroxy-6-({[2-(4-morpholinyl)ethyl]amino}-sulfonyl)-3-quinolinecarboxamide;
(213) N-(4-chlorobenzyl)-8-fluoro-4-hydroxy-6-({[3-(4-morpholinyl)propyl]amino}-sulfonyl)-3-quinolinecarboxamide;
(214) N-(4-chlorobenzyl)-8-fluoro-4-hydroxy-6-[({2-[(5-nitro-2-pyridinyl)amino]ethyl}-amino)sulfonyl]-3-quinolinecarboxamide;
(215) N-(4-chlorobenzyl)-8-fluoro-4-hydroxy-6-{[(2-pyridinylmethyl)amino]sulfonyl}-3-quinolinecarboxamide;
(216) N-(4-chlorobenzyl)-8-fluoro-4-hydroxy-6-({[2-(2-pyridinyl)ethyl]amino}sulfonyl)-3-quinolinecarboxamide;
(217) N-(4-chlorobenzyl)-8-fluoro-4-hydroxy-6-{[(3-pyridinylmethyl)amino]sulfonyl}-3-quinolinecarboxamide;
(218) N-(4-chlorobenzyl)-8-fluoro-4-hydroxy-6-{[(4-pyridinylmethyl)amino]sulfonyl}-3-quinolinecarboxamide;
(219) N-(4-chlorobenzyl)-6-{[(4-chlorobenzyl)amino]sulfonyl}-8-fluoro-4-hydroxy-3-quinolinecarboxamide;
(220) N-(4-chlorobenzyl)-8-fluoro-4-hydroxy-6-{[(4-methoxybenzyl)amino]sulfonyl}-3-quinolinecarboxamide;
(221) N-(4-chlorobenzyl)-8-fluoro-4-hydroxy-6-[(neopentylamino)sulfonyl]-3-quinolinecarboxamide;
(222) N-(4-chlorobenzyl)-8-fluoro-4-hydroxy-6-{[(2-hydroxypropyl)amino]sulfonyl}-3-quinolinecarboxamide;
(223) N-(4-chlorobenzyl)-6-{[(2,3-dihydroxypropyl)amino]sulfonyl}-8-fluoro-4-hydroxy-3-quinolinecarboxamide;
(224) N-(4-chlorobenzyl)-6-{[(2,2-diphenylethyl)amino]sulfonyl}-8-fluoro-4-hydroxy-3-quinolinecarboxamide;
(225) 11-{[(3-{[(4-chlorobenzyl)amino]carbonyl}-8-fluoro-4-hydroxy-6-quinolinyl)sulfonyl]amino}undecanoic acid;
(226) 6-({[2-(acetylamino)ethyl]amino}sulfonyl)-N-(4-chlorobenzyl)-8-fluoro-4-hydroxy-3-quinolinecarboxamide;
(227) N-(4-chlorobenzyl)-8-fluoro-4-hydroxy-6-({[2-(2-hydroxyethoxy)ethyl]amino}-sulfonyl)-3-quinolinecarboxamide;
(228) N-(4-chlorobenzyl)-8-fluoro-4-hydroxy-6-{[(2-hydroxyethyl)amino]sulfonyl}-3-quinolinecarboxamide;
(229) N-(4-chlorobenzyl)-8-fluoro-4-hydroxy-6-[(phenethylamino)sulfonyl]-3-quinolinecarboxamide;
(230) N-(4-chlorobenzyl)-6-{[(4-chlorophenethyl)amino]sulfonyl}-8-fluoro-4-hydroxy-3-quinolinecarboxamide;
(231) N-(4-chlorobenzyl)-8-fluoro-4-hydroxy-6-[(2-propynylamino)sulfonyl]-3-quinolinecarboxamide;
(232) N-(4-chlorobenzyl)-8-fluoro-4-hydroxy-6-[(isopentylamino)sulfonyl]-3-quinolinecarboxamide; p1 (233) N-(4-chlorobenzyl)-8-fluoro-4-hydroxy-6-{[(3-phenylpropyl)amino]sulfonyl}-3-quinolinecarboxamide;
(234) N-(4-chlorobenzyl)-8-fluoro-4-hydroxy-6-[(pentylamino)sulfonyl]-3-quinolinecarboxamide;
(235) 6-({[3,5-bis(trifluoromethyl)benzyl]amino}sulfonyl)-N-(4-chlorobenzyl)-8-fluoro-4-hydroxy-3-quinolinecarboxamide;
(236) N-(4-chlorobenzyl)-6-({[2-(1-cyclohexen-1-yl)ethyl]amino}sulfonyl)-8-fluoro-4-hydroxy-3-quinolinecarboxamide;
(237) N-(4-chlorobenzyl)-8-fluoro-4-hydroxy-6-({[2-(1-naphthylamino)ethyl]amino}-sulfonyl)-3-quinolinecarboxamide;
(238) N-(4-chlorobenzyl)-8-fluoro-4-hydroxy-6-[(methylamino)sulfonyl]-3-quinolinecarboxamide;
(239) N-(4-chlorobenzyl)-6-{[(cyanomethyl)amino]sulfonyl}-8-fluoro-4-hydroxy-3-quinolinecarboxamide;
(240) N-(4-chlorobenzyl)-6-{[(2,4-dimethoxybenzyl)amino]sulfonyl}-8-fluoro-4-hydroxy-3-quinolinecarboxamide;
(241) N-(4-chlorobenzyl)-8-fluoro-4-hydroxy-6-{[(3-iodobenzyl)amino]sulfonyl}-3-quinolinecarboxamide;
(242) N-(4-chlorobenzyl)-8-fluoro-4-hydroxy-6-{[(2,2,2-trifluoroethyl)amino]sulfonyl}-3-quinolinecarboxamide;
(243) 6-{[(2-bromoethyl)amino]sulfony}-N-(4-chlorobenzyl)-8-fluoro-4-hydroxy-3-quinolinecarboxamide;
(244) N-(4-chlorobenzyl)-6-{[(2-chloroethyl)amino]sulfonyl}-8-fluoro-4-hydroxy-3-quinolinecarboxamide;
(245) N-(4-chlorobenzyl)-6-{[(3,4-dihydroxyphenethyl)amino]sulfonyl}-8-fluoro-4-hydroxy-3-quinolinecarboxamide;
(246) N-(4-chlorobenzyl)-6-({[2-(ethylsulfanyl)ethyl]amino}sulfonyl)-8-fluoro-4-hydroxy-3-quinolinecarboxamide;
(247) 6-{[(3-bromopropyl)amino]sulfonyl}-N-(4-chlorobenzyl)-8-fluoro-4-hydroxy-3-quinolinecarboxamide;
(248) 6-({[4-(aminosulfonyl)benzyl]amino}sulfonyl)-N-(4-chlorobenzyl)-8-fluoro-4-hydroxy-3-quinolinecarboxamide;
(249) 6-[({2-[bis(2-hydroxyethyl)amino]ethyl}amino)sulfonyl]-N-(4-chlorobenzyl)-8-fluoro-4-hydroxy-3-quinolinecarboxamide;
(250) N-(4-chlorobenzyl)-6-({[2-(ethylsulfanyl)ethyl]amino}sulfonyl)-8-fluoro-4-hydroxy-3-quinolinecarboxamide;
(251) N-(4-chlorobenzyl)-6-{[(3,4-dimethylbenzyl)amino]sulfonyl}-8-fluoro-4-hydroxy-3-quinolinecarboxamide;
(252) N-(4-chlorobenzyl)-6-{[(cyclopropylmethyl)amino]sulfonyl}-8-fluoro-4-hydroxy-3-quinolinecarboxamide;
(253) 6-{[(4-bromobenzyl)amino]sulfonyl}-N-(4-chlorobenzyl)-8-fluoro-4-hydroxy-3-quinolinecarboxamide;
(254) N-(4-chlorobenzyl)-8-fluoro-4-hydroxy-6-({[2-(2-thienyl)ethyl]amino}sulfonyl)-3-quinolinecarboxamide;
(255) N-(4-chlorobenzyl)-8-fluoro-4-hydroxy-6-{[(2-phenoxyethyl)amino]sulfonyl}-3-quinolinecarboxamide;
(256) tert-butyl 2-{[(3-{[(4-chlorobenzyl)amino]carbonyl}-8-fluoro-4-hydroxy-6-quinolinyl)sulfonyl]amino}acetate;
(257) tert-butyl 3-{[(3-{[(4-chlorobenzyl)amino]carbonyl}-8-fluoro-4-hydroxy-6-quinolinyl)sulfonyl]amino}propanoate;
(258) N-(4-chlorobenzyl)-8-fluoro-4-hydroxy-6-({[3-(trifluoromethoxy)benzyl]amino}-sulfonyl)-3-quinolinecarboxamide;
(259) N-(4-chlorobenzyl)-8-fluoro-4-hydroxy-6-{[(2-{[2-(hydroxymethyl)phenyl]sulfanyl}-benzyl)amino]sulfonyl}-3-quinolinecarboxamide;
(260) N-(4-chlorobenzyl)-8-fluoro-4-hydroxy-6-({[4-(1,2,3-thiadiazol-4-yl)benzyl]amino}sulfonyl)-3-quinolinecarboxamide;
(261) N-(4-chlorobenzyl)-6-{[(4-chloro-2-fluorobenzyl)amino]sulfonyl}-8-fluoro-4-hydroxy-3-quinolinecarboxamide;
(262) N-(4-chlorobenzyl)-8-fluoro-4-hydroxy-6-[({2-[(2-hydroxyethyl)sulfanyl]ethyl}-amino)sulfonyl]-3-quinolinecarboxamide;
(263) 6-{[(2-amino-2-methylpropyl)amino]sulfonyl}-N-(4-chlorobenzyl)-8-fluoro-4-hydroxy-3-quinolinecarboxamide;
(264) 6-{[(2-amino-2-oxoethyl)amino]sulfonyl}-N-(4-chlorobenzyl)-8-fluoro-4-hydroxy-3-quinolinecarboxamide;
(265) 6-{[(4-aminobenzyl)amino]sulfonyl}-N-(4-chlorobenzyl)-8-fluoro-4-hydroxy-3-quinolinecarboxamide;
(266) di(tert-butyl) 3-(3-{[(4-chlorobenzyl)amino]carbonyl}-8-fluoro-4-hydroxy-6-quinolinyl)propyl phosphate;
(267) 3-(3-{[(4-chlorobenzyl)amino]carbonyl}-8-fluoro-4-hydroxy-6-quinolinyl)propyl dihydrogen phosphate;
(268) 3-(3-{[(4-chlorobenzyl)amino]carbonyl}-4-hydroxy-6-quinolinyl)propyl dihydrogen phosphate;
(269) tert-butyl 3-(3-{[(4-chlorobenzyl)amino]carbonyl}-4-hydroxy-6-quinolinyl)propyl phosphonate;
(270) tert-butyl 3-(3-{[(4-chlorobenzyl)amino]carbonyl}-8-fluoro-4-hydroxy-6-quinolinyl)propyl phosphonate;
(271) (E)-3-(3-{[(4-chlorobenzyl)amino]carbonyl}-8-fluoro-4-hydroxy-6-quinolinyl)-2-propenoic acid; or
(272) N-[(4-chlorophenyl)methyl]-4-hydroxy-7-iodo-3-quinolinecarboxamide.

4. The method of claim 2, wherein the compound administered is
(1) 7-amino-N-(4-chlorobenzyl)-4-hydroxy-3-quinolinecarboxamide;
(2) N-(4-chlorobenzyl)-4-hydroxy-7-methoxy-3-quinolinecarboxamide;
(3) N-(4-chlorobenzyl)-7-fluoro-4-hydroxy-3-quinolinecarboxamide;
(4) N-(4-chlorobenzyl)-8-fluoro-4-hydroxy-3-quinolinecarboxamide;
(5) 6-chloro-N-(4-chlorobenzyl)-4-hydroxy-3-quinolinecarboxamide;
(6) N-(4-chlorobenzyl)-4-hydroxy-6-methyl-3-quinolinecarboxamide;
(7) N-(4-chlorobenzyl)-4-hydroxy-6-methoxy-3-quinolinecarboxamide;
(8) N-(4-chlorobenzyl)-8-fluoro-4-hydroxy-6-methyl-3-quinolinecarboxamide;
(9) N-(4-chlorobenzyl)-4-hydroxy-6-iodo-3-quinolinecarboxamide;
(10) N-(4-chlorobenzyl)-4-hydroxy-6-phenyl-3-quinolinecarboxamide;
(11) N-(4-chlorobenzyl)-4-hydroxy-6,8-dimethoxy-3-quinolinecarboxamide;
(12) 6-(tert-butyl)-N-(4-chlorobenzyl)-4-hydroxy-3-quinolinecarboxamide;
(13) N-(4-chlorobenzyl)-6-(cyanomethyl)-4-hydroxy-3-quinolinecarboxamide;
(14) N-(4-chlorobenzyl)-9-hydroxy[1,3]thiazolo[5,4-f]quinoline-8-carboxamide;
(15) N-(4-chlorobenzyl)-8-fluoro-4-hydroxy-6-(3-hydroxy-1-propynyl)-3-quinolinecarboxamide;
(16) N-(4-chlorobenzyl)-8-fluoro-4-hydroxy-6-(1,3-thiazol-2-yl)-3-quinolinecarboxamide;
(17) N-(4-chlorobenzyl)-8-fluoro-4-hydroxy-6-(2-thienyl)-3-quinolinecarboxamide;
(18) N-(4-chlorobenzyl)-8-fluoro-4-hydroxy-6-(3-methoxy-1-propynyl)-3-quinolinecarboxamide;
(19) 6-(4-bromo-2-thienyl)-N-(4-chlorobenzyl)-8-fluoro-4-hydroxy-3-quinolinecarboxamide;
(20) N-(4-chlorobenzyl)-8-fluoro-4-hydroxy-6-(4-hydroxybutyl)-3-quinolinecarboxamide;
(21) N-(4-chlorobenzyl)-8-fluoro-4-hydroxy-6-[(Z)-4-hydroxy-1-butenyl]-3-quinolinecarboxamide;
(22) N-((4-chlorobenzyl)-6,8-difluoro-4-hydroxy-7-[4-(hydroxymethyl)phenoxy]-3-quinolinecarboxamide;
(23) N-((4-chlorobenzyl)-6,8-difluoro-4-hydroxy-7-methoxy-3-quinolinecarboxamide;
(24) N-(4-chlorobenzyl)-8-fluoro-4-hydroxy-6-(5-hydroxy-1-pentynyl)-3-quinolinecarboxamide;
(25) 3-(3-{[(4-chlorobenzyl)amino]carbonyl}-8-fluoro-4-hydroxy-6-quinolinyl)-2-propynyl formate;
(26) N-(4-chlorobenzyl)-8-fluoro-4-hydroxy-6-(3-hydroxypropyl)-3-quinolinecarboxamide;
(27) N-(4-chlorobenzyl)-4-hydroxy-7-(3-hydroxypropyl)-3-quinolinecarboxamide;
(28) N-((4-chlorobenzyl)-8-fluoro-4-hydroxy-6-(3-hydroxybutyl)-3-quinolinecarboxamide;
(29) N-((4-chlorobenzyl)-6-[(E)-2-cyanoethenyl]-8-fluoro-4-hydroxy-3-quinolinecarboxamide;
(30) N-((4-chlorobenzyl)-4-hydroxy-6-(3-hydroxy-1-propynyl)-8-methoxy-3-quinolinecarboxamide;
(31) N-(4-chlorobenzyl)-4-hydroxy-6-(3-hydroxy-1-propynyl)-3-quinolinecarboxamide;
(32) N-(4-chlorobenzyl)-4-hydroxy-8-methoxy-6-(3-methoxy-1-propynyl)-3-quinolinecarboxamide;
(33) N-(4-chlorobenzyl)-8-fluoro-4-hydroxy-6-[(Z)-3-hydroxy-1-propenyl]-3-quinolinecarboxamide;
(34) N-(4-chlorobenzyl)-8-fluoro-4-hydroxy-6-(3-oxopropyl)-3-quinolinecarboxamide;
(35) N-(4-chlorobenzyl)-4-hydroxy-7-(4-hydroxybutyl)-3-quinolinecarboxamide;
(36) N-(4-chlorobenzyl)-4-hydroxy-6-iodo-8-methoxy-3-quinolinecarboxamide;
(37) N-(4-chlorobenzyl)-8-fluoro-6-(3-fluoro-1-propynyl)-4-hydroxy-3-quinolinecarboxamide;
(38) N-(4-chlorobenzyl)-4-hydroxy-6-(3-hydroxypropyl)-3-quinolinecarboxamide;
(39) 3-(3-{[(4-chlorobenzyl)amino]carbonyl}-8-fluoro-4-hydroxy-6-quinolinyl)propyl 2-bromoacetate;
(40) 3-(3-{[(4-chlorobenzyl)amino]carbonyl}-8-fluoro-4-hydroxy-6-quinolinyl)propyl 2-[(tert-butoxycarbonyl)amino]-3-methylbutanoate;
(41) 3-(3-{[(4-chlorobenzyl)amino]carbonyl}-8-fluoro-4-hydroxy-6-quinolinyl)propyl 2-amino-3-methylbutanoate;
(42) 3-(3-{[(4-chlorobenzyl)amino]carbonyl}-8-fluoro-4-hydroxy-6-quinolinyl)-2-propynyl phenylcarbamate;
(43) N-((4-chlorobenzyl)-4-hydroxy-6-propyl-3-quinolinecarboxamide;
(44) N-((4-chlorobenzyl)-4-hydroxy-6-(3-hydroxypropyl)-8-methoxy-3-quinolinecarboxamide;
(45) N-(4-chlorobenzyl)-8-fluoro-4-hydroxy-6-(methylsulfanyl)-3-quinolinecarboxamide;
(46) N-(4-chlorobenzyl)-4-hydroxy-7-{[(1-naphthylmethyl)amino]sulfonyl}-3-quinolinecarboxamide;
(47) N-(4-chlorobenzyl)-8-fluoro-4-hydroxy-6-[(E)-3-oxo-1-butenyl]-3-quinolinecarboxamide;
(48) N-(4-chlorobenzyl)-4-hydroxy-6-(3-hydroxy-1-propynyl)-8-(trifluoromethoxy)-3-quinolinecarboxamide;
(49) sodium 2-[{8-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;
(50) 3-(3-{[(4-chlorobenzyl)amino]carbonyl}-8-fluoro-4-hydroxy-6-quinolinyl)propyl dihydrogen phosphate;
(51) 3-(3-{[(4-chlorobenzyl)amino]carbonyl}-8-fluoro-4-hydroxy-6-quinolinyl)propyl 2,6-bis[(tert-butoxycarbonyl)amino]hexanoate;
(52) tert-butyl 3-(3-{[(4-chlorobenzyl)amino]carbonyl}-8-fluoro-4-hydroxy-6-quinolinyl)propyl phosphonate;
(53) sodium 2-[(8-{[3-(3-{[(4-chlorobenzyl)amino]carbonyl}-8-fluoro-4-hydroxy-6-quinolinyl)-2-propynyl]oxy}-8-oxooctanoyl)(methyl)amino]-1-ethanesulfonate;
(54) N-(4-chlorobenzyl)-4-hydroxy-6-(2-hydroxyethoxy)-3-quinolinecarboxamide;
(55) 3-(3-{[(4-chlorobenzyl)amino]carbonyl}-8-fluoro-4-hydroxy-6-quinolinyl)propyl 2,6-diaminohexanoate trifluoroacetic acid salt;
(56) N-(4-chlorobenzyl)-4-hydroxy-6-[(2-hydroxyethyl)amino]-3-quinolinecarboxamide;
(57) N-(4-chlorobenzyl)-4-hydroxy-6-(3-hydroxybutyl)-3-quinolinecarboxamide;
(58) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;
(59) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;
(60) tert-butyl 3-(3-{[(4-chlorobenzyl)amino]carbonyl}-4-hydroxy-6-quinolinyl)propyl phosphonate;
(61) 3-(3-{[(4-chlorobenzyl)amino]carbonyl}-4-hydroxy-6-quinolinyl)propyl dihydrogen phosphate;
(62) N-(4-chlorobenzyl)-4-hydroxy-8-(2-hydroxyethoxy)-6-(3-hydroxypropyl)-3-quinolinecarboxamide;
(63) N-(4-chlorobenzyl)-8-fluoro-4-hydroxy-6-(4-morpholinylmethyl)-3-quinolinecarboxamide;
(64) N-(4-chlorobenzyl)-8-fluoro-4-hydroxy-6-(3-oxobutyl)-3-quinolinecarboxamide;
(65) N-(4-chlorobenzyl)-4-hydroxy-6-(2-oxo-1,3-oxazolidin-3-yl)-3-quinolinecarboxamide;
(66) 3-(3-{[(4-chlorobenzyl)amino]carbonyl)-4-hydroxy-6-quinolinyl)-2-propynyl 2,6-bis[(tert-butoxycarbonyl)amino]hexanoate;
(67) N-(4-chlorobenzyl)-6-[(Z)-3-hydroxy-1-propenyl]-4-oxo-1,4-dihydro-3-quinolinecarboxamide;
(68) N-(4-chlorobenzyl)-6-[(E)-3-hydroxy-1-propenyl]-4-oxo-1,4-dihydro-3-quinolinecarboxamide;
(69) 3-(3-{[(4-chlorobenzyl)amino]carbonyl}-4-hydroxy-6-quinolinyl)-2-propynyl 2,6-diaminohexanoate trifluoroacetic acid salt;
(70) 3-(3-{[(4-chlorobenzyl)amino]carbonyl}-4-hydroxy-6-quinolinyl)propyl hydrogen phosphonate;
(71) N-(4-chlorobenzyl)-4-hydroxy-6-(2-thienyl)-3-quinolinecarboxamide;
(72) N-((4-chlorobenzyl)-4-hydroxy-6-[(2-hydroxyethoxy)methyl]-3-quinolinecarboxamide;
(73) N-(4-chlorobenzyl)-4-hydroxy-6-(3-hydroxy-1,1-dimethylpropyl)-3-quinolinecarboxamide;
(74) N-(4-chlorobenzyl)-4-hydroxy-6-{[2-hydroxy-1-(hydroxymethyl)ethoxy]methyl}-3-quinolinecarboxamide;
(75) N-((4-chlorobenzyl)-4-hydroxy-6-(hydroxymethyl)-3-quinolinecarboxamide;
(76) Methyl 3-{[(4-chlorobenzyl)amino]carbonyl}-4-hydroxy-6-quinolinecarboxylate; or (77) N-(4-chlorobenzyl)-4-hydroxy-6-(4-morpholinylmethyl)-3-quinolinecarboxamide.

5. The method of claim 2, wherein the compound administered is
(1) N-(4-chlorobenzyl)-4-hydroxy-7-methoxy-3-quinolinecarboxamide;
(2) N-(4-chlorobenzyl)-7-fluoro-4-hydroxy-3-quinolinecarboxamide;
(3) N-(4-chlorobenzyl)-8-fluoro-4-hydroxy-3-quinolinecarboxamide;
(4) N-(4-chlorobenzyl)-4-hydroxy-6-methoxy-3-quinolinecarboxamide;
(5) N-(4-chlorobenzyl)-8-fluoro-4-hydroxy-6-(2-thienyl)-3-quinolinecarboxamide;
(6) N-(4-chlorobenzyl)-8-fluoro-4-hydroxy-6-(4-hydroxybutyl)-3-quinolinecarboxamide;
(7) N-(4-chlorobenzyl)-8-fluoro-4-hydroxy-6-[(Z)-4-hydroxy-1-butenyl]-3-quinolinecarboxamide;
(8) N-(4-chlorobenzyl)-8-fluoro-4-hydroxy-6-(3-hydroxypropyl)-3-quinolinecarboxamide;
(9) N-(4-chlorobenzyl)-4-hydroxy-7-(3-hydroxypropyl)-3-quinolinecarboxamide;
(10) N-(4-chlorobenzyl)-4-hydroxy-6-(3-hydroxy-1-propynyl)-8-methoxy-3-quinolinecarboxamide;
(11) N-(4-chlorobenzyl)-4-hydroxy-6-(3-hydroxy-1-propynyl)-3-quinolinecarboxamide;
(12) N-(4-chlorobenzyl)-4-hydroxy-8-methoxy-6-(3-methoxy-1-propynyl)-3-quinolinecarboxamide;
(13) N-(4-chlorobenzyl)-4-hydroxy-7-(4-hydroxybutyl)-3-quinolinecarboxamide;
(14) N-(4-chlorobenzyl)-4-hydroxy-6-(3-hydroxypropyl)-3-quinolinecarboxamide;
(15) 3-(3-{[(4-chlorobenzyl)amino]carbonyl}-8-fluoro-4-hydroxy-6-quinolinyl)propyl 2-bromoacetate;
(16) 3-(3-{[(4-chlorobenzyl)amino]carbonyl}-8-fluoro-4-hydroxy-6-quinolinyl)propyl 2-amino-3-methylbutanoate;
(17) N-(4-chlorobenzyl)-4-hydroxy-6-(3-hydroxypropyl)-8-methoxy-3-quinolinecarboxamide;
(18) sodium 2-[}8-[3-(3-{[(4-chlorobenzyl)amino]carbonyl}-8-fluoro-4-hydroxy-6-quinolinyl)propoxy]-8-oxooctanoyl}(methyl)amino]-1-ethanesulfonate;
(19) 3-(3-{[(4-chlorobenzyl)amino]carbonyl}-8-fluoro-4-hydroxy-6-quinolinyl)propyl dihydrogen phosphate;
(20) 3-(3-{[(4-chlorobenzyl)amino]carbonyl}-8-fluoro-4-hydroxy-6-quinolinyl)propyl 2,6-bis[(tert-butoxycarbonyl)amino]hexanoate;
(21) tert-butyl 3-(3-{[(4-chlorobenzyl)amino]carbonyl}-8-fluoro-4-hydroxy-6-quinolinyl)propyl phosphonate;
(22) 3-(3-{[(4-chlorobenzyl)amino]carbonyl}-8-fluoro-4-hydroxy-6-quinolinyl)propyl 2,6-diaminohexanoate trifluoroacetic acid salt;
(23) N-(4-chlorobenzyl)-4-hydroxy-6-[(2-hydroxyethyl)amino]-3-quinolinecarboxamide;
(24) N-(4-chlorobenzyl)-4-hydroxy-6-(3-hydroxybutyl)-3-quinolinecarboxamide;
(25) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;
(26) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;
(27) tert-butyl 3-(3-{[(4-chlorobenzyl)amino]carbonyl}-4-hydroxy-6-quinolinyl)propyl phosphonate;
(28) 3-(3-{[(4-chlorobenzyl)amino]carbonyl}-4-hydroxy-6-quinolinyl)propyl dihydrogen phosphate;
(29) N-(4-chlorobenzyl)-4-hydroxy-8-(2-hydroxyethoxy)-6-(3-hydroxypropyl)-3-quinolinecarboxamide;
(30) N-(4-chlorobenzyl)-8-fluoro-4-hydroxy-6-(4-morpholinylmethyl)-3-quinolinecarboxamide;
(31) 3-(3-{[(4-chlorobenzyl)amino]carbonyl}-4-hydroxy-6-quinolinyl)-2-propynyl 2,6-bis[(tert-butoxycarbonyl)amino]hexanoate;
(32) N-(4-chlorobenzyl)-6-[(Z)-3-hydroxy-1-propenyl]-4-oxo-1,4-dihydro-3-quinolinecarboxamide;
(33) N-(4-chlorobenzyl)-6-[(E)-3-hydroxy-1-propenyl]-4-oxo-1,4-dihydro-3-quinolinecarboxamide;
(34) 3-(3-{[(4-chlorobenzyl)amino]carbonyl}-4-hydroxy-6-quinolinyl)-2-propynyl 2,6-diaminohexanoate trifluoroacetic acid salt;
(35) 3-(3-{[(4-chlorobenzyl)amino]carbonyl}-4-hydroxy-6-quinolinyl)propyl hydrogen phosphonate;
(36) N-(4-chlorobenzyl)-4-hydroxy-6-(2-thienyl)-3-quinolinecarboxamide;
(37) N-(4-chlorobenzyl)-4-hydroxy-6-[(2-hydroxyethoxy)methyl]-3-quinolinecarboxamide;
(38) N-(4-chlorobenzyl)-4-hydroxy-6-(3-hydroxy-1,1-dimethylpropyl)-3-quinolinecarboxamide;
(39) methyl 3-{[(4-chlorobenzyl)amino]carbonyl}-4-hydroxy-6-quinolinecarboxylate; or
(40) N-(4-chlorobenzyl)-4-hydroxy-6-(4-morpholinylmethyl)-3-quinolinecarboxamide.

6. The method of claim 2, wherein the compound administered is
(1) N-(4-chlorobenzyl)-8-fluoro-4-hydroxy-6-(3-hydroxypropyl)-3-quinolinecarboxamide;
(2) N-(4-chlorobenzyl)-4-hydroxy-6-(3-hydroxy-1-propynyl)-8-methoxy-3-quinolinecarboxamide;
(3) N-(4-chlorobenzyl)-4-hydroxy-8-methoxy-6-(3-methoxy-1-propynyl)-3-quinolinecarboxamide;
(4) N-(4-chlorobenzyl)-4-hydroxy-6-(3-hydroxypropyl)-3-quinolinecarboxamide;
(5) N-(4-chlorobenzyl)-4-hydroxy-6-(3-hydroxypropyl)-8-methoxy-3-quinolinecarboxamide;
(6) sodium 2-[(8-{[3-(3-{[(4-chlorobenzyl)amino]carbonyl}-4-hydroxy-6-quinolinyl)-2-propynyl]oxy}-8-oxooctanoyl)(methyl)amino]-1-ethanesulfonate;
(7) sodium 2-[{8-[3-(3-{[(4-chlorobenzyl)amino]carbonyl}-4-hydroxy-6-quinolinyl)propoxy]-8-oxooctanoyl}(methyl)amino]-1-ethanesulfonate;
(8) 3-(3-{[(4-chlorobenzyl)amino]carbonyl}-4-hydroxy-6-quinolinyl)propyl dihydrogen phosphate;
(9) N-(4-chlorobenzyl)-6-[(Z)-3-hydroxy-1-propenyl]-4-oxo-1,4-dihydro-3-quinolinecarboxamide;
(10) N-(4-chlorobenzyl)-6-[(E)-3-hydroxy-1-propenyl]-4-oxo-1,4-dihydro-3-quinolinecarboxamide;
(11) 3-(3-{[(4-chlorobenzyl)amino]carbonyl}-4-hydroxy-6-quinolinyl)-2-propynyl 2,6-diaminohexanoate trifluoroacetic acid salt;
(12) 3-(3-{[(4-chlorobenzyl)amino]carbonyl}-4-hydroxy-6-quinolinyl)propyl hydrogen phosphonate;
(13) N-(4-chlorobenzyl)-4-hydroxy-6-(2-thienyl)-3-quinolinecarboxamide;
(14) N-((4-chlorobenzyl)-4-hydroxy-6-[(2-hydroxyethoxy)methyl]-3-quinolinecarboxamide; or (15) N-(4-chlorobenzyl)-4-hydroxy-6-(4-morpholinylmethyl)-3-quinolinecarboxamide.

7. A method of claim 1, wherein the compound administered has the Formula II 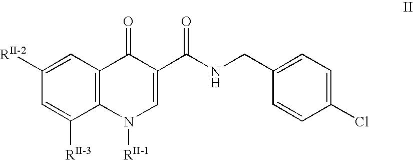
or a pharmaceutically acceptable salt thereof,
wherein
R^{II-1} is C₁₋₇ alkyl, optionally substituted by hydroxy or NR^{II-4}R^{II-5};
R^{II-2} is C₁₋₇ alkyl substituted by hydroxy or NR^{II-4}R^{II-5};
R^{II-3} is H, F or C₁₋₇ alkoxy;
R^{II-4} and R^{II-5} together with N are a 5- or 6-membered heterocyclic moiety having 1-3 heteroatoms selected from the group consisting of nitrogen, oxygen and sulfur in which sulfur may be substituted by one (1) or two (2) oxygen atoms;
and a pharmaceutically acceptable salt thereof.

8. The method of claim 7, wherein the compound administered is
(a) N-(4-chlorobenzyl)-6-(3-hydroxy-1,1-dimethylpropyl)-1-methyl-4-oxo-1,4-dihydro-3-quinolinecarboxamide;
(b) N-(4-chlorobenzyl)-1-methyl-6-(4-morpholinylmethyl)-4-oxo-1,4-dihydro-3-quinolinecarboxamide;
(c) N-(4-chlorobenzyl)-1-(2-hydroxyethyl)-6-(3-hydroxypropyl)-4-oxo-1,4-dihydro-3-quinolinecarboxamide;
(d) N-(4-chlorobenzyl)-6-(3-hydroxypropyl)-1-methyl-4-oxo-1,4-dihydro-3-quinolinecarboxamide;
(e) 1-(tert-butyl)-N-(4-chlorobenzyl)-6-(3-hydroxypropyl)-4-oxo-1,4-dihydro-3-quinolinecarboxamide;
(f) N-(4-chlorobenzyl)-6-[(1,1-dioxo-1′,4-thiazinan-4-yl)methyl]-1-methyl-4-oxo-1,4-dihydro-3-quinolinecarboxamide;
(g) N-(4-chlorobenzyl)-1-methyl-4-oxo-6-[(1-oxo-1,4-thiazinan-4-yl)methyl]-1′,4-dihydro-3-quinolinecarboxamide;
(h) N-(4-chlorobenzyl)-8-fluoro-1-methyl-6-(4-morpholinylmethyl)-4-oxo-1,4-dihydro-3-quinolinecarboxamide;
or a pharmaceutically acceptable salt thereof.

9. The method of claim 7, wherein the compound administered is
(a) N-(4-chlorobenzyl)-6-(3-hydroxy-1,1-dimethylpropyl)-1-methyl-4-oxo-1,4-dihydro-3-quinolinecarboxamide;
(b) N-(4-chlorobenzyl)-1-methyl-6-(4-morpholinylmethyl)-4-oxo-1,4-dihydro-3-quinolinecarboxamide;
(c) N-(4-chlorobenzyl)-1-(2-hydroxyethyl)-6-(3-hydroxypropyl)-4-oxo-1,4-dihydro-3-quinolinecarboxamide;
(d) N-(4-chlorobenzyl)-6-(3-hydroxypropyl)-1-methyl-4-oxo-1,4-dihydro-3-quinolinecarboxamide;
(e) N-(4-chlorobenzyl)-6-[(1,1-dioxo-1′,4-thiazinan-4-yl)methyl]-1-methyl-4-oxo-1,4-dihydro-3-quinolinecarboxamide;
(f) N-(4-chlorobenzyl)-1-methyl-4-oxo-6-[(1-oxo-1′,4-thiazinan-4-yl)methyl]-1,4-dihydro-3-quinolinecarboxamide;
(g) N-(4-chlorobenzyl)-8-fluoro-1-methyl-6-(4-morpholinylmethyl)-4-oxo-1,4-dihydro-3-quinolinecarboxamide;
or a pharmaceutically acceptable salt thereof.

10. The method of claim 7, wherein the compound administered is
(a) N-(4-chlorobenzyl)-1-methyl-6-(4-morpholinylmethyl)-4-oxo-1,4-dihydro-3-quinolinecarboxamide;
(b) N-(4-chlorobenzyl)-6-(3-hydroxypropyl)-1-methyl-4-oxo-1,4-dihydro-3-quinolinecarboxamide;
(c) N-(4-chlorobenzyl)-6-[(1,1-dioxo-1′,4-thiazinan-4-yl)methyl]-1-methyl-4-oxo-1,4-dihydro-3-quinolinecarboxamide;
(d) N-(4-chlorobenzyl)-1-methyl-4-oxo-6-[(1-oxo-1′,4-thiazinan-4-yl)methyl]-1,4-dihydro-3-quinolinecarboxamide;
(e) N-(4-chlorobenzyl)-8-fluoro-1-methyl-6-(4-morpholinylmethyl)-4-oxo-1,4-dihydro-3-quinolinecarboxamide;
or a pharmaceutically acceptable salt thereof.

11. The method of claim 7, wherein the compound administered is N-(4-chlorobenzyl)-1-methyl-6-(4-morpholinylmethyl)-4-oxo-1,4-dihydro-3-quinolinecarboxamide, or a pharmaceutically acceptable salt thereof.

12. The method of claim 1, wherein the compound administered is Formula III 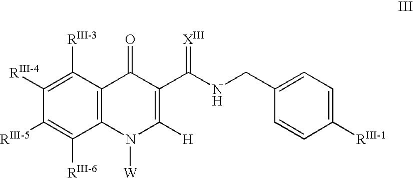
or a pharmaceutically acceptable salt thereof
wherein,
X^{III} is
a) O, or
b) S;
W is
a) R^{III-2};
b) NR^{XIII-7}R^{III-8},
c) OR^{III-9}, or
d) SOᵢR^{III-9};
R^{III-1} is
a) Cl,
b) F,
c) Br,
d) CN, or
e) NO₂;
R^{III-2} is
a) (CH₂CH₂O)ₘR^{III-10},
b) het^{III}, wherein said het^{III} is bonded via a carbon atom,
c) C₁₋₇ alkyl which may be partially unsaturated and is optionally substituted by one or more substituents selected from a group consisting of NR^{III-7}R^{III-8}, R^{III-11}, CN, SOᵢR^{III-9}, or OC₂₋₄ alkyl which is further substituted by het^{III}, OR^{III-10}, OC(═O)aryl^{III}, or NR^{III-7}R^{III-8}, or
d) C₃₋₈ cycloalkyl, which may be partially unsaturated and is optionally substituted by R^{III-11}, NR^{III-7}R^{III-8}, SOᵢR^{III-9}, or C₁₋₇ alkyl optionally substituted by R^{III-11}, NR^{III-7}R^{III-8}, or SOᵢR^{III-9};
R^{III-3} is
a) H,
b) halo, or
c) C₁₋₄ alkyl, optionally substituted by one to three halo;
R^{III-4} is
a) H,
b) aryl^{III},
c) het^{III},
d) SO₂NHR^{III-12},
e) CONHR^{III-12},
f) NR^{III-7}R^{III-8},
g) NHCOR^{III-12},
h) NHSO₂R^{III-12},
i) OC₂₋₇ alkyl optionally substituted by —OH,
j) SC₂₋₇ alkyl optionally substituted by OH, or
k) C₁₋₈ alkyl which may be partially unsaturated and is optionally substituted by one or more substituents selected from a group consisting of N₃, OR^{III-10}, NR^{III-7}R^{III-8}, halo, SOᵢR^{III-9}, OR^{III-13} or R^{III-11};
R^{III-5} is
a) H,
b) halo,
c) C≡CR^{III-14},
d) NR^{III-7}R^{III-8},
e) SO₂NHR^{III-12},
f) het^{III}, or
g) C₁₋₇ alkyl, optionally substituted by OH;
R^{III-6} is
a) H,
b) halo,
c) SC₁₋₇ alkyl,
d) C₁₋₇ alkoxy, optionally substituted by one or more halo or OH, or
e) C₁₋₇ alkyl, which may be partially unsaturated and is optionally substituted by halo, NR^{III-10}R^{III-10}, (CH₂)ₙOR^{III-13}, R^{III-11}, OC₁₋₇ alkyl which is further substituted with het^{III}, NR^{III-7}R^{III-8}, or SOᵢR^{III-9};
R^{III-7} and R^{III-8} are independently
a) H,
b) aryl^{III},
c) C₁₋₇alkyl which may be partially unsaturated and is optionally substituted by one or more substituents selected from a group consisting of N^{III-10}R^{III-10}, CONR^{III-10}R^{III-10}, R^{III-11}, SOᵢR^{III-9}, halo; or
d) R^{III-7} and R^{III-8} together with the nitrogen to which they are attached to form a het^{III};
R^{III-9} is
a) aryl^{III},
b) het^{III},
c) C₃₋₈cycloalkyl, or
d) C₁₋₇alkyl which may be partially unsaturated and optionally substituted by one or more OR^{III-10}, Oaryl^{III}, het^{III}, aryl^{III}, NR^{III-10}R^{III-10}, CN, SH, SOᵢC₁₋₆ alkyl, SOᵢ aryl^{III}, halo, or CONR^{III-10}R^{III-10};
R^{III-10} is
a) H, or
b) C₁₋₇ alkyl, optionally substituted by OH;
R^{III-11} is
a) OR^{III-10},
b) Ohet^{III},
c) Oaryl^{III},
d) CO₂R^{III-10},
e) het^{III},
f) aryl^{III}, or
g) CN;
R^{III-12} is
a) H,
b) het^{III},
c) aryl^{III},
d) C₃₋₈ cycloalkyl, or
e) C₁₋₇ alkyl optionally substituted by NR^{III-7}R^{III-8}, or R^{III-11};
R^{III-13} is
a) (P═O)(OH)₂,
b) (P═O)(C₁₋₇ alkoxy)₂,
c) CO(CH₂)ₙCON(CH₃)(CH₂)ₙSO₃⁻M^{III+},
d) an amino acid,
e) C(═O)aryl^{III},
f) C(═O)C₁₋₆alkyl, optionally substituted by NR^{III-10}R^{III-10}, or
g) CO(CH₂)ₙCO₂H;
R^{III-14} is
a) het^{III},
b) (CH₂)ₙOR^{III-13}, or
c) C₁₋₇ alkyl substituted by one or more substituents selected from a group consisting of R^{III-11}, OC₁₋₇ alkyl which is further substituted with het^{III}, NR^{III-7}R^{III-8}, or SOᵢR^{III-9};
aryl^{III} is a phenyl radical or an ortho-fused bicyclic carbocyclic radical wherein at least one ring is aromatic;
het^{III} is a four- (4), five- (5), six- (6), or seven- (7) membered saturated or unsaturated heterocyclic ring having 1, 2, or 3 heteroatoms selected from the group consisting of oxygen, sulfur, and nitrogen, which is optionally fused to a benzene ring, or any bicyclic heterocycle group;
wherein any aryl^{III} is optionally substituted with one or more substituents selected from the group consisting of halo, OH, CF₃, C₁₋₆alkoxy, and C₁₋₆ alkyl which maybe further substituted by one to three SR^{III-10}, N^{III-10}R^{III-10}, OR^{III-10}, or CO₂R^{III-10};
wherein any ^{III-}het is optionally substituted with one or more substituents selected from the group consisting of halo, OH, CF₃, C₁₋₆alkoxy, oxo, oxine, and C₁₋₆ alkyl which maybe further substituted by one to three SR^{III-10}, NR^{III-10}R^{III-10}, OR^{III-10}, or CO₂R^{III-10};
i^{III} is 0, 1, or 2;
m^{III} is 1, 2, or 3;
n^{III} is 1, 2, 3, 4, 5, or 6; and
M^{III} is sodium, potassium, or lithium.

13. The method of claim 12, wherein the compound administered is 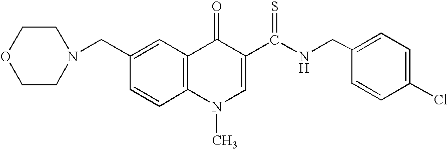
or a pharmaceutically acceptable salt thereof.

14. The method of claim 12, wherein the compound administered is 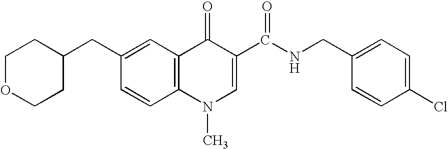
or a pharmaceutically acceptable salt thereof.

15. The method of claim 12, wherein the compound administered is 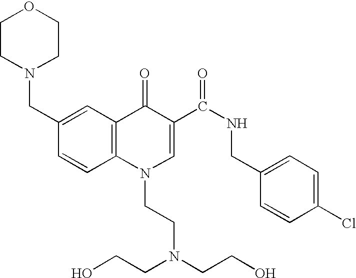
or a pharmaceutically acceptable salt thereof.

16. The method of claim 12, wherein the compound administered is 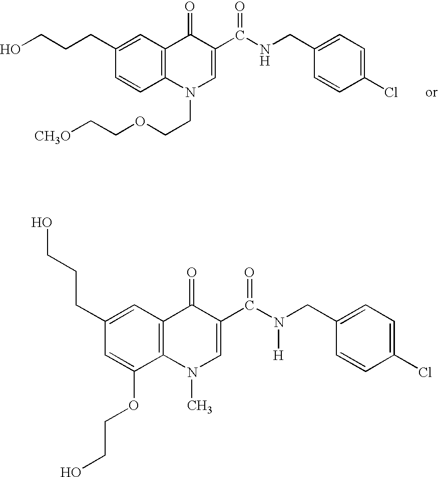
or a pharmaceutically acceptable salt thereof.

17. The method of claim 12, wherein the compound administered is 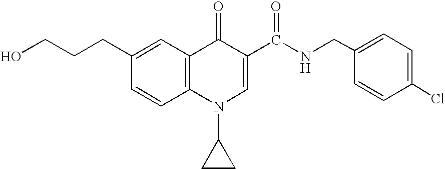
or a pharmaceutically acceptable salt thereof.

18. The method of claim 12, wherein the compound administered is 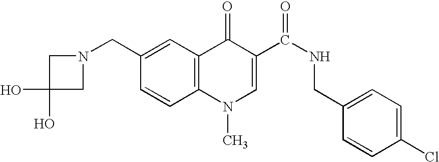
or a pharmaceutically acceptable salt thereof.

19. The method of claim 12, wherein the compound administered is 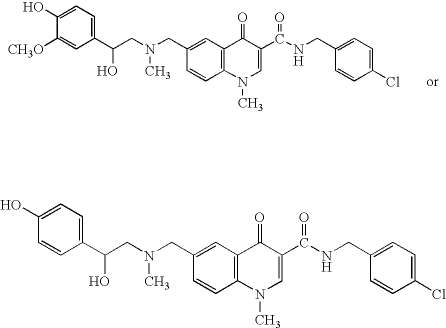
or a pharmaceutically acceptable salt thereof.

20. The method of claim 12, wherein the compound administered is 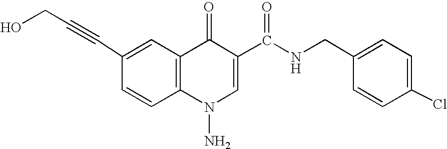
or a pharmaceutically acceptable salt thereof.

21. The method of claim 12, wherein the compound administered is 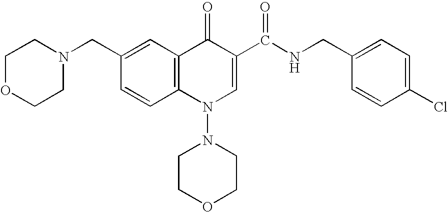
or a pharmaceutically acceptable salt thereof.

22. The method of claim 12, wherein the compound administered is
(1) N-(4-chlorobenzyl)-6-(3-hydroxy-1-propynyl)-1-isopropyl-4-oxo-1,4-dihydro-3-quinolinecarboxamide;
(2) 1-(sec-butyl)-N-(4-chlorobenzyl)-6-(3-hydroxy-1-propynyl)-4-oxo-1,4-dihydro-3-quinolinecarboxamide;
(3) 1-(sec-butyl)-N-(4-chlorobenzyl)-6-(3-hydroxy-1-propynyl)-8-methoxy-4-oxo-1,4-dihydro-3-quinolinecarboxamide;
(4) N-(4-chlorobenzyl)-6-[3-hydroxy-1-propenyl]-1-[2-(4-morpholinyl)ethyl]-4-oxo-1,4-dihydro-3-quinolinecarboxamide;
(5) N-(4-chlorobenzyl)-8-fluoro-6-(3-hydroxy-1-propynyl)-1-methyl-4-oxo-1,4-dihydro-3-quinolinecarboxamide;
(6) N-(4-chlorobenzyl)-8-fluoro-6-[(Z)-3-hydroxy-1-propenyl]-1-methyl-4-oxo-1,4-dihydro-3-quinolinecarboxamide;
(7) N-(4-chlorobenzyl)-1-[2-(diethylamino)ethyl]-8-fluoro-6-(3-hydroxy-1-propynyl)-4-oxo-1,4-dihydro-3-quinolinecarboxamide;
(8) N-(4-chlorobenzyl)-6-(3-hydroxy-1-propynyl)-4-oxo-1-propyl-1,4-dihydro-3-quinolinecarboxamide;
(9) N-(4-chlorobenzyl)-1-[2-(diethylamino)ethyl]-6-(3-hydroxy-1-propynyl)-4-oxo-1,4-dihydro-3-quinolinecarboxamide;
(10) N-(4-chlorobenzyl)-1-[2-(dimethylamino)ethyl]-6-(3-hydroxy-1-propynyl)-4-oxo-1,4-dihydro-3-quinolinecarboxamide hydrochloride;
(11) N-(4-chlorobenzyl)-6-(3-hydroxy-1-propynyl)-4-oxo-1-[2-(1-piperidinyl)ethyl]-1,4-dihydro-3-quinolinecarboxamide;
(12) N-(4-chlorobenzyl)-6-(3-hydroxy-1-propynyl)-4-oxo-1-[3-(1-piperidinyl)propyl]-1,4-dihydro-3-quinolinecarboxamide;
(13) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;
(14) N-(4-chlorobenzyl)-1-[2-(diisopropylamino)ethyl]-6-(3-hydroxy-1-propynyl)-4-oxo-1,4-dihydro-3-quinolinecarboxamide;
(15) N-(4-chlorobenzyl)-6-(3-hydroxy-1-propynyl)-4-oxo-1-[2-(1-pyrrolidinyl)ethyl]1,4-dihydro-3-quinolinecarboxamide;
(16) N-(4-chlorobenzyl)-6-(3-hydroxy-1-propynyl)-1-[2-(4-morpholinyl)ethyl]-4-oxo-1,4-dihydro-3-quinolinecarboxamide;
(17) N-(4-chlorobenzyl)-1-[3-(dimethylamino)propyl]-6-(3-hydroxy-1-propynyl)-4-oxo-1,4-dihydro-3-quinolinecarboxamide;
(18) N-(4-chlorobenzyl)-6-(3-hydroxy-1-propynyl)-4-oxo-1-vinyl-1,4-dihydro-3-quinolinecarboxamide;
(19) N-(4-chlorobenzyl)-6-[(E)-3-hydroxy-1-propenyl]-1-methyl-4-oxo-1,4-dihydro-3-quinolinecarboxamide;
(20) N-(4-chlorobenzyl)-6-[(Z)-3-hydroxy-1-propenyl]-1-methyl-4-oxo-1,4-dihydro-3-quinolinecarboxamide;
(21) N-(4-chlorobenzyl)-1-cyclopropyl-6-(3-hydroxy-1-propynyl)-4-oxo-1,4-dihydro-3-quinolinecarboxamide;
(22) tert-butyl 2-[3-{[(4-chlorobenzyl)amino]carbonyl}-6-(3-hydroxy-1-propynyl)-4-oxo-1(4H)-quinolinyl]acetate;
(23) 2-[3-{[(4-chlorobenzyl)amino]carbonyl}-6-(3-hydroxy-1-propynyl)-4-oxo-1(4H)-quinolinyl]acetic acid;
(24) N-(4-chlorobenzyl)-1-(2-hydroxyethyl)-6-(3-hydroxy-1-propynyl)-4-oxo-1,4-dihydro-3-quinolinecarboxamide;
(25) N-(4-chlorobenzyl)-6-(3-hydroxy-1-propynyl)-1-methyl-4-oxo-1,4-dihydro-3-quinolinecarboxamide;
(26) di(tert-butyl) 3-(3-{[(4-chlorobenzyl)amino]carbonyl}-1-methyl-4-oxo-1,4-dihydro-6-quinolinyl)propyl phosphate;
(27) 3-(3-{[(4-chlorobenzyl)amino]carbonyl}-1-methyl-4-oxo-1,4-dihydro-6-quinolinyl)propyl dihydrogen phosphate;
(28) di(tert-butyl) 3-(3-{[(4-chlorobenzyl)amino]carbonyl}-1-cyclopropyl-4-oxo-1,4-dihydro-6-quinolinyl)propyl phosphate;
(29) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;
(30) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;
(31) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;
(32) sodium 2-[(8-{[3-(3-{[(4-chlorobenzyl)amino]carbonyl}-1-cyclopropyl-4-oxo-1,4-dihydro-6-quinolinyl)-2-propynyl]oxy}-8-oxooctanoyl)(methyl)amino]-1-ethanesulfonate;
(33) 1-(tert-butyl)-N-(4-chlorobenzyl)-6-(3-hydroxy-1-propynyl)-4-oxo-1,4-dihydro-3-quinolinecarboxamide;
(34) sodium 2-[{8-[3-(1-(tert-butyl)-3-{[(4-chlorobenzyl)amino]-carbonyl}-4-oxo-1,4-dihydro-6-quinolinyl)propoxy]-8-oxooctanoyl}(methyl)amino]-1-ethanesulfonate;
(35) sodium 2-[(8-{[3-(1-(tert-butyl)-3-{[(4-chlorobenzyl)amino]-carbonyl}-4-oxo-1,4-dihydro-6-quinolinyl)-2-propynyl]oxy}-8-oxooctanoyl)(methyl)amino]-1-ethanesulfonate;
(36) N-(4-chlorobenzyl)-6-(3-hydroxy-1-propynyl)-1-[2-(2-methoxyethoxy)-ethyl]-4-oxo-1,4-dihydro-3-quinolinecarboxamide;
(37) N-(4-cyanobenzyl)-6-(3-hydroxy-1-propynyl)-1-methyl-4-oxo-1,4-dihydro-3-quinolinecarboxamide;
(38) N-(4-chlorobenzyl)-1-methyl-6-(1,4-oxazepan-4-ylmethyl)-4-oxo-1,4-dihydro-3-quinolinecarboxamide;
(39) N-(4-chlorobenzyl)-1-methyl-4-oxo-6-(1,4-thiazepan-4-ylmethyl)-1,4-dihydro-3-quinolinecarboxamide;
(40) N-(4-chlorobenzyl)-1-methyl-6-(2-oxa-5-azabicyclo[2.2.1]hept-5-ylmethyl)-4-oxo-1,4-dihydro-3-quinolinecarboxamide;
(41) N-(4-chlorobenzyl)-6-(2,3-dihydro-4H-1,4-benzoxazin-4-ylmethyl)-1-methyl-4-oxo-1,4-dihydro-3-quinolinecarboxamide;
(42) 6-(azidomethyl)-N-(4-chlorobenzyl)-1-methyl-4-oxo-1,4-dihydro-3-quinoline-carboxamide;
(43) N-(4-chlorobenzyl)-1-methyl-4-oxo-6-vinyl-1,4-dihydro-3-quinoline-carboxamide;
(44) N-(4-chlorobenzyl)-1-[2-(2-hydroxyethoxy)ethyl]-6-(3-hydroxy-1-propynyl)-4-oxo-1,4-dihydro-3-quinolinecarboxamide;
(45) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;
(46) N-(4-chlorobenzyl)-1-[2-(2-hydroxyethoxy)ethyl]-6-(4-morpholinylmethyl)-4-oxo-1,4-dihydro-3-quinolinecarboxamide;
(47) N-(4-chlorobenzyl)-1-[2-(2-ethoxyethoxy)ethyl]-6-(4-morpholinylmethyl)-4-oxo-1,4-dihydro-3-quinolinecarboxamide;
(48) N-(4-chlorobenzyl)-6-(4-morpholinylmethyl)-4-oxo-1-(2-propynyl)-1,4-dihydro-3-quinolinecarboxamide;
(49) N-(4-chlorobenzyl)-1-[2-(ethylsulfanyl)ethyl]-6-(4-morpholinylmethyl)-4-oxo-1,4-dihydro-3-quinolinecarboxamide;
(50) N-(4-chlorobenzyl)-1-[3-(methylsulfanyl)propyl]-6-(4-morpholinylmethyl)-4-oxo-1,4-dihydro-3-quinolinecarboxamide;
(51) N-(4-chlorobenzyl)-1-(4-hydroxy-2-butynyl)-6-(4-morpholinylmethyl)-4-oxo-1,4-dihydro-3-quinolinecarboxamide;
(52) N-(4-chlorobenzyl)-6-{[(2-hydroxy-2-phenylethyl)(methyl)amino]methyl}-1-methyl-4-oxo-1,4-dihydro-3-quinolinecarboxamide;
(53) N-(4-chlorobenzyl)-1-[3-(methylsulfinyl)propyl]-6-(4-morpholinylmethyl)-4-oxo-1,4-dihydro-3-quinolinecarboxamide;
(54) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;
(55) N-(4-chlorobenzyl)-1-[3-(methylsulfonyl)propyl]-6-(4-morpholinylmethyl)-4-oxo-1,4-dihydro-3-quinolinecarboxamide;
(56) N-(4-chlorobenzyl)-1-[2-(ethylsulfinyl)ethyl]-6-(4-morpholinylmethyl)-4-oxo-1,4-dihydro-3-quinolinecarboxamide;
(57) N-(4-chlorobenzyl)-1-[2-(ethylsulfonyl)ethyl]-6-(4-morpholinylmethyl)-4-oxo-1,4-dihydro-3-quinolinecarboxamide;
(58) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;
(59) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;
(60) N-(4-chlorobenzyl)-6-(4-morpholinylmethyl)-4-oxo-1-[2-(phenylsulfanyl)ethyl]-1,4-dihydro-3-quinolinecarboxamide;
(61) N-(4-chlorobenzyl)-1-[(methylsulfanyl)methyl]-6-(4-morpholinylmethyl)-4-oxo-1,4-dihydro-3-quinolinecarboxamide;
(62) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;
(63) N-(4-chlorobenzyl)-6-[(3-hydroxy-1-azetidinyl)methyl]-1-methyl-4-oxo-1,4-dihydro-3-quinolinecarboxamide;
(64) N-(4-chlorobenzyl)-6-(4-morpholinylmethyl)-4-oxo-1-[(phenylsulfanyl)-methyl]-1,4-dihydro-3-quinolinecarboxamide;
(65) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;
(66) N-(4-chlorobenzyl)-6-[(3,3-dihydroxy-1-azetidinyl)methyl]-1-methyl-4-oxo-1,4-dihydro-3-quinolinecarboxamide;
(67) N-(4-chlorobenzyl)-1-[(methylsulfinyl)methyl]-6-(4-morpholinylmethyl)-4-oxo-1,4-dihydro-3-quinolinecarboxamide;
(68) N-(4-chlorobenzyl)-1-[(methylsulfonyl)methyl]-6-(4-morpholinylmethyl)-4-oxo-1,4-dihydro-3-quinolinecarboxamide;
(69) N-(4-chlorobenzyl)-6-(4-morpholinylmethyl)-4-oxo-1-[(phenylsulfinyl)-methyl]-1,4-dihydro-3-quinolinecarboxamide;
(70) N-(4-chlorobenzyl)-6-(4-morpholinylmethyl)-4-oxo-1-[(phenylsulfonyl)-methyl]-1,4-dihydro-3-quinolinecarboxamide;
(71) N-(4-chlorobenzyl)-6-(3-hydroxypropyl)-1-[2-(2-methoxyethoxy)ethyl]-4-oxo-1,4-dihydro-3-quinolinecarboxamide;
(72) N-(4-chlorobenzyl)-1-[2-(2-methoxyethoxy)ethyl]-6-(4-morpholinylmethyl)-4-oxo-1,4-dihydro-3-quinolinecarboxamide;
(73) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;
(74) N-(4-chlorobenzyl)-6-{[(cyanomethyl)(methyl)amino]methyl}-1-[2-(2-methoxyethoxy)ethyl]4-oxo-1,4-dihydro-3-quinolinecarboxamide;
(75) N-(4-chlorobenzyl)-6-{[(3R)-3-hydroxypyrrolidinyl]methyl}-1-[2-(2-methoxyethoxy)ethyl]-4-oxo-1,4-dihydro-3-quinolinecarboxamide;
(76) N-(4-chlorobenzyl)-1-[2-(2-methoxyethoxy)ethyl]-6-[(methylsulfanyl)methyl]-4-oxo-1,4-dihydro-3-quinolinecarboxamide;
(77) N-(4-chlorobenzyl)-6-{[[(1R,2S)-2-hydroxy-1-methyl-2-phenylethyl](methyl)-amino]methyl}-1-[2-(2-methoxyethoxy)ethyl]-4-oxo-1,4-dihydro-3-quinoline-carboxamide;
(78) N-(4-chlorobenzyl)-6-{[(2-hydroxy-2-phenylethyl)(methyl)amino]methyl}-1-[2-(2-methoxyethoxy)ethyl]-4-oxo-1,4-dihydro-3-quinolinecarboxamide;
(79) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;
(80) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;
(81) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinoline-carboxamide;
(82) N-(4-chlorobenzyl)-6-(3-hydroxy-1-propynyl)-1-methyl-4-oxo-1,4-dihydro-3-quinolinecarbothioamide;
(83) N-(4-chlorobenzyl)-8-(3-hydroxy-1-propynyl)-1-methyl-4-oxo-1,4-dihydro-3-quinolinecarboxamide;
(84) N-(4-chlorobenzyl)-8-(4-hydroxy-1-butynyl)-1-methyl-4-oxo-1,4-dihydro-3-quinolinecarboxamide;
(85) N-(4-chlorobenzyl)-6-{[3-(hydroxyimino)-1-azetidinyl]methyl}-1-methyl-4-oxo-1,4-dihydro-3-quinolinecarboxamide;
(86) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;
(87) N-(4-chlorobenzyl)-1-([(4-chlorophenyl)sulfanyl]methyl)-6-(4-morpholinylmethyl)-4-oxo-1,4-dihydro-3-quinolinecarboxamide;
(88) N-(4-chlorobenzyl)-1-([(4-chlorophenyl)sulfinyl]methyl)-6-(4-morpholinylmethyl)-4-oxo-1,4-dihydro-3-quinolinecarboxamide;
(89) N-(4-chlorobenzyl)-1-([(4-chlorophenyl)sulfonyl]methyl)-6-(4-morpholinylmethyl)-4-oxo-1,4-dihydro-3-quinolinecarboxamide;
(90) N-(4-chlorobenzyl)-1-[(4-chlorophenoxy)methyl]-6-(4-morpholinylmethyl)-4-oxo-1,4-dihydro-3-quinolinecarboxamide;
(91) N-(4-chlorobenzyl)-1-[(2-methoxyethoxy)methyl]-6-(4-morpholinylmethyl)-4-oxo-1,4-dihydro-3-quinolinecarboxamide;
(92) 2-([3-1[(4-chlorobenzyl)amino]carbonyl}-6-(4-morpholinylmethyl)-4-oxo-1(4H)-quinolinyl]methoxy}ethyl benzoate;
(93) N-(4-chlorobenzyl)-1-[(2-hydroxyethoxy)methyl]-6-(4-morpholinylmethyl)-4-oxo-1,4-dihydro-3-quinolinecarboxamide;
(94) N-(4-chlorobenzyl)-6-(4-morpholinylmethyl)-4-oxo-1-tetrahydro-2H-pyran-4-yl-1,4-dihydro-3-quinolinecarboxamide;
(95) N-(4-chlorobenzyl)-1-(1-methyl-4-piperidinyl)-6-(4-morpholinylmethyl)-4-oxo-1,4-dihydro-3-quinolinecarboxamide;
(96) N-(4-chlorobenzyl)-6-(4-morpholinylmethyl)-4-oxo-1-(4-piperidinyl)-1,4-dihydro-3-quinolinecarboxamide;
(97) N-(4-chlorobenzyl)-1-(1,1-dioxohexahydrothiopyran-4-yl)-6-(4-morpholinylmethyl)-4-oxo-1,4-dihydro-3-quinolinecarboxamide;
(98) N-(4-chlorobenzyl)-1-(4-morpholinyl)-6-(4-morpholinylmethyl)-4-oxo-1,4-dihydro-3-quinolinecarboxamide;
(99) N-(4-chlorobenzyl)-1-(4-methyl-1-piperazinyl)-6-(4-morpholinylmethyl)-4-oxo-1,4-dihydro-3-quinolinecarboxamide;
(100) N-(4-chlorobenzyl)-6-(4-morpholinylmethyl)-4-oxo-1-(1-piperidinyl)-1,4-dihydro-3-quinolinecarboxamide;
(101) N-(4-chlorobenzyl)-6-(4-morpholinylmethyl)-4-oxo-1-(1-pyrrolidinyl)-1,4-dihydro-3-quinolinecarboxamide;
(102) N-(4-chlorobenzyl)-1-[(2R)-2-(methoxymethyl)pyrrolidinyl]-6-(4-morpholinylmethyl)-4-oxo-1,4-dihydro-3-quinolinecarboxamide;
(103) N-(4-chlorobenzyl)-1-(dimethylamino)-6-(4-morpholinylmethyl)-4-oxo-1,4-dihydro-3-quinolinecarboxamide;
(104) 1-Amino-N-(4-chlorobenzyl)-6-(3-hydroxy-1-propynyl)-4-oxo-1,4-dihydro-3-quinolinecarboxamide;
(105) 1-Amino-N-(4-chlorobenzyl)-6-(3-hydroxypropyl)-4-oxo-1,4-dihydro-3-quinolinecarboxamide;
(106) N-(4-chlorobenzyl)-1-(dimethylamino)-6-(3-hydroxy-1-propynyl)-4-oxo-1,4-dihydro-3-quinolinecarboxamide;
(107) N-(4-chlorobenzyl)-1-(dimethylamino)-4-oxo-1,4-dihydro-3-quinolinecarboxamide;
(108) 1-(allyloxy)-N-(4-chlorobenzyl)-4-oxo-1,4-dihydro-3-quinolinecarboxamide;
(109) N-(4-chlorobenzyl)-1-methoxy-4-oxo-1,4-dihydro-3-quinolinecarboxamide;
(110) N-(4-bromobenzyl)-1-(4-morpholinyl)-6-(4-morpholinylmethyl)-4-oxo-1,4-dihydro-3-quinolinecarboxamide;
(111) N-(4-fluorobenzyl)-1-(4-morpholinyl)-6-(4-morpholinylmethyl)-4-oxo-1,4-dihydro-3-quinolinecarboxamide;
(112) N-(4-chlorobenzyl)-1-{[2-(4-morphoiinyl)ethoxy]methyl}-6-(4-morpholinylmethyl)-4-oxo-1,4-dihydro-3-quinolinecarboxamide;
(113) N-(4-chlorobenzyl)-1-{[2-(dimethylamino)ethoxy]methyl}-6-(4-morpholinylmethyl)-4-oxo-1,4-dihydro-3-quinolinecarboxamide;
(114) N-(4-chlorobenzyl)-1-{[2-(4-methyl-1-piperazinyl)ethoxy]methyl}-6-(4-morpholinylmethyl)-4-oxo-1,4-dihydro-3-quinolinecarboxamide;
(115) N-(4-chlorobenzyl)-6-(4-morpholinylmethyl)-4-oxo-1-{[2-(1-piperidinyl)ethoxy]methyl}-1,4-dihydro-3-quinolinecarboxamide;
(116) N-(4-chlorobenzyl)-6-(4-morpholinylmethyl)-4-oxo-1-{[2-(1-pyrrolidinyl)ethoxy]methyl}-1,4-dihydro-3-quinolinecarboxamide;
or a pharmaceutically acceptable salt thereof.

23. The method of claim 12, wherein the compound administered is:
(1) 1-(sec-butyl)-N-(4-chlorobenzyl)-6-(3-hydroxy-1-propynyl)-4-oxo-1,4-dihydro-3-quinolinecarboxamide;
(2) 1-(sec-butyl)-N-(4-chlorobenzyl)-6-(3-hydroxy-1-propynyl)-8-methoxy-4-oxo-1,4-dihydro-3-quinolinecarboxamide;
(3) N-(4-chlorobenzyl)-6-[3-hydroxy-1-propenyl]-1-[2-(4-morpholinyl)ethyl]-4-oxo-1,4-dihydro-3-quinolinecarboxamide;
(4) N-(4-chlorobenzyl)-8-fluoro-6-(3-hydroxy-1-propynyl)-1-methyl-4-oxo-1,4-dihydro-3-quinolinecarboxamide;
(5) N-(4-chlorobenzyl)-8-fluoro-6-[(Z)-3-hydroxy-1-propenyl]-1-methyl-4-oxo-1,4-dihydro-3-quinolinecarboxamide;
(6) N-(4-chlorobenzyl)-1-[2-(diethylamino)ethyl]-6-(3-hydroxy-1-propynyl)-4-oxo-1,4-dihydro-3-quinolinecarboxamide;
(7) N-(4-chlorobenzyl)-1-[2-(dimethylamino)ethyl]-6-(3-hydroxy-1-propynyl)-4-oxo-1,4-dihydro-3-quinolinecarboxamide hydrochloride;
(8) N-(4-chlorobenzyl)-6-(3-hydroxy-1-propynyl)-4-oxo-1-[2-(1-piperidinyl)ethyl]-1,4-dihydro-3-quinolinecarboxamide;
(9) N-(4-chlorobenzyl)-6-(3-hydroxy-1-propynyl)-4-oxo-1-[3-(1-piperidinyl)propyl]-1,4-dihydro-3-quinolinecarboxamide;
(10) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;
(11) N-(4-chlorobenzyl)-1-[2-(diisopropylamino)ethyl]-6-(3-hydroxy-1-propynyl)-4-oxo-1,4-dihydro-3-quinolinecarboxamide;
(12) N-(4-chlorobenzyl)-6-(3-hydroxy-1-propynyl)-4-oxo-1-[2-(1-pyrrolidinyl)ethyl]-1,4-dihydro-3-quinolinecarboxamide;
(13) N-(4-chlorobenzyl)-6-(3-hydroxy-1-propynyl)-1-[2-(4-morpholinyl)ethyl]-4-oxo-1,4-dihydro-3-quinolinecarboxamide;
(14) N-(4-chlorobenzyl)-1-[3-(dimethylamino)propyl]-6-(3-hydroxy-1-propynyl)-4-oxo-1,4-dihydro-3-quinolinecarboxamide;
(15) N-(4-chlorobenzyl)-6-(3-hydroxy-1-propynyl)-4-oxo-1-vinyl-1,4-dihydro-3-quinolinecarboxamide;
(16) N-(4-chlorobenzyl)-6-[(E)-3-hydroxy-1-propenyl]-1-methyl-4-oxo-1,4-dihydro-3-quinolinecarboxamide;
(17) N-(4-chlorobenzyl)-6-[(Z)-3-hydroxy-1-propenyl]-1-methyl-4-oxo-1,4-dihydro-3-quinolinecarboxamide;
(18) N-(4-chlorobenzyl)-1-cyclopropyl-6-(3-hydroxy-1-propynyl)-4-oxo-1,4-dihydro-3-quinolinecarboxamide;
(19) tert-butyl 2-[3-{[(4-chlorobenzyl)amino]carbonyl}-6-(3-hydroxy-1-propynyl)-4-oxo-1(4H)-quinolinyl]acetate;
(20) N-(4-chlorobenzyl)-1-(2-hydroxyethyl)-6-(3-hydroxy-1-propynyl)-4-oxo-1,4-dihydro-3-quinolinecarboxamide;
(21) N-(4-chlorobenzyl)-6-(3-hydroxy-1-propynyl)-1-methyl-4-oxo-1,4-dihydro-3-quinolinecarboxamide;
(22) 3-(3-{[(4-chlorobenzyl)amino]carbonyl}-1-methyl-4-oxo-1,4-dihydro-6-quinolinyl)propyl dihydrogen phosphate;
(23) di(tert-butyl) 3-(3-{[(4-chlorobenzyl)amino]carbonyl}-1-cyclopropyl-4-oxo-1,4-dihydro-6-quinolinyl)propyl phosphate;
(24) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;
(25) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;
(26) N-(4-chlorobenzyl)-6-(3-hydroxy-1-propynyl)-1-[2-(2-methoxyethoxy)-ethyl]-4-oxo-1,4-dihydro-3-quinolinecarboxamide;
(27) N-(4-cyanobenzyl)-6-(3-hydroxy-1-propynyl)-1-methyl-4-oxo-1,4-dihydro-3-quinolinecarboxamide;
(28) N-(4-chlorobenzyl)-6-(3-hydroxy-1-propynyl)-4-oxo-1-propyl-1,4-dihydro-3-quinolinecarboxamide;
(29) N-(4-chlorobenzyl)-1-methyl-6-(1,4-oxazepan-4-ylmethyl)-4-oxo-1,4-dihydro-3-quinolinecarboxamide;
(30) N-(4-chlorobenzyl)-1-methyl-4-oxo-6-(1,4-thiazepan-4-ylmethyl)-1,4-dihydro-3-quinolinecarboxamide;
(31) 6-(azidomethyl)-N-(4-chlorobenzyl)-1-methyl-4-oxo-1,4-dihydro-3-quinolinecarboxamide;
(32) N-(4-chlorobenzyl)-6-[(4,4-difluoro-1-piperidinyl)methyl]-1-methyl-4-oxo-1,4-dihydro-3-quinolinecarboxamide;
(33) N-(4-chlorobenzyl)-4-hydroxy-6-iodo-3-quinolinecarbothioamide;
(34) N-(4-chlorobenzyl)-6-(2,3-dihydro-4H-1,4-benzoxazin-4-ylmethyl)-1-methyl-4-oxo-1,4-dihydro-3-quinolinecarboxamide.
(35) N-(4-chlorobenzyl)-1-methyl-4-oxo-6-vinyl-1,4-dihydro-3-quinoline-carboxamide;
(36) N-(4-chlorobenzyl)-1-[2-(2-hydroxyethoxy)ethyl]-6-(3-hydroxy-1-propynyl)-4-oxo-1,4-dihydro-3-quinolinecarboxamide;
(37) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;
(38) N-(4-chlorobenzyl)-1-[2-(2-hydroxyethoxy)ethyl]-6-(4-morpholinylmethyl)-4-oxo-1,4-dihydro-3-quinolinecarboxamide;
(39) N-(4-chlorobenzyl)-1-[2-(2-ethoxyethoxy)ethyl]-6-(4-morpholinylmethyl)-4-oxo-1,4-dihydro-3-quinolinecarboxamide;
(40) N-(4-chlorobenzyl)-1-[2-(ethylsulfanyl)ethyl]-6-(4-morpholinylmethyl)-4-oxo-1,4-dihydro-3-quinolinecarboxamide;
(41) N-(4-chlorobenzyl)-1-[3-(methylsulfanyl)propyl]-6-(4-morpholinylmethyl)-4-oxo-1,4-dihydro-3-quinolinecarboxamide;
(42) N-(4-chlorobenzyl)-1-(4-hydroxy-2-butynyl)-6-(4-morpholinylmethyl)-4-oxo-1,4-dihydro-3-quinolinecarboxamide;
(43) N-(4-chlorobenzyl)-6-{[(2-hydroxy-2-phenylethyl)(methyl)amino]methyl}-1-methyl-4-oxo-1,4-dihydro-3-quinolinecarboxamide;
(44) N-(4-chlorobenzyl)-1-[3-(methylsulfinyl)propyl]-6-(4-morpholinylmethyl)-4-oxo-1,4-dihydro-3-quinolinecarboxamide;
(45) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;
(46) N-(4-chlorobenzyl)-1-[3-(methylsulfonyl)propyl]-6-(4-morpholinylmethyl)-4-oxo-1,4-dihydro-3-quinolinecarboxamide;
(47) N-(4-chlorobenzyl)-1-[2-(ethylsulfinyl)ethyl]-6-(4-morpholinylmethyl)-4-oxo-1,4-dihydro-3-quinolinecarboxamide;
(48) N-(4-chlorobenzyl)-1-[2-(ethylsulfonyl)ethyl]-6-(4-morpholinylmethyl)-4-oxo-1,4-dihydro-3-quinolinecarboxamide;
(49) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;
(50) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;
(51) N-(4-chlorobenzyl)-6-(4-morpholinylmethyl)-4-oxo-1-[2-(phenylsulfanyl)ethyl]-1,4-dihydro-3-quinolinecarboxamide;
(52) N-(4-chlorobenzyl)-1-[(methylsulfanyl)methyl]-6-(4-morpholinylmethyl)-4-oxo-1,4-dihydro-3-quinolinecarboxamide;
(53) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;
(54) N-(4-chlorobenzyl)-6-[(3-hydroxy-1-azetidinyl)methyl]-1-methyl-4-oxo-1,4-dihydro-3-quinolinecarboxamide;
(55) N-(4-chlorobenzyl)-6-(4-morpholinylmethyl)-4-oxo-1-[(phenylsulfanyl)-methyl]-1,4-dihydro-3-quinolinecarboxamide
(56) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;
(57) N-(4-chlorobenzyl)-6-[(3,3-dihydroxy-1-azetidinyl)methyl]-1-methyl-4-oxo-1,4-dihydro-3-quinolinecarboxamide;
(58) N-(4-chlorobenzyl)-1-[(methylsulfinyl)methyl]-6-(4-morpholinylmethyl)-4-oxo-1,4-dihydro-3-quinolinecarboxamide;
(59) N-(4-chlorobenzyl)-1-[(methylsulfonyl)methyl]-6-(4-morpholinylmethyl)-4-oxo-1,4-dihydro-3-quinolinecarboxamide;
(60) N-(4-chlorobenzyl)-6-(4-morpholinylmethyl)-4-oxo-1-[(phenylsulfinyl)-methyl]-1,4-dihydro-3-quinolinecarboxamide;
(61) N-(4-chlorobenzyl)-6-(4-morpholinylmethyl)-4-oxo-1-[(phenylsulfonyl)-methyl]-1,4-dihydro-3-quinolinecarboxamide;
(62) N-(4-chlorobenzyl)-6-(3-hydroxypropyl)-1-[2-(2-methoxyethoxy)ethyl]-4-oxo-1,4-dihydro-3-quinolinecarboxamide;
(63) N-(4-chlorobenzyl)-1-[2-(2-methoxyethoxy)ethyl]-6-(4-morpholinylmethyl)-4-oxo-1,4-dihydro-3-quinolinecarboxamide;
(64) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;
(65) N-(4-chlorobenzyl)-6-{[(3R)-3-hydroxypyrrolidinyl]methyl}-1-[2-(2-methoxyethoxy)ethyl]-4-oxo-1,4-dihydro-3-quinolinecarboxamide;
(66) N-(4-chlorobenzyl)-6-{[[(1R,2S)-2-hydroxy-1-methyl-2-phenylethyl](methyl)-amino]methyl}-1-[2-(2-methoxyethoxy)ethyl]-4-oxo-1,4-dihydro-3-quinolinecarboxamide;
(67) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;
(68) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;
(69) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinolinecarboxamide;
(70) N-(4-chlorobenzyl)-6-(3-hydroxy-1-propynyl)-1-methyl-4-oxo-1,4-dihydro-3-quinolinecarbothioamide;
(71) N-(4-chlorobenzyl)-8-(3-hydroxy-1-propynyl)-1-methyl-4-oxo-1,4-dihydro-3-quinolinecarboxamide;
(72) N-(4-chlorobenzyl)-8-(4-hydroxy-1-butynyl)-1-methyl-4-oxo-1,4-dihydro-3-quinolinecarboxamide;
(73) N-(4-chlorobenzyl)-6-{[3-(hydroxyimino)-1-azetidinyl]methyl}-1-methyl-4-oxo-1,4-dihydro-3-quinolinecarboxamide;
(74) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;
(75) N-(4-chlorobenzyl)-1-([(4-chlorophenyl)sulfanyl]methyl)-6-(4-morpholinylmethyl)-4-oxo-1,4-dihydro-3-quinolinecarboxamide;
(76) N-(4-chlorobenzyl)-1-([(4-chlorophenyl)sulfinyl]methyl)-6-(4-morpholinylmethyl)-4-oxo-1,4-dihydro-3-quinolinecarboxamide;
(77) N-(4-chlorobenzyl)-1-([(4-chlorophenyl)sulfonyl]methyl)-6-(4-morpholinylmethyl)-4-oxo-1,4-dihydro-3-quinolinecarboxamide;
(78) N-(4-chlorobenzyl)-1-[(4-chlorophenoxy)methyl]-6-(4-morpholinylmethyl)-4-oxo-1,4-dihydro-3-quinolinecarboxamide;
(79) N-(4-chlorobenzyl)-1-[(2-methoxyethoxy)methyl]-6-(4-morpholinylmethyl)-4-oxo-1,4-dihydro-3-quinolinecarboxamide;
(80) 2-{[3-1[(4-chlorobenzyl)amino]carbonyl}-6-(4-morpholinylmethyl)-4-oxo-1(4H)-quinolinyl]methoxy}ethylbenzoate;
(81) N-(4-chlorobenzyl)-1-[(2-hydroxyethoxy)methyl]-6-(4-morpholinylmethyl)-4-oxo-1,4-dihydro-3-quinolinecarboxamide;
(82) N-(4-chlorobenzyl)-6-(4-morpholinylmethyl)-4-oxo-1-tetrahydro-2H-pyran-4-yl-1,4-dihydro-3-quinolinecarboxamide;
(83) N-(4-chlorobenzyl)-1-(1-methyl-4-piperidinyl)-6-(4-morpholinylmethyl)-4-oxo-1,4-dihydro-3-quinolinecarboxamide;
(84) N-(4-chlorobenzyl)-6-(4-morpholinylmethyl)-4-oxo-1-(4-piperidinyl)-1,4-dihydro-3-quinolinecarboxamide;
(85) N-(4-chlorobenzyl)-1-(1,1-dioxohexahydrothiopyran-4-yl)-6-(4-morpholinylmethyl)-4-oxo-1,4-dihydro-3-quinolinecarboxamide;
(86) N-(4-chlorobenzyl)-1-(4-morpholinyl)-6-(4-morpholinylmethyl)-4-oxo-1,4-dihydro-3-quinolinecarboxamide;
(87) N-(4-chlorobenzyl)-1-(4-methyl-1-piperazinyl)-6-(4-morpholinylmethyl)-4-oxo-1,4-dihydro-3-quinolinecarboxamide;
(88) N-(4-chlorobenzyl)-6-(4-morpholinylmethyl)-4-oxo-1-(1-piperidinyl)-1,4-dihydro-3-quinolinecarboxamide;
(89) N-(4-chlorobenzyl)-6-(4-morpholinylmethyl)-4-oxo-1-(1-pyrrolidinyl)-1,4-dihydro-3-quinolinecarboxamide;
(90) N-(4-chlorobenzyl)-1-[(2R)-2-(methoxymethyl)pyrrolidinyl]-6-(4-morpholinylmethyl)-4-oxo-1,4-dihydro-3-quinolinecarboxamide;
(91) N-(4-chlorobenzyl)-1-(dimethylamino)-6-(4-morpholinylmethyl)-4-oxo-1,4-dihydro-3-quinolinecarboxamide;
(92) 1-amino-N-(4-chlorobenzyl)-6-(3-hydroxy-1-propynyl)-4-oxo-1,4-dihydro-3-quinolinecarboxamide;
(93) 1-amino-N-(4-chlorobenzyl)-6-(3-hydroxypropyl)-4-oxo-1,4-dihydro-3-quinolinecarboxamide;
(94) N-(4-chlorobenzyl)-1-(dimethylamino)-6-(3-hydroxy-1-propynyl)-4-oxo-1,4-dihydro-3-quinolinecarboxamide;
(95) 1-(allyloxy)-N-(4-chlorobenzyl)-4-oxo-1,4-dihydro-3-quinolinecarboxamide;
or a pharmaceutically acceptable salt thereof.

24. The method of claim 12, wherein the compound administered is
(1) 1-(sec-butyl)-N-(4-chlorobenzyl)-6-(3-hydroxy-1-propynyl)-4-oxo-1,4-dihydro-3-quinolinecarboxamide;
(2) 1-(sec-butyl)-N-(4-chlorobenzyl)-6-(3-hydroxy-1-propynyl)-8-methoxy-4-oxo-1,4-dihydro-3-quinolinecarboxamide;
(3) N-(4-chlorobenzyl)-6-[3-hydroxy-1-propenyl]-1-[2-(4-morpholinyl)ethyl]-4-oxo-1,4-dihydro-3-quinolinecarboxamide;
(4) N-(4-chlorobenzyl)-8-fluoro-6-(3-hydroxy-1-propynyl)-1-methyl-4-oxo-1,4-dihydro-3-quinolinecarboxamide;
(5) N-(4-chlorobenzyl)-8-fluoro-6-[(Z)-3-hydroxy-1-propenyl]-1-methyl-4-oxo-1,4-dihydro-3-quinolinecarboxamide;
(6) N-(4-chlorobenzyl)-1-[2-(dimethylamino)ethyl]-6-(3-hydroxy-1-propynyl)-4-oxo-1,4-dihydro-3-quinolinecarboxamide hydrochloride;
(7) N-(4-chlorobenzyl)-6-(3-hydroxy-1-propynyl)-4-oxo-1-[2-(1-piperidinyl)ethyl]-1,4-dihydro-3-quinolinecarboxamide;
(8) N-(4-chlorobenzyl)-6-(3-hydroxy-1-propynyl)-4-oxo-1-[3-(1-piperidinyl)propyl]-1,4-dihydro-3-quinolinecarboxamide;
(9) N-(4-chlorobenzyl)-6-(3-hydroxy-1-propynyl)-1-[2-(1-methyl-2-pyrrolidinyl)ethyl]-4-oxo-1,4-dihydro-3-quinolinecarboxamide;
(10) N-(4-chlorobenzyl)-1-[2-(diisopropylamino)ethyl]-6-(3-hydroxy-1-propynyl)-4-oxo-1,4-dihydro-3-quinolinecarboxamide;
(11) N-(4-chlorobenzyl)-6-(3-hydroxy-1-propynyl)-4-oxo-1-[2-(1-pyrrolidinyl)ethyl]-1,4-dihydro-3-quinolinecarboxamide;
(12) N-(4-chlorobenzyl)-6-(3-hydroxy-1-propynyl)-1-[2-(4-morpholinyl)ethyl]-4-oxo-1,4-dihydro-3-quinolinecarboxamide;
(13) N-(4-chlorobenzyl)-1-[3-(dimethylamino)propyl]-6-(3-hydroxy-1-propynyl)-4-oxo-1,4-dihydro-3-quinolinecarboxamide;
(14) N-(4-chlorobenzyl)-6-(3-hydroxy-1-propynyl)-4-oxo-1-vinyl-1,4-dihydro-3-quinolinecarboxamide;
(15) N-(4-chlorobenzyl)-6-[(E)-3-hydroxy-1-propenyl]-1-methyl-4-oxo-1,4-dihydro-3-quinolinecarboxamide;
(16) N-(4-chlorobenzyl)-6-[(Z)-3-hydroxy-1-propenyl]-1-methyl-4-oxo-1,4-dihydro-3-quinolinecarboxamide;
(17) N-(4-chlorobenzyl)-1-cyclopropyl-6-(3-hydroxy-1-propynyl)-4-oxo-1,4-dihydro-3-quinolinecarboxamide;
(18) tert-butyl 2-[3-{[(4-chlorobenzyl)amino]carbonyl}-6-(3-hydroxy-1-propynyl)-4-oxo-1(4H)-quinolinyl]acetate;
(19) N-(4-chlorobenzyl)-1-(2-hydroxyethyl)-6-(3-hydroxy-1-propynyl)-4-oxo-1,4-dihydro-3-quinolinecarboxamide;
(20) N-(4-chlorobenzyl)-6-(3-hydroxy-1-propynyl)-1-methyl-4-oxo-1,4-dihydro-3-quinolinecarboxamide;
(21) 3-(3-{[(4-chlorobenzyl)amino]carbonyl}-1-methyl-4-oxo-1,4-dihydro-6-quinolinyl)propyl dihydrogen phosphate;
(22) di(tert-butyl) 3-(3-{[(4-chlorobenzyl)amino]carbonyl}-1-cyclopropyl-4-oxo-1,4-dihydro-6-quinolinyl)propyl phosphate;
(23) sodium 2-[{8-[3-(3-{[(4-chlorobenzyl)amino]carbonyl}-1-cyclopropyl-4-oxo-1,4-dihydro-6-quinolinyl)propoxy]-8-oxooctanoyl}(methyl)amino]-1-ethanesulfonate;
(24) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;
(25) N-(4-chlorobenzyl)-6-(3-hydroxy-1-propynyl)-1-[2-(2-methoxyethoxy)ethyl]-4-oxo-1,4-dihydro-3-quinolinecarboxamide;
(26) N-(4-cyanobenzyl)-6-(3-hydroxy-1-propynyl)-1-methyl-4-oxo-1,4-dihydro-3-quinolinecarboxamide;
(27) N-(4-chlorobenzyl)-6-(3-hydroxy-1-propynyl)-4-oxo-1-propyl-1,4-dihydro-3-quinolinecarboxamide;
(28) 6-(azidomethyl)-N-(4-chlorobenzyl)-1-methyl-4-oxo-1,4-dihydro-3-quinolinecarboxamide;
(29) N-(4-chlorobenzyl)-6-(2,3-dihydro-4H-1,4-benzoxazin-4-ylmethyl)-1-methyl-4-oxo-1,4-dihydro-3-quinolinecarboxamide;
(30) N-(4-chlorobenzyl)-1-methyl-4-oxo-6-vinyl-1,4-dihydro-3-quinolinecarboxamide;
(31) N-(4-chlorobenzyl)-1-[2-(2-hydroxyethoxy)ethyl]-6-(3-hydroxy-1-propynyl)-4-oxo-1,4-dihydro-3-quinolinecarboxamide;
(32) N-(4-chlorobenzyl)-1-{2-[2-(2-methoxyethoxy)ethoxy]ethyl}-6-(4-morpholinylmethyl)-4-oxo-1,4-dihydro-3-quinolinecarboxamide;
(33) N-(4-chlorobenzyl)-1-[2-(2-hydroxyethoxy)ethyl]-6-(4-morpholinylmethyl)-4-oxo-1,4-dihydro-3-quinolinecarboxamide;
(34) N-(4-chlorobenzyl)-1-[2-(2-ethoxyethoxy)ethyl]-6-(4-morpholinylmethyl)-4-oxo-1,4-dihydro-3-quinolinecarboxamide;
(35) N-(4-chlorobenzyl)-1-[2-(ethylsulfanyl)ethyl]-6-(4-morpholinylmethyl)-4-oxo-1,4-dihydro-3-quinolinecarboxamide;
(36) N-(4-chlorobenzyl)-1-[3-(methylsulfanyl)propyl]-6-(4-morpholinylmethyl)-4-oxo-1,4-dihydro-3-quinolinecarboxamide;
(37) N-(4-chlorobenzyl)-1-(4-hydroxy-2-butynyl)-6-(4-morpholinylmethyl)-4-oxo-1,4-dihydro-3-quinolinecarboxamide;
(38) N-(4-chlorobenzyl)-1-[3-(methylsulfinyl)propyl]-6-(4-morpholinylmethyl)-4-oxo-1,4-dihydro-3-quinolinecarboxamide;
(39) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;
(40) N-(4-chlorobenzyl)-1-[3-(methylsulfonyl)propyl]-6-(4-morpholinylmethyl)-4-oxo-1,4-dihydro-3-quinolinecarboxamide;
(41) N-(4-chlorobenzyl)-1-[2-(ethylsulfinyl)ethyl]-6-(4-morpholinylmethyl)-4-oxo-1,4-dihydro-3-quinolinecarboxamide;
(42) N-(4-chlorobenzyl)-1-[2-(ethylsulfonyl)ethyl]-6-(4-morpholinylmethyl)-4-oxo-1,4-dihydro-3-quinolinecarboxamide;
(43) N-(4-chlorobenzyl)-1-{3-[(3-hydroxypropyl)sulfinyl]propyl}-6-(4-morpholinylmethyl)-4-oxo-1,4-dihydro-3-quinolinecarboxamide;
(44) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;
(45) N-(4-chlorobenzyl)-6-(4-morpholinylmethyl)-4-oxo-1-[2-(phenylsulfanyl)ethyl]-1,4-dihydro-3-quinolinecarboxamide;
(46) N-(4-chlorobenzyl)-1-[(methylsulfanyl)methyl]-6-(4-morpholinylmethyl)-4-oxo-1,4-dihydro-3-quinolinecarboxamide;
(47) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;
(48) N-(4-chlorobenzyl)-6-[(3-hydroxy-1-azetidinyl)methyl]-1-methyl-4-oxo-1,4-dihydro-3-quinolinecarboxamide;
(49) N-(4-chlorobenzyl)-6-(4-morpholinylmethyl)-4-oxo-1-[(phenylsulfanyl)-methyl]-1,4-dihydro-3-quinolinecarboxamide;
(50) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;
(51) N-(4-chlorobenzyl)-6-[(3,3-dihydroxy-1-azetidinyl)methyl]-1-methyl-4-oxo-1,4-dihydro-3-quinolinecarboxamide;
(52) N-(4-chlorobenzyl)-1-[(methylsulfinyl)methyl]-6-(4-morpholinylmethyl)-4-oxo-1,4-dihydro-3-quinolinecarboxamide;
(53) N-(4-chlorobenzyl)-1-[(methylsulfonyl)methyl]-6-(4-morpholinylmethyl)-4-oxo-1,4-dihydro-3-quinolinecarboxamide;
(54) N-(4-chlorobenzyl)-6-(4-morpholinylmethyl)-4-oxo-1-[(phenylsulfinyl)-methyl]-1,4-dihydro-3-quinolinecarboxamide;
(55) N-(4-chlorobenzyl)-6-(4-morpholinylmethyl)-4-oxo-1-[(phenylsulfonyl)-methyl]-1,4-dihydro-3-quinolinecarboxamide;
(56) N-(4-chlorobenzyl)-6-(3-hydroxypropyl)-1-[2-(2-methoxyethoxy)ethyl]-4-oxo-1,4-dihydro-3-quinolinecarboxamide;
(57) N-(4-chlorobenzyl)-1-[2-(2-methoxyethoxy)ethyl]-6-(4-morpholinylmethyl)-4-oxo-1,4-dihydro-3-quinolinecarboxamide;
(58) N-(4-chlorobenzyl)-1-[2-(2-methoxyethoxy)ethyl]-4-oxo-6-[(4-oxo-1-piperidinyl)methyl]-1,4-dihydro-3-quinolinecarboxamide;
(59) N-(4-chlorobenzyl)-6-{[(3R)-3-hydroxypyrrolidinyl]methyl}-1-[2-(2-methoxyethoxy)ethyl]-4-oxo-1,4-dihydro-3-quinolinecarboxamide;
(60) N-(4-chlorobenzyl)-6-{[[2-hydroxy-2-(4-hydroxyphenyl)ethyl](methyl)amino]-methyl}-1-[2-(2-methoxyethoxy)ethyl]-4-oxo-1,4-dihydro-3-quinolinecarboxamide;
(61) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;
(62) 1-{2-[2-(tert-butoxy)ethoxy]ethyl-N-(4-chlorobenzyl)-6-{[[2-hydroxy-2-(4-hydroxyphenyl)ethyl](methyl)amino]methyl-4-oxo-1,4-dihydro-3-quinolinecarboxamide;
(63) N-(4-chlorobenzyl)-6-(3-hydroxy-1-propynyl)-1-methyl-4-oxo-1,4-dihydro-3-quinolinecarbothioamide;
(64) N-(4-chlorobenzyl)-8-(3-hydroxy-1-propynyl)-1-methyl-4-oxo-1,4-dihydro-3-quinolinecarboxamide;
(65) N-(4-chlorobenzyl)-8-(4-hydroxy-1-butynyl)-1-methyl-4-oxo-1,4-dihydro-3-quinolinecarboxamide;
(66) N-(4-chlorobenzyl)-6-{[3-(hydroxyimino)-1-azetidinyl]methyl}-1-methyl-4-oxo-1,4-dihydro-3-quinolinecarboxamide;
(67) N-(4-chlorobenzyl)-1-{2-[2-(4-morpholinyl)ethoxy]ethyl}-6-(4-morpholinylmethyl)-4-oxo-1,4-dihydro-3-quinolinecarboxamide;
(68) N-(4-chlorobenzyl)-1-([(4-chlorophenyl)sulfanyl]methyl)-6-(4-morpholinylmethyl)-4-oxo-1,4-dihydro-3-quinolinecarboxamide;
(69) N-(4-chlorobenzyl)-1-([(4-chlorophenyl)sulfinyl]methyl)-6-(4-morpholinylmethyl)-4-oxo-1,4-dihydro-3-quinolinecarboxamide;
(70) N-(4-chlorobenzyl)-1-([(4-chlorophenyl)sulfonyl]methyl)-6-(4-morpholinylmethyl)-4-oxo-1,4-dihydro-3-quinolinecarboxamide;
(71) N-(4-chlorobenzyl)-1-[(2-methoxyethoxy)methyl]-6-(4-morpholinylmethyl)-4-oxo-1,4-dihydro-3-quinolinecarboxamide;
(72) 2-{[3-{[(4-chlorobenzyl)amino]carbonyl}-6-(4-morpholinylmethyl)-4-oxo-1(4H)-quinolinyl]methoxy}ethyl benzoate;
(73) N-(4-chlorobenzyl)-1-[(2-hydroxyethoxy)methyl]-6-(4-morpholinylmethyl)-4-oxo-1,4-dihydro-3-quinolinecarboxamide;
(74) N-(4-chlorobenzyl)-6-(4-morpholinylmethyl)-4-oxo-1-tetrahydro-2H-pyran-4-yl-1,4-dihydro-3-quinolinecarboxamide;
(75) N-(4-chlorobenzyl)-1-(1-methyl-4-piperidinyl)-6-(4-morpholinylmethyl)-4-oxo-1,4-dihydro-3-quinolinecarboxamide;
(76) N-(4-chlorobenzyl)-6-(4-morpholinylmethyl)-4-oxo-1-(4-piperidinyl)-1,4-dihydro-3-quinolinecarboxamide;
(77) N-(4-chlorobenzyl)-1-(1,1-dioxohexahydrothiopyran-4-yl)-6-(4-morpholinylmethyl)-4-oxo-1,4-dihydro-3-quinolinecarboxamide;
(78) N-(4-chlorobenzyl)-1-(4-morpholinyl)-6-(4-morpholinylmethyl)-4-oxo-1,4-dihydro-3-quinolinecarboxamide;
(79) N-(4-chlorobenzyl)-1-(4-methyl-1-piperazinyl)-6-(4-morpholinylmethyl)-4-oxo-1,4-dihydro-3-quinolinecarboxamide;
(80) N-(4-chlorobenzyl)-6-(4-morpholinylmethyl)-4-oxo-1-(1-piperidinyl)-1,4-dihydro-3-quinolinecarboxamide;
(81) N-(4-chlorobenzyl)-1-(dimethylamino)-6-(4-morpholinylmethyl)-4-oxo-1,4-dihydro-3-quinolinecarboxamide;
(82) 1-amino-N-(4-chlorobenzyl)-6-(3-hydroxy-1-propynyl)-4-oxo-1,4-dihydro-3-quinolinecarboxamide;
(83) 1-amino-N-(4-chlorobenzyl)-6-(3-hydroxypropyl)-4-oxo-1,4-dihydro-3-quinolinecarboxamide;
or a pharmaceutically acceptable salt thereof.

25. The method of claim 12, wherein the compound administered is
(1) N-(4-chlorobenzyl)-8-fluoro-6-[(Z)-3-hydroxy-1-propenyl]-1-methyl-4-oxo-1,4-dihydro-3-quinolinecarboxamide;
(2) N-(4-chlorobenzyl)-6-(3-hydroxy-1-propynyl)-1-[2-(4-morpholinyl)ethyl]-4-oxo-1,4-dihydro-3-quinolinecarboxamide;
(3) N-(4-chlorobenzyl)-6-(3-hydroxy-1-propynyl)-4-oxo-1-vinyl-1,4-dihydro-3-quinolinecarboxamide;
(4) N-(4-chlorobenzyl)-6-[(Z)-3-hydroxy-1-propenyl]-1-methyl-4-oxo-1,4-dihydro-3-quinolinecarboxamide;
(5) N-(4-chlorobenzyl)-1-cyclopropyl-6-(3-hydroxy-1-propynyl)-4-oxo-1,4-dihydro-3-quinolinecarboxamide;
(6) N-(4-chlorobenzyl)-1-(2-hydroxyethyl)-6-(3-hydroxy-1-propynyl)-4-oxo-1,4-dihydro-3-quinolinecarboxamide;
(7) N-(4-chlorobenzyl)-6-(3-hydroxy-1-propynyl)-1-methyl-4-oxo-1,4-dihydro-3-quinolinecarboxamide;
(8) 3-(3-{[(4-chlorobenzyl)amino]carbonyl}-1-methyl-4-oxo-1,4-dihydro-6-quinolinyl)propyl dihydrogen phosphate;
(9) sodium 2-[(8-{[3-(3-{[(4-chlorobenzyl)amino]carbonyl}-1-methyl-4-oxo-1,4-dihydro-6-quinolinyl)-2-propynyl]oxy}-8-oxooctanoyl)(methyl)amino]-1-ethanesulfonate;
(10) N-(4-chlorobenzyl)-6-(3-hydroxy-1-propynyl)-1-[2-(2-methoxyethoxy)-ethyl]-4-oxo-1,4-dihydro-3-quinolinecarboxamide;
(11) N-(4-chlorobenzyl)-1-methyl-4-oxo-6-vinyl-1,4-dihydro-3-quinolinecarboxamide;
(12) N-(4-chlorobenzyl)-1-[2-(2-hydroxyethoxy)ethyl]-6-(3-hydroxy-1-propynyl)-4-oxo-1,4-dihydro-3-quinolinecarboxamide;
(13) N-(4-chlorobenzyl)-1-[2-(2-hydroxyethoxy)ethyl]-6-(4-morpholinylmethyl)-4-oxo-1,4-dihydro-3-quinolinecarboxamide;
(14) N-(4-chlorobenzyl)-1-[2-(2-ethoxyethoxy)ethyl]-6-(4-morpholinylmethyl)-4-oxo-1,4-dihydro-3-quinolinecarboxamide;
(15) N-(4-chlorobenzyl)-1-[2-(ethylsulfanyl)ethyl]-6-(4-morpholinylmethyl)-4-oxo-1,4-dihydro-3-quinolinecarboxamide;
(16) N-(4-chlorobenzyl)-1-[3-(methylsulfanyl)propyl]-6-(4-morpholinylmethyl)-4-oxo-1,4-dihydro-3-quinolinecarboxamide;
(17) N-(4-chlorobenzyl)-1-(4-hydroxy-2-butynyl)-6-(4-morpholinylmethyl)-4-oxo-1,4-dihydro-3-quinolinecarboxamide;
(18) N-(4-chlorobenzyl)-1-[3-(methylsulfinyl)propyl]-6-(4-morpholinylmethyl)-4-oxo-1,4-dihydro-3-quinolinecarboxamide;
(19) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;
(20) N-(4-chlorobenzyl)-1-[3-(methylsulfonyl)propyl]-6-(4-morpholinylmethyl)-4-oxo-1,4-dihydro-3-quinolinecarboxamide;
(21) N-(4-chlorobenzyl)-1-[2-(ethylsulfinyl)ethyl]-6-(4-morpholinylmethyl)-4-oxo-1,4-dihydro-3-quinolinecarboxamide;
(22) N-(4-chlorobenzyl)-1-[2-(ethylsulfonyl)ethyl]-6-(4-morpholinylmethyl)-4-oxo-1,4-dihydro-3-quinolinecarboxamide;
(23) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;
(24) N-(4-chlorobenzyl)-6-(4-morpholinylmethyl)-4-oxo-1-[2-(phenylsulfanyl)ethyl]-1,4-dihydro-3-quinolinecarboxamide;
(25) N-(4-chlorobenzyl)-1-[(methylsulfanyl)methyl]-6-(4-morpholinylmethyl)-4-oxo-1,4-dihydro-3-quinolinecarboxamide;
(26) N-(4-chlorobenzyl)-6-{[[2-hydroxy-2-(4-hydroxyphenyl)ethyl](methyl)-amino]methyl}-1-methyl-4-oxo-1,4-dihydro-3-quinolinecarboxamide;
(27) N-(4-chlorobenzyl)-6-(4-morpholinylmethyl)-4-oxo-1-[(phenylsulfanyl)-methyl]-1,4-dihydro-3-quinolinecarboxamide;
(28) N-(4-chlorobenzyl)-6-{[[2-hydroxy-2-(4-hydroxy-3-methoxyphenyl)ethyl]-(methyl)amino]methyl}-1-methyl-4-oxo-1,4-dihydro-3-quinolinecarboxamide;
(29) N-(4-chlorobenzyl)-6-[(3,3-dihydroxy-1-azetidinyl)methyl]-1-methyl-4-oxo-1,4-dihydro-3-quinolinecarboxamide;
(30) N-(4-chlorobenzyl)-1-[(methylsulfinyl)methyl]-6-(4-morpholinylmethyl)-4-oxo-1,4-dihydro-3-quinolinecarboxamide;
(31) N-(4-chlorobenzyl)-1-[(methylsulfonyl)methyl]-6-(4-morpholinylmethyl)-4-oxo-1,4-dihydro-3-quinolinecarboxamide;
(32) N-(4-chlorobenzyl)-6-(4-morpholinylmethyl)-4-oxo-1-[(phenylsulfinyl)-methyl]-1,4-dihydro-3-quinolinecarboxamide;
(33) N-(4-chlorobenzyl)-6-(4-morpholinylmethyl)-4-oxo-1-[(phenylsulfonyl)-methyl]-1,4-dihydro-3-quinolinecarboxamide;
(34) N-(4-chlorobenzyl)-6-(3-hydroxypropyl)-1-[2-(2-methoxyethoxy)ethyl]-4-oxo-1,4-dihydro-3-quinolinecarboxamide;
(35) N-(4-chlorobenzyl)-1-[2-(2-methoxyethoxy)ethyl]-6-(4-morpholinylmethyl)-4-oxo-1,4-dihydro-3-quinolinecarboxamide;
(36) 1-{2-[2-(tert-butoxy)ethoxy]ethyl-N-(4-chlorobenzyl)-6-(4-morpholinyl-methyl)-4-oxo-1,4-dihydro-3-quinolinecarboxamide;
(37) N-(4-chlorobenzyl)-6-(3-hydroxy-1-propynyl)-1-methyl-4-oxo-1,4-dihydro-3-quinolinecarbothioamide;
(38) N-(4-chlorobenzyl)-8-(3-hydroxy-1-propynyl)-1-methyl-4-oxo-1,4-dihydro-3-quinolinecarboxamide;
(39) N-(4-chlorobenzyl)-8-(4-hydroxy-1-butynyl)-1-methyl-4-oxo-1,4-dihydro-3-quinolinecarboxamide;
(40) N-(4-chlorobenzyl)-1-([(4-chlorophenyl)sulfanyl]methyl)-6-(4-morpholinylmethyl)-4-oxo-1,4-dihydro-3-quinolinecarboxamide;
(41) N-(4-chlorobenzyl)-1-([(4-chlorophenyl)sulfinyl]methyl)-6-(4-morpholinylmethyl)-4-oxo-1,4-dihydro-3-quinolinecarboxamide;
(42) N-(4-chlorobenzyl)-1-[(2-methoxyethoxy)methyl]-6-(4-morpholinylmethyl)-4-oxo-1,4-dihydro-3-quinolinecarboxamide;
(43) 2-{[3-{[(4-chlorobenzyl)amino]carbonyl}-6-(4-morpholinylmethyl)-4-oxo-1(4H)-quinolinyl]methoxy}ethyl benzoate;
(44) N-(4-chlorobenzyl)-1-[(2-hydroxyethoxy)methyl]-6-(4-morpholinylmethyl)-4-oxo-1,4-dihydro-3-quinolinecarboxamide;
(45) N-(4-chlorobenzyl)-6-(4-morpholinylmethyl)-4-oxo-1-tetrahydro-2H-pyran-4-yl-1,4-dihydro-3-quinolinecarboxamide;
(46) N-(4-chlorobenzyl)-1-(1-methyl-4-piperidinyl)-6-(4-morpholinylmethyl)-4-oxo-1,4-dihydro-3-quinolinecarboxamide;
(47) N-(4-chlorobenzyl)-6-(4-morpholinylmethyl)-4-oxo-1-(4-piperidinyl)-1,4-dihydro-3-quinolinecarboxamide;
(48) N-(4-chlorobenzyl)-1-(1,1-dioxohexahydrothiopyran-4-yl)-6-(4-morpholinylmethyl)-4-oxo-1,4-dihydro-3-quinolinecarboxamide;
(49) N-(4-chlorobenzyl)-1-(4-morpholinyl)-6-(4-morpholinylmethyl)-4-oxo-1,4-dihydro-3-quinolinecarboxamide;
(50) 1-amino-N-(4-chlorobenzyl)-6-(3-hydroxy-1-propynyl)-4-oxo-1,4-dihydro-3-quinolinecarboxamide;
(51) 1-amino-N-(4-chlorobenzyl)-6-(3-hydroxypropyl)-4-oxo-1,4-dihydro-3-quinolinecarboxamide;
or a pharmaceutically acceptable salt thereof.

26. The method of claim 12, wherein the compound administered is
(1) N-(4-chlorobenzyl)-1-methyl-4-oxo-6-(tetrahydro-2H-pyran-4-ylmethyl)-1,4-dihydro-3-quinolinecarboxamide;
(2) N-(4-chlorobenzyl)-1-methyl-6-(4-morpholinylmethyl)-4-oxo-1,4-dihydro-3-quinolinecarbothioamide;
(3) N-(4-chlorobenzyl)-8-(2-hydroxyethoxy)-6-(3-hydroxypropyl)-1-methyl-4-oxo-1,4-dihydro-3-quinolinecarboxamide;
(4) N-(4-chlorobenzyl)-1-cyclopropyl-6-(3-hydroxypropyl)-4-oxo-1,4-dihydro-3-quinolinecarboxamide;
(5) 1-{2-[bis(2-hydroxyethyl)amino]ethyl}-N-(4-chlorobenzyl)-6-(4-morpholinyl-methyl)-4-oxo-1,4-dihydro-3-quinolinecarboxamide;
or a pharmaceutically acceptable salt thereof.

27. The method of claim 12, wherein the compound administered is
(1) N-(4-chlorobenzyl)-8-[2-hydroxy-1-(hydroxymethyl)ethoxy]-6-(3-hydroxypropyl)-1-methyl-4-oxo-1,4-dihydro-3-quinolinecarboxamide;
(2) N-(4-chlorobenzyl)-8-fluoro-6-(hydroxymethyl)-4-oxo-1-[3-(tetrahydro-2H-pyran-2-yloxy)propyl]-1,4-dihydro-3-quinolinecarboxamide;
(3) N-(4-chlorobenzyl)-6-[ethyl(2-hydroxyethyl)amino]-1-methyl-4-oxo 1,4-dihydro-3-quinolinecarboxamide;
(4) N-(4-chlorobenzyl)-1-cyclopropyl-6-(4-morpholinylmethyl)-4-oxo-1,4-dihydro-3-quinolinecarboxamide;
(5) 6-{[bis(2-hydroxyethyl)amino]methyl}-N-(4-chlorobenzyl)-1-methyl-4-oxo-1,4-dihydro-3-quinolinecarboxamide;
(6) N-(4-chlorobenzyl)-6-{[(2-hydroxyethyl)(methyl)amino]methyl}-1-methyl-4-oxo-1,4-dihydro-3-quinolinecarboxamide;
(7) 6-((benzyl(2-hydroxyethyl)amino)methyl)-N-(4-chlorobenzyl)-1-methyl-4-oxo-1,4-dihydro-3-quinolinecarboxamide;
(8) N-(4-chlorobenzyl)-6-[(4,4-difluoro-1-piperidinyl)methyl]-1-methyl-4-oxo-1,4-dihydro-3-quinolinecarboxamide;
(9) N-(4-chlorobenzyl)-6-{[4-fluoro-3,6-dihydro-1(2H)-pyridinyl]methyl}-1-methyl-4-oxo-1,4-dihydro-3-quinolinecarboxamide;
or a pharmaceutically acceptable salt thereof.

28. The method of claim 12, wherein the compound administered is
(1) N-(4-chlorobenzyl)-1-[2-(2-hydroxyethoxy)ethyl]-6-(3-hydroxy-1-propynyl)-4-oxo-1,4-dihydro-3-quinolinecarboxamide;
(2) N-(4-chlorobenzyl)-6-{[[2-hydroxy-2-(4-hydroxyphenyl)ethyl](methyl) amino]methyl}-1-methyl-4-oxo-1,4-dihydro-3-quinolinecarboxamide;
(3) N-(4-chlorobenzyl)-6-{[[2-hydroxy-2-(4-hydroxy-3-methoxyphenyl)ethyl](methyl)amino]methyl}-1-methyl-4-oxo-1,4-dihydro-3-quinolinecarboxamide;
(4) N-(4-chlorobenzyl)-6-[(3,3-dihydroxy-1-azetidinyl)methyl]-1-methyl-4-oxo-1,4-dihydro-3-quinolinecarboxamide;
(5) N-(4-chlorobenzyl)-8-fluoro-6-[(Z)-3-hydroxy-1-propenyl]-1-methyl-4-oxo-1,4-dihydro-3-quinolinecarboxamide;
(6) N-(4-chlorobenzyl)-6-(3-hydroxy-1-propynyl)-1-[2-(4-morpholinyl)ethyl]-4-oxo-1,4-dihydro-3-quinolinecarboxamide;
(7) N-(4-chlorobenzyl)-6-[(Z)-3-hydroxy-1-propenyl]-1-methyl-4-oxo-1,4-dihydro-3-quinolinecarboxamide;
(8) N-(4-chlorobenzyl)-1-cyclopropyl-6-(3-hydroxy-1-propynyl)-4-oxo-1,4-dihydro-3-quinolinecarboxamide;
(9) N-(4-chlorobenzyl)-1-(2-hydroxyethyl)-6-(3-hydroxy-1-propynyl)-4-oxo-1,4-dihydro-3-quinolinecarboxamide;
(10) N-(4-chlorobenzyl)-6-(3-hydroxy-1-propynyl)-1-methyl-4-oxo-1,4-dihydro-3-quinolinecarboxamide;
(11) N-(4-chlorobenzyl)-6-(3-hydroxy-1-propynyl)-1-[2-(2-methoxyethoxy)ethyl]-4-oxo-1,4-dihydro-3-quinolinecarboxamide;
(12) N-(4-chlorobenzyl)-6-(3-hydroxy-1-propynyl)-1-methyl-4-oxo-1,4-dihydro-3-quinolinecarbothioamide;
(13) N-(4-chlorobenzyl)-8-(3-hydroxy-1-propynyl)-1-methyl-4-oxo-1,4-dihydro-3-quinolinecarboxamide;
(14) N-(4-chlorobenzyl)-8-(4-hydroxy-1-butynyl)-1-methyl-4-oxo-1,4-dihydro-3-quinolinecarboxamide;
(15) N-(4-chlorobenzyl)-1-methyl-4-oxo-6-(tetrahydro-2H-pyran-4-ylmethyl)-1,4-dihydro-3-quinolinecarboxamide;
or a pharmaceutically acceptable salt thereof.

29. The method of claim 12, wherein the compound administered is
(1) N-(4-chlorobenzyl)-1-(4-morpholinyl)-6-(4-morpholinylmethyl)-4-oxo-1,4-dihydro-3-quinolinecarboxamide;
(2) 1-amino-N-(4-chlorobenzyl)-6-(3-hydroxy-1-propynyl)-4-oxo-1,4-dihydro-3-quinolinecarboxamide;
(3) 1-amino-N-(4-chlorobenzyl)-6-(3-hydroxypropyl)-4-oxo-1,4-dihydro-3-quinolinecarboxamide;
(4) N-(4-bromobenzyl)-1-(4-morpholinyl)-6-(4-morpholinylmethyl)-4-oxo-1,4-dihydro-3-quinolinecarboxamide;
(5) N-(4-fluorobenzyl)-1-(4-morpholinyl)-6-(4-morpholinylmethyl)-4-oxo-1,4-dihydro-3-quinolinecarboxamide;
or a pharmaceutically acceptable salt thereof.

30. The method of claim 12, wherein the compound administered is
(1) N-(4-chlorobenzyl)-1-{[(4-chlorophenyl)sulfanyl]methyl}-6-(4-morpholinylmethyl)-4-oxo-1,4-dihydro-3-quinolinecarboxamide;
(2) N-(4-chlorobenzyl)-1-{[(4-chlorophenyl)sulfinyl]methyl}-6-(4-morpholinylmethyl)-4-oxo-1,4-dihydro-3-quinolinecarboxamide;
(3) N-(4-chlorobenzyl)-1-[(2-methoxyethoxy)methyl]-6-(4-morpholinylmethyl)-4-oxo-1,4-dihydro-3-quinolinecarboxamide;
(4) 2-{[3-{[(4-chlorobenzyl)amino]carbonyl}-6-(4-morpholinylmethyl)-4-oxo-1(4H)-quinolinyl]methoxy}ethyl benzoate;
(5) N-(4-chlorobenzyl)-1-[(2-hydroxyethoxy)methyl]-6-(4-morpholinylmethyl)-4-oxo-1,4-dihydro-3-quinolinecarboxamide;
(6) N-(4-chlorobenzyl)-6-(4-morpholinylmethyl)-4-oxo-1-tetrahydro-2H-pyran-4-yl-1,4-dihydro-3-quinolinecarboxamide;
(7) N-(4-chlorobenzyl)-1-(1-methyl-4-piperidinyl)-6-(4-morpholinylmethyl)-4-oxo-1,4-dihydro-3-quinolinecarboxamide;
(8) N-(4-chlorobenzyl)-6-(4-morpholinylmethyl)-4-oxo-1-(4-piperidinyl)-1,4-dihydro-3-quinolinecarboxamide;
(9) N-(4-chlorobenzyl)-1-(1,1-dioxohexahydro-llambda˜6˜-thiopyran-4-yl)-6-(4-morpholinylmethyl)-4-oxo-1,4-dihydro-3-quinolinecarboxamide;
(10) N-(4-chlorobenzyl)-1-(4-morpholinyl)-6-(4-morpholinylmethyl)-4-oxo-1,4-dihydro-3-quinolinecarboxamide;
(11) N-(4-chlorobenzyl)-1-[2-(2-hydroxyethoxy)ethyl]-6-(4-morpholinylmethyl)-4-oxo-1,4-dihydro-3-quinolinecarboxamide;
(12) N-(4-chlorobenzyl)-1-[2-(2-ethoxyethoxy)ethyl]-6-(4-morpholinylmethyl)-4-oxo-1,4-dihydro-3-quinolinecarboxamide;
(13) N-(4-chlorobenzyl)-1-[2-(ethylsulfanyl)ethyl]-6-(4-morpholinylmethyl)-4-oxo-1,4-dihydro-3-quinolinecarboxamide;
(14) N-(4-chlorobenzyl)-1-[3-(methylsulfanyl)propyl]-6-(4-morpholinylmethyl)-4-oxo-1,4-dihydro-3-quinolinecarboxamide;
(15) N-(4-chlorobenzyl)-1-(4-hydroxy-2-butynyl)-6-(4-morpholinylmethyl)-4-oxo-1,4-dihydro-3-quinolinecarboxamide;
(16) 1-{2-[bis(2-hydroxyethyl)amino]ethyl}-N-(4-chlorobenzyl)-6-(4-morpholinylmethyl)-4-oxo-1,4-dihydro-3-quinolinecarboxamide;
(17) N-(4-chlorobenzyl)-1-[3-(methylsulfinyl)propyl]-6-(4-morpholinylmethyl)-4-oxo-1,4-dihydro-3-quinolinecarboxamide;
(18) N-(4-chlorobenzyl)-1-{3-[(3-hydroxypropyl)sulfanyl]propyl}-6-(4-morpholinylmethyl)-4-oxo-1,4-dihydro-3-quinolinecarboxamide;
(19) N-(4-chlorobenzyl)-1-[3-(methylsulfonyl)propyl]-6-(4-morpholinylmethyl)-4-oxo-1,4-dihydro-3-quinolinecarboxamide;
(20) N-(4-chlorobenzyl)-1-[2-(ethylsulfinyl)ethyl]-6-(4-morpholinylmethyl)-4-oxo-1,4-dihydro-3-quinolinecarboxamide;
(21) N-(4-chlorobenzyl)-1-[2-(ethylsulfonyl)ethyl]-6-(4-morpholinylmethyl)-4-oxo-1,4-dihydro-3-quinolinecarboxamide;
(22) N-(4-chlorobenzyl)-1-{3-[(3-hydroxypropyl)sulfonyl]propyl}-6-(4-morpholinylmethyl)-4-oxo-1,4-dihydro-3-quinolinecarboxamide;
(23) N-(4-chlorobenzyl)-6-(4-morpholinylmethyl)-4-oxo-1-[2-(phenylsulfanyl)ethyl]-1,4-dihydro-3-quinolinecarboxamide;
(24) N-(4-chlorobenzyl)-1-[(methylsulfanyl)methyl]-6-(4-morpholinylmethyl)-4-oxo-1,4-dihydro-3-quinolinecarboxamide;
(25) N-(4-chlorobenzyl)-6-(4-morpholinylmethyl)-4-oxo-1-[(phenylsulfanyl)methyl]-1,4-dihydro-3-quinolinecarboxamide;
(26) N-(4-chlorobenzyl)-1-[(methylsulfinyl)methyl]-6-(4-morpholinylmethyl)-4-oxo-1,4-dihydro-3-quinolinecarboxamide;
(27) N-(4-chlorobenzyl)-1-[(methylsulfonyl)methyl]-6-(4-morpholinylmethyl)-4-oxo-1,4-dihydro-3-quinolinecarboxamide;
(28) N-(4-chlorobenzyl)-6-(4-morpholinylmethyl)-4-oxo-1-[(phenylsulfinyl)methyl]-1,4-dihydro-3-quinolinecarboxamide;
(29) N-(4-chlorobenzyl)-6-(4-morpholinylmethyl)-4-oxo-1-[(phenylsulfonyl)methyl]-1,4-dihydro-3-quinolinecarboxamide;
(30) N-(4-chlorobenzyl)-1-[2-(2-methoxyethoxy)ethyl]-6-(4-morpholinylmethyl)-4-oxo-1,4-dihydro-3-quinolinecarboxamide;
(31) 1-{2-[2-(tert-butoxy)ethoxy]ethyl}-N-(4-chlorobenzyl)-6-(4-morpholinylmethyl)-4-oxo-1,4-dihydro-3-quinolinecarboxamide;
(32) N-(4-chlorobenzyl)-1-cyclopropyl-6-(4-morpholinylmethyl)-4-oxo-1,4-dihydro-3-quinolinecarboxamide;
or a pharmaceutically acceptable salt thereof.

31. The method of claim 1, wherein the compound administered has the Formula IV 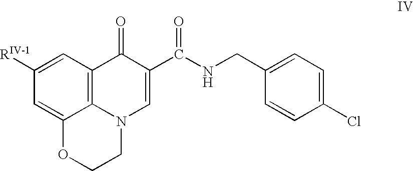
or a pharmaceutically acceptable salt, racemate, solvate, tautomer or optical isomer or prodrug derivative thereof.
wherein R^{IV-1} is C₁₋₆ alkyl, optionally substituted with —OH, —OC₁₋₄ alkyl or het^{IV};
wherein C₁₋₆ alkyl is optionally partially unsaturated;
wherein het^{IV} is a radical of a five- or six-membered heterocyclic ring having one or two heteroatoms selected from the group consisting of oxygen, sulfur and N.

32. The method of claim 31, wherein the compound administered is N-(4-Chlorobenzyl)-9-(4-morpholinylmethyl)-7-oxo-2,3-dihydro-7H-[1,4]oxazino[2,3,4-ij]quinoline-6-carboxamide; or a pharmaceutically acceptable salt thereof.

33. The method of claim 1, wherein the compound administered has the Formula V 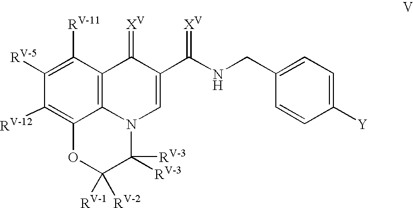
or a pharmaceutically acceptable salt, racemate, solvate, tautomer or optical isomer or prodrug derivative thereof,
wherein
each X^{V} is independently O or S;
Y is Cl, F, Br, CN or NO₂;
R^{V-1}, R^{V-2}, R^{V-3} and R^{V-4} are independently
a) hydrogen,
b) N₃,
c) CN,
d) fluoro,
e) trifluoromethyl,
f) aryl^{V},
g) het^{V},
h) C₁₋₈ alkyl, optionally substituted with R^{V-6} or OR^{V-7}, or
i) R^{V-1} and R^{V-2} or R^{V-3} and R^{V-4} together with the carbon to which they are attached form C₃₋₈cycloalkyl or het^{V};
R^{V-5} is C₁₋₈alkyl, which may be partially unsaturated and optionally substituted with one to three N₃, halo, CN, R^{V-6} or R^{V-7};
R^{V-6} is
a) aryl^{V},
b) het^{V},
c) SOᵢR^{V-8},
d) OR^{V-8},
e) C(═O)OR^{V-8},
f) C(═O)R^{V-8}, or
g) NR^{V-8}R^{V-9};
R^{V-7} is
a) P(═O) (OR^{V-10})₂,
b) CO(CH₂)ⱼCON(CH₃)(CH₂)ₖSO₃⁻M^{V+},
c) an amino acid,
d) C(═O)C₁₋₆alkyl, optionally substituted by NR^{V-10}R^{V-10}, or
e) CO(CH₂)ₙCO₂H;
R^{V-8} and R^{V-9} are independently
a) hydrogen,
b) C₃₋₈cycloalkyl,
c) aryl^{V},
d) het^{V}, or
e) C₁₋₈alkyl which is further optionally substituted with one or more aryl^{V}, het^{V}, halo, CN, CO₂R^{V-10}, SOᵢR^{V-10}, OR^{V-10}, NR^{V-10}R^{V-10}, CF₃, or C₃₋₈cycloalkyl;
R^{V-10} is
a) H or
b) C₁₋₈alkyl, optionally substituted with OH or OC₁₋₄alkyl;
R^{V-11} and R^{V-12} are independently
a) hydrogen,
b) halo,
c) NO₂,
d) CN,
e) R^{V-6},
f) SOᵢNR^{V-8}R^{V-9}, or
g) C₁₋₈alkyl, which may be partially unsaturated and optionally substituted with one to three N₃, halo, CN, R^{V-6} or OR^{V-7};
aryl^{V} is
a phenyl radical, optionally fused with a saturated or unsaturated carbocyclic or hetero cyclic ring; at each occurrence, aryl^{V} may be substituted with one or more halo, CN, CO₂R^{V-10}, SOᵢR^{V-10}, OR^{V-10}, NR^{V-10}R^{V-10}, CF₃, C₃₋₈cycloalkyl, or C₁₋₄alkyl wherein C₁₋₄alkyl is optionally substituted with OR^{V-10};
het^{V} is
a four- (4), five- (5), six- (6), or seven- (7) membered saturated or unsaturated heterocyclic ring having 1, 2, or 3 heteroatoms selected from the group consisting of O, S, and NW, wherein W is hydrogen, C₁₋₄alkyl, C(═O)OC₁₋₄alkyl or absent, wherein het^{v} is optionally fused with a benzene ring, a carbcyclic or a heterocyclic ring; at each occurrence, het^{V} may be substituted with one or more halo, CN, CO₂R^{V-10}, SOᵢR^{V-10}, OR^{V-10}, NR^{V-10}R^{V-10}, C₁₋₄alkyl, CF₃, C₃₋₈cycloalkyl, oxo or oxine; at each occurrence, a cycloalkyl may be substituted with C₁₋₄alkyl, OR^{V-10}, oxo, oxine, or a Spiro fused het^{V};
i^{V} is 0, 1 or 2;
j^{V} is 1, 2, 3, 4, 5, or 6;
k^{V} is 1, 2, 3, 4, 5, or 6;
n^{V} is 1, 2, 3, 4, 5, or 6;
M^{V} is sodium, potassium, or lithium.

34. The method of claim 33, wherein the compound administered is
(a) N-(4-Chlorobenzyl)-2-(hydroxymethyl)-9-(morpholin-4-ylmethyl)-7-oxo-2,3-dihydro-7H-[1,4]oxazino[2,3,4-ij]quinoline-6-carboxamide,
(b) N-(4-Chlorobenzyl)-2-(R or S)-(hydroxymethyl)-9-(morpholin-4-ylmethyl)-7-oxo-2,3-dihydro-7H-[1,4]oxazino[2,3,4-ij]quinoline-6-carboxamide,
(c) N-(4-Chlorobenzyl)-9-(morpholin-4-ylmethyl)-7-oxo-2-pyridin-3-yl-2,3-dihydro-7H-[1,4]oxazino[2,3,4-ij]quinoline-6-carboxamide,
(d) N-(4-Chlorobenzyl)-9-(morpholin-4-ylmethyl)-7-oxo-2-pyridin-4-yl-2,3-dihydro-7H-[1,4]oxazino[2,3,4-ij]quinoline-6-carboxamide,
(e) N-(4-Chlorobenzyl)-9-(morpholin-4-ylmethyl)-7-oxo-2-pyridin-2-yl-2,3-dihydro-7H-[1,4]oxazino[2,3,4-ij]quinoline-6-carboxamide,
(f) N-(4-Chlorobenzyl)-9-(morpholin-4-ylmethyl)-7-oxo-2-(R or S)-pyridin-3-yl-2,3-dihydro-7H-[1,4]oxazino[2,3,4-ij]quinoline-6-carboxamide,
(g) N-(4-Chlorobenzyl)-2,9-bis(morpholin-4-ylmethyl)-7-oxo-2,3-dihydro-7H-[1,4]oxazino[2,3,4-ij]quinoline-6-carboxamide,
(h) 2-[(tert-Butylsulfanyl)methyl]-N-(4-chlorobenzyl)-9-(morpholin-4-ylmethyl)-7-oxo-2,3-dihydro-7H-[1,4]oxazino[2,3,4-ij]quinoline-6-carboxamide,
(i) N-(4-Chlorobenzyl)-2-{[(2-hydroxyethyl)sulfanyl]methyl}-9-(morpholin-4-ylmethyl)-7-oxo-2,3-dihydro-7H-[1,4]oxazino[2,3,4-ij]quinoline-6-carboxamide,
(j) N-(4-Chlorobenzyl)-2-{[(1-methyl-1H-imidazol-2-yl)sulfanyl]methyl}-9-(morpholin-4-ylmethyl)-7-oxo-2,3-dihydro-7H-[1,4]oxazino[2,3,4-ij]quinoline-6-carboxamide,
(k) N-(4-Chlorobenzyl)-9-(morpholin-4-ylmethyl)-7-oxo-2-{[(3-pyridinylmethyl)amino]methyl}-2,3-dihydro-7H-[1,4]oxazino[2,3,4-ij]quinoline-6-carboxamide,
(l) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl acetate,
(m) N-(4-Chlorobenzyl)-9-(morpholin-4-ylmethyl)-7-oxo-2-(R or S)-{[(3-pyridinylmethyl)amino]methyl}-2,3-dihydro-7H-[1,4]oxazino[2,3,4-ij]quinoline-6-carboxamide,
(n) N-(4-Chlorobenzyl)-2-(3-hydroxyphenyl)-9-(morpholin-4-ylmethyl)-7-oxo-2,3-dihydro-7H-[1,4]oxazino[2,3,4-ij]quinoline-6-carboxamide,
(o) N-(4-Chlorobenzyl)-9-(morpholin-4-ylmethyl)-7-oxo-2-(R or S)-pyridin-2-yl-2,3-dihydro-7H-[1,4]oxazino[2,3,4-ij]quinoline-6-carboxamide,
(p) N-(4-Chlorobenzyl)-2-[3-(hydroxymethyl)phenyl]-9-(morpholin-4-ylmethyl)-7-oxo-2,3-dihydro-7H-[1,4]oxazino[2,3,4-ij]quinoline-6-carboxamide,
(q) N-(4-Chlorobenzyl)-2-[2-(hydroxymethyl)phenyl]-9-(morpholin-4-ylmethyl)-7-oxo-2,3-dihydro-7H-[1,4]oxazino[2,3,4-ij]quinoline-6-carboxamide,
(r) N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide,
(s) N-(4-Chlorobenzyl)-2-(2-furyl)-9-(morpholin-4-ylmethyl)-7-oxo-2,3-dihydro-7H-[1,4]oxazino[2,3,4-ij]quinoline-6-carboxamide,
(t) N-(4-Chlorobenzyl)-2-(3-cyanophenyl)-9-(morpholin-4-ylmethyl)-7-oxo-2,3-dihydro-7H-[1,4]oxazino[2,3,4-ij]quinoline-6-carboxamide,
(u) N-(4-Chlorobenzyl)-2-(3-furyl)-9-(morpholin-4-ylmethyl)-7-oxo-2,3-dihydro-7H-[1,4]oxazino[2,3,4-ij]quinoline-6-carboxamide,
(v) N-(4-Chlorobenzyl)-9-(morpholin-4-ylmethyl)-7-oxo-2-thien-2-yl-2,3-dihydro-7H-[1,4]oxazino[2,3,4-ij]quinoline-6-carboxamide,
(w) N-(4-Chlorobenzyl)-2-(3,5-difluorophenyl)-9-(morpholin-4-ylmethyl)-7-oxo-2,3-dihydro-7H-[1,4]oxazino[2,3,4-ij]quinoline-6-carboxamide,
(x) 2-(1,3-Benzodioxol-5-yl)-N-(4-chlorobenzyl)-9-(morpholin-4-ylmethyl)-7-oxo-2,3-dihydro-7H-[1,4]oxazino[2,3,4-ij]quinoline-6-carboxamide,
(y) N-(4-Chlorobenzyl)-2-(2,3-dihydro-1,4-benzodioxin-6-yl)-9-(morpholln-4-ylmethyl)-7-oxo-2,3-dihydro-7H-[1,4]oxazino[2,3,4-ij]quinoline-6-carboxamide,
(z) 2-(1,3-Benzodioxol-4-yl)-N-(4-chlorobenzyl)-9-(morpholin-4-ylmethyl)-7-oxo-2,3-dihydro-7H-[1,4]oxazino[2,3,4-ij]quinoline-6-carboxamide,
(aa) 2-[3,5-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,
(bb) N-(4-Chlorobenzyl)-9-(morpholin-4-ylmethyl)-7-oxo-2-thien-3-yl-2,3-dihydro-7H-[1,4]oxazino[2,3,4-ij]quinoline-6-carboxamide,
(cc) N-(4-Chlorobenzyl)-2,2-bis[(methoxymethoxy)methyl]-9-(morpholin-4-ylmethyl)-7-oxo-2,3-dihydro-7H-[1,4]oxazino[2,3,4-ij]quinoline-6-carboxamide,
(dd) N-[(4-Chlorophenyl)methyl]-9′-(4-morpholinylmethyl)-4,7′-dioxospiro[cyclohexane-1,2′(3′H)-[7H]pyrido[1,2,3-de][1,4]benzoxazine]-6′-carboxamide,
(ee) N-[(4-Chlorophenyl)methyl]-4-hydroxy-9′-(4-morpholinylmethyl)-7′-oxospiro[cyclohexane-1,2′(3′H)-[7H]pyrido[1,2,3-de][1,4]benzoxazine]-6′-carboxamide,
(ff) N-(4-Chlorobenzyl)-3,9-bis(morpholin-4-ylmethyl)-7-oxo-2,3-dihydro-7H-[1,4]oxazino[2,3,4-ij]quinoline-6-carboxamide,
(gg) N-(4-Chlorobenzyl)-9-(morpholin-4-ylmethyl)-7-oxo-2-phenyl-2,3-dihydro-7H-[1,4]oxazino[2,3,4-ij]quinoline-6-carboxamide,
(hh) N-(4—Chlorobenzyl)-2,2-difluoro-9-(morpholin-4-ylmethyl)-7-oxo-2,3-dihydro-7H-[1,4]oxazino[2,3,4-ij]quinoline-6-carboxamide,
(ii) N-(4-Chlorobenzyl)-2-[(methylsulfanyl)methyl]-9-(morpholin-4-ylmethyl)-7-oxo-2,3-dihydro-7H-[1,4]oxazino[2,3,4-ij]quinoline-6-carboxamide,
(jj) N-(4-Chlorobenzyl)-2-[(dimethylamino)methyl]-9-(morpholin-4-ylmethyl)-7-oxo-2,3-dihydro-7H-[1,4]oxazino[2,3,4-ij]quinoline-6-carboxamide,
(kk) N-(4-Chlorobenzyl)-2-[(4-methyl-1-piperazinyl)methyl]-9-(morpholin-4-ylmethyl)-7-oxo-2,3-dihydro-7H-[1,4]oxazino[2,3,4-ij]quinoline-6-carboxamide,
(ll) Methyl ({[6-{[(4-chlorobenzyl)amino]carbonyl}-9-(morpholin-4-ylmethyl)-7-oxo-2,3-dihydro-7H-[1,4]oxazino[2,3,4-ij]quinolin-2-yl]methyl}thio)acetate,
(mm) N-(4-Chlorobenzyl)-9-(morpholin-4-ylmethyl)-7-oxo-2-(1-pyrrolidinylmethyl)-2,3-dihydro-7H-[1,4]oxazino[2,3,4-ij]quinoline-6-carboxamide,
(nn) N-(4-Chlorobenzyl)-2-{[(2,3-dihydroxypropyl)sulfanyl]methyl}-9-(morpholin-4-ylmethyl)-7-oxo-2,3-dihydro-7H-[1,4]oxazino[2,3,4-ij]quinoline-6-carboxamide,
(oo) N-(4-Chlorobenzyl)-2-{[(2,3-dihydroxypropyl)amino]methyl}-9-(morpholin-4-ylmethyl)-7-oxo-2,3-dihydro-7H-[1,4]oxazino[2,3,4-ij]quinoline-6-carboxamide,
(pp) N-(4-Chlorobenzyl)-2-{[(2-hydroxyethyl)amino]methyl}-9-(morpholin-4-ylmethyl)-7-oxo-2,3-dihydro-7H-[1,4]oxazino[2,3,4-ij]quinoline-6-carboxamide,
(qq) N-(4-Chlorobenzyl)-9-(morpholin-4-ylmethyl)-7-oxo-2-(1-piperidinylmethyl)-2,3-dihydro-7H-[1,4]oxazino[2,3,4-ij]quinoline-6-carboxamide,
(rr) 2-{[bis(2-Hydroxyethyl)amino]methyl}-N-(4-chlorobenzyl)-9-(morpholin-4-ylmethyl)-7-oxo-2,3-dihydro-7H-[1,4]oxazino[2,3,4-ij]quinoline-6-carboxamide,
(ss) N-(4-Chlorobenzyl)-9-(morpholin-4-ylmethyl)-7-oxo-2-{[(2-pyridinylmethyl)amino]methyl}-2,3-dihydro-7H-[1,4]oxazino[2,3,4-ij]quinoline-6-carboxamide,
(tt) 2-[(8-{[6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methoxy}-8-oxooctanoyl)(methyl)amino]ethanesulfonic acid sodium salt,
(uu) [6-{[(4-Chlorobenzyl)amino]carbonyl}-9-(morpholin-4-ylmethyl)-7-oxo-2,3-dihydro-7H-[1,4]oxazino[2,3,4-ij]quinolin-2-yl]methyl dimethyl phosphate,
(vv) N-(4-Chlorobenzyl)-9-(morpholin-4-ylmethyl)-7-oxo-2-{[(4pyridinylmethyl)amino]methyl}-2,3-dihydro-7H-[1,4]oxazino[2,3,4-ij]quinoline-6-carboxamide,
(ww) N-(4-Chlorobenzyl)-2-(1H-imidazol-1-ylmethyl)-9-(morpholin-4-ylmethyl)-7-oxo-2,3-dihydro-7H-[1,4]oxazino[2,3,4-ij]quinoline-6-carboxamide,
(xx) N-(4-Chlorobenzyl)-2-{[(4-chlorobenzyl)amino]methyl}-9-(morpholin-4-ylmethyl)-7-oxo-2,3-dihydro-7H-[1,4]oxazino[2,3,4-ij]quinoline-6-carboxamide,
(yy) N-(4-Chlorobenzyl)-3-(hydroxymethyl)-9-(morpholin-4-ylmethyl)-7-oxo-2,3-dihydro-7H-[1,4]oxazino[2,3,4-ij]quinoline-6-carboxamide,
(zz) N-(4-Chlorobenzyl)-2-(4-hydroxyphenyl)-9-(morpholin-4-ylmethyl)-7-oxo-2,3-dihydro-7H-[1,4]oxazino[2,3,4-ij]quinoline-6-carboxamide,
(aaa) N-(4-Chlorobenzyl)-2-{3-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,
(bbb) N-(4-Chlorobenzyl)-2-{2-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,
(ccc) N-(4-Chlorobenzyl)-2-(2-hydroxyphenyl)-9-(morpholin-4-ylmethyl)-7-oxo-2,3-dihydro-7H-[1,4]oxazino[2,3,4-ij]quinoline-6-carboxamide,
(ddd) N-[(4-Chlorophenyl)methyl]-2,3,5,6-tetrahydro-9′-(4-morpholinylmethyl)-7′-oxospiro[4H-pyran-4,2′(3′H)-[7H]pyrido[1,2,3-de][1,4]benzoxazine]-6′-carboxamide,
(eee) 1,1-Dimethylethyl 6-[[[(4-chlorophenyl)methyl]amino]carbonyl]-9′-(4-morpholinylmethyl)-7′-oxospiro[piperidine-4,2′(3′H)-[7H]pyrido[1,2,3-de][1,4]benzoxazine]-1-carboxylate,
(fff) N-[(4-Chlorophenyl)methyl]-9′-(4-morpholinylmethyl)-7′-oxospiro[piperidine-4,2′(3′H)-[7H]pyrido[1,2,3-de][1,4]benzoxazine]-6′-carboxamide,
(ggg) N-(4-Chlorobenzyl)-2,2-bis(hydroxymethyl)-9-(morpholin-4-ylmethyl)-7-oxo-2,3-dihydro-7H-[1,4]oxazino[2,3,4-ij]quinoline-6-carboxamide,
(hhh) N-[(4-Chlorophenyl)methyl]-2′,3′,5′,6′-tetrahydro-9-(4-morpholinylmethyl)-7-oxospiro[7H-pyrido[1,2,3-de]-1,4-benzoxazine-2(3H),4′-[4H]thiopyran]-6-carboxamide,
(iii) N-(4-Chlorobenzyl)-9-(morpholin-4-ylmethyl)-7-oxo-3-phenyl-2,3-dihydro-7H-[1,4]oxazino[2,3,4-ij]quinoline-6-carboxamide,
(jjj) N-(4-Chlorobenzyl)-3,3-bis(hydroxymethyl)-9-(3-hydroxy-1-propynyl)-7-oxo-2,3-dihydro-7H-[1,4]oxazino[2,3,4-ij]quinoline-6-carboxamide,
(kkk) N-(4-Chlorobenzyl)-3,3-bis(hydroxymethyl)-9-(3-hydroxypropyl)-7-oxo-2,3-dihydro-7H-[1,4]oxazino[2,3,4-ij]quinoline-6-carboxamide,
(lll) N-(4-Chlorobenzyl)-2-[2-(methoxymethoxy)phenyl]-9-(morpholin-4-ylmethyl)-7-oxo-2,3-dihydro-7H-[1,4]oxazino[2,3,4-ij]quinoline-6-carboxamide,
(mmm) N-(4-Chlorobenzyl)-2-{4-[(methoxymethoxy)methyl]phenyl}-9-(morpholin-4-ylmethyl)-7-oxo-2,3-dihydro-7H-[1,4]oxazino[2,3,4-ij]quinoline-6-carboxamide,
(nnn) 2-[2,3-bis(Methoxymethoxy)phenyl]-N-(4-chlorobenzyl)-9-(morpholin-4-ylmethyl)-7-oxo-2,3-dihydro-7H-[1,4]oxazino[2,3,4-ij]quinoline-6-carboxamide,
(ooo) N-[(4-Chlorophenyl)methyl]-1-methyl-9′-(4-morpholinylmethyl)-7′-oxospiro[piperidine-4,2′(3′H)-[7H]pyrido[1,2,3-de][1,4]benzoxazine]-6′-carboxamide,
(ppp) N-[(4-Chlorophenyl)methyl]-9″-(4-morpholinylmethyl)dispiro[1,3-dioxolane-2,1′-cyclohexane-4′,2″(3″H)-[7H]pyrido[1,2,3-de][1,4]benzoxazine]-6″-carboxamide, or a pharmaceutically acceptable salt thereof.

35. The method of claim 33, wherein the compound administered is
(a) N-(4-Chlrobenzyl)-2-(hydroxymethyl)-9-(morpholin-4-ylmethyl)-7-thioxo-2,3-dihydro-7H-[1,4]oxazino[2,3,4-ij]quinoline-6-carboxamide,
(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,
(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,
(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,
(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxamide,
(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxamide,
(g) N-(4-Chlorobenzyl)-2,9-bis(morpholin-4-ylmethyl)-7-thioxo-2,3-dihydro-7H-[1,4]oxazino[2,3,4-ij]quinoline-6-carboxamide,
(h) 2-[(tert-Butylsulfanyl)methyl]-N-(4-chlorobenzyl)-9-(morpholin-4-ylmethyl)-7-thioxo-2,3-dihydro-7H-[1,4]oxazino[2,3,4-ij]quinoline-6-carboxamide,
(i) N-(4-Chlorobenzyl)-2-{[(2-hydroxyethyl)sulfanyl]methyl}-9-(morpholin-4-ylmethyl)-7-thioxo-2,3-dihydro-7H-[1,4]oxazino[2,3,4-ij]quinoline-6-carboxamide,
(j) N-(4-Chlorobenzyl)-2-{[(1-methyl-1H-imidazol-2-yl)sulfanyl]methyl}-9-(morpholin-4-ylmethyl)-7-thioxo-2,3-dihydro-7H-[1,4]oxazino[2,3,4-ij]quinoline-6-carboxamide,
(k) N-(4-Chlorobenzyl)-9-(morpholin-4-ylmethyl)-7-thioxo-2-{[(3-pyridinylmethyl)amino]methyl}-2,3-dihydro-7H-[1,4]oxazino[2,3,4-ij]quinoline-6-carboxamide,
(l) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl acetate,
(m) N-(4-Chlorobenzyl)-9-(morpholin-4-ylmethyl)-7-thioxo-2-(R or S)-{[(3-pyridinylmethyl)amino]methyl}-2,3-dihydro-7H-[1,4]oxazino[2,3,4-ij]quinoline-6-carboxamide,
(n) N-(4-Chlorobenzyl)-2-(3-hydroxyphenyl)-9-(morpholin-4-ylmethyl)-7-thioxo-2,3-dihydro-7H-[1,4]oxazino[2,3,4-ij]quinoline-6-carboxamide,
(o) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,
(p) N-(4-Chlorobenzyl)-2-[3-(hydroxymethyl)phenyl]-9-(morpholin-4-ylmethyl)-7-thioxo-2,3-dihydro-7H-[1,4]oxazino[2,3,4-ij]quinoline-6-carboxamide,
(q) N-(4-Chlorobenzyl)-2-[2-(hydroxymethyl)phenyl]-9-(morpholin-4-ylmethyl)-7-thioxo-2,3-dihydro-7H-[1,4]oxazino[2,3,4-ij]quinoline-6-carboxamide,
(r) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide,
(s) N-(4-Chlorobenzyl)-2-(2-furyl)-9-(morpholin-4-ylmethyl)-7-thioxo-2,3-dihydro-7H-[1,4]oxazino[2,3,4-ij]quinoline-6-carboxamide,
(t) N-(4-Chlorobenzyl)-2-(3-cyanophenyl)-9-(morpholin-4-ylmethyl)-7-thioxo-2,3-dihydro-7H-[1,4]oxazino[2,3,4-ij]quinoline-6-carboxamide,
(u) N-(4-Chlorobenzyl)-2-(3-furyl)-9-(morpholin-4-ylmethyl)-7-thioxo-2,3-dihydro-7H-[1,4]oxazino[2,3,4-ij]quinoline-6-carboxamide,
(v) N-(4-Chlorobenzyl)-9-(morpholin-4-ylmethyl)-7-thioxo-2-thien-2-yl-2,3-dihydro-7H-[1,4]oxazino[2,3,4-ij]quinoline-6-carboxamide,
(w) N-(4-Chlorobenzyl)-2-(3,5-difluorophenyl)-9-(morpholin-4-ylmethyl)-7-thioxo-2,3-dihydro-7H-[1,4]oxazino[2,3,4-ij]quinoline-6-carboxamide,
(x) 2-(1,3-Benzodioxol-5-yl)-N-(4-chlorobenzyl)-9-(morpholin-4-ylmethyl)-7-thioxo-2,3-dihydro-7H-[1,4]oxazino[2,3,4-ij]quinoline-6-carboxamide,
(y) N-(4-Chlorobenzyl)-2-(2,3-dihydro-1,4-benzodioxin-6-yl)-9-(morpholin-4-ylmethyl)-7-thioxo-2,3-dihydro-7H-[1,4]oxazino[2,3,4-ij]quinoline-6-carboxamide,
(z) 2-(1,3-Benzodioxol-4-yl)-N-(4-chlorobenzyl)-9-(morpholin-4-ylmethyl)-7-thioxo-2,3-dihydro-7H-[1,4]oxazino[2,3,4-ij]quinoline-6-carboxamide,
(aa) 2-[3,5-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,
(bb) N-(4-Chlorobenzyl)-9-(morpholin-4-ylmethyl)-7-thioxo-2-thien-3-yl-2,3-dihydro-7H-[1,4]oxazino[2,3,4-ij]quinoline-6-carboxamide,
(cc) N-(4-Chlorobenzyl)-2,2-bis[(methoxymethoxy)methyl]-9-(morpholin-4-ylmethyl)-7-thioxo-2,3-dihydro-7H-[1,4]oxazino[2,3,4-ij]quinoline-6-carboxamide,
(dd) N-[(4-Chlorophenyl)methyl]-9′-(4-morpholinylmethyl)-4-oxo-7′-thioxospiro[cyclohexane-1,2′(3′H)-[7H]pyrido[1,2,3-de][1,4]benzoxazine]-6′-carboxamide,
(ee) N-[(4-Chlorophenyl)methyl]-4-hydroxy-9′-(4-morpholinylmethyl)-7′-thioxospiro[cyclohexane-1,2′(3′H)-[7H]pyrido[1,2,3-de][1,4]benzoxazine]-6′-carboxamide,
(ff) N-(4-Chlorobenzyl)-3,9-bis(morpholin-4-ylmethyl)-7-thioxo-2,3-dihydro-7H-[1,4]oxazino[2,3,4-ij]quinoline-6-carboxamide,
(gg) N-(4-Chlorobenzyl)-9-(morpholin-4-ylmethyl)-7-thioxo-2-phenyl-2,3-dihydro-7H-[1,4]oxazino[2,3,4-ij]quinoline-6-carboxamide,
(hh) N-(4-Chlorobenzyl)-2,2-difluoro-9-(morpholin-4-ylmethyl)-7-thioxo-2,3-dihydro-7H-[1,4]oxazino[2,3,4-ij]quinoline-6-carboxamide,
(ii) N-(4-Chlorobenzyl)-2-[(methylsulfanyl)methyl]-9-(morpholin-4-ylmethyl)-7-thioxo-2,3-dihydro-7H-[1,4]oxazino[2,3,4-ij]quinoline-6-carboxamide,
(jj) N-(4-Chlorobenzyl)-2-[(dimethylamino)methyl]-9-(morpholin-4-ylmethyl)-7-thioxo-2,3-dihydro-7H-[1,4]oxazino[2,3,4-ij]quinoline-6-carboxamide,
(kk) N-(4-Chlorobenzyl)-2-[(4-methyl-1-piperazinyl)methyl]-9-(morpholin-4-ylmethyl)-7-thioxo-2,3-dihydro-7H-[1,4]oxazino[2,3,4-ij]quinoline-6-carboxamide,
(ll) Methyl ({[6-{[(4-chlorobenzyl)amino]carbonyl}-9-(morpholin-4-ylmethyl)-7-thioxo-2,3-dihydro-7H-[1,4]oxazino[2,3,4-ij]quinolin-2-yl]methyl}thio)acetate,
(mm) N-(4-Chlorobenzyl)-9-(morpholin-4-ylmethyl)-7-thioxo-2-(1-pyrrolidinylmethyl)-2,3-dihydro-7H-[1,4]oxazino[2,3,4-ij]quinoline-6-carboxamide,
(nn) N-(4-Chlorobenzyl)-2-{[(2,3-dihydroxypropyl)sulfanyl]methyl}-9-(morpholin-4-ylmethyl)-7-thioxo-2,3-dihydro-7H-[1,4]oxazino[2,3,4-ij]quinoline-6-carboxamide,
(oo) N-(4-Chlorobenzyl)-2-{[(2,3-dihydroxypropyl)amino]methyl}-9-(morpholin-4-ylmethyl)-7-thioxo-2,3-dihydro-7H-[1,4]oxazino[2,3,4-ij]quinoline-6-carboxamide,
(pp) N-(4-Chlorobenzyl)-2-{[(2-hydroxyethyl)amino]methyl}-9-(morpholin-4-ylmethyl)-7-thioxo-2,3-dihydro-7H-[1,4]oxazino[2,3,4-ij]quinoline-6-carboxamide,
(qq) N-(4-Chlorobenzyl)-9-(morpholin-4-ylmethyl)-7-thioxo-2-(1-piperidinylmethyl)-2,3-dihydro-7H-[1,4]oxazino[2,3,4-ij]quinoline-6-carboxamide,
(rr) 2-{[bis(2—Hydroxyethyl)amino]methyl}-N-(4-chlorobenzyl)-9-(morpholin-4-ylmethyl)-7-thioxo-2,3-dihydro-7H-[1,4]oxazino[2,3,4-ij]quinoline-6-carboxamide,
(ss) N-(4-Chlorobenzyl)-9-(morpholin-4-ylmethyl)-7-thioxo-2-{[(2-pyridinylmethyl)amino]methyl}-2,3-dihydro-7H-[1,4]oxazino[2,3,4-ij]quinoline-6-carboxamide,
(tt) 2-[(8-{[6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methoxy}-8-oxooctanoyl)(methyl)amino]ethanesulfonic acid sodium salt,
(uu) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl dimethyl phosphate,
(vv) N-(4-Chlorobenzyl)-9-(morpholin-4-ylmethyl)-7-thioxo-2-{[(4-pyridinylmethyl)amino]methyl}-2,3-dihydro-7H-[1,4]oxazino[2,3,4-ij]quinoline-6-carboxamide,
(ww) N-(4-Chlorobenzyl)-2-(1H-imidazol-1-ylmethyl)-9-(morpholin-4-ylmethyl)-7-thioxo-2,3-dihydro-7H-[1,4]oxazino[2,3,4-ij]quinoline-6-carboxamide,
(xx) N-(4-Chlorobenzyl)-2-{[(4-chlorobenzyl)amino]methyl}-9-(morpholin-4-ylmethyl)-7-thioxo-2,3-dihydro-7H-[1,4]oxazino[2,3,4-ij]quinoline-6-carboxamide,
(yy) N-(4-Chlorobenzyl)-3-(hydroxymethyl)-9-(morpholin-4-ylmethyl)-7-thioxo-2,3-dihydro-7H-[1,4]oxazino[2,3,4-ij]quinoline-6-carboxamide,
(zz) N-(4-Chlorobenzyl)-2-(4-hydroxyphenyl)-9-(morpholin-4-ylmethyl)-7-thioxo-2,3-dihydro-7H-[1,4]oxazino[2,3,4-ij]quinoline-6-carboxamide,
(aaa) N-(4-Chlorobenzyl)-2-{3-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,
(bbb) N-(4-Chlorobenzyl)-2-{2-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,
(ccc) N-(4-Chlorobenzyl)-2-(2-hydroxyphenyl)-9-(morpholin-4-ylmethyl)-7-thioxo-2,3-dihydro-7H-[1,4]oxazino[2,3,4-ij]quinoline-6-carboxamide,
(ddd) N-[(4-Chlorophenyl)methyl]-2,3,5,6-tetrahydro-9′-(4-morpholinylmethyl)-7′-thioxospiro[4H-pyran-4,2′(3′H)-[7H]pyrido[1,2,3-de][1,4]benzoxazine]-6′-carboxamide,
(eee) 1,1-Dimethylethyl 6-[[[(4-chlorophenyl)methyl]amino]carbonyl]-9′-(4-morpholinylmethyl)-7′-thioxospiro[piperidine-4,2′(3′H)-[7H]pyrido[1,2,3-de][1,4]-1-carboxylate,
(fff) N-[(4-Chlorophenyl)methyl]-9′-(4-morpholinylmethyl)-7′-thioxospiro[piperidine-4,2′(3′H)-[7H]pyrido[1,2,3-de][1,4]benzoxazine]-6′-carboxamide,
(ggg) N-(4-Chlorobenzyl)-2,2-bis(hydroxymethyl)-9-(morpholin-4-ylmethyl)-7-thioxo-2,3-dihydro-7H-[1,4]oxazino[2,3,4-ij]quinoline-6-carboxamide,
(hhh) N-[(4-Chlorophenyl)methyl]-2′,3′,5′,6′-tetrahydro-9-(4-morpholinylmethyl)-7-thioxospiro[7H-pyrido[1,2,3-de]-1,4-benzoxazine-2(3H),4′-[4H]thiopyran]-6-caboxamide,
(iii) N-(4-Chlorobenzyl)-9-(morpholin-4-ylmethyl)-7-thioxo-3-phenyl-2,3-dihydro-7H-[1,4]oxazino[2,3,4-ij]quinoline-6-carboxamide,
(jjj) N-(4-Chlorobenzyl)-3,3-bis(hydroxymethyl)-9-(3-hydroxy-1-propynyl)-7-thioxo-2,3-dihydro-7H-[1,4]oxazino[2,3,4-ij]quinoline-6-carboxamide,
(kkk) N-(4-Chlorobenzyl)-3,3-bis(hydroxymethyl)-9-(3-hydroxypropyl)-7-thioxo-2,3-dihydro-7H-[1,4]oxazino[2,3,4-ij]quinoline-6-carboxamide,
(lll) N-(4-Chlorobenzyl)-2-[2-(methoxymethoxy)phenyl]-9-(morpholin-4-ylmethyl)-7-thioxo-2,3-dihydro-7H-[1,4]oxazino[2,3,4-ij]quinoline-6-carboxamide,
(mmm) N-(4-Chlorobenzyl)-2-{4-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,
(nnn) 2-[2,3-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,
(ooo) N-[(4-Chlorophenyl)methyl]-1-methyl-9′-(4-morpholinylmethyl)-7′-thioxospiro[piperidine-4,2′(3′H)-[7H]pyrido[1,2,3-de][1,4]benzoxazine]-6′-carboxamide; or
a pharmaceutically acceptable salt thereof.

36. The method of claim 33, wherein the compound administered is selected from the group consisting of
(a) N-(4-Chlorobenzyl)-2-(hydroxymethyl)-9-(morpholin-4-ylmethyl)-7-thioxo-2,3-dihydro-7H-[1,4]oxazino[2,3,4-ij]quinoline-6-carboxamide,
(b) N-(4-Chlorobenzyl)-2-(R or S)-(hydroxymethyl)-9-(morpholin-4-ylmethyl)-7-thioxo-2,3-dihydro-7H-[1,4]oxazino[2,3,4-ij]quinoline-6-carboxamide,
(c) N-(4-Chlorobenzyl)-9-(morpholin-4-ylmethyl)-7-thioxo-2-pyridin-3-yl-2,3-dihydro-7H-[1,4]oxazino[2,3,4-ij]quinoline-6-carboxamide,
(d) N-(4-Chlorobenzyl)-9-(morpholin-4-ylmethyl)-7-thioxo-2-pyridin-4-yl-2,3-dihydro-7H-[1,4]oxazino[2,3,4-ij]quinoline-6-carboxamide,
(e) N-(4-Chlorobenzyl)-9-(morpholin-4-ylmethyl)-7-thioxo-2-pyridin-2-yl-2,3-dihydro-7H-[1,4]oxazino[2,3,4-ij]quinoline-6-carboxamide,
(f) N-(4-Chlorobenzyl)-9-(morpholin-4-ylmethyl)-7-thioxo-2-(R or S)-pyridin-3-yl-2,3-dihydro-7H-[1,4]oxazino[2,3,4-ij]quinoline-6-carboxamide,
(g) N-(4-Chlorobenzyl)-2,9-bis(morpholin-4-ylmethyl)-7-thioxo-2,3-dihydro-7H-[1,4]oxazino[2,3,4-ij]quinoline-6-carboxamide,
(h) 2-[(tert-Butylsulfanyl)methyl]-N-(4-chlorobenzyl)-9-(morpholin-4-ylmethyl)-7-thioxo-2,3-dihydro-7H-[1,4]oxazino[2,3,4-ij]quinoline-6-carboxamide,
(i) N-(4-Chlorobenzyl)-2-{[(2-hydroxyethyl)sulfanyl]methyl}-9-(morpholin-4-ylmethyl)-7-thioxo-2,3-dihydro-7H-[1,4]oxazino[2,3,4-ij]quinoline-6-carboxamide,
(j) N-(4-Chlorobenzyl)-2-{[(1-methyl-1H-imidazol-2-yl)sulfanyl]methyl}-9-(morpholin-4-ylmethyl)-7-thioxo-2,3-dihydro-7H-[1,4]oxazino[2,3,4-ij]quinoline-6-carboxamide,
(k) N-(4-Chlorobenzyl)-9-(morpholin-4-ylmethyl)-7-thioxo-2-{[(3-pyridinylmethyl)amino]methyl}-2,3-dihydro-7H-[1,4]oxazino[2,3,4-ij]quinoline-6-carboxamide,
(l) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl acetate,
(m) N-(4-Chlorobenzyl)-9-(morpholin-4-ylmethyl)-7-thioxo-2-(R or S)-{[(3-pyridinylmethyl)amino]methyl}-2,3-dihydro-7H-[1,4]oxazino[2,3,4-ij]quinoline-6-carboxamide,
(n) N-(4-Chlorobenzyl)-2-(3-hydroxyphenyl)-9-(morpholin-4-ylmethyl)-7-thioxo-2,3-dihydro-7H-[1,4]oxazino[2,3,4-ij]quinoline-6-carboxamide,
(o) N-(4-Chlorobenzyl)-9-(morpholin-4-ylmethyl)-7-thioxo-2-(R or S)-pyridin-2-yl-2,3-dihydro-7H-[1,4]oxazino[2,3,4-ij]quinoline-6-carboxamide,
(p) N-(4-Chlorobenzyl)-2-[3-(hydroxymethyl)phenyl]-9-(morpholin-4-ylmethyl)-7-thioxo-2,3-dihydro-7H-[1,4]oxazino[2,3,4-ij]quinoline-6-carboxamide,
(q) N-(4-Chlorobenzyl)-2-[2-(hydroxymethyl)phenyl]-9-(morpholin-4-ylmethyl)-7-thioxo-2,3-dihydro-7H-[1,4]oxazino[2,3,4-ij]quinoline-6-carboxamide,
(r) N-(4-Chlorobenzyl)-2-(1-methyl-1H-imidazol-2-yl)-9-(morpholin-4-ylmethyl)-7-thioxo-2,3-dihydro-7H-[1,4]oxazino[2,3,4-ij]quinoline-6-carboxamide,
(s) N-(4-Chlorobenzyl)-2-(2-furyl)-9-(morpholin-4-ylmethyl)-7-thioxo-2,3-dihydro-7H-[1,4]oxazino[2,3,4-ij]quinoline-6-carboxamide,
(t) N-(4-Chlorobenzyl)-2-(3-cyanophenyl)-9-(morpholin-4-ylmethyl)-7-thioxo-2,3-dihydro-7H-[1,4]oxazino[2,3,4-ij]quinoline-6-carboxamide,
(u) N-(4-Chlorobenzyl)-2-(3-furyl)-9-(morpholin-4-ylmethyl)-7-thioxo-2,3-dihydro-7H-[1,4]oxazino[2,3,4-ij]quinoline-6-carboxamide,
(v) N-(4-Chlorobenzyl)-9-(morpholin-4-ylmethyl)-7-thioxo-2-thien-2-yl-2,3-dihydro-7H-[1,4]oxazino[2,3,4-ij]quinoline-6-carboxamide,
(w) N-(4-Chlorobenzyl)-2-(3,5-difluorophenyl)-9-(morpholin-4-ylmethyl)-7-thioxo-2,3-dihydro-7H-[1,4]oxazino[2,3,4-ij]quinoline-6-carboxamide,
(x) 2-(1,3-Benzodioxol-5-yl)-N-(4-chlorobenzyl)-9-(morpholin-4-ylmethyl)-7-thioxo-2,3-dihydro-7H-[1,4]oxazino[2,3,4-ij]quinoline-6-carboxamide,
(y) N-(4-Chlorobenzyl)-2-(2,3-dihydro-1,4-benzodioxin-6-yl)-9-(morpholin-4-ylmethyl)-7-thioxo-2,3-dihydro-7H-[1,4]oxazino[2,3,4-ij]quinoline-6-carboxamide,
(z) 2-(1,3-Benzodioxol-4-yl)-N-(4-chlorobenzyl)-9-(morpholin-4-ylmethyl)-7-thioxo-2,3-dihydro-7H-[1,4]oxazino[2,3,4-ij]quinoline-6-carboxamide,
(aa) 2-[3,5-bis(Methoxymethoxy)phenyl]-N-(4-chlorobenzyl)-9-(morpholin-4-ylmethyl)-7-thioxo-2,3-dihydro-7H-[1,4]oxazino[2,3,4-ij]quinoline-6-carboxamide,
(bb) N-(4-Chlorobenzyl)-9-(morpholin-4-ylmethyl)-7-thioxo-2-thien-3-yl-2,3-dihydro-7H-[1,4]oxazino[2,3,4-ij]quinoline-6-carboxamide,
(cc) N-(4-Chlorobenzyl)-2,2-bis[(methoxymethoxy)methyl]-9-(morpholin-4-ylmethyl)-7-thioxo-2,3-dihydro-7H-[1,4]oxazino[2,3,4-ij]quinoline-6-carboxamide,
(dd) N-[(4-Chlorophenyl)methyl]-9′-(4-morpholinylmethyl)-4-oxo-7′-thioxospiro[cyclohexane-1,2′(3′H)-[7H]pyrido[1,2,3-de][1,4]benzoxazine]-6′-carboxamide,
(ee) N-[(4-Chlorophenyl)methyl]-4-hydroxy-9′-(4-morpholinylmethyl)-7′-thioxospiro[cyclohexane-1,2′(3′H)-[7H]pyrido[1,2,3-de][1,4]benzoxazine]-6′-carboxamide,
(ff) N-(4-Chlorobenzyl)-3,9-bis(morpholin-4-ylmethyl)-7-thioxo-2,3-dihydro-7H-[1,4]oxazino[2,3,4-ij]quinoline-6-carboxamide,
(gg) N-(4-Chlorobenzyl)-9-(morpholin-4-ylmethyl)-7-thioxo-2-phenyl-2,3-dihydro-7H-[1,4]oxazino[2,3,4-ij]quinoline-6-carboxamide,
(hh) N-(4-Chlorobenzyl)-2,2-difluoro-9-(morpholin-4-ylmethyl)-7-thioxo-2,3-dihydro-7H-[1,4]oxazino[2,3,4-ij]quinoline-6-carboxamide,
(ii) N-(4-Chlorobenzyl)-2-[(methylsulfanyl)methyl]-9-(morpholin-4-ylmethyl)-7-thioxo-2,3-dihydro-7H-[1,4]oxazino[2,3,4-ij]quinoline-6-carboxamide,
(jj) N-(4-Chlorobenzyl)-2-[(dimethylamino)methyl]-9-(morpholin-4-ylmethyl)-7-thioxo-2,3-dihydro-7H-[1,4]oxazino[2,3,4-ij]quinoline-6-carboxamide,
(kk) N-(4-Chlorobenzyl)-2-[(4-methyl-1-piperazinyl)methyl]-9-(morpholin-4-ylmethyl)-7-thioxo-2,3-dihydro-7H-[1,4]oxazino[2,3,4-ij]quinoline-6-carboxamide,
(ll) Methyl({[6-{[(4-chlorobenzyl)amino]carbonyl}-9-(morpholin-4-ylmethyl)-7-thioxo-2,3-dihydro-7H-[1,4]oxazino[2,3,4-ij]quinolin-2-yl]methyl}thio)acetate,
(mm) N-(4-Chlorobenzyl)-9-(morpholin-4-ylmethyl)-7-thioxo-2-(1-pyrrolidinylmethyl)-2,3-dihydro-7H-[1,4]oxazino[2,3,4-ij]quinoline-6-carboxamide,
(nn) N-(4-Chlorobenzyl)-2-{[(2,3-dihydroxypropyl)sulfanyl]methyl}-9-(morpholin-4-ylmethyl)-7-thioxo-2,3-dihydro-7H-[1,4]oxazino[2,3,4-ij]quinoline-6-carboxamide,
(oo) N-(4-Chlorobenzyl)-2-{[(2,3-dihydroxypropyl)amino]methyl}-9-(morpholin-4-ylmethyl)-7-thioxo-2,3-dihydro-7H-[1,4]oxazino[2,3,4-ij]quinoline-6-carboxamide,
(pp) N-(4-Chlorobenzyl)-2-{[(2-hydroxyethyl)amino]methyl}-9-(morpholin-4-ylmethyl)-7-thioxo-2,3-dihydro-7H-[1,4]oxazino[2,3,4-ij]quinoline-6-carboxamide,
(qq) N-(4-Chlorobenzyl)-9-(morpholin-4-ylmethyl)-7-thioxo-2-(1-piperidinylmethyl)-2,3-dihydro-7H-[1,4]oxazino[2,3,4-ij]quinoline-6-carboxamide,
(rr) 2-{[bis(2-Hydroxyethyl)amino]methyl}-N-(4-chlorobenzyl)-9-(morpholin-4-ylmethyl)-7-thioxo-2,3-dihydro-7H-[1,4]oxazino[2,3,4-ij]quinoline-6-carboxamide,
(ss) N-(4-Chlorobenzyl)-9-(morpholin-4-ylmethyl)-7-thioxo-2-{[(2-pyridinylmethyl)amino]methyl}-2,3-dihydro-7H-[1,4]oxazino[2,3,4-ij]quinoline-6-carboxamide,
(tt) 2-[(8-{[6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methoxy}-8-oxooctanoyl)(methyl)amino]ethanesulfonic acid sodium salt,
(uu) [6-{[(4-Chlorobenzyl)amino]carbonyl}-9-(morpholin-4-ylmethyl)-7-thioxo-2,3-dihydro-7H-[1,4]oxazino[2,3,4-ij]quinolin-2-yl]methyl dimethyl phosphate,
(vv) N-(4-Chlorobenzyl)-9-(morpholin-4-ylmethyl)-7-thioxo-2-{[(4-pyridinylmethyl)amino]methyl}-2,3-dihydro-7H-[1,4]oxazino[2,3,4-ij]quinoline-6-carboxamide,
(ww) N-(4-Chlorobenzyl)-2-(1H-imidazol-1-ylmethyl)-9-(morpholin-4-ylmethyl)-7-thioxo-2,3-dihydro-7H-[1,4]oxazino[2,3,4-ij]quinoline-6-carboxamide,
(xx) N-(4-Chlorobenzyl)-2-{[(4-chlorobenzyl)amino]methyl}-9-(morpholin-4-ylmethyl)-7-thioxo-2,3-dihydro-7H-[1,4]oxazino[2,3,4-ij]quinoline-6-carboxamide,
(yy) N-(4-Chlorobenzyl)-3-(hydroxymethyl)-9-(morpholin-4-ylmethyl)-7-thioxo-2,3-dihydro-7H-(1,4oxazino[2,3,4-ij]quinoline-6-carboxamide,
(zz) N-(4-Chlorobenzyl)-2-(4-hydroxyphenyl)-9-(morpholin-4-ylmethyl)-7-thioxo-2,3-dihydro-7H-[1,4]oxazino[2,3,4-ij]quinoline-6-carboxamide,
(aaa) N-(4-Chlorobenzyl)-2-{3-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,
(bbb) N-(4-Chlorobenzyl)-2-{2-[(methoxymethoxy)methyl]phenyl}-9-(morpholin-4-ylmethyl)-7-thioxo-2,3-dihydro-7H-[1,4]oxazino[2,3,4-ij]quinoline-6-carboxamide,
(ccc) N-(4-Chlorobenzyl)-2-(2-hydroxyphenyl)-9-(morpholin-4-ylmethyl)-7-thioxo-2,3-dihydro-7H-[1,4]oxazino[2,3,4-ij]quinoline-6-carboxamide,
(ddd) N-[(4-Chlorophenyl)methyl]-2,3,5,6-tetrahydro-9′-(4-morpholinylmethyl)-7′-thioxospiro[4H-pyran-4,2′(3′H)-[7H]pyrido[1,2,3-de][1,4]benzoxazine]-6′-carboxamide,
(eee) 1,1-Dimethylethyl 6-[[[(4-chlorophenyl)methyl]amino]carbonyl]-9′-(4-morpholinylmethyl)-7′-thioxospiro[piperidine-4,2′(3′H)-[7H]pyrido[1,2,3-de][1,4benzoxazine]-1-carboxylate,
(fff) N-[(4-Chlorophenyl)methyl]-9′-(4-morpholinylmethyl)-7′-thioxospiro[piperidine-4,2′(3′H)-[7H]pyrido[1,2,3-de][1,4]benzoxazine]-6′-carboxamide,
(ggg) N-(4-Chlorobenzyl)-2,2-bis[(hydroxymethoxy)methyl]-3-9-(morpholin-4-ylmethyl)-7-thioxo-2,3-dihydro-7H-[1,4]oxazino[2,3,4-ij]quinoline-6-carboxamide,
(hhh) N-[(4-Chlorophenyl)methyl]-2′,3′,5′,6′-tetrahydro-9-(4-morpholinylmethyl)-7-thioxospiro[7H-pyrido[1,2,3-de]-1,4-benzoxazine-2(3H),4′-[4H]thiopyran]-6-carboxamide; or a pharmaceutically acceptable salt thereof.

37. The method of claim 33, wherein the compound administered is N-(4-chlorobenzyl)-9-(morpholin-4-ylmethyl)-7-oxo-2-(S)-pyridin-2-yl-2,3-dihydro-7H-[1,4]oxazino[2,3,4-ij]quinoline-6-carboxamide; or a pharmaceutically acceptable salt thereof.

38. The method of claim 33, wherein the compound administered is N-(4-chlorobenzyl)-9-(morpholin-4-ylmethyl)-7-oxo-2-(R)-pyridin-2-yl-2,3-dihydro-7H-[1,4]oxazino[2,3,4-ij]quinoline-6-carboxamide; or a pharmaceutically acceptable salt thereof.

39. The method of claim 33, wherein the compound administered is N-(4-chlorobenzyl)-9-(morpholin-4-ylmethyl)-7-oxo-2-(R)-pyridin-3-yl-2,3-dihydro-7H-[1,4]oxazino[2,3,4-ij]quinoline-6-carboxamide; or a pharmaceutically acceptable salt thereof.

40. The method of claim 33, wherein the compound administered is N-(4-chlorobenzyl)-9-(morpholin-4-ylmethyl)-7-oxo-2-(S)-pyridin-3-yl-2,3-dihydro-7H-[1,4]oxazino[2,3,4-ij]quinoline-6-carboxamide; or a pharmaceutically acceptable salt thereof.

41. The method of claim 33, wherein the compound administered is N-(4-Chlorobenzyl)-2-(1-methyl-1H-imidazol-2-yl)-9-(morpholin-4-ylmethyl)-7-oxo-2,3-dihydro-7H-[1,4]oxazino[2,3,4-ij]quinoline-6-carboxamide; or a pharmaceutically acceptable salt thereof.

42. The method of claim 1, wherein the compound administered has the compound of Formula XI wherein Formula XI is 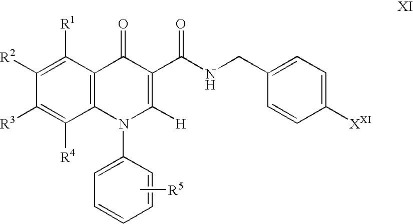
or a pharmaceutically acceptable salt thereof;
wherein,
X^{XI} is Cl, F, Br, CN, or NO₂;
R^{XI-1} is H, halo, or C₁₋₄alkyl optionally substituted by one to three halo;
R^{XI-2} is
a) H,
b) halo,
c) aryl^{XI},
d) het^{XI}, wherein said het^{XI} is bound via a carbon atom,
e) C₁₋₇alkyl which may be partially unsaturated and optionally substituted by one or more substituents of the group R^{XI-10}, NR^{XI-7}R^{XI-8}, halo, (C═O)R^{XI-6}, or S(O)ₘR^{XI-6},
f) NR^{XI-7}R^{XI-8},
g) OR^{XI-11},
h) SR^{XI-11},
i) NHSO₂R^{XI-6},
j) S(O)ₘR^{XI-6},
k) (C═O)R^{XI-6},
l) (C═O)OR^{XI-11},
m) CHO,
n) cyano, or
o) C₃₋₈cycloalkyl which may be partially unsaturated and is optionally substituted by one or more substituents selected from halo, oxo, R^{XI-10}, C₁₋₇alkyl, or NR^{XI-7}R^{XI-8};
R^{XI-3} is
a) H,
b) halo,
c) OR¹¹, or
d) C₁₋₇alkyl which may be partially unsaturated and optionally substituted by one or more substituents of the group OR^{XI-11}, SR^{XI-11}, NR^{XI-7}R^{XI-8}, or halo, or
R^{XI-2} together with R^{XI-3} form a carbocyclic or saturated 5 or 6 membered het^{XI} which may be optionally substituted by NR^{XI-7}R^{XI-8}, het^{XI} attached through a carbon atom, or C₁₋₇alkyl which may be optionally substituted by OR^{XI-12};
R^{XI-4} is
a) H,
b) halo,
c) OR^{XI-11}, or
d) C₁₋₇alkyl which may be partially unsaturated and optionally substituted by one or more substituents of the group OR^{XI-11}, SR^{XI-11}, NR^{XI-7}R^{XI-8}, aryl^{XI}, halo, C₃₋₈cycloalkyl optionally substituted by OR^{XI-12}, or het^{XI} attached through a carbon atom, or
e) NR^{XI-7}R^{XI-8};
R^{XI-5} is
a) H,
b) halo,
c) OR^{XI-11},
d) O(CH₂CH₂O)ₙR^{XI-12},
e) C₃₋₈cycloalkyl which may be partially unsaturated and is optionally substituted by one or more substituents selected from halo, OR^{XI-12}, SR^{XI-12}, oxo, C₁₋₇alkyl or NR^{XI-12}R^{XI-12},
f) het^{XI},
g) aryl^{XI},
h) NHSO₂R^{XI-6},
i) S(O)ₘR^{XI-6},
j) (C═O)R^{XI-6},
k) (C═O)OR^{XI-11},
l) nitro,
m) cyano,
n) SR^{XI-11},
o) NR^{XI-7}R^{XI-8},
p) C₁₋₇alkyl which may be partially unsaturated and is optionally substituted by one or more substituents selected from NR^{XI-7}R^{XI-8}, R^{XI-10}, S(O)ₘR^{XI-6}, (P═O)(OR^{XI-12})₂, (C═O)R^{XI-6}, or halo,
q) CHO,
r) SCN,
s) Any two adjacent R^{XI-5} substituents taken with the bond connecting them form an aryl^{XI}, or het^{XI}, or
t) Any two adjacent R^{XI-5} substituents taken together constitute a C₃₋₆alkyl chain which may be optionally substituted by R^{XI-9}, NR^{XI-7}R^{XI-8}, cyano, CO₂R^{XI-12}, OR^{XI-11}, SR^{XI-11}, or (═O);
R^{XI-6} is
a) C₁₋₇alkyl,
b) NR^{XI-11}R^{XI-11},
c) aryl^{XI}, or
d) het^{XI};
R^{XI-7} and R^{XI-8} are independently
a) H,
b) aryl^{XI},
c) C₁₋₇alkyl which may be partially unsaturated and is optionally substituted by one or more substituents selected from S(O)ₘR^{XI-6}, CONR^{XI-12}R^{XI-12}, CO₂R^{XI-12}, (C═O)R^{XI-9}, het^{XI}, aryl^{XI}, cyano, or halo,
d) C₂₋₇alkyl which may be partially unsaturated and is substituted by one or more substituents selected from NR^{XI-12}R^{XI-12}, OR^{XI-11}, or SR^{XI-11},
e) C₃₋₈cycloalkyl which may be partially unsaturated and is optionally substituted by one or more substituents selected from halo, OR^{XI-12}, SR^{XI-12}, oxo, or NR^{XI-12}R^{XI-12},
f) (C═O)R^{XI-9}, or
g) R^{XI-7} and R^{XI-8} together with the nitrogen to which they are attached for a het^{XI};
R^{XI-9} is
a) aryl^{XI},
b) het^{XI}, wherein said het^{XI} is bound through a carbon atom,
c) C₁₋₇alkyl optionally substituted by aryl^{XI}, het^{XI}, cyano, OR^{XI-12}, SR^{XI-12}, NR^{XI-12}R^{XI-12}, or halo, or
d) C₃₋₈cycloalkyl which may be partially unsaturated and is optionally substituted by one or more substituents selected from halo, OR^{XI-12}, SR^{XI-12}, or NR^{XI-12}R^{XI-12};
R^{XI-10} is
a) OR^{XI-11},
b) SR^{XI-11},
c) CO₂R^{XI-12},
d) het^{XI},
e) aryl^{XI}, or
f) cyano;
R^{XI-11} is
a) H,
b) aryl^{XI},
c) het^{XI}, wherein said het^{XI} is bound through a carbon atom,
d) C₁₋₇alkyl optionally substituted by aryl^{XI}, het^{XI}, wherein said het^{XI} is bound through a carbon atom, C₃₋₈cycloalkyl optionally substituted by OR^{XI-12}, or halo,
e) C₂₋₇alkyl substituted by OR^{XI-12}, SR^{XI-2}, or NR^{XI-12}R^{XI-12}, or
f) C₃₋₈cycloalkyl which may be partially unsaturated and is optionally substituted by one or more substituents selected from halo, OR^{XI-12}, SR^{XI-12}, or NR^{XI-12}R^{XI-12},
R^{XI-12} is H, or C₁₋₇alkyl;
each m^{XI} is independently 1 or 2;
each n^{XI} is independently 1, 2, or 3;
wherein aryl^{XI} is a phenyl radical or an ortho-fused bicyclic carbocyclic radical wherein at least one ring is aromatic and is optionally substituted with one or more substituents selected from halo, OH, cyano, CO₂R^{XI-12}, CF₃, C₁₋₆alkoxy, or C₁₋₆ alkyl which may be further substituted by one to three SR^{XI-12}, NR^{XI-12}R^{XI-12}, OR^{XI-12}, or CO₂R^{XI-12} groups;
wherein het^{XI} is a four- (4), five- (5), six- (6), or seven- (7) membered saturated or unsaturated heterocyclic ring having 1, 2, or 3 heteroatoms selected from oxygen, sulfur, or nitrogen, which is optionally fused to a benzene ring, or any bicyclic heterocyclic group and wherein any het^{XI} is optionally substituted with one or more substituents selected from halo, OH, cyano, phenyl, CO₂R^{XI-12}, CF₃, C₁₋₆alkoxy, oxo, oxime, or C₁₋₆ alkyl which may be further substituted by one to three SR^{XI-12}, NR^{XI-12}R^{XI-12}, OR^{XI-12}, or CO₂R^{XI-12}, groups; and
wherein halo is F, Cl, Br, I;
or a pharmaceutically acceptable salt thereof.

43. The method according to claim 42, wherein
X^{XI} is Cl;
R^{XI-1} is H;
R^{XI-12} is C₁₋₇alkyl which may be partially unsaturated and is substituted by one or more substituents of the group OH, NR^{XI-7}R^{XI-8}, or ^{XI-}het bound through a carbon atom; and
R^{XI-3} is H.

44. The method according to claim 42, wherein R^{XI-2} is C₁₋₇alkyl which may be partially unsaturated and is substituted by one or more substituents of the group OH, NR^{XI-7}R^{XI-8}, or het^{XI} bound through a carbon atom.

45. The method according to claim 42, wherein R^{XI-2} is C₁₋₇alkyl which is fully saturated and is substituted by one or more substituents of the group OH or NR^{XI-7}R^{XI-8}.

46. The method according to claim 42, wherein R^{XI-2} is 3-hydroxypropyl.

47. The method according to claim 42, wherein R^{XI-2} is 3-hydroxy-1-propynyl.

48. The method according to claim 42, wherein R^{XI-2} is tetrahydro-2H-pyran-4-ylmethyl.

49. The method according to claim 42, wherein R^{XI-2} is 4-morpholinylmethyl.

50. The method according to claim 42, wherein the compound administered is selected from the group consisting of
(1) N-(4-chlorobenzyl)-6-(4-morpholinylmethyl)-4-oxo-1-phenyl-1,4-dihydro-3-quinolinecarboxamide;
(2) N-(4-chlorobenzyl)-6-(3-hydroxypropyl)-4-oxo-1-phenyl-1,4-dihydro-3-quinolinecarboxamide;
(3) N-(4-chlorobenzyl)-6-(3-hydroxy-1-propynyl)-4-oxo-1-phenyl-1,4-dihydro-3-quinolinecarboxamide;
(4) N-(4-chlorobenzyl)-4-oxo-1-phenyl-6-(tetrahydro-2H-pyran-4-ylmethyl)-1,4-dihydro-3-quinolinecarboxamide;
(5) N-(4-chlorobenzyl)-1-(2-methylphenyl)-6-(4-morpholinylmethyl)-4-oxo-1,4-dihydro-3-quinolinecarboxamide;
(6) N-(4-chlorobenzyl)-1-(3-iodophenyl)-6-(4-morpholinylmethyl)-4-oxo-1,4-dihydro-3-quinolinecarboxamideN-(4-chlorobenzyl)-1-(4-chlorophenyl)-6-(4-morpholinylmethyl)-4-oxo-1,4-dihydro-3-quinolinecarboxamide;
(7) N-(4-chlorobenzyl)-1-(4-isopropylphenyl)-6-(4-morpholinylmethyl)-4-oxo-1,4-dihydro-3-quinolinecarboxamide;
(8) N-(4-chlorobenzyl)-1-(4-methoxyphenyl)-6-(4-morpholinylmethyl)-4-oxo-1,4-dihydro-3-quinolinecarboxamide;
(9) N-(4-fluorobenzyl)-1-(4-chlorophenyl)-6-(4-morpholinylmethyl)-4-oxo-1,4-dihydro-3-quinolinecarboxamide;
(10) N-(4-chlorobenzyl)-6-(4-morpholinylmethyl)-4-oxo-1-(2,4-difluorophenyl)-1,4-dihydro-3-quinolinecarboxamide;
(11) N-(4-chlorobenzyl)-6-(3-hydroxypropyl)-4-oxo-1-(2,4-difluorophenyl)-1,4-dihydro-3-quinolinecarboxamide;
(12) N-(4-chlorobenzyl)-6-(3-hydroxy-1-propynyl)-4-oxo-1-(2,4-difluorophenyl)-1,4-dihydro-3-quinolinecarboxamide;
(13) N-(4-chlorobenzyl)-4-oxo-1-(2,4-difluorophenyl)-6-(tetrahydro-2H-pyran-4-yl-methyl)-1,4-dihydro-3-quinolinecarboxamide;
(14) N-(4-Chlorobenzyl)-1-(2-(hydroxymethyl)phenyl)-6-(4-morpholinylmethyl)-4-oxo-1,4-dihydro-3-quinolinecarboxamide;
(15) N-(4-Chlorobenzyl)-1-(2,3-dihydro-1H-inden-5-yl)-6-(4-morpholinylmethyl)-4-oxo-1,4-dihydro-3-quinolinecarboxamide;
(16) 1-(1,3-Benzodioxol-5-yl)-N-(4-chlorobenzyl)-6-(4-morpholinylmethyl)-4-oxo-1,4-dihydro-3-quinolinecarboxamide;
(17) N-(4-Chlorobenzyl)-1-(1H-indol-5-yl)-6-(4-morpholinylmethyl)-4-oxo-1,4-dihydro-3-quinolinecarboxamide;
(18) N-(4-Fluorobenzyl)-1-(1H-indol-5-yl)-6-(4-morpholinylmethyl)-4-oxo-1,4-dihydro-3-quinolinecarboxamide;
(19) N-(4-Chlorobenzyl)-1-(3-hydroxyphenyl)-6-(4-morpholinylmethyl)-4-oxo-1,4-dihydro-3-quinolinecarboxamide;
(20) N-(4-Chlorobenzyl)-1-(3-(2-hydroxyethyl)phenyl)-6-(4-morpholinylmethyl)-4-oxo-1,4-dihydro-3-quinolinecarboxamide;
(21) N-(4-Fluorobenzyl)-1-(3-(2-hydroxyethyl)phenyl)-6-(4-morpholinylmethyl)-4-oxo-1,4-dihydro-3-quinolinecarboxamide;
(22) N-(4-chlorobenzyl)-1-(3-methoxyphenyl)-6-(4-morpholinylmethyl)-4-oxo-1,4-dihydro-3-quinolinecarboxamide;
(23) N-(4-chlorobenzyl)-1-(3-(hydroxymethyl)phenyl)-6-(4-morpholinylmethyl)-4-oxo-1,4-dihydro-3-quinolinecarboxamide;
(24) N-(4-chlorobenzyl)-6-(4-morpholinylmethyl)-1-(4-(4-morpholinyl)phenyl)-4-oxo-1,4-dihydro-3-quinolinecarboxamide;
(25) N-(4-chlorobenzyl)-1-(3,4-difluorophenyl)-6-(4-morpholinylmethyl)-4-oxo-1,4-dihydro-3-quinolinecarboxamide;
(26) N-(4-chlorobenzyl)-1-(3-(3-hydroxy-1-propynyl)phenyl)-6-(4-morpholinylmethyl)-4-oxo-1,4-dihydro-3-quinolinecarboxamide;
(27) N-(4-chlorobenzyl)-1-(3-(4-hydroxy-1-butynyl)phenyl)-6-(4-morpholinylmethyl)-4-oxo-1,4-dihydro-3-quinolinecarboxamide;
(28) N-(4-chlorobenzyl)-1-(3-(4-hydroxy-1-butynyl)phenyl)-6-(4-morpholinylmethyl)-4-oxo-1,4-dihydro-3-quinolinecarboxamide;
(29) N-(4-chlorobenzyl)-1-(3-(5-hydroxypentyl)phenyl)-6-(4-morpholinylmethyl)-4-oxo-1,4-dihydro-3-quinolinecarboxamide;
(30) N-(4-chlorobenzyl)-1-(3-(4-hydroxybutyl)phenyl)-6-(4-morpholinylmethyl)-4-oxo-1,4-dihydro-3-quinolinecarboxamide;
(31) N-(4-chlorobenzyl)-1-[3-(3-hydroxypropyl)phenyl]-6-(4-morpholinylmethyl)-4-oxo-1,4-dihydro-3-quinolinecarboxamide;
or a pharmaceutically acceptable salt thereof.

51. The method according to claim 42, which is N-(4-chlorobenzyl)-6-(4-morpholinylmethyl)-4-oxo-1-phenyl-1,4-dihydro-3-quinolinecarboxamide or a pharmaceutically acceptable salt thereof.

52. The method according to claim 42, wherein ^{XI-}X is Cl.

53. The method according to claim 42, wherein either R^{XI-2}, or R^{XI-4} or both R^{XI-2} and R^{XI-4} do not represent H.

54. A method according to claim 1, wherein said mammal is a human.

55. A method according to claim 1, wherein said mammal is a livestock or companion animal.

56. A method according to claim 1, wherein the effective dose is from about 0.1 to about 300 mg/kg of body weight.

57. A method according to claim 1, wherein the effective dose is about 1 to about 30 mg/kg of body weight.

58. A method according to claim 1, wherein the compound is administered parenterally, topically, intravaginally, orally or rectally.
